(19) United States
(12) Patent Application Publication
(10) Pub. No.: US 2005/0009136 A1
(43) Pub. Date: Jan. 13, 2005

(54) PAPP-A ligands

(75) Inventors: Andrew Nixon, Hanover, MA (US); Shannon Hogan, Arlington, MA (US)

Correspondence Address: FISH & RICHARDSON PC, 225 FRANKLIN ST, BOSTON, MA, 02110, US

(73) Assignee: DYAX CORPORATION

(21) Appl. No.: 10/783,311

(22) Filed: Feb. 19, 2004

Related U.S. Application Data
(60) Provisional application No. 60/448,515, filed on Feb. 19, 2003.

(51) Int. Cl. C07K 16/18; C07H 21/04; C12P 21/04; C12N 5/06

(52) U.S. Cl. 435/69.1; 435/320.1; 435/326; 530/387.1; 536/23.53

(57) ABSTRACT

The invention provides, inter alia, proteins that bind to PAPP-A, an 1547 amino acid glycoprotein which can form an ˜200 kDa monomer or an ˜400 kDa dimer. In one form, the proteins are antibodies. In one embodiment, the proteins can inhibit the ability of PAPP-A to interact (e.g., cleave) substrates such as IGFBP-4, IGFBP-5, and IGFBP-2.

### CROSS-REFERENCE TO RELATED APPLICATIONS

This application claims priority to U.S. Provisional Patent Application Serial No. 60/448,515, filed on Feb. 19, 2003, the contents of which are hereby incorporated by reference in their entireties.

### BACKGROUND

Pregnancy-associated plasma protein-A (PAPP-A) was first identified from the serum of pregnant women. PAPP-A is a metalloproteinase that can cleave insulin-like growth factor binding proteins (including IGFBP-2, IGFBP-4, and IGFBP-5), inhibitors or potentiators of insulin-like growth factor (IGF) action. IGFBP-4 inhibits IGF by binding to it and preventing its activity. PAPP-A cleaves IGF-bound IGFBP-4, thus releasing IGF from IGFBP-4. The released IGF is active and can bind to its receptor. PAPP-A is also able to cleave IGFBP-5, although this cleavage is IGF-independent. Thus, PAPP-A can regulate the biologically relevant concentration of IGF and as such is an important regulator in a number of disease states and tumor progression.

Vascular injury, e.g., as occurs in balloon angioplasty surgery, can cause overgrowth of vascular smooth muscle cells which leads to narrowing of the blood vessel, also referred to as restenosis. It has been observed that PAPP-A expression is increased in animal models of restenosis and that the activity of PAPP-A to release IGF suggests a role for PAPP-A in vascular smooth muscle cell proliferation and migration (Bayes-Genis et al., (2001) Arterioscler. Thromb. Vasc. Biol. 21:335-341). PAPP-A may further serve as a marker that can identify patients with unstable atherosclerotic plaques (Bayes-Genis (2001) New England Journal of Medicine 345:1022-1029).

### SUMMARY

This invention provides, inter alia, protein ligands that bind to PAPP-A. In one embodiment, the protein ligands include one or more immunoglobulin variable domains, e.g., the proteins are antibodies, or antigen-binding fragments thereof. For example, the invention provides anti-PAPP-A antibodies, antibody fragments, and pharmaceutical compositions thereof, as well as nucleic acids, recombinant expression vectors and host cells for making such antibodies and fragments. Methods of using the antibodies of the invention to detect PAPP-A, or regulate IGF axis activity, e.g., by regulating IGFBP, either in vitro or in vivo, are also encompassed by the invention. An anti-PAPP-A ligand that binds to human PAPP-A with high affinity and specificity, e.g., can be used as a diagnostic, prophylactic, or therapeutic agent in vivo and in vitro.

Human PAPP-A can regulate IGF levels, and thereby control behavior (e.g., growth, proliferation, or differentiation) of IGF-responsive cells or cells expressing PAPP-A. The antibodies can bind to the PAPP-A expressed from various cell types. The protein ligands of the invention can be used, for example, to target living normal, benign hyperplastic, and cancerous cells, e.g., cells whose growth or proliferation is regulated by IGF and cells that include PAPP-A associated with their cell surface.

Accordingly, in one aspect, the invention features an isolated protein that includes a first and second immunoglobulin variable domain (e.g., a light chain immunoglobulin variable domain (LC) and a heavy chain immunoglobulin variable domain (HC)), wherein the isolated protein binds to a PAPP-A molecule with an affinity constant of at least 10⁵ M⁻¹. In one embodiment, the protein has one or more of the following properties:
a. the isolated protein binds to an epitope within the PAPP-A molecule, e.g., an epitope bound by a ligand described herein;
b. the isolated protein competes with a protein described herein for binding to PAPP-A or competitively inhibits binding of a protein described herein to PAPP-A;
c. the isolated protein inhibits PAPP-A cleavage of an IGFBP;
d. the first and/or second immunoglobulin domain is at least 70, 80, 85, 90, 95, 96, 97, 98, 99% identical to an immunoglobulin domain sequence described herein;
e. the first and/or second immunoglobulin domain comprises one, two, or three of the CDRs of an immunoglobulin domain sequence described herein;
f. the first and/or second immunoglobulin domain comprises one, two or three CDRs that have an amino acid sequence that differs by no more than 3, 2.5, 2, 1.5, 1, 0.7, 0.5, or 0.2 substitutions, insertions or deletions for every 10 amino acids relative to an immunoglobulin domain sequence described herein;
g. the first and/or second immunoglobulin domain is at least 70, 80, 85, 90, 95, 96, 97, 98, 99% identical in the CDR regions to an immunoglobulin domain sequence described herein; or
h. the first and/or second immunoglobulin domain is at least 70, 80, 85, 90, 95, 96, 97, 98, 99% identical in the framework regions to an immunoglobulin domain sequence described herein.

In one embodiment, CDR1 of the LC variable region includes:

| | |
|---|---|
| R-A-S-[QR]-[DGRS]-[VI]-[RSN]- | (SEQ ID NO:358) |
| [NRHST]-[YDEWNS]-[LVY]- | |
| [AGNL]; | |
| R-A-S-Q-X1-[VI]-X2-X3-[YDEWNS]-X4, | (SEQ ID NO:359) |

wherein X1, X2, and X3 are any amino acid, e.g., a hydrophilic amino acid and X4 is hydrophobic, e.g., aliphatic;

X1-X2-X3-X4-X5-X6-X7-X8, wherein X1 is N, Q, R, or K, X2 is hydrophilic, A, or G, X3 is aliphatic, X4 and X5 are hydrophilic, X6 is any amino acid, or aromatic or hydrophilic, and X7 is hydrophobic;

| | |
|---|---|
| S-G-S-S-S-N-I-[GEDA]-[SRV]-[NY]- | (SEQ ID NO:360) |
| [TLFD]-V-[YT]; | |
| S-G-S-S-S-N-I-[GEDA]-[SRV]-[ANY]- | (SEQ ID NO:389) |
| [TLFD]-V-[NYT]; | |
| T-G-T-S-S-D-[IV]-G-[DGY]-Y-[NED]- | (SEQ ID NO:361) |
| Y-V-S; | |
| T-G-T-S-S-D-[IV]-G-[ADGY]-Y- | (SEQ ID NO:387) |
| [NKED]-[YF]-V-S; | |
| or | |
| X1-X2-X3-G-X4-Y-X5-X6-X7-X8, | |

wherein X1 is T or S, X2 is D or E, X3 is aliphatic, X4 is hydrophilic or G, and X5 is hydrophilic or N, E, D, or Q.

In one embodiment, CDR2 of the LC variable region includes:

| | |
|---|---|
| [ADEG]-[AVDNE]-[ASTNV]-[STNQ]- | |
| [LRN]-[AQPR]-[TFSKP]; | |
| [ADENG]-[AVDNE]-[ASTRNV]-[STENQ]- | |
| [LRN]-[AQPR]-[TFSKP] | |
| [ADE]-[AV]-[AST]-[ST]-[LR]-[AQ]- | |
| [TFSK]; | |
| [ST]-X1-X2-X3-[LRN]-[PRQ]-S, | (SEQ ID NO:382) |
| wherein X1, X2, and X3 are | |
| hydrophilic; | |
| [NST]-X1-X2-X3-[LRN]-[PRQ]-S, | (SEQ ID NO:388) |
| wherein X1, X2, and X3 are | |
| hydrophilic; | |
| [ST]-[DN]-[DN]-Q-R-P-S; | (SEQ ID NO:362) |
| [HST]-[DN]-[DN]-[QY]-R-P; | (SEQ ID NO:389) |
| or | |
| G-A-S-[ST]-[LR]-[QA]. | (SEQ ID NO:363) |

In one embodiment, CDR3 of the LC variable region includes:
[QL]-Q-X1-X2-X3-X4-P-X5 (SEQ ID NO:364), wherein X1, X2, X3, X4, and X5 are any amino acid, or X1 is hydrophilic, A, or G, X2 is hydrophilic, X3 is hydrophilic, X4 is aromatic, T, R, or K, X5 is hydrophobic, and the sequence can optionally be followed by T;
Q-Q-Y-X1-X2-X3-P-[PLR]-T (SEQ ID NO:365), wherein X1 and X2 are any amino acid, and X3 is hydrophobic (e.g., aromatic);
[AGQSV]-[ATS]-X1-X2-X3-[STGA]-X4-[STRG]-[GPNF]-X5-V (SEQ ID NO:381), wherein X2, X3, and X4 are any amino acid, and X1 is aromatic, or X2 is E, D, R, T, or S, X3 is D, N, Q, K, R, or S, and X4 is S, L, T, or N;
A-W-D-D-S-L-S-G-X1-V (SEQ ID NO:366), wherein X1 is hydrophobic;
A-W-D-D-S-L-S-G-[VW]-V (SEQ ID NO:367);
A-[AT]-W-D-[DNEQ]-[ST]-L-X1-G-X2-V (SEQ ID NO:), wherein X1 is any amino acid (e.g., S, R, T, H, N) and X2 is any amino acid, e.g., hydrophobic, e.g., V, Y, or W; or
A-[AT]-W-D-[DNEQ]-[ST]-L-[SRT]-G-[VW]-V (SEQ ID NO:368).

In one embodiment, CDR1 of the HC variable region includes:
Y-X1-M-X2 (SEQ ID NO:369), wherein X1 and X2 are any amino acid, or X1 is W, D, K, T, R, H, or P, and X2 is N, W, D, E, P, T, R, S, V, or F;
X1-Y-X2-M-X3 (SEQ ID NO:370), wherein X1 is aromatic, X2 is any amino acid, and X3 is N, W, D, E, P, T, R, S, V, or F;
W-Y-X1-M (SEQ ID NO:371), wherein X1 is any amino acid, or X1 is W, H, or T; or
Q-Y-X1-M (SEQ ID NO:372), wherein X1 is any amino acid.

In one embodiment, CDR2 of the HC variable region includes

I-X1-[PS]-S-G-G (SEQ ID NO:373), wherein X1 is any amino acid, hydrophobic or V, Y, W, R, S, or G;

I-X1-[PS]-S-G-G-X2-T (SEQ ID NO:374), wherein X1 and X2 are any amino acid;

I-X1-[PS]-S-G-G-X2-T (SEQ ID NO:375), wherein X1 is S, V, Y, W, R, or G, and X2 is G, K, L, R, H, F, Y, T, G, Q, D, M, I, or N; or

I-X1-[PS]-S-G-G-X2-T-X3-Y-A-D-S-V-K-G (SEQ ID NO:376), wherein X1 is S, V, Y, W, R, or G, and X2 and X3 are any amino acid.

In one embodiment, CDR3 of the HC variable region includes:

D-F-G-S;

at least two, three, or four consecutive tyrosines;

| | |
|---|---|
| [SG]-[SG]-W-Y; | (SEQ ID NO:377) |
| S-S-[SG]-W-Y; | (SEQ ID NO:378), |
| [RHWY]-Y-Y-Y-G-M; | (SEQ ID NO:379) |
| S-S-[SG]-W-[SY]; | |
| or | |
| [YSG]-[RHWY]-Y-Y-Y-G-M-D. | (SEQ ID NO:380) |

In one embodiment, the protein is at least 50, 60, 70, 80, 85, 90, or 95% identical to a immunoglobulin region encoded by at least one human germline sequence.

In one embodiment, the first and second immunoglobulin domain are components of separate polypeptide chains. In another embodiment, the first and second immunoglobulin domain are components of the same polypeptide chain. The protein can be physically associated with an agent, e.g., coupled or bound to an agent, e.g., a label or a cytotoxic agent. In one embodiment, the cytotoxic agent includes an Fc domain.

In one embodiment, the protein can inhibit a PAPP-A-mediated activity, e.g., PAPP-A-mediated cleavage of an IGFBP. In one embodiment, the protein can bind to a PAPP-A containing structure, e.g., a plaque, e.g., an atherosclerotic plaque. In another embodiment, the PAPP-A containing structure is a tumor.

In one embodiment, the protein can bind to PAPP-A associated with the surface of a cell.

In one embodiment, the protein can alter a property of an IGF-responsive cell in vivo. For example, the protein may alter a property of a tumor cell in vivo. In some embodiments, the protein can impair or kill a tumor cell that has PAPP-A associated on the tumor cell surface.

The protein can also be used, e.g., to detect PAPP-A. For example, the protein can be used in a method that includes: providing a PAPP-A binding protein described herein; and detecting binding of the protein to a sample or detecting binding of the protein within a subject, e.g., a patient. In another example, the protein can be used to evaluate a subject. The method includes: providing the protein; administering the protein to a subject; and detecting location of the protein within the subject.

A protein described herein can be used, e.g., therapeutically to prevent activity of PAPP-A in a patient, it can be used diagnostically, e.g., to detect atherosclerotic lesions or to localize PAPP-A, it can be used on a patient, e.g., with or suspected of having an atherosclerotic lesion.

A protein described herein can be used therapeutically, e.g., on a patient with an acute coronary syndrome, or a patient at risk for developing a coronary artery occlusion, a patient undergoing angioplasty, or at risk for restenosis. For example, the ligand can be used to reduce or prevent the overgrowth of smooth muscle cells that contributes to restenosis.

In another aspect, the invention features a method of treating a subject. The method includes providing a pharmaceutical composition that includes a PAPP-A binding protein described herein; and administering the pharmaceutical composition to a subject in an amount effective to treat a disease or disorder. For example, the disease or disorder is a proliferative disease. The disease or disorder can include IGF-1 regulated growth. In one embodiment, the subject has a glioblastoma or an osteosarcoma.

In one embodiment, the protein is administered by application during a surgical procedure, e.g., during brain or other neurosurgery. In another embodiment, the protein is administered to a lumbar puncture.

In another aspect, the invention features a method of modulating IGF activity in a subject. The method can include: providing a pharmaceutical composition that includes a PAPP-A binding protein, e.g., a PAPP-A binding protein described herein; identifying a subject having a disease or disorder associated with aberrant IGF activity; and administering the pharmaceutical composition to a subject in an amount effective to modulate (e.g., reduce) IGF activity in the subject.

In yet another aspect, the invention features a method of modulating IGF activity in a subject. The method can include: providing a pharmaceutical composition that includes a PAPP-A binding protein, e.g., a PAPP-A binding protein described herein; identifying a subject having a proliferative disorder, e.g., a glioblastoma; and administering the pharmaceutical composition to a subject in an amount effective to reduce proliferation associated with the proliferative disorder in the subject.

In another aspect, the invention features a method of reducing activity of PAPP-A in a subject. The method can include: identifying a subject having a disease or disorder associated with aberrant PAPP-A activity; and administering a pharmaceutical composition that includes a PAPP-A binding protein, e.g., a PAPP-A binding protein described herein to the subject in an amount effective to reduce PAPP-A activity in the subject.

In still another aspect, the invention features a method of altering a cellular activity. The method includes: providing a PAPP-A binding protein, e.g., a PAPP-A binding protein described herein, to the extracellular milieu of a cell, under conditions that enable the protein to interact with PAPP-A to thereby alter the IGF signalling in the cell.

In one embodiment, the anti-PAPP-A ligand binds to human PAPP-A with high affinity and specificity, and thus can be used as diagnostic, prophylactic, or therapeutic agents in vivo and in vitro. Preferably the ligands specifically bind to the PAPP-A. As used herein, “specific binding” refers to the property of the antibody: (1) to bind to PAPP-A, e.g., human PAPP-A, with an affinity of at least 1×10^{≡}M⁻¹, 1×10⁶ M⁻¹, 1×10⁷ M⁻¹, 1×10⁸ M⁻¹, or 1×10⁹ M⁻¹ and (2) to preferentially bind to PAPP-A, e.g., human PAPP-A, with an affinity that is at least two-fold, 50-fold, 100-fold, or greater than its affinity for binding to a non-specific antigen other than PAPP-A (e.g., BSA, casein, a non-metzincin protease, or a protease that cannot cleave a IGFBP).

The protein ligands of the invention interact with, e.g., bind to PAPP-A, preferably human PAPP-A, with high affinity and specificity. For example, the protein ligand binds to human PAPP-A with an affinity constant of at least 10⁷ M⁻¹, preferably, at least 10⁸ M⁻¹, 10⁹ M⁻¹, or 10¹⁰ M⁻¹. Exemplary protein ligands can have an IC₅₀ of between about 0.1-200 nM, 0.1-5 nM, 1-20 nM, e.g., about 2 nM or about 11 nM. IC50 can be evaluated using standard methods, e.g., obtained from an in vitro biochemical peptide cleavage assay in the presence of a ligand of interest. Exemplary protein ligands can have a Kᵢ between 10^{31 6} and 10⁻¹¹ M, or 10⁻⁷ and 10⁻¹⁰ M, or less than 10⁻⁶, 10⁻⁷, 10⁻⁹.

In one embodiment, the protein ligand interacts with, e.g., binds to, the protease domain of human PAPP-A (e.g., about amino acids 81-1214 of SEQ ID NO: 1). In one embodiment, the anti-PAPP-A ligand binds all or part of the epitope of an antibody described herein. The anti-PAPP-A ligand can inhibit, e.g., competitively inhibit, the binding of an antibody described herein to human PAPP-A. An anti-PAPP-A ligand may bind to an epitope, e.g., a conformational or a linear epitope, which when bound, prevents binding of an antibody described herein. The epitope can be in close proximity spatially (e.g., within 5 Angstroms) or functionally-associated, e.g., an overlapping or adjacent epitope in linear sequence or conformationally to the one recognized by an antibody described herein. In one embodiment, the anti-PAPP-A ligand binds to an epitope located wholly or partially within the protease domain (e.g., about amino acids 81-1214 of SEQ ID NO:1) of human PAPP-A. In one embodiment, the ligand bind to an epitope that includes amino acid 562, 563, or 566 of SEQ ID NO:1, or an epitope that overlaps with 520-535, 557-574, or 615-632 of SEQ ID NO:1, or short consensus repeats of complement proteins and selectins (SCRs), e.g., SCR1-5, epitopes between SCR3 and SCR4), the sequence of these SCRs is referenced in Kristensen et al. (1994) Biochemistry. 33, 1592-1598.

In a preferred embodiment, the protein ligand is an antibody. As used herein, the term “antibody” refers to a protein comprising at least one, and preferably two, heavy (H) chain immunoglobulin variable regions (abbreviated herein as VH), and at least one and preferably two light (L) chain immunoglobulin variable regions (abbreviated herein as VL). Accordingly, the term “antibody” encompasses, and is not limited to F″abs, single chain antibodies, other antibody fragments, IgG's, IgM's, and other immunoglobulin variable domain-containing structures. The VH and VL regions can be further subdivided into regions of hypervariability, termed “complementarity determining regions” (“CDR”), interspersed with regions that are more conserved, termed “framework regions” (FR). The extent of the framework region and CDRs has been precisely defined (see, Kabat, E. A., et al. (1991) Sequences of Proteins of Immunological Interest, Fifth Edition, U.S. Department of Health and Human Services, NIH Publication No. 91-3242, and Chothia, C. et al. (1987) J. Mol. Biol. 196:901-917). For the purposes of a definition herein, an explicit recitation of a CDR sequence controls, and in the absence of an explicit recitation, the Kabat definition is used. Each VH and VL is composed of three CDRs and four FRs, arranged from amino-terminus to carboxy-terminus in the following order: FR1, CDR1, FR2, CDR2, FR3, CDR3, FR4.

The VH or VL chain of the antibody can further include all or part of a heavy or light chain constant region, to thereby form a heavy or light immunoglobulin chain, respectively. In one embodiment, the antibody is a tetramer of two heavy immunoglobulin chains and two light immunoglobulin chains, wherein the heavy and light immunoglobulin chains are inter-connected by, e.g., disulfide bonds. The heavy chain constant region is comprised of three domains, CH1, CH2 and CH3. The light chain constant region is comprised of one domain, CL. The variable region of the heavy and light chains contains a binding domain that interacts with an antigen. The constant regions of the antibodies typically mediate the binding of the antibody to host tissues or factors, including various cells of the immune system (e.g., effector cells) and the first component (Clq) of the classical complement system. The term “antibody” includes intact immunoglobulins of isotypes IgA, IgG, IgE, IgD, IgM (as well as subtypes thereof), wherein the light chains of the immunoglobulin may be of types kappa or lambda.

As used herein, the term “immunoglobulin” refers to a protein consisting of one or more polypeptides substantially encoded by immunoglobulin genes. The recognized human immunoglobulin genes include the kappa, lambda, alpha (IgA1 and IgA2), gamma (IgG1, IgG2, IgG3, IgG4), delta, epsilon and mu constant region genes, as well as the myriad variable region genes. Full-length immunoglobulin “light chains” (about 25 Kd or 214 amino acids) are encoded by a variable region gene at the NH2-terminus (about 110 amino acids) and a kappa or lambda constant region gene at the COOH—terminus. Full-length immunoglobulin “heavy chains” (about 50 Kd or 446 amino acids), are similarly encoded by a variable region gene (about 116 amino acids) and one of the other aforementioned constant region genes, e.g., gamma (encoding about 330 amino acids).

The term “antigen-binding fragment” of an antibody (or simply “antibody portion,” or “fragment”), as used herein, refers to one or more fragments of a full-length antibody that retain the ability to specifically bind to PAPP-A (e.g., human PAPP-A). Examples of binding fragments encompassed within the term “antigen-binding fragment” of an antibody include (i) a Fab fragment, a monovalent fragment consisting of the VL, VH, CL and CH domains; (ii) a F(ab′)₂ fragment, a bivalent fragment comprising two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR). Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding fragment” of an antibody. These antibody fragments are obtained using conventional techniques known to those with skill in the art, and the fragments are screened for utility in the same manner as are intact antibodies.

The antibody is preferably monospecific, e.g., a monoclonal antibody, or antigen-binding fragment thereof. The term “monospecific antibody” refers to an antibody that displays a single binding specificity and affinity for a particular target, e.g., epitope. This term includes a “monoclonal antibody” or “monoclonal antibody composition,” which as used herein refer to a preparation of antibodies or fragments thereof of single molecular composition.

The anti-PAPP-A antibodies can be full-length (e.g., an IgG (e.g., an IgG1, IgG2, IgG3, IgG4), IgM, IgA (e.g., IgA1, IgA2), IgD, and IgE, but preferably an IgG) or can include only an antigen-binding fragment (e.g., a Fab, F(ab′)₂ or scFv fragment). The antibody, or antigen-binding fragment thereof, can include two heavy chain immunoglobulins and two light chain immunoglobulins, or can be a single chain antibody. The antibodies can, optionally, include a constant region chosen from a kappa, lambda, alpha, gamma, delta, epsilon or a mu constant region gene. A preferred anti-PAPP-A antibody includes a heavy and light chain constant region substantially from a human antibody, e.g., a human IgG1 constant region or a portion thereof and a kappa or lambda light chain constant region or portion thereof. As used herein, “isotype” refers to the antibody class (e.g., IgM or IgG1) that is encoded by heavy chain constant region genes.

In a preferred embodiment, the antibody is a recombinant or modified anti-PAPP-A antibody, e.g., a chimeric, a humanized, a deimmunized, or an in vitro generated antibody. The term antibody encompasses antigen binding fragments thereof. The term “recombinant” or “modified” human antibody, as used herein, is intended to include all antibodies that are prepared, expressed, created or isolated by recombinant means, such as antibodies expressed using a recombinant expression vector transfected into a host cell, antibodies isolated from a recombinant, combinatorial antibody library, antibodies isolated from an animal (e.g., a mouse) that is transgenic for human immunoglobulin genes or antibodies prepared, expressed, created or isolated by any other means that involves splicing of human immunoglobulin gene sequences to other DNA sequences. Such recombinant antibodies include humanized, CDR grafted, chimeric, deimmunized, in vitro generated antibodies, and may optionally include constant regions derived from human germline immunoglobulin sequences.

In other embodiments, the anti-PAPP-A antibody is a human antibody or an effectively human antibody. Also within the scope of the invention are antibodies, or antigen-binding fragments thereof, which bind overlapping epitopes of, or competitively inhibit, the binding of the anti-PAPP-A antibodies disclosed herein to PAPP-A, e.g., antibodies which bind overlapping epitopes of, or competitively inhibit, the binding of monospecific antibodies described herein to PAPP-A. Any combination of anti-PAPP-A antibodies is within the scope of the invention, e.g., two or more antibodies that bind to different regions of PAPP-A, e.g., antibodies that bind to two different epitopes on PAPP-A, e.g., a bispecific antibody.

In one embodiment, the anti-PAPP-A antibody, or antigen-binding fragment thereof, includes at least one light or heavy chain immunoglobulin (or preferably, at least one light chain immunoglobulin and at least one heavy chain immunoglobulin). Preferably, each immunoglobulin includes a light or a heavy chain variable region having at least one, two and, preferably, three complementarity determining regions (CDRs) substantially identical to a CDR from an anti-PAPP-A light or heavy chain variable region, respectively, i.e., from a variable region of an immunoglobulin variable domain described herein.

In a preferred embodiment, the antibody (or fragment thereof) includes at least one, two and preferably three CDRs from the light or heavy chain variable region of an immunoglobulin variable domain described herein. In other embodiments, the antibody (or fragment thereof) can have at least one, two and preferably three CDRs from the light or heavy chain variable region of an immunoglobulin variable domain pair as produced by clone described herein. In one preferred embodiment, the antibody, or antigen-binding fragment thereof, includes all six CDRs from an anti-PAPP-A antibody produced by a clone described herein.

In another preferred embodiment, the antibody (or fragment thereof) includes at least one, two and preferably three CDRs from the light and/or heavy chain variable region of a clone described herein, e.g., having an amino acid sequence that differs by no more than 3, 2.5, 2, 1.5, or 1, 0.5 substitutions, insertions or deletions for every 10 amino acids relative to a heavy chain CDRs described herein, or a light chain CDRs described herein, or a sequence substantially identical thereto. Further, the antibody, or antigen-binding fragment thereof, can include six CDRs, each of which differs by no more than 3, 2.5, 2, 1.5, or 1, 0.5 substitutions, insertions or deletions for every 10 amino acids relative to the corresponding CDRs of an anti-PAPP-A antibody described herein.

In another embodiment, the light or heavy chain immunoglobulin of the anti-PAPP-A antibody, or antigen-binding fragment thereof, can further include a light or a heavy chain variable framework that has no more than 3, 2.5, 2, 1.5, or 1, 0.5 substitutions, insertions or deletions for every 10 amino acids in FR1, FR2, FR3, or FR4 relative to the corresponding frameworks of an antibody described herein. In a preferred embodiment, the light or heavy chain immunoglobulin of the anti-PAPP-A antibody, or antigen-binding fragment thereof, further includes a light or a heavy chain variable framework, e.g., FR1, FR2, FR3, or FR4, that is identical to a framework of an antibody described herein.

In one embodiment, the light or the heavy chain variable framework can be chosen from: (a) a light or heavy chain variable framework including at least 70%, 80%, 90%, 95%, or preferably 100% of the amino acid residues from a human light or heavy chain variable framework, e.g., a light or heavy chain variable framework residue from a human mature antibody or a human germline sequence, or a consensus sequence; (b) a light or heavy chain variable framework including from 20% to 80%, 40% to 80%, or 60% to 90% of the amino acid residues from a human light or heavy chain variable framework, e.g., a light or heavy chain variable framework residue from a human mature antibody or a human germline sequence, or a consensus sequence; (c) a non-human framework (e.g., a rodent framework); or (d) a non-human framework that has been modified, e.g., to remove antigenic or cytotoxic determinants, e.g., deimmunized, or partially humanized.

In one embodiment, the heavy or light chain framework includes an amino acid sequence that is at least 80%, 85%, 90%, 95%, 97%, 98%, or 99% in identity to an amino acid sequence described herein, or to the heavy or light chain framework sequence of the antibody produced by a clone described herein; or which differs by at least 1, 2 or 5 but less than 40, 30, 20, or 10 residues from an amino acid sequence described herein.

In other embodiments, the modified heavy and/or light chain variable region of the PAPP-A antibody has an amino acid sequence, which is at least 80%, 85%, 90%, 95%, 97%, 98%, 99% or higher identical to an amino acid sequence described herein, or the heavy and/or light chain variable region sequence of the antibody produced by a clone described herein; or which differs by at least 1 or 5 but less than 40, 30, 20, or 10 residues from an amino acid sequence described herein.

Preferred anti-PAPP-A antibodies include at least one, preferably two, light and at least one, preferably two, heavy chain variable regions of a clone described herein.

In other embodiments, the light or heavy chain variable framework of the anti-PAPP-A antibody, or antigen-binding fragment thereof, includes at least one, two, three, four, five, six, seven, eight, nine, ten, fifteen, sixteen, or seventeen amino acid residues from a human light or heavy chain variable framework, e.g., a light or heavy chain variable framework residue from a human mature antibody, a human germline sequence, or a consensus sequence. In one embodiment, the amino acid residue from the human light chain variable framework is the same as the residue found at the same position in a human germline. Preferably, the amino acid residue from the human light chain variable framework is the most common residue in the human germline at the same position.

An anti-PAPP-A ligand described herein can be used alone, e.g., can be administered to a subject or used in vitro in non-derivatized or unconjugated forms. In other embodiments, the anti-PAPP-A ligand can be derivatized, modified or linked to another functional molecule, e.g., another peptide, protein, isotope, cell, or insoluble support. For example, the anti-PAPP-A ligand can be functionally linked (e.g., by chemical coupling, genetic fusion, non-covalent association or otherwise) to one or more other molecular entities, such as an antibody (e.g., if the ligand is an antibody to form a bispecific or a multispecific antibody), a toxin, a radioisotope, a therapeutic (e.g., a cytotoxic or cytostatic) agent or moiety, among others. For example, the anti-PAPP-A ligand can be coupled to a radioactive ion (e.g., an α-, γ-, or β-emitter), e.g., iodine (¹³¹I or ¹²⁵I), yttrium (⁹⁰Y), lutetium (¹⁷⁷Lu), actinium (²²⁵Ac), rhenium (¹⁸⁶Re), or bismuth (²¹² or ²¹³Bi).

In another aspect, the invention provides, compositions, e.g., pharmaceutical compositions, which include a pharmaceutically acceptable carrier, excipient or stabilizer, and at least one of the anti-PAPP-A ligands (e.g., antibodies or fragments thereof) described herein. In one embodiment, the compositions, e.g., the pharmaceutical compositions, comprise a combination of two or more of the aforesaid anti-PAPP-A ligands.

In another aspect, the invention features a kit that includes an anti-PAPP-A antibody (or fragment thereof), e.g., an anti-PAPP-A antibody (or fragment thereof) as described herein, for use alone or in combination with other therapeutic modalities, e.g., a cytotoxic or labeling agent, e.g., a cytotoxic or labeling agent as described herein, along with instructions on how to use the PAPP-A antibody or the combination of such agents to treat, prevent or detect cancerous lesions.

The invention also features nucleic acid sequences that encode a heavy and light chain immunoglobulin or immunoglobulin fragment described herein. For example, the invention features, a first and second nucleic acid encoding a heavy and light chain variable region, respectively, of an anti-PAPP-A antibody molecule as described herein. In another aspect, the invention features host cells and vectors containing the nucleic acids of the invention.

In another aspect, the invention features a method of producing an anti-PAPP-A antibody, or antigen-binding fragment thereof. The method includes: providing a first nucleic acid encoding a heavy chain variable region, e.g., a heavy chain variable region as described herein; providing a second nucleic acid encoding a light chain variable region, e.g., a light chain variable region as described herein; and expressing said first and second nucleic acids in a host cell under conditions that allow assembly of said light and heavy chain variable regions to form an antigen binding protein. The first and second nucleic acids can be linked or unlinked, e.g., expressed on the same or different vector, respectively.

The host cell can be a eukaryotic cell, e.g., a mammalian cell, an insect cell, a yeast cell, or a prokaryotic cell, e.g., E. coli. For example, the mammalian cell can be a cultured cell or a cell line. Exemplary mammalian cells include lymphocytic cell lines (e.g., NSO), Chinese hamster ovary cells (CHO), COS cells, oocyte cells, and cells from a transgenic animal, e.g., mammary epithelial cell. For example, nucleic acids encoding the antibodies described herein can be expressed in a transgenic animal. In one embodiment, the nucleic acids are placed under the control of a tissue-specific promoter (e.g., a mammary specific promoter) and the antibody is produced in the transgenic animal. For example, the antibody molecule is secreted into the milk of the transgenic animal, such as a transgenic cow, pig, horse, sheep, goat or rodent.

The invention also features a method of treating, e.g., ablating or killing, a cell, e.g., a normal, benign or hyperplastic cell (e.g., a cell found in pulmonary, brain, ovary, breast, renal, urothelial, colonic, prostatic, or hepatic cancer and/or metastasis). Methods of the invention include contacting the cell with a anti-PAPP-A ligand, in an amount sufficient to treat, e.g., ablate or kill, the cell. The ligand can include a cytotoxic entity. Methods of the invention can be used, for example, to treat or prevent a disorder, e.g., a cancerous (e.g., a malignant or metastatic disorder), or non-cancerous disorder (e.g., a benign or hyperplastic disorder) by administering to a subject a anti-PAPP-A ligand in an amount effective to treat or prevent such disorder.

The subject method can be used on cells in culture, e.g. in vitro or ex vivo. For example, cancerous or metastatic cells (e.g., pulmonary, breast, brain, ovary, renal, urothelial, colonic, prostatic, or hepatic cancer or metastatic cells) can be cultured in vitro in culture medium and the contacting step can be effected by adding the anti-PAPP-A ligand to the culture medium. The method can be performed on cells (e.g., cancerous or metastatic cells) present in a subject, as part of an in vivo (e.g., therapeutic or prophylactic) protocol. For in vivo embodiments, the contacting step is effected in a subject and includes administering the anti-PAPP-A ligand to the subject under conditions effective to permit both binding of the ligand to the cell, and the treating, e.g., the killing or ablating of the cell.

The method of the invention can be used to treat or prevent cancerous disorders, e.g., including but are not limited to, solid tumors, soft tissue tumors, and metastatic lesions. Examples of solid tumors include malignancies, e.g., sarcomas, adenocarcinomas, and carcinomas, of the various organ systems, such as those affecting lung, breast, brain, ovary, lymphoid, gastrointestinal (e.g., colon), and genitourinary tract (e.g., renal, urothelial cells), pharynx, as well as adenocarcinomas which include malignancies such as most colon cancers, rectal cancer, renal-cell carcinoma, liver cancer, non-small cell carcinoma of the lung, cancer of the small intestine and cancer of the esophagus. Another exemplary tumor is a glioblastoma multiforme (GBM), e.g., GBM Grade IV astrocytoma. Metastatic lesions of the aforementioned cancers can also be treated or prevented using the methods and compositions of the invention.

The subject can be a mammal, e.g., a primate, preferably a higher primate, e.g., a human (e.g., a patient having, or at risk of, a disorder described herein, e.g., cancer).

The anti-PAPP-A antibody or fragment thereof, e.g., an anti-PAPP-A antibody or fragment thereof as described herein, can be administered to the subject systemically (e.g., orally, parenterally, subcutaneously, intravenously, intramuscularly, intraperitoneally, intranasally, transdermally, or by inhalation), topically, by application to mucous membranes, such as the nose, throat and bronchial tubes, by application during a medical procedure, e.g., a surgery or lumbar puncture.

The methods of the invention can further include the step of monitoring the subject, e.g., for a reduction in one or more of: a reduction in tumor size; reduction in cancer markers, e.g., levels of PAPP-A; reduction in the appearance of new lesions, e.g., in a bone scan; a reduction in the appearance of new disease-related symptoms; or decreased or stabilization of size of soft tissue mass; or any parameter related to improvement in clinical outcome. The subject can be monitored in one or more of the following periods: prior to beginning of treatment; during the treatment; or after one or more elements of the treatment have been administered. Monitoring can be used to evaluate the need for further treatment with the same anti-PAPP-A ligand or for additional treatment with additional agents. Generally, a decrease in one or more of the parameters described above is indicative of the improved condition of the subject.

The anti-PAPP-A ligand can be used alone in unconjugated form to thereby ablate or kill the PAPP-A-associated cells. For example, if the ligand is an antibody, the ablation or killing can be mediated by an antibody-dependent cell killing mechanisms such as complement-mediated cell lysis and/or effector cell-mediated cell killing. In other embodiments, the anti-PAPP-A ligand can be bound to a substance, e.g., a cytotoxic agent or moiety, effective to kill or ablate the PAPP-A-expressing cells. For example, the anti-PAPP-A ligand can be coupled to a radioactive ion (e.g., an α, γ-, or β-emitter), e.g., iodine (¹³¹I or ¹²⁵I), yttrium (⁹⁰Y), lutetium (¹⁷⁷Lu), actinium (²²⁵Ac), or bismuth (²¹³Bi). The methods and compositions of the invention can be used in combination with other therapeutic modalities. In one embodiment, the methods of the invention include administering to the subject a anti-PAPP-A ligand, e.g., a anti-PAPP-A antibody or fragment thereof, in combination with a cytotoxic agent, in an amount effective to treat or prevent said disorder. The ligand and the cytotoxic agent can be administered simultaneously or sequentially. In other embodiments, the methods and compositions of the invention are used in combination with surgical and/or radiation procedures.

In another aspect, the invention features methods for detecting the presence of a PAPP-A protein, in a sample, in vitro (e.g., a biological sample, such as a tissue biopsy of a cancerous lesion). The subject method can be used to evaluate, e.g., diagnose or stage a disorder described herein, e.g., a cancerous disorder. The method includes: (i) contacting the sample (and optionally, a reference, e.g., control, sample) with an anti-PAPP-A ligand, as described herein, under conditions that allow interaction of the anti-PAPP-A ligand and the PAPP-A protein to occur; and (ii) detecting formation of a complex between the anti-PAPP-A ligand, and the sample (and optionally, the reference, e.g., control, sample). Formation of the complex is indicative of the presence of PAPP-A protein, and can indicate the suitability or need for a treatment described herein, e.g., a statistically significant change in the formation of the complex in the sample relative to the reference sample, e.g., the control sample, is indicative of the presence of PAPP-A in the sample

In yet another aspect, the invention provides a method for detecting the presence of PAPP-A in vivo (e.g., in vivo imaging in a subject). The subject method can be used to evaluate, e.g., diagnose, localize, or stage a disorder described herein, e.g., a cancerous disorder. The method includes: (i) administering to a subject (and optionally a control subject) an anti-PAPP-A ligand (e.g., an antibody or antigen binding fragment thereof), under conditions that allow interaction of the anti-PAPP-A ligand and the PAPP-A protein to occur; and (ii) detecting formation of a complex between the ligand and PAPP-A, wherein a statistically significant change in the formation of the complex in the subject relative to the reference, e.g., the control subject or subject's baseline, is indicative of the presence of the PAPP-A.

In other embodiments, a method of diagnosing or staging, a disorder as described herein (e.g., a cancerous disorder), is provided. The method includes: (i) identifying a subject having, or at risk of having, the disorder; (ii) obtaining a sample of a tissue or cell affected with the disorder; (iii) contacting said sample or a control sample with an anti-PAPP-A ligand, under conditions that allow interaction of the binding agent and the PAPP-A protein to occur, and (iv) detecting formation of a complex. A statistically significant increase in the formation of the complex between the ligand with respect to a reference sample, e.g., a control sample, is indicative of the disorder or the stage of the disorder.

Preferably, the anti-PAPP-A ligand used in the in vivo and in vitro diagnostic methods is directly or indirectly labeled with a detectable substance to facilitate detection of the bound or unbound binding agent. Suitable detectable substances include various enzymes, prosthetic groups, fluorescent materials, luminescent materials and radioactive materials. In one embodiment, the anti-PAPP-A ligand is coupled to a radioactive ion, e.g., indium (¹¹¹In), iodine (¹³¹I or ¹²⁵I), yttrium (90Y), actinium (²²⁵Ac), bismuth (²¹³Bi), sulfur (³⁵S), carbon (¹⁴C), tritium (³H), rhodium (⁸⁸Rh), or phosphorous (³²P). In another embodiment, the ligand is labeled with an NMR contrast agent.

The invention also provides polypeptides and nucleic acids that encompass a range of amino acid and nucleic acid sequences.

In some embodiments, the protein ligands are modified scaffold polypeptides (or peptides), cyclic peptides or linear peptides, e.g., of less than 25 amino acids. Whereas many examples described herein refer to antibody ligands or fragments thereof, it is understood, that some embodiments can be practiced using any protein ligand (e.g., an antibody and non-antibody ligand) or even a non-protein ligand. It is possible in some cases to use non-natural amino acids or other chemical features not naturally present to produce ligands. For example, the ligand can be in part or in whole a pepetidomimetic, e.g., a peptoid.

As used herein, the term “substantially identical” (or “substantially homologous”) is used herein to refer to a first amino acid or nucleotide sequence that contains a sufficient number of identical or equivalent (e.g., with a similar side chain, e.g., conserved amino acid substitutions) amino acid residues or nucleotides to a second amino acid or nucleotide sequence such that the first and second amino acid or nucleotide sequences have similar activities. In the case of antibodies, the second antibody has the same specificity and has at least 50% of the affinity of the same.

Sequences similar or homologous (e.g., at least about 85% sequence identity) to the sequences disclosed herein are also part of this application. In some embodiment, the sequence identity can be about 85%, 90%, 91%, 92%, 93%, 94%, 95%, 96%, 97%, 98%, 99% or higher. Alternatively, substantial identity exists when the nucleic acid segments will hybridize under selective hybridization conditions (e.g., highly stringent hybridization conditions), to the complement of the strand. The nucleic acids may be present in whole cells, in a cell lysate, or in a partially purified or substantially pure form.

Calculations of “homology” or “sequence identity” between two sequences (the terms are used interchangeably herein) are performed as follows. The sequences are aligned for optimal comparison purposes (e.g., gaps can be introduced in one or both of a first and a second amino acid or nucleic acid sequence for optimal alignment and non-homologous sequences can be disregarded for comparison purposes). In a preferred embodiment, the length of a reference sequence aligned for comparison purposes is at least 30%, preferably at least 40%, more preferably at least 50%, even more preferably at least 60%, and even more preferably at least 70%, 80%, 90%, 100% of the length of the reference sequence. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences, taking into account the number of gaps, and the length of each gap, which need to be introduced for optimal alignment of the two sequences.

The comparison of sequences and determination of percent identity between two sequences can be accomplished using a mathematical algorithm. In a preferred embodiment, the percent identity between two amino acid sequences is determined using the Needleman and Wunsch ((1970) J. Mol. Biol. 48:444-453) algorithm which has been incorporated into the GAP program in the GCG software package, using either a Blossum 62 matrix or a PAM250 matrix, and a gap weight of 16, 14, 12, 10, 8, 6, or 4 and a length weight of 1, 2, 3, 4, 5, or 6. In yet another preferred embodiment, the percent identity between two nucleotide sequences is determined using the GAP program in the GCG software package, using a NWSgapdna.CMP matrix and a gap weight of 40, 50, 60, 70, or 80 and a length weight of 1, 2, 3, 4, 5, or 6. A particularly preferred set of parameters (and the one that should be used if the practitioner is uncertain about what parameters should be applied to determine if a molecule is within a sequence identity or homology limitation of the invention) are a Blossum 62 scoring matrix with a gap penalty of 12, a gap extend penalty of 4, and a frameshift gap penalty of 5.

As used herein, the term “homologous” is synonymous with “similarity” and means that a sequence of interest differs from a reference sequence by the presence of one or more amino acid substitutions (although modest amino acid insertions or deletions) may also be present. Presently preferred means of calculating degrees of homology or similarity to a reference sequence are through the use of BLAST algorithms (available from the National Center of Biotechnology Information (NCBI), National Institutes of Health, Bethesda Md.), in each case, using the algorithm default or recommended parameters for determining significance of calculated sequence relatedness.

As used herein, the term “hybridizes under low stringency, medium stringency, high stringency, or very high stringency conditions” describes conditions for hybridization and washing. Guidance for performing hybridization reactions can be found in Current Protocols in Molecular Biology, John Wiley & Sons, N.Y. (1989), 6.3.1-6.3.6, which is incorporated by reference. Aqueous and nonaqueous methods are described in that reference and either can be used. Specific hybridization conditions referred to herein are as follows: 1) low stringency hybridization conditions in 6× sodium chloride/sodium citrate (SSC) at about 45° C., followed by two washes in 0.2×SSC, 0.1% SDS at least at 50° C. (the temperature of the washes can be increased to 55° C. for low stringency conditions); 2) medium stringency hybridization conditions in 6×SSC at about 45° C., followed by one or more washes in 0.2×SSC, 0.1% SDS at 60° C.; 3) high stringency hybridization conditions in 6×SSC at about 45° C., followed by one or more washes in 0.2×SSC, 0.1% SDS at 65° C.; and preferably 4) very high stringency hybridization conditions are 0.5M sodium phosphate, 7% SDS at 65° C., followed by one or more washes at 0.2×SSC, 1% SDS at 65° C. Very high stringency conditions (4) are the preferred conditions and the ones that should be used unless otherwise specified. The invention includes nucleic acid that hybridize under one or more of the above conditions to a nucleic acid described herein. The nucleic acid can encode an immunoglobulin variable domain sequence, e.g., a heavy chain or light chain of an immunoglobulin.

It is understood that the binding agent polypeptides of the invention may have additional conservative or non-essential amino acid substitutions, which do not have a substantial effect on the polypeptide functions. Whether or not a particular substitution will be tolerated, i.e., will not adversely affect desired biological properties, such as binding activity can be determined as described in Bowie, et al. (1990) Science 247:1306-1310. A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine).

A “non-essential” amino acid residue is a residue that can be altered from the wild-type sequence of the binding agent, e.g., the antibody, without abolishing or more preferably, without substantially altering a biological activity, whereas an “essential” amino acid residue results in such a change. Any nucleic acid or protein described herein can be provided in isolated form, or in a cell or organism.

An “effectively human” immunoglobulin variable region is an immunoglobulin variable region that includes a sufficient number of human framework amino acid positions such that the immunoglobulin variable region does not elicit an immunogenic response in a normal human. An “effectively human” antibody is an antibody that includes a sufficient number of human amino acid positions such that the antibody does not elicit an immunogenic response in a normal human.

A “humanized” immunoglobulin variable region is an immunoglobulin variable region that is modified to include a sufficient number of human framework amino acid positions such that the immunoglobulin variable region does not elicit an immunogenic response in a normal human. Descriptions of “humanized” immunoglobulins include, for example, U.S. Pat. No. 6,407,213 and U.S. Pat. No. 5,693,762.

An exemplary PAPP-A molecule includes a polypeptide chain having the following sequence:

| | |
|---|---|
| MRLWSWVLHLGLLSAALGCGLAERPRPARRDPRAGRPPRPAAGPATCATRGPRPPRLAAAAAAAG | (SEQ ID NO:1) |
| RAWEAVRVPRRRQQREARGATEEPSPPSRALYFSGRGEQLRVLRADLELPRDAFTLQVWLRAEGGQRSPAV | |
| ITGLYDKCSYISRDRGWVVGIHTISDQDNKDPRYFFSLKTDRARQVTTINAHRSYLPGQWVYLAATYDGQF | |
| MKLYVNGAQVATSGEQVGGIFSPLTQKCKVLMLGGSALNHNYRGYIEHFSLWKVARTQREILSDMETHGAH | |
| TALPQLLLQENWDNVKHAWSPMKDGSSPKVEFSNAHGFLLDTSLEPPLCGQTLCDNTEVIASYNQLSSFRQ | |
| PKVVRYRVVNLYEDDHKNPTVTREQVDFQHHQLAEAFKQYNISWELDVLEVSNSSLRRRLILANCDISKIG | |
| DENCDPECNHTLTGHDGGDCRHLRHPAFVKKQHNGVCDMDCNYERFNFDGGECCDPEITNVTQTCFDPDSP | |
| HRAYLDVNELKNILKLDGSTHLNIFFAKSSEEELAGVATWPWDKEALMHLGGIVLNPSFYGMPGHTHTMIH | |
| EIGHSLGLYHVFRGISEIQSCSDPCMETEPSFETGDLCNDTNPAPKHKSCGDPGPGNDTCGFHSFFNTPYN | |
| NFMSYADDDCTDSFTPNQVARMHCYLDLVYQGWQPSRKPAPVALAPQVLGHTTDSVTLEWFPPIDGHFFER | |
| ELGSACHLCLEGRILVQYASNASSPMPCSPSGHWSPREAEGHPDVEQPCKSSVRTWSPNSAVNPHTVPPAC | |
| PEPQGCYLELEFLYPLVPESLTIWVTFVSTDWDSSGAVNDIKLLAVSGKNISLGPQNVFCDVPLTIRLWDV | |
| GEEVYGIQIYTLDEHLEIDAAMLTSTADTPLCLQCKPLKYKVVRDPPLQMDVASILHLNRKFVDMDLNLGS | |
| VYQYWVITISGTEESEPSPAVTYIHGRGYCGDGIIQKDQGEQCDDMNKINGDGCSLFCRQEVSFNCIDEPS | |
| RCYFHDGDGVCEEFEQKTSIKDCGVYTPQGFLDQWASNASVSHQDQQCPGWVIIGQPAASQVCRTKVIDLS | |
| EGISQHAWYPCTISYPYSQLAQTTFWLRAYFSQPMVAAAVIVHLVTDGTYYGDQKQETISVQLLDTKDQSH | |
| DLGLHVLSCRNNPLIIPVVHDLSQPFYHSQAVRVSFSSPLVAISGVALRSFDNFDPVTLSSCQRGETYSPA | |
| EQSCVHFACEKTDCPELAVENASLNCSSSDRYHGAQCTVSCRTGYVLQIRRDDELIKSQTGPSVTVTCTEG | |
| KWNKQVACEPVDCSIPDHHQVYAASFSCPEGTTFGSQCSFQCRHPAQLKGNNSLLTCMEDGLWSFPEALCE | |
| LMCLAPPPVPNADLQTARCRENKHKVGSFCKYKCKPGYHVPGSSRKSKKRAFKTQCTQDGSWQEGACVPVT | |
| CDPPPPKFHGLYQCTNGFQFNSECRIKCEDSDASQGLGSNVIHCRKDGTWNGSFHVCQEMQGQCSVPNELN | |
| SNLKLQCPDGYAIGSECATSCLDHNSESIILPMNVTVRDIPHWLNPTRVERVVCTAGLKWYPHPALIHCVK | |
| GCEPFMGDNYCDAINNRAFCNYDGGDCCTSTVKTKKVTPFPMSCDLQGDCACRDPQAQEHSRKDLRGYSHG | |

An exemplary mature PAPP-A molecule includes a polypeptide chain having the following sequence:

| | |
|---|---|
| EARGATEEPSPPSRALYFSGRGEQLRVLRADLELPRDAFTLQVWLRAEGGQRSPAVITGLYDKCS | (SEQ ID NO:2) |
| YISRDRGWVVGIHTISDQDNKDPRYFFSLKTDRARQVTTINAHRSYLPGQWVYLAATYDGQFMKLYVNGAQ | |
| VATSGEQVGGIFSPLTQKCKVLMLGGSALNHNYRGYIEHFSLWKVARTQREILSDMETHGAHTALPQLLLQ | |
| ENWDNVKHAWSPMKDGSSPKVEFSNAHGFLLDTSLEPPLCGQTLCDNTEVIASYNQLSSFRQPKVVRYRVV | |
| NLYEDDHKNPTVTREQVDFQHHQLAEAFKQYNISWELDVLEVSNSSLRRRLILANCDISKIGDENCDPECN | |
| HTLTGHDGGDCRHLRHPAFVKKQHNGVCDMDCNYERFNFDGGECCDPEITNVTQTCFDPDSPHRAYLDVNE | |
| LKNILKLDGSTHLNIFFAKSSEEELAGVATWPWDKEALMHLGGIVLNPSFYGMPGHTHTMIHEIGHSLGLY | |
| HVFRGISEIQSCSDPCMETEPSFETGDLCNDTNPAPKHKSCGDPGPGNDTCGFHSFFNTPYNNFMSYADDD | |
| CTDSFTPNQVARMHCYLDLVYQGWQPSRKPAPVALAPQVLGHTTDSVTLEWFPPIDGHFFERELGSACHLC | |
| LEGRILVQYASNASSPMPCSPSGHWSPREAEGHPDVEQPCKSSVRTWSPWSAVNPHTVPPACPEPQGCYLE | |
| LEFLYPLVPESLTIWVTFVSTDWDSSGAVNDIKLLAVSGKNISLGPQNVFCDVPLTIRLWDVGEEVYGIQI | |
| YTLDEHLEIDAAMLTSTADTPLCLQCKPLKYKVVRDPPLQMDVASILHLNRKFVDMDLNLGSVYQYWVITI | |
| SGTEESEPSPAVTYIHGRGYCGDGIIQKDQGEQCDDMNKINGDGCSLFCRQEVSFNCIDEPSRCYFHDGDG | |
| VCEEFEQKTSIKDCGVYTPQGFLDQWASNASVSHQDQQCPGWVIIGQPAASQVCRTKVIDLSEGISQHAWY | |
| PCTISYPYSQLAQTTFWLRAYFSQPMVAAAVIVHLVTDGTYYGDQKQETISVQLLDTKDQSHDLGLHVLSC | |
| RNNPLIIPVVHDLSQPFYHSQAVRVSFSSPLVAISGVALRSFDNFDPVTLSSCQRGETYSPAEQSCVHFAC | |
| EKTDCPELAVENASLNCSSSDRYHGAQCTVSCRTGYVLQIRRDDELIKSQTGPSVTVTCTEGKWNKQVACE | |
| PVDCSIPDHHQVYAASFSCPEGTTFGSQCSFQCRHPAQLKGNNSLLTCMEDGLWSFPEALCELMCLAPPPV | |
| PNADLQTARCRENKHKVGSFCKYKCKPGYHVPGSSRKSKKRAFKTQCTQDGSWQEGACVPVTCDPPPPKFH | |
| GLYQCTNGFQFNSECRIKCEDSDASQGLGSNVIHCRKDGTWNGSFHVCQEMQGQCSVPNELNSNLKLQCPD | |
| GYAIGSECATSCLDHNSESIILPMNVTVRDIPHWLNPTRVERVVCTAGLKWYPHPALIHCVKGCEPFMGDN | |
| YCDAINNRAFCNYDGGDCCTSTVKTKKVTPFPMSCDLQGDCACRDPQAQEHSRKDLRGYSHG | |

Naturally occurring variants of PAPP-A are also known. For example, arginine R944 in the prepro-PAPP-A sequence (SEQ ID NO:1) can be substituted with serine.

An “inactivated” form of PAPP-A refers to a PAPP-A molecule that is unable to efficiently proteolyze its substrate (e.g., has less than 80, 70, 50, 40, 25, 10, 5, or 1% of normal activity). The PAPP-A molecule can be inactivated by an amino acid change (e.g., substitution, deletion, or insertion) or by an exogenous agent. For example, a PAPP-A molecule can be inactivated by an inhibitor (e.g., a ligand that binds to PAPP-A). An inhibitor may cause steric inhibition of the active site, allosteric inhibition, inhibition of substrate binding, or inability to be localized with its substrate. Its substrate can include, among others, itself (e.g., a PAPP-A molecule) and an IGFBP molecule. It is also possible for an inhibitor to function by other mechanisms, e.g., altering PAPP-A translation, processing, secretion, clearance, and so forth.

An inactive form of PAPP-A that cannot autodigest can be useful in the purification of PAPP-A and in a screen for ligands that bind PAPP-A, e.g., during a display library selection.

Exemplary amino acid changes that cause PAPP-A inactivation are changes that alter conserved residues (e.g., residues conserved between PAPP-A proteins from different mammals). An exemplary inactivated PAPP-A molecule can include an amino acid change that alters E483 (numbering with reference to SEQ ID NO:2). The amino acid change can be substitution with another amino acid, e.g., a non-acidic amino acid, e.g., alanine. An exemplary inactive PAPP-A molecule includes the mutation E483A (glutamate at position 483 is substituted with alanine). Other mutations which impair PAPP-A mediated cleavage of IGFBP-4 include M556L, Y558F, Y558A, deletion of S498-Y552, and C563A (numbering with reference to SEQ ID NO:2).

A “PAPP-A-containing structure” refers to a discrete mass present in a subject or obtained from a subject, that contains PAPP-A. Exemplary PAPP-A containing structures include unstable, e.g. vulnerable, atherosclerotic plaques or actively proliferating vascular smooth muscle cells in which PAPP-A has been found to be elevated, as well as stable atherosclerotic plaques and non-actively proliferating vascular smooth muscle cells in which PAPP-A levels are detectable. A PAPP-A containing structure may also include the serum, blood, or other biological fluid of a patient. “Circulating PAPP-A” refers to PAPP-A molecules that are present in a biological fluid of a subject, e.g., in serum, blood, or an interstitial fluid. Patients with certain conditions may have elevated circulating PAPP-A levels. The term “IGF” refers to insulin-like growth factor, and is inclusive of IGF-1 and IGF-2.

Implementations of the invention can include one or more features described herein. Other features and advantages of the instant invention will become more apparent from the following detailed description and claims. All patents, patent applications, and references cited herein are incorporated by reference in their entirety, inclusive of U.S. Provisional Patent Application Serial No. 60/448,515, filed on Feb. 19, 2003, and PCT US04/______, filed Feb. 19, 2004, attorney docket number 10280-059WO1, titled “PAPP-A Ligands.”

### DETAILED DESCRIPTION

The invention provides, inter alia, methods for identifying proteins that bind to PAPP-A. In one form, PAPP-A is a 1547 amino acid glycoprotein which can form an ˜200 kDa monomer or an ˜400 kDa dimer. PAPP-A can interact (e.g., cleave) substrates such as IGFBP-4, IGFBP-5, and IGFBP-2.

The methods can include: providing a library and screening the library to identify a member that encodes a protein that binds to the PAPP-A. The screening can be performed in a number of ways. For example, the library can be a display library. The PAPP-A can be tagged and recombinantly expressed. The PAPP-A is purified and attached to a support, e.g., to paramagnetic beads or other magnetically responsive particle. The PAPP-A can also be associated with the surface of a cell. The display library can be screened to identify members that specifically bind to the cell, e.g., only if the PAPP-A is expressed.

The invention also provides, inter alia, ligands that bind (e.g., specifically bind) to PAPP-A. Exemplary ligands include those described herein and those identified by a method described herein. In one embodiment, the ligand includes one, or two immunoglobulin variable domains, e.g., a single-chain antibody, Fab, IgG, and so forth.

Display Libraries

A display library can be used to identify proteins that bind to a PAPP-A target, e.g., a mature PAPP-A molecule or proteolytically inactive mutant PAPP-A molecule (e.g., E483A). A display library is a collection of entities; each entity includes an accessible protein component and a recoverable component that encodes or identifies the protein component. The protein component can be of any length, e.g. from three amino acids to over 300 amino acids. In a selection, the protein component of each member of the library is probed with the PAPP-A target, and if the protein component binds to the PAPP-A, the display library member is identified, typically by retention on a support.

Retained display library members are recovered from the support and analyzed. The analysis can include amplification and a subsequent selection under similar or dissimilar conditions. For example, positive and negative selections can be alternated. The analysis can also include determining the amino acid sequence of the protein component and purification of the protein component for detailed characterization.

A variety of formats can be used for display libraries. Examples include the following.

Phage Display. One format utilizes viruses, particularly bacteriophages. This format is termed “phage display.” The protein component is typically covalently linked to a bacteriophage coat protein. The linkage results form translation of a nucleic acid encoding the protein component fused to the coat protein. The linkage can include a flexible peptide linker, a protease site, or an amino acid incorporated as a result of suppression of a stop codon. Phage display is described, for example, in Ladner et al., U.S. Pat. No. 5,223,409; Smith (1985) Science 228:1315-1317; WO 92/18619; WO 91/17271; WO 92/20791; WO 92/15679; WO 93/01288; WO 92/01047; WO 92/09690; WO 90/02809; de Haard et al. (1999) J. Biol. Chem 274:18218-30; Hoogenboom et al. (1998) Immunotechnology 4:1-20; Hoogenboom et al. (2000) Immunol Today 2:371-8; Fuchs et al. (1991) Bio/Technology 9:1370-1372; Hay et al. (1992) Hum Antibod Hybridomas 3:81-85; Huse et al. (1989) Science 246:1275-1281; Griffiths et al. (1993) EMBO J 12:725-734; Hawkins et al. (1992) J Mol Biol 226:889-896; Clackson et al. (1991) Nature 352:624-628; Gram et al. (1992) PNAS 89:3576-3580; Garrard et al. (1991) Bio/Technology 9:1373-1377; Rebar et al. (1996) Methods Enzymol. 267:129-49; Hoogenboom et al. (1991) Nuc Acid Res 19:4133-4137; and Barbas et al. (1991) PNAS 88:7978-7982.

Phage display systems have been developed for filamentous phage (phage fl, fd, and M13) as well as other bacteriophage (e.g. T7 bacteriophage and lambdoid phages; see, e.g., Santini (1998) J. Mol. Biol. 282:125-135; Rosenberg et al. (1996) Innovations 6:1-6; Houshmet al. (1999) Anal Biochem 268:363-370). The filamentous phage display systems typically use fusions to a minor coat protein, such as gene III protein, and gene VIII protein, a major coat protein, but fusions to other coat proteins such as gene VI protein, gene VII protein, gene IX protein, or domains thereof can also been used (see, e.g., WO 00/71694). In one embodiment, the fusion is to a domain of the gene III protein, e.g., the anchor domain or “stump,” (see, e.g., U.S. Pat. No. 5,658,727 for a description of the gene III protein anchor domain). It is also possible to physically associate the protein being displayed to the coat using a non-peptide linkage, e.g., a non-covalent bond or a non-peptide covalent bond. For example, a disulfide bond and/or c-fos and c-jun coiled-coils can be used for physical associations (see, e.g., Crameri et al. (1993) Gene 137:69 and WO 01/05950).

The valency of the protein component can also be controlled. Cloning of the sequence encoding the protein component into the complete phage genome results in multivariant display since all replicates of the gene III protein are fused to the protein component. For reduced valency, a phagemid system can be utilized. In this system, the nucleic acid encoding the protein component fused to gene III is provided on a plasmid, typically of length less than 7000 nucleotides. The plasmid includes a phage origin of replication so that the plasmid is incorporated into bacteriophage particles when bacterial cells bearing the plasmid are infected with helper phage, e.g. M13K07. The helper phage provides an intact copy of gene III and other phage genes required for phage replication and assembly. The helper phage has a defective origin such that the helper phage genome is not efficiently incorporated into phage particles relative to the plasmid that has a wild type origin.

Bacteriophage displaying the protein component can be grown and harvested using standard phage preparatory methods, e.g. PEG precipitation from growth media.

After selection of individual display phages, the nucleic acid encoding the selected protein components, by infecting cells using the selected phages. Individual colonies or plaques can be picked, the nucleic acid isolated and sequenced.

Cell-based Display. In still another format the library is a cell-display library. Proteins are displayed on the surface of a cell, e.g., a eukaryotic or prokaryotic cell. Exemplary prokaryotic cells include E. coli cells, B. subtilis cells, spores (see, e.g., Lu et al. (1995) Biotechnology 13:366). Exemplary eukaryotic cells include yeast (e.g., Saccharomyces cerevisiae, Schizosaccharomyces pombe, Hanseula, or Pichia pastoris). Yeast surface display is described, e.g., in Boder and Wittrup (1997) Nat. Biotechnol. 15:553-557 and U.S. Provisional Patent Application No. Serial No. 60/326,320, filed Oct. 1, 2001, titled “MULTI-CHAIN EUKARYOTIC DISPLAY VECTORS AND THE USES THEREOF.” This application describes a yeast display system that can be used to display immunoglobulin proteins such as Fab fragments, and the use of mating to generate combinations of heavy and light chains.

In one embodiment, variegate nucleic acid sequences are cloned into a vector for yeast display. The cloning joins the variegated sequence with a domain (or complete) yeast cell surface protein, e.g., Aga2, Agal, Flo1, or Gas1. A domain of these proteins can anchor the polypeptide encoded by the variegated nucleic acid sequence by a transmembrane domain (e.g., Flo1) or by covalent linkage to the phospholipid bilayer (e.g., Gas1). The vector can be configured to express two polypeptide chains on the cell surface such that one of the chains is linked to the yeast cell surface protein. For example, the two chains can be immunoglobulin chains.

Ribosome Display. RNA and the polypeptide encoded by the RNA can be physically associated by stabilizing ribosomes that are translating the RNA and have the nascent polypeptide still attached. Typically, high divalent Mg₂₊ concentrations and low temperature are used. See, e.g., Mattheakis et al. (1994) Proc. Natl. Acad. Sci. USA 91:9022 and Hanes et al. (2000) Nat Biotechnol. 18:1287-92; Hanes et al. (2000) Methods Enzymol. 328:404-30. and Schaffitzel et al. (1999) J Immunol Methods. 231(1-2):119-35.

Peptide-Nucleic Acid Fusions. Another format utilizes peptide-nucleic acid fusions. Polypeptide-nucleic acid fusions can be generated by the in vitro translation of mRNA that include a covalently attached puromycin group, e.g., as described in Roberts and Szostak (1997) Proc. Natl. Acad. Sci. USA 94:12297-12302, and U.S. Pat. No. 6,207,446. The mRNA can then be reverse transcribed into DNA and crosslinked to the polypeptide.

Other Display Formats. Yet another display format is a non-biological display in which the protein component is attached to a non-nucleic acid tag that identifies the polypeptide. For example, the tag can be a chemical tag attached to a bead that displays the polypeptide or a radiofrequency tag (see, e.g., U.S. Pat. No. 5,874,214).

Scaffolds. Scaffolds for display can include: antibodies (e.g., Fab fragments, single chain Fv molecules (scFV), single domain antibodies, camelid antibodies, and camelized antibodies); T-cell receptors; MHC proteins; extracellular domains (e.g., fibronectin Type III repeats, EGF repeats); protease inhibitors (e.g., Kunitz domains, ecotin, BPTI, and so forth); TPR repeats; trifoil structures; zinc finger domains; DNA-binding proteins; particularly monomeric DNA binding proteins; RNA binding proteins; enzymes, e.g., proteases (particularly inactivated proteases), RNase; chaperones, e.g., thioredoxin, and heat shock proteins; and intracellular signaling domains (such as SH2 and SH3 domains).

Appropriate criteria for evaluating a scaffolding domain can include: (1) amino acid sequence, (2) sequences of several homologous domains, (3) 3-dimensional structure, and/or (4) stability data over a range of pH, temperature, salinity, organic solvent, oxidant concentration. In one embodiment, the scaffolding domain is a small, stable protein domains, e.g., a protein of less than 100, 70, 50, 40 or 30 amino acids. The domain may include one or more disulfide bonds or may chelate a metal, e.g., zinc.

Examples of small scaffolding domains include: Kunitz domains (58 amino acids, 3 disulfide bonds), Cucurbida maxima trypsin inhibitor domains (31 amino acids, 3 disulfide bonds), domains related to guanylin (14 amino acids, 2 disulfide bonds), domains related to heat-stable enterotoxin IA from gram negative bacteria (18 amino acids, 3 disulfide bonds), EGF domains (50 amino acids, 3 disulfide bonds), kringle domains (60 amino acids, 3 disulfide bonds), fungal carbohydrate-binding domains (35 amino acids, 2 disulfide bonds), endothelin domains (18 amino acids, 2 disulfide bonds), and Streptococcal G IgG-binding domain (35 amino acids, no disulfide bonds).

Examples of small intracellular scaffolding domains include SH2, SH3, and EVH domains. Generally, any modular domain, intracellular or extracellular, can be used.

Another useful type of scaffolding domain is the immunoglobulin (Ig) domain. Methods using immunoglobulin domains for display are described below (see, e.g., “Antibody Display Libraries”).

Display technology can also be used to obtain ligands, e.g., antibody ligands, particular epitopes of a target. This can be done, for example, by using competing non-target molecules that lack the particular epitope or are mutated within the epitope, e.g., with alanine. Such non-target molecules can be used in a negative selection procedure as described below, as competing molecules when binding a display library to the target, or as a pre-elution agent, e.g., to capture in a wash solution dissociating display library members that are not specific to the target.

Iterative Selection. In one preferred embodiment, display library technology is used in an iterative mode. A first display library is used to identify one or more ligands that bind to PAPP-A. These identified ligands are then varied using a mutagenesis method to form a second display library. Higher affinity ligands are then selected from the second library e.g., by using higher stringency or more competitive binding and washing conditions.

In some implementations, the mutagenesis is targeted to regions known or likely to be at the binding interface. If, for example, the identified ligands are antibodies, then mutagenesis can be directed to the CDR regions of the heavy or light chains as described herein. Further, mutagenesis can be directed to framework regions near or adjacent to the CDRs. In the case of antibodies, mutagenesis can also be limited to one or a few of the CDRs, e.g., to make precise step-wise improvements. Likewise, if the identified ligands are enzymes, mutagenesis can be directed to the active site and vicinity.

In one embodiment, mutagenesis is used to make an antibody more similar to one or more germline sequences. One exemplary germlining method can include: identifying one or more germline sequences that are similar (e.g., most similar in a particular database) to the sequence of the isolated antibody. Then mutations (at the amino acid level) can be made in the isolated antibody, either incrementally, in combination, or both. For example, a nucleic acid library that includes sequences encoding some or all possible germline mutations is made. The mutated antibodies are then evaluated, e.g., to identify an antibody that has one or more additional germline residues relative to the isolated antibody and that is still useful (e.g., has a functional activity). In one embodiment, as many germline residues are introduced into an isolated antibody as possible.

In one embodiment, mutagenesis is used to substitute or insert one or more germline residues into a CDR region. For example, the germline CDR residue can be from a germline sequence that is similar (e.g., most similar) to the variable region being modified. After mutagenesis, activity (e.g., binding or other functional activity) of the antibody can be evaluated to determine if the germline residue or residues are tolerated. Similar mutagenesis can be performed in the framework regions. In the case of CDR1 and CDR2, identifying a similar germline sequence can include selecting one such sequence. In the case of CDR3, identifying a similar germline sequence can include selecting one such sequence, but may including using two germline sequences that separately contribute to the amino-terminal portion and the carboxy-terminal portion. In other implementations more than one or two germline sequences are used, e.g., to form a consensus sequence.

Selecting a germline sequence can be performed in different ways. For example, a germline sequence can be selected if it meets a predetermined criteria for selectivity or similarity, e.g., at least a certain percentage identity, e.g., at least 75, 80, 85, 90, 91, 92, 93, 94, 95, 96, 97, 98, 99, or 99.5% identity. The selection can be performed using at least 2, 3, 5, or 10 germline sequences.

In one embodiment, with respect to a particular reference variable domain sequence, e.g., a sequence described herein, a related variable domain sequence has at at least 30, 40, 50, 60, 70, 80, 90, 95 or 100% of the CDR amino acid positions that are not identical to residues in the reference CDR sequences, residues that are identical to residues at corresponding positions in a human germline sequence (i.e., an amino acid sequence encoded by a human germline nucleic acid).

In one embodiment, with respect to a particular reference variable domain sequence, e.g., a sequence described herein, a related variable domain sequence has at at least 30, 50, 60, 70, 80, 90 or 100% of the FR regions are identical to FR sequence from a human germline sequence, e.g., a germline sequence related to the reference variable domain sequence.

Accordingly, it is possible to isolate an antibody which has similar activity to a given antibody of interest, but is more similar to one or more germline sequences, particularly one or more human germline sequences. For example, an antibody can be at least 90, 91, 92, 93, 94, 95, 96, 97, 98, 99, or 99.5% identical to a gernline sequence in a region outside the CDRs (e.g., framework regions). Further an antibody can include at least 1, 2, 3, 4, or 5 germline residues in a CDR region, the germline residue being from a germline sequence of similar (e.g., most similar) to the variable region being modified. Germline sequences of primary interest are human germline sequences. The activity of the antibody (e.g., the binding activity) can be within a factor or 100, 10, 5, 2, 0.5, 0.1, and 0.001 of the original antibody. An exemplary germline sequences include VKI-O2, VL2-1, VKIII-L2::JK2, vg3-23, V3-23::JH4, and V3-23::JK6.

Some exemplary mutagenesis techniques include: error-prone PCR (Leung et al. (1989) Technique 1:11-15), recombination, DNA shuffling using random cleavage (Stemmer (1994) Nature 389-391; termed “nucleic acid shuffling”), RACHITT™ (Coco et al. (2001) Nature Biotech. 19:354), site-directed mutagenesis (Zooler et al. (1987) Nucl Acids Res 10:6487-6504), cassette mutagenesis (Reidhaar-Olson (1991) Methods Enzymol. 208:564-586) and incorporation of degenerate oligonucleotides (Griffiths et al. (1994) EMBO J 13:3245).

In one example of iterative selection, the methods described herein are used to first identify a protein ligand from a display library that binds a PAPP-A with at least a minimal binding specificity for a target or a minimal activity, e.g., an equilibrium dissociation constant for binding of greater than 1 nM, 10 nM, or 100 nM. The nucleic acid sequence encoding the initial identified protein ligand is used as a template nucleic acid for the introduction of variations, e.g., to identify a second protein ligand that has enhanced properties (e.g., binding affinity, kinetics, or stability) relative to the initial protein ligand.

Off-Rate Selection. Since a slow dissociation rate can be predictive of high affinity, particularly with respect to interactions between polypeptides and their targets, the methods described herein can be used to isolate ligands with a desired kinetic dissociation rate (i.e. reduced) for a binding interaction to PAPP-A.

To select for slow dissociating ligands from a display library, the library is contacted to an immobilized PAPP-A target. The immobilized PAPP-A is then washed with a first solution that removes non-specifically or weakly bound biomolecules. Then the immobilized PAPP-A is eluted with a second solution that includes a saturation amount of free PAPP-A, i.e., PAPP-A molecules that are not attached to the particle or fragments thereof. The free PAPP-A binds to biomolecules that dissociate from the target. Rebinding is effectively prevented by the saturating amount of free PAPP-A relative to the much lower concentration of immobilized PAPP-A.

The second solution can have solution conditions that are substantially physiological or that are stringent. Typically, the solution conditions of the second solution are identical to the solution conditions of the first solution. Fractions of the second solution are collected in temporal order to distinguish early from late fractions. Later fractions include biomolecules that dissociate at a slower rate from the PAPP-A target than biomolecules in the early fractions.

Further, it is also possible to recover display library members that remain bound to the PAPP-A target even after extended incubation. These can either be dissociated using chaotropic conditions or can be amplified while attached to the target. For example, phage bound to the target can be contacted to bacterial cells.

Selecting or Screening for Specificity. The display library screening methods described herein can include a selection or screening process that discards display library members that bind to a non-target molecule. Examples of non-target molecules include: (i) a metzincin family member other than PAPP-A, e.g., an astacin or a distintegrin metalloproteinase (e.g., an ADAMs family member); (ii) a protease outside the metzincin family; and (iii) a serum albumin.

In one implementation, a so-called “negative selection” step is used to discriminate between the target and related non-target molecule and a related, but distinct non-target molecules, e.g. PAPP-A:proMBP complex or the E483A mutant PAPP-A. The display library or a pool thereof is contacted to the non-target molecule. Members of the sample that do not bind the non-target are collected and used in subsequent selections for binding to the target molecule or even for subsequent negative selections. The negative selection step can be prior to or after selecting library members that bind to the target molecule.

In another implementation, a screening step is used. After display library members are isolated for binding to the target molecule, each isolated library member is tested for its ability to bind to a non-target molecule (e.g., a non-target listed above). For example, a high-throughput ELISA screen can be used to obtain this data. The ELISA screen can also be used to obtain quantitative data for binding of each library member to the target. The non-target and target binding data are compared (e.g., using a computer and software) to identify library members that specifically bind to the target.

Diversity

Display libraries include variation at one or more positions in the displayed polypeptide. The variation at a given position can be synthetic or natural. For some libraries, both synthetic and natural diversity are included.

Synthetic Diversity. Libraries can include regions of diverse nucleic acid sequence that originate from artificially synthesized sequences. Typically, these are formed from degenerate oligonucleotide populations that include a distribution of nucleotides at each given position. The inclusion of a given sequence is random with respect to the distribution. One example of a degenerate source of synthetic diversity is an oligonucleotide that includes NNN wherein N is any of the four nucleotides in equal proportion.

Synthetic diversity can also be more constrained, e.g., to limit the number of codons in a nucleic acid sequence at a given trinucleotide to a distribution that is smaller than NNN. For example, such a distribution can be constructed using less than four nucleotides at some positions of the codon. In addition, trinucleotide addition technology can be used to further constrain the distribution.

So-called “trinucleotide addition technology” is described, e.g., in Wells et al. (1985) Gene 34:315-323, U.S. Pat. Nos. 4,760,025 and 5,869,644. Oligonucleotides are synthesized on a solid phase support, one codon (i.e., trinucleotide) at a time. The support includes many functional groups for synthesis such that many oligonucleotides are synthesized in parallel. The support is first exposed to a solution containing a mixture of the set of codons for the first position. The unit is protected so additional units are not added. The solution containing the first mixture is washed away and the solid support is deprotected so a second mixture containing a set of codons for a second position can be added to the attached first unit. The process is iterated to sequentially assemble multiple codons. Trinucleotide addition technology enables the synthesis of a nucleic acid that at a given position can encoded a number of amino acids. The frequency of these amino acids can be regulated by the proportion of codons in the mixture. Further the choice of amino acids at the given position is not restricted to quadrants of the codon table as is the case if mixtures of single nucleotides are added during the synthesis.

Natural Diversity. Libraries can include regions of diverse nucleic acid sequence that originate (or are synthesized based on) from different naturally-occurring sequences. An example of natural diversity that can be included in a display library is the sequence diversity present in immune cells (see also below). Nucleic acids are prepared from these immune cells and are manipulated into a format for polypeptide display. Another example of naturally diversity is the diversity of sequences among different species of organisms. For example, diverse nucleic acid sequences can be amplified from environmental samples, such as soil, and used to construct a display library.

Antibody Display Libraries

In one embodiment, the display library presents a diverse pool of polypeptides, each of which includes an immunoglobulin domain, e.g., an immunoglobulin variable domain. Display libraries are particular useful, for example for identifying human or “humanized” antibodies that recognize human antigens. Such antibodies can be used as therapeutics to treat human disorders such as cancer. Since the constant and framework regions of the antibody are human, these therapeutic antibodies may avoid themselves being recognized and targeted as antigens. The constant regions are also optimized to recruit effector functions of the human immune system. The in vitro display selection process surmounts the inability of a normal human immune system to generate antibodies against self-antigens.

A typical antibody display library displays a polypeptide that includes a VH domain and a VL domain. An “immunoglobulin domain” refers to a domain from the variable or constant domain of immunoglobulin molecules. Immunoglobulin domains typically contain two β-sheets formed of about seven β-strands, and a conserved disulphide bond (see, e.g., A. F. Williams and A. N. Barclay 1988 Ann. Rev Immunol. 6:381-405). The display library can display the antibody as a Fab fragment (e.g., using two polypeptide chains) or a single chain Fv (e.g., using a single polypeptide chain). Other formats can also be used.

As used herein, an “immunoglobulin variable domain sequence” refers to an amino acid sequence which can form the structure of an immunoglobulin variable domain. For example, the sequence may include all or part of the amino acid sequence of a naturally-occurring variable domain. For example, the sequence may omit one, two or more N- or C-terminal amino acids, internal amino acids, may include one or more insertions or additional terminal amino acids, or may include other alterations. In one embodiment, a polypeptide that includes immunoglobulin variable domain sequence can associate with another immunoglobulin variable domain sequence to form a target binding structure (or “antigen binding site”), e.g., a structure that preferentially interacts with an activated integrin structure or a mimic of an activated integrin structure, e.g., relative to an non-activated structure.

As in the case of the Fab and other formats, the displayed antibody can include a constant region as part of a light or heavy chain. In one embodiment, each chain includes one constant region, e.g., as in the case of a Fab. In other embodiments, additional constant regions are displayed.

Antibody libraries can be constructed by a number of processes (see, e.g., de Haard et al. (1999) J. Biol. Chem 274:18218-30; Hoogenboom et al. (1998) Immunotechnology 4:1-20. and Hoogenboom et al. (2000) Immunol Today 21:371-8. Further, elements of each process can be combined with those of other processes. The processes can be used such that variation is introduced into a single immunoglobulin domain (e.g., VH or VL) or into multiple immunoglobulin domains (e.g., VH and VL). The variation can be introduced into an immunoglobulin variable domain, e.g., in the region of one or more of CDR1, CDR2, CDR3, FR1, FR2, FR3, and FR4, referring to such regions of either and both of heavy and light chain variable domains. In one embodiment, variation is introduced into all three CDRs of a given variable domain. In another preferred embodiment, the variation is introduced into CDR1 and CDR2, e.g., of a heavy chain variable domain. Any combination is feasible. In one process, antibody libraries are constructed by inserting diverse oligonucleotides that encode CDRs into the corresponding regions of the nucleic acid. The oligonucleotides can be synthesized using monomeric nucleotides or trinucleotides. For example, Knappik et al. (2000) J. Mol. Biol. 296:57-86 describe a method for constructing CDR encoding oligonucleotides using trinucleotide synthesis and a template with engineered restriction sites for accepting the oligonucleotides.

In another process, antibody libraries are constructed from nucleic acid amplified from naïve germline immunoglobulin genes. The amplified nucleic acid includes nucleic acid encoding the VH and/or VL domain. Sources of immunoglobulin-encoding nucleic acids are described below. Amplification can include PCR, e.g., with primers that anneal to the conserved constant region, or another amplification method. (It is also possible to prepare any antibody library from nucleic acid from a subject animal immunized with a human PAPP-A).

Nucleic acid encoding immunoglobulin domains can be obtained from the immune cells of, e.g., a human, a primate, mouse, rabbit, camel, or rodent. In one example, the cells are selected for a particular property. B cells at various stages of maturity can be selected. In another example, the B cells are naïve.

In one embodiment, fluorescent-activated cell sorting (FACS) is used to sort B cells that express surface-bound IgM, IgD, or IgG molecules. Further, B cells expressing different isotypes of IgG can be isolated. In another preferred embodiment, the B or T cell is cultured in vitro. The cells can be stimulated in vitro, e.g., by culturing with feeder cells or by adding mitogens or other modulatory reagents, such as antibodies to CD40, CD40 ligand or CD20, phorbol myristate acetate, bacterial lipopolysaccharide, concanavalin A, phytohemagglutinin or pokeweed mitogen.

In still another embodiment, the cells are isolated from a subject that has an immunological disorder, e.g., systemic lupus erythematosus (SLE), rheumatoid arthritis, vasculitis, Sjogren syndrome, systemic sclerosis, or anti-phospholipid syndrome. The subject can be a human, or an animal, e.g., an animal model for the human disease, or an animal having an analogous disorder. In yet another embodiment, the cells are isolated from a transgenic non-human animal that includes a human immunoglobulin locus.

In one preferred embodiment, the cells have activated a program of somatic hypermutation. Cells can be stimulated to undergo somatic mutagenesis of immunoglobulin genes, for example, by treatment with anti-immunoglobulin, anti-CD40, and anti-CD38 antibodies (see, e.g., Bergthorsdottir et al. (2001) J Immunol. 166:2228). In another embodiment, the cells are naïve.

The nucleic acid encoding an immunoglobulin variable domain can be isolated from a natural repertoire by the following exemplary method. First, RNA is isolated from the immune cell. The RNA mixture is treated with calf intestinal phosphatase (CIP). Non-mRNAs and truncated RNAs are dephosphorylated to disallow binding of the RNA oligo in the next step of the RACE procedure. The 5′ cap on the full length mRNAs is then removed with tobacco acid pyrophosphatase and after RNA oligo hybridization, reverse transcription is used to produce the cDNAs.

The reverse transcription of the first (antisense) strand can be done in any manner with any suitable primer. See, e.g., de Haard et al. (1999) J. Biol. Chem 274:18218-30. The primer binding region can be constant among different immunoglobulins, e.g., in order to reverse transcribe different isotypes of immunoglobulin. The primer binding region can also be specific to a particular isotype of immunoglobulin. Typically, the primer is specific for a region that is 3′ to a sequence encoding at least one CDR. In another embodiment, poly-dT primers may be used (and may be preferred for the heavy-chain genes).

A synthetic sequence can be ligated to the 3′ end of the reverse transcribed strand. The synthetic sequence can be used as a primer binding site for binding of the forward primer during PCR amplification after reverse transcription. The use of the synthetic sequence can obviate the need to use a pool of different forward primers to fully capture the available diversity.

The variable domain-encoding gene is then amplified, e.g., using one or more rounds. If multiple rounds are used, nested primers can be used for increased fidelity. The amplified nucleic acid is then cloned into a display library vector.

Any method for amplifying nucleic acid sequences may be used for amplification. Methods that maximize, and do not bias, diversity are preferred. A variety of techniques can be used for nucleic acid amplification. The polymerase chain reaction (PCR; U.S. Pat. Nos. 4,683,195 and 4,683,202, Saiki, et al. (1985) Science 230, 1350-1354) utilizes cycles of varying temperature to drive rounds of nucleic acid synthesis. Transcription-based methods utilize RNA synthesis by RNA polymerases to amplify nucleic acid (U.S. Pat. No 6,066,457; U.S. Pat. No 6,132,997; U.S. Pat. No 5,716,785; Sarkar et. al., Science (1989) 244: 331-34; Stofler et al., Science (1988) 239: 491). NASBA (U.S. Pat. Nos. 5,130,238; 5,409,818; and 5,554,517) utilizes cycles of transcription, reverse-transcription, and RnaseH-based degradation to amplify a DNA sample. Still other amplification methods include rolling circle amplification (RCA; U.S. Pat. Nos. 5,854,033 and 6,143,495) and strand displacement amplification (SDA; U.S. Pat. Nos. 5,455,166 and 5,624,825).

Secondary Screening Methods

After selecting candidate display library members that bind to a target, each candidate ligand from a candidate display library member can be further analyzed, e.g., to further characterize its interaction with the target. Candidate ligands obtained by other methods can be similarly evaluated. Each candidate ligand can be subjected to one or more secondary screening assays. The assay can be for a binding property, a catalytic property (e.g., proteolysis), a physiological property (e.g., cytotoxicity, renal clearance, immunogenicity), a structural property (e.g., stability, conformation, oligomerization state) or another functional property. The same assay can be used repeatedly, but with varying conditions, e.g., to determine pH, ionic, or thermal sensitivities.

As appropriate, the assays can use the display library member directly, a recombinant polypeptide produced from the nucleic acid encoding a displayed polypeptide, or a synthetic polypeptide synthesized based on the sequence of a displayed ligand.

In one embodiment, the secondary assay evaluates the ability of the candidate ligand to alter PAPP-A activity (e.g., proteolysis activity). In one example, enzymatic inhibition of PAPP-A is evaluated using a non-labeled substrate, e.g., a synthetic substrate, e.g., a synthetic peptide substrate. After the cleavage reaction, the substrate is analyzed to determine if it was cleaved. For example, the substrate can be analyzed by a discontinuous technique such as HPLC. In another example, enzymatic inhibition of PAPP-A is evaluated using a labeled substrate, e.g., fluorescently labeled substrate, e.g., a fluorescently labeled synthetic peptide substrate. Cleavage can be monitored in real-time, e.g., during the cleavage reaction.

Exemplary activity assays also include the use of internally quenched fluorescent peptide substrates (see, e.g., C. G. Knight, Methods in Enzymol. 248, 18-34) which demonstrate an increase in fluorescence upon cleavage, fluorophor overlabeled macromolecular substrates such as IGFBP-4 or IGFBP-5 which may either be used for fluorescence intensity experiments or for fluorescence polarization measurements (J. Bio. mol. Screening 1, 33 (1996); BioTechniques 17, 585 (1994)), or Western blot analyses.

For example, a labeled peptide substrate that includes a fluorophore e.g., 7-methoxycoumarin and derivatives thereof and a quencher e.g. 2,4-dinitrophenyl can be contacted to PAPP-A in the presence of a candidate ligand. If the ligand inhibits PAPP-A proteolytic activity, the substrate remains quenched. If however, PAPP-A proteolytic activity is not inhibited, the substrate is cleaved and the fluorophore is separated from the quencher causing a detectable increase in fluorescence. Determination of the rate of cleavage of the labeled peptide substrate at fixed concentrations of labeled peptide substrate and PAPP-A and varied concentrations of candidate ligand allows a determination of the efficacy of the candidate ligand to inhibiti PAPP-A activity.

In one embodiment a natural protein substrate e.g. IGFBP-2, IGFBP-4 or IGFBP-5 may be preferred to a peptide. A continuous method to measure cleavage of these proteins can include labeling (e.g., heavily labeling or overlabeling) with a suitable fluorophore e.g. BODIPY FL, BODIPY TR-X or Fluorescein. The heavy labeling results in almost total quenching of the conjugate's fluorescence. PAPP-A mediated cleavage of the protein substrate relieves this quenching, yielding brightly fluorescent labeled peptides. The increase in fluorescence can be measured in a spectrofluorometer, minifluorometer or fluorescence microplate reader and is proportional to PAPP-A activity. This system may be used to determine the efficacy of a candidate ligand to prevent the cleavage of the natural substrates by PAPP-A. Determination of the rate of cleavage of the labeled protein substrate at fixed concentrations of labeled protein substrate and PAPP-A and varied concentrations of candidate ligand allows a determination of the efficacy of the candidate ligand to inhibit PAPP-A activity.

In one embodiment a natural protein substrate e.g. IGFBP-2, IGFBP-4 or IGFBP-5 may be optimally labeled but not heavily labeled or overlabeled with a suitable fluorophore e.g. BODIPY FL, BODIPY TR-X or Fluorescein. When the tethered fluorophore is excited by polarized fluorescent light, the polarization of fluorescence emission is dependent upon the rate of molecular tumbling. Upon PAPP-A mediated cleavage of the fluorescently labeled protein substrate, the resulting smaller peptides tumble faster, and the emitted light is depolarized relative to that measured with the intact protein. The change in fluorescence polarization may be measured in real time with any suitably equipped fluorometer including a spectrofluorometer, minifluorometer or fluorescence microplate reader and is proportional to PAPP-A activity. This system may be used to determine the efficacy of a candidate ligand to prevent the cleavage of the natural substrates by PAPP-A. Determination of the rate of cleavage of the labeled protein substrate at fixed concentrations of labeled protein substrate and PAPP-A and varied concentrations of candidate ligand allows a determination of the efficacy of the candidate ligand to inhibit PAPP-A activity.

In one embodiment, the candidate ligand is evaluated for its ability to alter PAPP-A cleavage of the natural substrates, e.g., IGFBP-2, IGFBP-4, and IGFBP-5 Cleavage of these substrates can be monitored, for example, by a separation (e.g., electrophoresis, a chromatographic assay, such as HPLC, centrifugation, and so forth) See, e.g., examples using western analysis with anti-IGFBP-2, anti-IGFBP-4, and anti-IGFBP-5 antibodies. Cleavage of natural ligands can also be monitored using labeled, e.g., fluorescently labeled, IGFBP-2, IGFBP-4, and IGFBP-5. For example, the cleavage reaction may increase the fluorescence of a labeled substrate, e.g., by separating a fluorophore from a quenching molecule. It may also be possible to follow the cleavage reaction using a sandwich ELISA style assay in which IGFBP-4, -5 is tagged and bound to an ELISA plate through the tag. This protein is then incubated with PAPP-A, −/+ candidate ligand for a given amount of time. The plate is then washed and evaluated, e.g., using an antibody to determine if a region of the substrate has separated from the plate. A signal will be obtained for those wells that contain functional inhibitors.

Exemplary assays for binding properties include the following.

ELISA. Polypeptides encoded by a display library can also be screened for a binding property using an ELISA assay. For example, each polypeptide is contacted to a microtitre plate whose bottom surface has been coated with the target, e.g., a limiting amount of the target. The plate is washed with buffer to remove non-specifically bound polypeptides. Then the amount of the polypeptide bound to the plate is determined by probing the plate with an antibody that can recognize the polypeptide, e.g., a tag or constant portion of the polypeptide. The antibody is linked to an enzyme such as alkaline phosphatase, which produces a colorimetric product when appropriate substrates are provided. The polypeptide can be purified from cells or assayed in a display library format, e.g., as a fusion to a filamentous bacteriophage coat. In another version of the ELISA assay, each polypeptide of a diversity strand library is used to coat a different well of a microtitre plate. The ELISA then proceeds using a constant target molecule to query each well.

Homogeneous Binding Assays. The binding interaction of candidate polypeptide with a target can be analyzed using a homogenous assay, i.e., after all components of the assay are added, additional fluid manipulations are not required. For example, fluorescence resonance energy transfer (FRET) can be used as a homogenous assay (see, for example, Lakowicz et al., U.S. Pat. No. 5,631,169; Stavrianopoulos, et al., U.S. Pat. No. 4,868,103). A fluorophore label on the first molecule (e.g., the molecule identified in the fraction) is selected such that its emitted fluorescent energy can be absorbed by a fluorescent label on a second molecule (e.g., the target) if the second molecule is in proximity to the first molecule. The fluorescent label on the second molecule fluoresces when it absorbs to the transferred energy. Since the efficiency of energy transfer between the labels is related to the distance separating the molecules, the spatial relationship between the molecules can be assessed. In a situation in which binding occurs between the molecules, the fluorescent emission of the ‘acceptor’ molecule label in the assay should be maximal. A binding event that is configured for monitoring by FRET can be conveniently measured through standard fluorometric detection means well known in the art (e.g., using a fluorimeter). By titrating the amount of the first or second binding molecule, a binding curve can be generated to estimate the equilibrium binding constant.

Another example of a homogenous assay is Alpha Screen (Packard Bioscience, Meriden Conn.). Alpha Screen uses two labeled beads. One bead generates singlet oxygen when excited by a laser. The other bead generates a light signal when singlet oxygen diffuses from the first bead and collides with it. The signal is only generated when the two beads are in proximity. One bead can be attached to the display library member, the other to the target. Signals are measured to determine the extent of binding.

The homogenous assays can be performed while the candidate polypeptide is attached to the display library vehicle, e.g., a bacteriophage.

Surface Plasmon Resonance (SPR). The binding interaction of a molecule isolated from a display library and a target can be analyzed using SPR. SPR or Biomolecular Interaction Analysis (BIA) detects biospecific interactions in real time, without labeling any of the interactants. Changes in the mass at the binding surface (indicative of a binding event) of the BIA chip result in alterations of the refractive index of light near the surface (the optical phenomenon of surface plasmon resonance (SPR)). The changes in the refractivity generate a detectable signal, which are measured as an indication of real-time reactions between biological molecules. Methods for using SPR are described, for example, in U.S. Pat. No. 5,641,640; Raether (1988) Surface Plasmons Springer Verlag; Sjolander and Urbaniczky (1991) Anal. Chem. 63:2338-2345; Szabo et al. (1995) Curr. Opin. Struct. Biol. 5:699-705 and on-line resources provide by BIAcore International AB (Uppsala, Sweden).

Information from SPR can be used to provide an accurate and quantitative measure of the equilibrium dissociation constant (K_{d}), and kinetic parameters, including Kₒₙ and K_{off}, for the binding of a biomolecule to a target. Such data can be used to compare different biomolecules. For example, proteins encoded by nucleic acid selected from a library of diversity strands can be compared to identify individuals that have high affinity for the target or that have a slow K_{off}. This information can also be used to develop structure-activity relationships (SAR). For example, the kinetic and equilibrium binding parameters of matured versions of a parent protein can be compared to the parameters of the parent protein. Variant amino acids at given positions can be identified that correlate with particular binding parameters, e.g., high affinity and slow K_{off}. This information can be combined with structural modeling (e.g., using homology modeling, energy minimization, or structure determination by crystallography or NMR). As a result, an understanding of the physical interaction between the protein and its target can be formulated and used to guide other design processes.

Protein Arrays. Polypeptides identified from the display library can be immobilized on a solid support, for example, on a bead or an array. For a protein array, each of the polypeptides is immobilized at a unique address on a support. Typically, the address is a two-dimensional address. Protein arrays are described below (see, e.g., Diagnostics).

Cellular Assays. A library of candidate polypeptides (e.g., previously identified by a display library or otherwise) can be screened by transforming the library into a host cell. For example, the library can include vector nucleic acid sequences that include segments that encode the polypeptides and that direct expression, e.g., such that the polypeptides are produced within the cell, secreted from the cell, or attached to the cell surface. The cells can be screened for polypeptides that bind to the PAPP-A, e.g., as detected by a change in a cellular phenotype or a cell-mediated activity. For example, in the case of an antibody that binds to the PAPP-A, the activity may be cell or complement-mediated cytotoxicity.

In one embodiment, at least some aspects of the screening method are automated. Automated methods can be used for a high throughput screen, e.g., to detect interactions with PAPP-A such as binding interactions or enzymatic interaction (e.g., inhibition of PAPP-A activity). For example, clones isolated from a primary screen and encoding candidate ligands are stored in an arrayed format (e.g., microtitre plates). A robotic device can automatically controlled to set up assays for each of the candidate ligands in a variety of formats, e.g., ELISA (using purified ligands or phage displaying the ligand), enzyme assays, cell based assays, and so forth. Enzymatic activity, for example, can be detected by any of a variety of methods, including spectroscopically, calorimetrically, using mass spectroscopy, and so forth.

Data indicate the performance of each clone for a particular assay, e.g., a binding assay, an activity assay, or a cell-based assay, can be stored in database. Software can be used to access the database and select clones that meet particular criteria, e.g., exceed a threshold for an assay. The software can then direct a robotic arm to pick the selected clones from the stored array, prepare nucleic acid encoding the ligand, prepare the ligand itself, and/or produce and screen secondary libraries that mutagenized the ligand.

Various robotic devices that can be employed in the automation process include multi-well plate conveyance systems, magnetic bead particle processors, liquid handling units, colony picking units. These devices can be built on custom specifications or purchased from commercial sources, such as Autogen (Framingham Mass.), Beckman Coulter (USA), Biorobotics (Woburn Mass.), Genetix (New Milton, Hampshire UK), Hamilton (Reno Nev.), Hudson (Springfield N.J.), Labsystems (Helsinki, Finland), Perkin Elmer Lifesciences (Wellseley Mass.), Packard Bioscience (Meriden Conn.), and Tecan (Mannedorf, Switzerland).

The activity of a protein ligand toward decreasing tumor volume and metastasis can be evaluated in model described in Rabbani et al., Int. J. Cancer 63 : 840-845 (1995). See also Xing et al., Canc. Res., 57: 3585-3593 (1997). There, Mat LyLu tumor cells were injected into the flank of Copenhagen rats. The animals were implanted with osmotic minipumps to continuously administer various doses of test compound for up to three weeks. The tumor mass and volume of experimental and control animals were evaluated during the experiment, as were metastatic growths. Evaluation of the resulting data permits a determination as to efficacy of the test compound, optimal dosing, and route of administration. Xing et al., Canc. Res., 57: 3585-3593 (1997) describes a related protocol.

The ligands described herein can be assayed for their ability to target sites of vascular injury as follows. Male New Zealand white rabbits (2 to 3 kg each) were obtained from ARI Breeding Labs, West Bridgewater, Mass. To induce vascular injury, their abdominal aortas are denuded of endothelium by a modification of the Baumgartner technique (Fischman et al., Arteriosclerosis 7:361, 1987). Briefly, after each animal is anesthetized with ketamine and ether or, alternatively, with xylazine (20 mg/ml) and Ketalar (50 mg/ml), the left femoral artery was isolated; a 4F Fogarty embolectomy catheter (Model 12-040-4F, Edwards Laboratories Incorporated, Santa Anna, Calif.) is introduced through an arterotomy in the femoral artery and is advanced under fluoroscopic visualization to the level of the diaphragm. The catheter is inflated to a pressure of about 3 psi above the balloon inflation pressure with radiographic contrast medium (Conray, Mallinkrodt, St. Louis, Mo.). Three passes can be made through the abdominal aorta with the inflated catheter to remove the aortic endothelium before removal of the catheter, ligation of the femoral artery, and closure of the wound. The animals are allowed to heal for a period of 4 to 5 weeks before injection of the labelled synthetic peptides.

Watanabe Heritable Hyperlipemic (WHHL) rabbits can also be used as animal models. They can be obtained from the WHHL Rabbit Program of the National Heart Lung and Blood Institute (Bethesda, Md.) at about 3 months of age and weighing about 1.5 kg. The animals can be raised until they were 3-4 kg in weight. At this weight, they exhibited marked aortic atherosclerosis. The ligands can be administered to the rabbits. One or more properties of the rabbits can be evaluated. For example, their arterial structure can be evaluated.

Ligand Production

Standard recombinant nucleic acid methods can be used to express a protein ligand that binds to PAPP-A. Generally, a nucleic acid sequence encoding the protein ligand is cloned into a nucleic acid expression vector. If the protein ligand includes multiple polypeptide chains, each chain must be cloned into an expression vector, e.g., the same or different vectors, that are expressed in the same or different cells. If the protein is sufficiently small, i.e., the protein is a peptide of less than 50 amino acids, the protein can be synthesized using automated organic synthetic methods. Methods for producing antibodies are also provided below.

The expression vector for expressing the protein ligand can include, in addition to the segment encoding the protein ligand or fragment thereof, regulatory sequences, including for example, a promoter, operably linked to the nucleic acid(s) of interest. Large numbers of suitable vectors and promoters are known to those of skill in the art and are commercially available for generating the recombinant constructs of the present invention. The following vectors are provided by way of example. Bacterial: pBS, phagescript, PsiX174, pBluescript SK, pBS KS, pNH8a, pNH16a, pNH18a, pNH46a (Stratagene); pTrc99A, pKK223-3, pKK233-3, pDR540, and pRIT5 (Pharmacia). Eukaryotic: pWLneo, pSV2cat, pOG44, PXTI, pSG (Stratagene) pSVK3, pBPV, pcDNA3.1 (Invitrogen), pMSG, and pSVL (Pharmacia). One preferred class of preferred libraries is the display library, which is described below.

Methods well known to those skilled in the art can be used to construct vectors containing a polynucleotide of the invention and appropriate transcriptional/translational control signals. These methods include in vitro recombinant DNA techniques, synthetic techniques and in vivo recombination/genetic recombination. See, for example, the techniques described in Sambrook & Russell, Molecular Cloning: A Laboratory Manual, 3^{rd} Edition, Cold Spring Harbor Laboratory, N.Y. (2001) and Ausubel et al., Current Protocols in Molecular Biology (Greene Publishing Associates and Wiley Interscience, N.Y. (1989). Promoter regions can be selected from any desired gene using CAT (chloramphenicol transferase) vectors or other vectors with selectable markers. Two appropriate vectors are pKK232-8 and pCM7. Particular named bacterial promoters include lacI, lacZ, T3, T7, gpt, lambda P, and trc. Eukaryotic promoters include CMV immediate early, HSV thymidine kinase, early and late SV40, LTRs from retrovirus, mouse metallothionein-I, and various art-known tissue specific promoters.

Generally, recombinant expression vectors will include origins of replication and selectable markers permitting transformation of the host cell, e.g., the ampicillin resistance gene of E. coli and S. cerevisiae auxotrophic markers (such as URA3, LEU2, HIS3, and TRPl genes), and a promoter derived from a highly expressed gene to direct transcription of a downstream structural sequence. Such promoters can be derived from operons encoding glycolytic enzymes such as 3-phosphoglycerate kinase (PGK), a-factor, acid phosphatase, or heat shock proteins, among others. The polynucleotide of the invention is assembled in appropriate phase with translation initiation and termination sequences, and preferably, a leader sequence capable of directing secretion of translated protein into the periplasmic space or extracellular medium. Optionally, a nucleic acid of the invention can encode a fusion protein including an N-terminal identification peptide imparting desired characteristics, e.g., stabilization or simplified purification of expressed recombinant product. Useful expression-vectors for bacteria are constructed by inserting a polynucleotide of the invention together with suitable translation initiation and termination signals, optionally in operable reading phase with a functional promoter. The vector will comprise one or more phenotypic selectable markers and an origin of replication to ensure maintenance of the vector and to, if desirable, provide amplification within the host. Suitable prokaryotic hosts for transformation include E. coli, Bacillus subtilis, Salmonella typhimurium and various species within the genera Pseudomonas, Streptomyces, and Staphylococcus, although others may also be employed as a matter of choice.

As a representative but nonlimiting example, useful expression vectors for bacteria can comprise a selectable marker and bacterial origin of replication derived from commercially available plasmids comprising genetic elements of the well known cloning vector pBR322 (ATCC 37017). Such commercial vectors include, for example, pKK223-3 (Pharmacia Fine Chemicals, Uppsala, Sweden) and pGEM1 (Promega, Madison, Wis., USA).

The present invention further provides host cells containing the vectors of the present invention, wherein the nucleic acid has been introduced into the host cell using known transformation, transfection or infection methods. For example, the host cells can include members of a library constructed from the diversity strand. The host cell can be a eukaryotic host cell, such as a mammalian cell, a lower eukaryotic host cell, such as a yeast cell, or the host cell can be a prokaryotic cell, such as a bacterial cell. Introduction of the recombinant construct into the host cell can be effected, for example, by calcium phosphate transfection, DEAE, dextran mediated transfection, or electroporation (Davis, L. et al., Basic Methods in Molecular Biology (1986)).

Any host/vector system can be used to identify one or more of the target elements of the present invention. These include, but are not limited to, eukaryotic hosts such as HeLa cells, CV-1 cell, COS cells, and Sf9 cells, as well as prokaryotic host such as E. coli and B. subtilis. The most preferred cells are those which do not normally express the particular reporter polypeptide or protein or which express the reporter polypeptide or protein at low natural level.

The host of the present invention may also be a yeast or other fungi. In yeast, a number of vectors containing constitutive or inducible promoters may be used. For a review see, Current Protocols in Molecular Biology, Vol. 2, Ed. Ausubel et al., Greene Publish. Assoc. & Wiley Interscience, Ch. 13 (1988); Grant et al., Expression and Secretion Vectors for Yeast, in Methods in Enzymology, Ed. Wu & Grossman, Acad. Press, N.Y. 153:516-544 (1987); Glover, DNA Cloning, Vol. II, IRL Press, Wash., D.C., Ch. 3 (1986); Bitter, Heterologous Gene Expression in Yeast, in Methods in Enzymology, Eds. Berger & Kimmel, Acad. Press, N.Y. 152:673-684 (1987); and The Molecular Biology of the Yeast Saccharomyces, Eds. Strathem et al., Cold Spring Harbor Press, Vols. I and 11 (1982).

The host of the invention may also be a prokaryotic cell such as E. coli, other enterobacteriaceae such as Serratia marcescans, bacilli, various pseudomonads, or other prokaryotes which can be transformed, transfected, infected.

The present invention further provides host cells genetically engineered to contain the polynucleotides of the invention. For example, such host cells may contain nucleic acids of the invention introduced into the host cell using known transformation, transfection or infection methods. The present invention still further provides host cells genetically engineered to express the polynucleotides of the invention, wherein such polynucleotides are in operative association with a regulatory sequence heterologous to the host cell that drives expression of the polynucleotides in the cell.

The host cell can be a higher eukaryotic host cell, such as a mammalian cell, a lower eukaryotic host cell, such as a yeast cell, or the host cell can be a prokaryotic cell, such as a bacterial cell.

Introduction of the recombinant construct into the host cell can be effected by calcium phosphate transfection, DEAE, dextran mediated transfection, or electroporation (Davis, L. et al., Basic Methods in Molecular Biology (1986)). The host cells containing one of polynucleotides of the invention can be used in conventional manners to produce the gene product encoded by the isolated fragment (in the case of an ORF).

Any host/vector system can be used to express one or more of the diversity strands of the present invention. These include, but are not limited to, eukaryotic hosts such as HeLa cells, CV-1 cell, COS cells, and Sf9 cells, as well as prokaryotic host such as E. coli and B. subtilis. The most preferred cells are those which do not normally express the particular polypeptide or protein or which expresses the polypeptide or protein at low natural level. Mature proteins can be expressed in mammalian cells, yeast, bacteria, or other cells under the control of appropriate promoters. Cell-free translation systems can also be employed to produce such proteins using RNAs derived from the DNA constructs of the present invention. Appropriate cloning and expression vectors for use with prokaryotic and eukaryotic hosts are described by Sambrook, et al., in Molecular Cloning: A Laboratory Manual, Second Edition, Cold Spring Harbor, N.Y. (1989).

Various mammalian cell culture systems can also be employed to express recombinant protein.

Examples of mammalian expression systems include the COS-7 lines of monkey kidney fibroblasts, described by Gluzman, Cell 23:175 (1981), and other cell lines capable of expressing a compatible vector, for example, the C127, 3T3, CHO, HeLa and BHK cell lines. Mammalian expression vectors will comprise an origin of replication, a suitable promoter and also any necessary ribosome-binding sites, polyadenylation site, splice donor and acceptor sites, transcriptional termination sequences, and 5′ flanking nontranscribed sequences.

DNA sequences derived from the SV40 viral genome, for example, SV40 origin, early promoter, enhancer, splice, and polyadenylation sites may be used to provide the required nontranscribed genetic elements. Recombinant polypeptides and proteins produced in bacterial culture are usually isolated by initial extraction from cell pellets, followed by one or more salting-out, aqueous ion exchange or size exclusion chromatography steps. In some embodiments, the template nucleic acid also encodes a polypeptide tag, e.g., penta- or hexa-histidine. The recombinant polypeptides encoded by a library of diversity strands can then be purified using affinity chromatography.

Microbial cells employed in expression of proteins can be disrupted by any convenient method, including freeze-thaw cycling, sonication, mechanical disruption, or use of cell lysing agents. A number of types of cells may act as suitable host cells for expression of the protein. Scopes (1994) Protein Purification: Principles and Practice, New York:Springer-Verlag provides a number of general methods for purifying recombinant (and non-recombinant) proteins. The method include, e.g., ion-exchange chromatography, size-exclusion chromatography, affinity chromatography, selective precipitation, dialysis, and hydrophobic interaction chromatography. These methods can be adapted for devising a purification strategy for the anti-PAPP-A protein ligand.

Mammalian host cells include, for example, monkey COS cells, Chinese Hamster Ovary (CHO) cells, human kidney 293 cells, human epidermal A431 cells, human Colo205 cells, 3T3 cells, CV-1 cells, other transformed primate cell lines, normal diploid cells, cell strains derived from in vitro culture of primary tissue, primary explants, HeLa cells, mouse L cells, BHK, HL-60, U937, HaK or Jurkat cells.

Alternatively, it may be possible to produce the protein in lower eukaryotes such as yeast or in prokaryotes such as bacteria. Potentially suitable yeast strains include Saccharomyces cerevisiae, Schizosaccharomyces pombe, Kluyveromyces strains, Candida, or any yeast strain capable of expressing heterologous proteins. Potentially suitable bacterial strains include Escherichia coli, Bacillus subtilis, Salmonella typhimurium, or any bacterial strain capable of expressing heterologous proteins. If the protein is made in yeast or bacteria, it may be necessary to modify the protein produced therein, for example by phosphorylation or glycosylation of the appropriate sites, in order to obtain the functional protein. Such covalent attachments may be accomplished using known chemical or enzymatic methods. In another embodiment of the present invention, cells and tissues may be engineered to express an endogenous gene comprising the polynucleotides of the invention under the control of inducible regulatory elements, in which case the regulatory sequences of the endogenous gene may be replaced by homologous recombination. As described herein, gene targeting can be used to replace a gene's existing regulatory region with a regulatory sequence isolated from a different gene or a novel regulatory sequence synthesized by genetic engineering methods.

Such regulatory sequences may be comprised of promoters, enhancers, scaffold-attachment regions, negative regulatory elements, transcriptional initiation sites, regulatory protein binding sites or combinations of said sequences. Alternatively, sequences which affect the structure or stability of the RNA or protein produced may be replaced, removed, added, or otherwise modified by targeting, including polyadenylation signals. MRNA stability elements, splice sites, leader sequences for enhancing or modifying transport or secretion properties of the protein, or other sequences which alter or improve the function or stability of protein or RNA molecules.

Antibody Production. Some antibodies, e.g., Fabs, can be produced in bacterial cells, e.g., E. coli cells. For example, if the Fab is encoded by sequences in a phage display vector that includes a suppressible stop codon between the display entity and a bacteriophage protein (or fragment thereof), the vector nucleic acid can be transferred into a bacterial cell that cannot suppress a stop codon. In this case, the Fab is not fused to the gene III protein and is secreted into the media.

Antibodies can also be produced in eukaryotic cells. In one embodiment, the antibodies (e.g., scFv's) are expressed in a yeast cell such as Pichia (see, e.g., Powers et al. (2001) J Immunol Methods. 251:123-35), Hanseula, or Saccharomyces.

In one preferred embodiment, antibodies are produced in mammalian cells. Preferred mammalian host cells for expressing the clone antibodies or antigen-binding fragments thereof include Chinese Hamster Ovary (CHO cells) (including dhfr- CHO cells, described in Urlaub and Chasin (1980) Proc. Natl. Acad. Sci. USA 77:4216-4220, used with a DHFR selectable marker, e.g., as described in Kaufman and Sharp (1982) Mol. Biol. 159:601-621), lymphocytic cell lines, e.g., NS0 myeloma cells and SP2 cells, COS cells, and a cell from a transgenic animal, e.g., a transgenic mammal. For example, the cell is a mammary epithelial cell.

In addition to the nucleic acid sequence encoding the diversified immunoglobulin domain, the recombinant expression vectors may carry additional sequences, such as sequences that regulate replication of the vector in host cells (e.g., origins of replication) and selectable marker genes. The selectable marker gene facilitates selection of host cells into which the vector has been introduced (see e.g., U.S. Pat. Nos. 4,399,216, 4,634,665 and 5,179,017). For example, typically the selectable marker gene confers resistance to drugs, such as G418, hygromycin or methotrexate, on a host cell into which the vector has been introduced. Preferred selectable marker genes include the dihydrofolate reductase (DHFR) gene (for use in dhfr host cells with methotrexate selection/amplification) and the neo gene (for G418 selection).

In an exemplary system for recombinant expression of an antibody, or antigen-binding portion thereof, of the invention, a recombinant expression vector encoding both the antibody heavy chain and the antibody light chain is introduced into dhfr- CHO cells by calcium phosphate-mediated transfection. Within the recombinant expression vector, the antibody heavy and light chain genes are each operatively linked to enhancer/promoter regulatory elements (e.g., derived from SV40, CMV, adenovirus and the like, such as a CMV enhancer/AdMLP promoter regulatory element or an SV40 enhancer/AdMLP promoter regulatory element) to drive high levels of transcription of the genes. The recombinant expression vector also carries a DHFR gene, which allows for selection of CHO cells that have been transfected with the vector using methotrexate selection/amplification. The selected transformant host cells are cultured to allow for expression of the antibody heavy and light chains and intact antibody is recovered from the culture medium. Standard molecular biology techniques are used to prepare the recombinant expression vector, transfect the host cells, select for transformants, culture the host cells and recover the antibody from the culture medium. For example, some antibodies can be isolated by affinity chromatography with a Protein A or Protein G resin.

For antibodies that include an Fc domain, the antibody production system preferably synthesizes antibodies in which the Fc region is glycosylated. For example, the Fc domain of IgG molecules is glycosylated at asparagine 297 in the CH2 domain. This asparagine is the site for modification with biantennary-type oligosaccharides. It has been demonstrated that this glycosylation is required for effector functions mediated by Fcγ receptors and complement C1q (Burton and Woof (1992) Adv. Immunol. 1:1-84; Jefferis et al. (1998) Immunol. Rev. 163:59-76). In a preferred embodiment, the Fc domain is produced in a mammalian expression system that appropriately glycosylates the residue corresponding to asparagine 297. The Fc domain can also include other eukaryotic post-translational modifications.

Antibodies can also be produced by a transgenic animal. For example, U.S. Pat. No. 5,849,992 describes a method of expressing an antibody in the mammary gland of a transgenic mammal. A transgene is constructed that includes a milk-specific promoter and nucleic acids encoding the antibody of interest and a signal sequence for secretion. The milk produced by females of such transgenic mammals includes, secreted-therein, the antibody of interest. The antibody can be purified from the milk, or for some applications, used directly.

Pharmaceutical Compositions

In another aspect, the present invention provides compositions, e.g., pharmaceutically acceptable compositions, which include an anti-PAPP-A ligand, e.g., an antibody molecule, other polypeptide or peptide identified as binding to PAPP-A, or described herein, formulated together with a pharmaceutically acceptable carrier. As used herein, “pharmaceutical compositions” encompass labeled ligands for in vivo imaging as well as therapeutic compositions.

As used herein, “pharmaceutically acceptable carrier” includes any and all solvents, dispersion media, coatings, antibacterial and antifingal agents, isotonic and absorption delaying agents, and the like that are physiologically compatible. Preferably, the carrier is suitable for intravenous, intramuscular, subcutaneous, parenteral, spinal or epidermal administration (e.g., by injection or infusion). Depending on the route of administration, the active compound, i.e., protein ligand may be coated in a material to protect the compound from the action of acids and other natural conditions that may inactivate the compound.

A “pharmaceutically acceptable salt” refers to a salt that retains the desired biological activity of the parent compound and does not impart any undesired toxicological effects (see e.g., Berge, S. M., et al. (1977) J. Pharm. Sci. 66:1-19). Examples of such salts include acid addition salts and base addition salts. Acid addition salts include those derived from nontoxic inorganic acids, such as hydrochloric, nitric, phosphoric, sulfuric, hydrobromic, hydroiodic, phosphorous and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

The compositions of this invention may be in a variety of forms. These include, for example, liquid, semi-solid and solid dosage forms, such as liquid solutions (e.g., injectable and infusible solutions), dispersions or suspensions, tablets, pills, powders, liposomes and suppositories. The preferred form depends on the intended mode of administration and therapeutic application. Typical preferred compositions are in the form of injectable or infusible solutions, such as compositions similar to those used for administration of humans with antibodies. The preferred mode of administration is parenteral (e.g., intravenous, subcutaneous, intraperitoneal, intramuscular). In a preferred embodiment, the anti-PAPP-A ligand is administered by intravenous infusion or injection. In another preferred embodiment, the anti-PAPP-A ligand is administered by intramuscular or subcutaneous injection.

The phrases “parenteral administration” and “administered parenterally” as used herein means modes of administration other than enteral and topical administration, usually by injection, and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal, epidural and intrastemal injection and infusion.

Pharmaceutical compositions typically must be sterile and stable under the conditions of manufacture and storage. A pharmaceutical composition can also be tested to insure it meets regulatory and industry standards for administration. For example, endotoxin levels in the preparation can be tested using the Limulus amebocyte lysate assay (e.g., using the kit from Bio Whittaker lot # 7L3790, sensitivity 0.125 EU/mL) according to the USP 24/NF 19 methods. Sterility of pharmaceutical compositions can be determined using thioglycollate medium according to the USP 24/NF 19 methods. For example, the preparation is used to inoculate the thioglycollate medium and incubated at 35° C. for 14 or more days. The medium is inspected periodically to detect growth of a microorganism.

The composition can be formulated as a solution, microemulsion, dispersion, liposome, or other ordered structure suitable to high drug concentration. Sterile injectable solutions can be prepared by incorporating the active compound (i.e., the ligand) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

In one embodiment, the anti-PAPP-A protein ligands are coupled to a carrier molecule. For example, the carrier molecule can improve bioavailability, providing a targeting activity, or a stabilizing activity. For example, the carrier molecule can be polyethylene glycol (PEG), an albumin (e.g., serum albumin, e.g., human serum albumin), or a peptide that associates with serum albumin. See, e.g., U.S. Ser. No. 10/094,401, filed Mar. 8, 2002.

The anti-PAPP-A protein ligands of the present invention can be administered by a variety of methods known in the art, although for many applications, the preferred route/mode of administration is intravenous injection or infusion. For example, for therapeutic applications, the anti-PAPP-A ligand can be administered by intravenous infusion at a rate of less than 30, 20, 10, 5, or 1 mg/min to reach a dose of about 1 to 100 mg/m² or 7 to 25 mg/m². The route and/or mode of administration will vary depending upon the desired results. In certain embodiments, the active compound may be prepared with a carrier that will protect the compound against rapid release, such as a controlled release formulation, including implants, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known. See, e.g., Sustained and Controlled Release Drug Delivery Systems, J. R. Robinson, ed., Marcel Dekker, Inc., New York, 1978.

In certain embodiments, the ligand may be orally administered, for example, with an inert diluent or an assimilable edible carrier. The compound (and other ingredients, if desired) may also be enclosed in a hard or soft shell gelatin capsule, compressed into tablets, or incorporated directly into the subject's diet. For oral therapeutic administration, the compounds may be incorporated with excipients and used in the form of ingestible tablets, buccal tablets, troches, capsules, elixirs, suspensions, syrups, wafers, and the like. To administer a compound of the invention by other than parenteral administration, it may be necessary to coat the compound with, or co-administer the compound with, a material to prevent its inactivation.

Pharmaceutical compositions can be administered with medical devices known in the art. For example, in a preferred embodiment, a pharmaceutical composition of the invention can be administered with a needle-less hypodermic injection device, such as the devices disclosed in U.S. Pat. Nos. 5,399,163, 5,383,851, 5,312,335, 5,064,413, 4,941,880, 4,790,824, or 4,596,556. Examples of well-known implants and modules useful in the present invention include: U.S. Pat. No. 4,487,603, which discloses an implantable micro-infusion pump for dispensing medication at a controlled rate; U.S. Pat. No. 4.,486,194, which discloses a therapeutic device for administering medicants through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. Of course, many other such implants, delivery systems, and modules are also known.

In certain embodiments, the compounds of the invention can be formulated to ensure proper distribution in vivo. For example, the blood-brain barrier (BBB) excludes many highly hydrophilic compounds. To ensure that the therapeutic compounds of the invention cross the BBB (if desired), they can be formulated, for example, in liposomes. For methods of manufacturing liposomes, see, e.g., U.S. Pat. Nos. 4,522,811; 5,374,548; and 5,399,331. The liposomes may comprise one or more moieties that are selectively transported into specific cells or organs, thus enhance targeted drug delivery (see, e.g., V. V. Ranade (1989) J. Clin. Pharmacol. 29:685).

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the subjects to be treated; each unit contains a predetermined quantity of active compound calculated to produce the desired therapeutic effect in association with the required pharmaceutical carrier. The specification for the dosage unit forms of the invention are dictated by and directly dependent on (a) the unique characteristics of the active compound and the particular therapeutic effect to be achieved, and (b) the limitations inherent in the art of compounding such an active compound for the treatment of sensitivity in individuals.

An exemplary, non-limiting range for a therapeutically or prophylactically effective amount of an antibody of the invention is 0.1-20 mg/kg, more preferably 1-10 mg/kg. The anti-PAPP-A antibody can be administered by intravenous infusion at a rate of less than 30, 20, 10, 5, or 1 mg/min to reach a dose of about 1 to 100 mg/m² or about 5 to 30 mg/m². For ligands smaller in molecular weight than an antibody, appropriate amounts can be proportionally less. It is to be noted that dosage values may vary with the type and severity of the condition to be alleviated. It is to be further understood that for any particular subject, specific dosage regimens should be adjusted over time according to the individual need and the professional judgment of the person administering or supervising the administration of the compositions, and that dosage ranges set forth herein are exemplary only and are not intended to limit the scope or practice of the claimed composition.

The pharmaceutical compositions of the invention may include a “therapeutically effective amount” or a “prophylactically effective amount” of an anti-PAPP-A ligand of the invention. A “therapeutically effective amount” refers to an amount effective, at dosages and for periods of time necessary, to achieve the desired therapeutic result. A therapeutically effective amount of the composition may vary according to factors such as the disease state, age, sex, and weight of the individual, and the ability of the protein ligand to elicit a desired response in the individual. A therapeutically effective amount is also one in which any toxic or detrimental effects of the composition is outweighed by the therapeutically beneficial effects. A “therapeutically effective dosage” preferably inhibits a measurable parameter, e.g., tumor growth rate by at least about 20%, 40%, 60, or 80% relative to untreated, matched subjects; plaque formation rate by at least about 20%, 40%, 60, or 80% relative to untreated, matched subjects; or IGF availability by at least about 20%, 40%, 60, or 80% relative to untreated, matched subjects. The ability of a compound to inhibit a measurable parameter, e.g., cancer, can be evaluated in an animal model system predictive of efficacy in human tumors. Alternatively, this property of a composition can be evaluated by examining the ability of the compound to inhibit, such inhibition in vitro by assays known to the skilled practitioner.

A “prophylactically effective amount” refers to an amount effective, at dosages and for periods of time necessary, to achieve the desired prophylactic result. Typically, since a prophylactic dose is used in subjects prior to or at an earlier stage of disease, the prophylactically effective amount will be less than the therapeutically effective amount.

Also within the scope of the invention are kits comprising the protein ligand that binds to PAPP-A and instructions for use, e.g., treatment, prophylactic, or diagnostic use. In one embodiment, the instructions for diagnostic applications include the use of the anti-PAPP-A ligand (e.g., antibody or antigen-binding fragment thereof, or other polypeptide or peptide) to detect PAPP-A, in vitro, e.g., in a sample, e.g., a biopsy or cells from a patient having a cancer or neoplastic disorder, or in vivo. In another embodiment, the instructions for therapeutic applications include suggested dosages and/or modes of administration in a patient with a cancer or neoplastic disorder. The kit can further contain a least one additional reagent, such as a diagnostic or therapeutic agent, e.g., a diagnostic or therapeutic agent as described herein, and/or one or more additional anti-PAPP-A ligands, formulated as appropriate, in one or more separate pharmaceutical preparations.

In another embodiment, it is possible to deliver an anti-PAPP-A ligand using a medical device, e.g., a catheter, a screw, balloon, or stent. See, e.g., U.S. Pat. No. 6,503,556 for a method of coating a stent. Stents can be used to maintain a body lumen. An exemplary stent has a tubular shape and an inner channel that allows flow through the body lumen. For example, the outer surface of the stent can be coated with an anti-PAPP-A ligand since this surface interacts with the body lumen. For example, U.S. Pat. No. 6,494,908 describes an exemplary stent.

Treatments

Protein ligands that bind to PAPP-A, e.g., identified by the method described herein and/or detailed herein, have therapeutic and prophylactic utilities. For example, these ligands can be administered to cells in culture, e.g. in vitro or ex vivo, or in a subject, e.g., in vivo, to treat, prevent, and/or diagnose a variety of disorders, such as cancers or a circulatory disorder, e.g., atherosclerosis.

As used herein, the term “treat” or “treatment” is defined as the application or administration of an anti-PAPP-A antibody, alone or in combination with, a second agent to a subject, e.g., a patient, or application or administration of the agent to an isolated tissue or cell, e.g., cell line, from a subject, e.g., a patient, who has a disorder (e.g., a disorder as described herein), a symptom of a disorder or a predisposition toward a disorder, with the purpose to cure, heal, alleviate, relieve, alter, remedy, ameliorate, improve or affect the disorder, the symptoms of the disorder or the predisposition toward the disorder.

Treating a cell refers to altering the behaviour of the cell (including, e.g., the inhibition of an activity, e.g., proliferation, growth, differentiation, ablation, killing of a cell in vitro or in vivo, or otherwise reducing capacity of a cell, e.g., an aberrant cell, to mediate a disorder, e.g., a disorder as described herein (e.g., a cancerous disorder, an inflammatory disorder, or a cardiovascular disorder). In one embodiment, “treating a cell” refers to a reduction in the activity of a cell, reduction in proliferation of a cell, e.g., a hyperproliferative cell, and/or reduction or cessation of cell differentiation. Such reduction does not necessarily indicate a total elimination of the cell, but a reduction, e.g., a statistically significant reduction, in the activity or the number of the cell.

As used herein, an amount of an anti-PAPP-A ligand effective to treat a disorder, or a “therapeutically effective amount” refers to an amount of the ligand which is effective, upon single or multiple dose administration to a subject, in treating a cell, e.g., a cancer cell, treating a PAPP-A containing structure, treating a plaque, or in prolonging curing, alleviating, relieving or improving a subject with a disorder as described herein beyond that expected in the absence of such treatment. As used herein, “inhibiting the growth” of the neoplasm refers to slowing, interrupting, arresting or stopping its growth and metastases and does not necessarily indicate a total elimination of the neoplastic growth.

As used herein, an amount of an anti-PAPP-A ligand effective to prevent a disorder, or a “a prophylactically effective amount” of the ligand refers to an amount of an anti-PAPP-A ligand, e.g., an anti-PAPP-A antibody described herein, which is effective, upon single- or multiple-dose administration to the subject, in preventing or delaying the occurrence of the onset or recurrence of a disorder, e.g., a cancer.

The terms “induce”, “inhibit”, “potentiate”, “elevate”, “increase”, “decrease” or the like, e.g., which denote quantitative differences between two states, refer to a difference, e.g., a statistically significant difference, between the two states. For example, “an amount effective to inhibit the proliferation of a cell” means that the rate of growth of the cells will be different, e.g., statistically significantly different, from the untreated cells. Statistical measures include the Student's T test, and Pearson's coefficient (e.g., P<0.05). A ligand described herein can be used, e.g., to inhibit the proliferation of an IGF-dependent cell.

As used herein, the term “subject” is intended to include human and non-human animals. Preferred human animals include a human patient having a disorder characterized by abnormal cell proliferation or cell differentiation. The term “non-human animals” of the invention includes all vertebrates, e.g., non-mammals (such as chickens, amphibians, reptiles) and mammals, such as non-human primates, sheep, dog, cow, pig, etc. In one embodiment, the subject is a human subject. Alternatively, the subject can be a mammal expressing a PAPP-A-like antigen with which an antibody of the invention cross-reacts. A protein ligand of the invention can be administered to a human subject for therapeutic purposes (discussed further below). Moreover, an anti-PAPP-A ligand can be administered to a non-human mammal expressing the PAPP-A-like antigen to which the ligand binds (e.g., a primate, pig or mouse) for veterinary purposes or as an animal model of human disease. Regarding the latter, such animal models may be useful for evaluating the therapeutic efficacy of the ligand (e.g., testing of dosages and time courses of administration).

In one embodiment, the invention provides a method of treating (e.g., ablating or killing) a cell (e.g., a non-cancerous cell, e.g., a normal, benign or hyperplastic cell, or a cancerous cell, e.g., a malignant cell, e.g., cell found in a solid tumor, a soft tissue tumor, or a metastatic lesion (e.g., a cell found in renal, urothelial, colonic, rectal, pulmonary, breast or hepatic, cancers and/or metastasis)). The method can include binding an anti-PAPP-A ligand to PAPP-A to alter the processing of PAPP-A substrate, e.g., thereby decreasing the availability of active IGF. For example, a method of altering the availability of active IGF can include administering an anti-PAPP-A ligand, e.g., a ligand described herein, in an amount effective to alter the availability of active IGF, e.g., 0.1-20 mg/kg/day, more preferably 1-10 mg/kg/day.

The methods can be used on cells in culture, e.g. in vitro or ex vivo. For example, cancerous or metastatic cells (e.g., renal, urothelial, colon, rectal, lung, breast, ovarian, prostatic, or liver cancerous or metastatic cells) can be cultured in vitro in culture medium and the contacting step can be effected by adding the anti-PAPP-A ligand to the culture medium. The methods can be performed on cells (e.g., cancerous or metastatic cells) present in a subject, as part of an in vivo (e.g., therapeutic or prophylactic) protocol. For in vivo embodiments, the contacting step is effected in a subject and includes administering the anti-PAPP-A ligand to the subject under conditions effective to permit both binding of the ligand to the cell and the treating, e.g., the killing or ablating of the cell.

The methods can be used to treat a cancer. As used herein, the terms “cancer”, “hyperproliferative”, “malignant”, and “neoplastic” are used interchangeably, and refer to those cells an abnormal state or condition characterized by rapid proliferation or neoplasm. The terms include all types of cancerous growths or oncogenic processes, metastatic tissues or malignantly transformed cells, tissues, or organs, irrespective of histopathologic type or stage of invasiveness. “Pathologic hyperproliferative” cells occur in disease states characterized by malignant tumor growth.

In one embodiment, the methods are used to treat neoplastic growth of cells that are responsive to IGF, e.g., cells that require IGF or cell whose growth rate is reduced at least 10%, 30%, or 60% in the absence of IGF. For example, the methods can be used to treat a glioblastoma multiforme (GBM), e.g., Grade IV astrocytoma. In one embodiment, the anti-PAPP-A ligand is applied during surgical intervention or during a lumbar puncture. In another embodiment, the anti-PAPP-A ligand is provided intravenously.

The common medical meaning of the term “neoplasia” refers to “new cell growth” that results as a loss of responsiveness to normal growth controls, e.g. to neoplastic cell growth. A “hyperplasia” refers to cells undergoing an abnormally high rate of growth. However, as used herein, the terms neoplasia and hyperplasia can be used interchangeably, as their context will reveal, referring generally to cells experiencing abnormal cell growth rates. Neoplasias and hyperplasias include “tumors,” which may be benign, premalignant or malignant.

Examples of cancerous disorders include, but are not limited to, solid tumors, soft tissue tumors, and metastatic lesions. Examples of solid tumors include malignancies, e.g., sarcomas, adenocarcinomas, and carcinomas, of the various organ systems, such as those affecting lung, breast, lymphoid, gastrointestinal (e.g., colon), and genitourinary tract (e.g., renal, urothelial cells), pharynx, prostate, ovary as well as adenocarcinomas which include malignancies such as most colon cancers, rectal cancer, renal-cell carcinoma, liver cancer, non-small cell carcinoma of the lung, cancer of the small intestine and so forth. Metastatic lesions of the aforementioned cancers can also be treated or prevented using the methods and compositions of the invention.

The methods can be useful in treating malignancies of the various organ systems, such as those affecting lung, breast, lymphoid, gastrointestinal (e.g., colon), and genitourinary tract, prostate, ovary, pharynx, as well as adenocarcinomas which include malignancies such as most colon cancers, renal-cell carcinoma, prostate cancer and/or testicular tumors, non-small cell carcinoma of the lung, cancer of the small intestine and cancer of the esophagus. Exemplary solid tumors that can be treated include: fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, non-small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, and retinoblastoma.

The term “carcinoma” is recognized by those skilled in the art and refers to malignancies of epithelial or endocrine tissues including respiratory system carcinomas, gastrointestinal system carcinomas, genitourinary system carcinomas, testicular carcinomas, breast carcinomas, prostatic carcinomas, endocrine system carcinomas, and melanomas. Exemplary carcinomas include those forming from tissue of the cervix, lung, prostate, breast, head and neck, colon and ovary. The term also includes carcinosarcomas, e.g., which include malignant tumors composed of carcinomatous and sarcomatous tissues. An “adenocarcinoma” refers to a carcinoma derived from glandular tissue or in which the tumor cells form recognizable glandular structures.

The term “sarcoma” is recognized by those skilled in the art and refers to malignant tumors of mesenchymal derivation.

The subject method can also be used to inhibit the proliferation of hyperplastic/neoplastic cells of hematopoietic origin, e.g., arising from myeloid, lymphoid or erythroid lineages, or precursor cells thereof. For instance, the present invention contemplates the treatment of various myeloid disorders including, but not limited to, acute promyeloid leukemia (APML), acute myelogenous leukemia (AML) and chronic myelogenous leukemia (CML) (reviewed in Vaickus, L. (1991) Crit Rev. in Oncol./Hemotol. 11:267-97). Lymphoid malignancies that may be treated by the subject method include, but are not limited to acute lymphoblastic leukemia (ALL), which includes B-lineage ALL and T-lineage ALL, chronic lymphocytic leukemia (CLL), prolymphocytic leukemia (PLL), hairy cell leukemia (HLL) and Waldenstrom's macroglobulinemia (WM). Additional forms of malignant lymphomas contemplated by the treatment method of the present invention include, but are not limited to, non-Hodgkin's lymphoma and variants thereof, peripheral T-cell lymphomas, adult T-cell leukemia/lymphoma (ATL), cutaneous T-cell lymphoma (CTCL), large granular lymphocytic leukemia (LGF) and Hodgkin's disease.

Methods of administering anti-PAPP-A ligands are described in “Pharmaceutical Compositions”. Suitable dosages of the molecules used will depend on the age and weight of the subject and the particular drug used. The ligands can be used as competitive agents to inhibit or reduce an interaction, e.g., between PAPP-A and a PAPP-A substrate, e.g., an IGFBP.

In one embodiment, the anti-PAPP-A ligands are used to kill or ablate cancerous cells and normal, benign hyperplastic, and cancerous cells in vivo. The ligands can be used by themselves or conjugated to an agent, e.g., a cytotoxic drug, radioisotope. This method includes: administering the ligand alone or attached to a cytotoxic drug, to a subject requiring such treatment.

The terms “cytotoxic agent” and “cytostatic agent” and “anti-tumor agent” are used interchangeably herein and refer to agents that have the property of inhibiting the growth or proliferation (e.g., a cytostatic agent), or inducing the killing, of hyperproliferative cells, e.g., an aberrant cancer cell. In cancer therapeutic embodiment, the term “cytotoxic agent” is used interchangeably with the terms “anti-cancer” or “anti-tumor” to mean an agent, which inhibits the development or progression of a neoplasm, particularly a solid tumor, a soft tissue tumor, or a metastatic lesion.

Nonlimiting examples of anti-cancer agents include, e.g., antimicrotubule agents, topoisomerase inhibitors, antimetabolites, mitotic inhibitors, alkylating agents, intercalating agents, agents capable of interfering with a signal transduction pathway, agents that promote apoptosis, radiation, and antibodies against other tumor-associated antigens (including naked antibodies, immunotoxins and radioconjugates). Examples of the particular classes of anti-cancer agents are provided in detail as follows: antitubulin/antimicrotubule, e.g., paclitaxel, vincristine, vinblastine, vindesine, vinorelbin, taxotere; topoisomerase I inhibitors, e.g., topotecan, camptothecin, doxorubicin, etoposide, mitoxantrone, daunorubicin, idarubicin, teniposide, amsacrine, epirubicin, merbarone, piroxantrone hydrochloride; antimetabolites, e.g., 5-fluorouracil (5-FU), methotrexate, 6-mercaptopurine, 6-thioguanine, fludarabine phosphate, cytarabine/Ara-C, trimetrexate, gemcitabine, acivicin, alanosine, pyrazofurin, N-Phosphoracetyl-L-Asparate=PALA, pentostatin, 5-azacitidine, 5-Aza 2′-deoxycytidine, ara-A, cladribine, 5-fluorouridine, FUDR, tiazofurin, N-[5-[N-(3,4-dihydro-2-methyl-4-oxoquinazolin-6-ylmethyl)-N-methylamino]-2-thenoyl]-L-glutamic acid; alkylating agents, e.g., cisplatin, carboplatin, mitomycin C, BCNU=Carmustine, melphalan, thiotepa, busulfan, chlorambucil, plicamycin, dacarbazine, ifosfamide phosphate, cyclophosphamide, nitrogen mustard, uracil mustard, pipobroman, 4-ipomeanol; agents acting via other mechanisms of action, e.g., dihydrolenperone, spiromustine, and desipeptide; biological response modifiers, e.g., to enhance anti-tumor responses, such as interferon; apoptotic agents, such as actinomycin D; and anti-hormones, for example anti-estrogens such as tamoxifen or, for example antiandrogens such as 4′-cyano-3-(4-fluorophenylsulphonyl)-2-hydroxy-2-methyl-3′-(trifluoromethyl) propionanilide.

Anti-PAPP-A ligands can be modified, e.g., by coupling or physical association with a cytotoxin or other bioactive agent. The ligands may be used to deliver a variety of cytotoxic drugs including therapeutic drugs, a compound emitting radiation, molecules of plants, fungal, or bacterial origin, biological proteins, and mixtures thereof. The cytotoxic drugs can be intracellularly acting cytotoxic drugs, such as short-range radiation emitters, including, for example, short-range, high-energy α-emitters, as described herein. The conjugate of the anti-PAPP-A ligand and the cytotoxin or other bioactive agent can be used to target, e.g., cells that have PAPP-A associated with their cell surface.

Enzymatically active toxins and fragments thereof are exemplified by diphtheria toxin A fragment, nonbinding active fragments of diphtheria toxin, exotoxin A (from Pseudomonas aeruginosa), ricin A chain, abrin A chain, modeccin A chain, α-sacrin, certain Aleurites fordii proteins, certain Dianthin proteins, Phytolacca americana proteins (PAP, PAPII and PAP-S), Morodica charantia inhibitor, curcin, crotin, Saponaria officinalis inhibitor, gelonin, mitogillin, restrictocin, phenomycin, and enomycin. Procedures for preparing enzymatically active polypeptides of the immunotoxins are described in WO84/03508 and WO85/03508. Examples of cytotoxic moieties that can be conjugated to ligands include adriamycin, chlorambucil, daunomycin, methotrexate, neocarzinostatin, and platinum.

In the case of polypeptide toxins, recombinant nucleic acid techniques can be used to construct a nucleic acid that encodes the ligand (or a protein component thereof) and the cytotoxin (or a protein component thereof) as translational fusions. The recombinant nucleic acid is then expressed, e.g., in cells and the encoded fusion polypeptide isolated.

Procedures for conjugating protein ligands (e.g., antibodies) with the cytotoxic agents have been previously described. Procedures for conjugating chlorambucil with antibodies are described by Flechner (1973) European Journal of Cancer, 9:741-745; Ghose et al. (1972) British Medical Journal, 3:495-499; and Szekerke, et al. (1972) Neoplasma, 19:211-215. Procedures for conjugating daunomycin and adriamycin to antibodies are described by Hurwitz, E. et al. (1975) Cancer Research, 35:1175-1181 and Arnon et al. (1982) Cancer Surveys, 1:429-449. Procedures for preparing antibody-ricin conjugates are described in U.S. Pat. No. 4,414,148 and by Osawa, T., et al. (1982) Cancer Surveys, 1:373-388 and the references cited therein. Coupling procedures as also described in EP 86309516.2.

In one embodiment, to kill or ablate normal, benign hyperplastic, or cancerous cells, a first protein ligand is conjugated with a prodrug that is activated only when in close proximity with a prodrug activator. The prodrug activator is conjugated with a second protein ligand, preferably one that binds to a non-competing site on the target molecule. Whether two protein ligands bind to competing or non-competing binding sites can be determined by conventional competitive binding assays. Drug-prodrug pairs suitable for use in the practice of the present invention are described in Blakely et al., (1996) Cancer Research, 56:3287-3292.

Alternatively, the anti-PAPP-A ligand can be coupled to high energy radiation emitters, for example, a radioisotope, such as ¹³¹I, a γ-emitter, which, when localized at the tumor site, results in a killing of several cell diameters. See, e.g., S. E. Order, “Analysis, Results, and Future Prospective of the Therapeutic Use of Radiolabeled Antibody in Cancer Therapy”, Monoclonal Antibodies for Cancer Detection and Therapy, R. W. Baldwin et al. (eds.), pp 303-316 (Academic Press 1985). Other suitable radioisotopes include α-emitters, such as ²¹²Bi, ²¹³Bi, and ²¹¹ At, and β-emitters, such as ¹⁸⁶Re and ⁹⁰Y. Moreover, Lu¹¹⁷ may also be used as both an imaging and cytotoxic agent.

Radioimmunotherapy (RIT) using antibodies labeled with ¹³¹I,⁹⁰Y, and ¹⁷⁷Lu is under intense clinical investigation. There are significant differences in the physical characteristics of these three nuclides and as a result, the choice of radionuclide is very critical in order to deliver maximum radiation dose to the tumor. The higher beta energy particles of ⁹⁰Y may be good for bulky tumors. The relatively low energy beta particles of ¹³¹I are ideal, but in vivo dehalogenation of radioiodinated molecules is a major disadvantage for internalizing antibody. In contrast, ¹⁷⁷Lu has low energy beta particle with only 0.2-0.3 mm range and delivers much lower radiation dose to bone marrow compared to ⁹⁰Y. In addition, due to longer physical half-life (compared to ⁹⁰Y), the tumor residence times are higher. As a result, higher activities (more mCi amounts) of ¹⁷⁷Lu labeled agents can be administered with comparatively less radiation dose to marrow. There have been several clinical studies investigating the use of ¹⁷⁷ Lu labeled antibodies in the treatment of various cancers. (Mulligan T et al. (1995) Clin Cancer Res. 1: 1447-1454; Meredith R F, et al. (1996) J Nucl Med 37:1491-1496; Alvarez R D, et al. (1997) Gynecologic Oncology 65: 94-101).

The anti-PAPP-A ligands can be used directly in vivo to eliminate antigen-expressing cells via natural complement-dependent cytotoxicity (CDC) or antibody-dependent cellular cytotoxicity (ADCC). The protein ligands of the invention, can include complement binding effector domain, such as the Fc portions from IgG1, −2, or −3 or corresponding portions of IgM which bind complement. In one embodiment, a population of target cells is ex vivo treated with a binding agent of the invention and appropriate effector cells. The treatment can be supplemented by the addition of complement or serum containing complement. Further, phagocytosis of target cells coated with a protein ligand of the invention can be improved by binding of complement proteins. In another embodiment target, cells coated with the protein ligand that includes a complement binding effector domain are lysed by complement.

Also encompassed by the present invention is a method of killing or ablating, e.g,. a cancer cell, which involves using the anti-PAPP-A ligand for prophylaxis. For example, these materials can be used to prevent or delay development or progression of cancers.

Use of the therapeutic methods of the present invention to treat cancers has a number of benefits. Since the protein ligands specifically recognize PAPP-A, other tissue is spared and high levels of the agent are delivered directly to the site where therapy is required. Treatment in accordance with the present invention can be effectively monitored with clinical parameters. Alternatively, these parameters can be used to indicate when such treatment should be employed.

Anti-PAPP-A ligands, e.g., ligands described herein, can be administered in combination with one or more of the existing modalities for treating cancers, including, but not limited to: surgery; radiation therapy, and chemotherapy.

Anti-PAPP-A ligands, e.g., ligands described herein, can be administered, to a patient who has experienced a cardiovascular event or cardiovascular disease or disorder, e.g., an acute coronary syndrome, e.g., a myocardial infarction or angina, e.g., stable or unstable angina). The ligand can be administered, e.g., before, during, or after, a cardiovascular event, e.g., a myocardial infarction, or angina, e.g., within 2, 4, 6, 10, 12, or 24 hours after such an event. In one embodiment, the ligand is conjugate to an agent which alters a property of a plaque, e.g., an enzyme, etc. which can modify, reduce, or destroy a plaque.

Diagnostic Uses

Protein ligands that bind to PAPP-A (e.g., ligands identified by a method described herein and/or described herein) have in vitro and in vivo diagnostic, therapeutic and prophylactic utilities.

In one aspect, the present invention provides a diagnostic method for detecting the presence of a PAPP-A, in vitro (e.g., a biological sample, such as tissue, biopsy, e.g., a cancerous tissue) or in vivo (e.g., in vivo imaging in a subject).

The method includes: (i) contacting a sample with anti-PAPP-A ligand; and (ii) detecting formation of a complex between the anti-PAPP-A ligand and the sample. The method can also include contacting a reference sample (e.g., a control sample) with the ligand, and determining the extent of formation of the complex between the ligand an the sample relative to the same for the reference sample. A change, e.g., a statistically significant change, in the formation of the complex in the sample or subject relative to the control sample or subject can be indicative of the presence of PAPP-A in the sample.

Another method includes: (i) administering the anti-PAPP-A ligand to a subject; and (iii) detecting formation of a complex between the anti-PAPP-A ligand, and the subject. The detecting can include determining location or time of formation of the complex.

The anti-PAPP-A ligand can be directly or indirectly labeled with a detectable substance to facilitate detection of the bound or unbound antibody. Suitable detectable substances include various enzymes, prosthetic groups, fluorescent materials, luminescent materials and radioactive materials.

Complex formation between the anti-PAPP-A ligand and PAPP-A can be detected by measuring or visualizing either the ligand bound to the PAPP-A or unbound ligand. Conventional detection assays can be used, e.g., an enzyme-linked immunosorbent assays (ELISA), a radioimmunoassay (RIA) or tissue immunohistochemistry. Further to labeling the anti-PAPP-A ligand, the presence of PAPP-A can be assayed in a sample by a competition immunoassay utilizing standards labeled with a detectable substance and an unlabeled anti-PAPP-A ligand. In one example of this assay, the biological sample, the labeled standards and the PAPP-A binding agent are combined and the amount of labeled standard bound to the unlabeled ligand is determined. The amount of PAPP-A in the sample is inversely proportional to the amount of labeled standard bound to the PAPP-A binding agent.

Fluorophore and chromophore labeled protein ligands can be prepared. Since antibodies and other proteins absorb light having wavelengths up to about 310 nm, the fluorescent moieties should be selected to have substantial absorption at wavelengths above 310 nm and preferably above 400 nm. A variety of suitable fluorescers and chromophores are described by Stryer (1968) Science, 162:526 and Brand, L. et al. (1972) Annual Review of Biochemistry, 41:843-868. The protein ligands can be labeled with fluorescent chromophore groups by conventional procedures such as those disclosed in U.S. Pat. Nos. 3,940,475, 4,289,747, and 4,376,110. One group of fluorescers having a number of the desirable properties described above is the xanthene dyes, which include the fluoresceins and rhodamines. Another group of fluorescent compounds are the naphthylamines. Once labeled with a fluorophore or chromophore, the protein ligand can be used to detect the presence or localization of the PAPP-A in a sample, e.g., using fluorescent microscopy (such as confocal or deconvolution microscopy).

Histological Analysis. Immunohistochemistry can be performed using the protein ligands described herein. For example, in the case of an antibody, the antibody can synthesized with a label (such as a purification or epitope tag), or can be detectably labeled, e.g., by conjugating a label or label-binding group. For example, a chelator can be attached to the antibody. The antibody is then contacted to a histological preparation, e.g., a fixed section of tissue that is on a microscope slide. After an incubation to allow binding, the preparation is washed to remove unbound antibody. The preparation is then analyzed, e.g., using microscopy, to identify if the antibody bound to the preparation.

Of course, the antibody (or other polypeptide or peptide) can be unlabeled at the time of binding. After binding and washing, the antibody is labeled in order to render it detectable.

Protein Arrays. The anti-PAPP-A ligand can also be immobilized on a protein array. The protein array can be used as a diagnostic tool, e.g., to screen medical samples (such as isolated cells, blood, sera, biopsies, and the like). Of course, the protein array can also include other ligands, e.g., other ligands that bind to the PAPP-A or to other target molecules, e.g., a cancer-associated antigen, a cardiovascular disease-associated protein, and so forth. Methods of producing polypeptide arrays are described, e.g., in De Wildt et al. (2000) Nat. Biotechnol. 18:989-994; Lueking et al. (1999) Anal. Biochem. 270:103-111; Ge (2000) Nucleic Acids Res. 28, e3, I-VII; MacBeath and Schreiber (2000) Science 289:1760-1763; WO 01/40803 and WO 99/51773A1. Polypeptides for the array can be spotted at high speed, e.g., using commercially available robotic apparati, e.g., from Genetic MicroSystems or BioRobotics. The array substrate can be, for example, nitrocellulose, plastic, glass, e.g., surface-modified glass. The array can also include a porous matrix, e.g., acrylamide, agarose, or another polymer.

For example, the array can be an array of antibodies, e.g., as described in De Wildt, supra. Cells that produce the protein ligands can be grown on a filter in an arrayed format. Polypeptide production is induced, and the expressed polypeptides are immobilized to the filter at the location of the cell.

A protein array can be contacted with a labeled target to determine the extent of binding of the target to each immobilized polypeptide from the diversity strand library. If the target is unlabeled, a sandwich method can be used, e.g., using a labeled probed, to detect binding of the unlabeled target.

Information about the extent of binding at each address of the array can be stored as a profile, e.g., in a computer database. The protein array can be produced in replicates and used to compare binding profiles, e.g., of a target and a non-target. Thus, protein arrays can be used to identify individual members of the diversity strand library that have desired binding properties with respect to one or more molecules.

In vivo Imaging. In still another embodiment, the invention provides a method for detecting the presence of PAPP-A-expressing cancerous tissues in vivo, detecting cells that have PAPP-A associated with their cell surface, and detecting PAPP-A containing structures, e.g., plaques. The method includes (i) administering to a subject (e.g., a patient having a cancer or neoplastic disorder) an anti-PAPP-A antibody, conjugated to a detectable marker; (ii) exposing the subject to a means for detecting said detectable marker to the PAPP-A-expressing tissues or cells. For example, the subject is imaged, e.g., by NMR or other tomographic means.

Examples of labels useful for diagnostic imaging in accordance with the present invention include radiolabels such as ₁₃₁I, ₁₁₁In, ₁₂₃I, ₉₉ₘTc, ₃₂P, ₁₂₅I, ₃H, ₁₄C, and ₁₈₈Rh, fluorescent labels such as fluorescein and rhodamine, nuclear magnetic resonance active labels, positron emitting isotopes detectable by a positron emission tomography (“PET”) scanner, chemiluminescers such as luciferin, and enzymatic markers such as peroxidase or phosphatase. Short-range radiation emitters, such as isotopes detectable by short-range detector probes can also be employed. The protein ligand can be labeled with such reagents using known techniques. For example, see Wensel and Meares (1983) Radioimmunoimaging and Radioimmunotherapy, Elsevier, N.Y. for techniques relating to the radiolabeling of antibodies and D. Colcher et al. (1986) Meth. Enzymol. 121: 802-816.

A radiolabeled ligand of this invention can also be used for in vitro diagnostic tests. The specific activity of a isotopically-labeled ligand depends upon the half-life, the isotopic purity of the radioactive label, and how the label is incorporated into the antibody.

Procedures for labeling polypeptides with the radioactive isotopes (such as ¹⁴C, ³H, ³⁵S, ¹²⁵I, ³²p, ¹³¹I) are generally known. For example, tritium labeling procedures are described in U.S. Pat. No. 4,302,438. Iodinating, tritium labeling, and ³⁵S labeling procedures, e.g., as adapted for murine monoclonal antibodies, are described, e.g., by Goding, J. W. (Monoclonal antibodies: principles and practice: production and application of monoclonal antibodies in cell biology, biochemistry, and immunology 2nd ed. London; Orlando : Academic Press, 1986. pp 124-126) and the references cited therein. Other procedures for iodinating polypeptides, such as antibodies, are described by Hunter and Greenwood (1962) Nature 144:945, David et al. (1974) Biochemistry 13:1014-1021, and U.S. Pat. Nos. 3,867,517 and 4,376,110. Radiolabeling elements that are useful in imaging include ¹²³I, ¹³¹I, ¹¹¹In, and ^{99m} Tc, for example. Procedures for iodinating antibodies are described by Greenwood, F. et al. (1963) Biochem. J. 89:114-123; Marchalonis, J. (1969) Biochem. J. 113:299-305; and Morrison, M. et al. (1971) Immunochemistry 289-297. Procedures for ^{99m} Tc-labeling are described by Rhodes, B. et al. in Burchiel, S. et al. (eds.), Tumor Imaging: The Radioimmunochemical Detection of Cancer, New York: Masson 111-123 (1982) and the references cited therein. Procedures suitable for ¹¹¹In-labeling antibodies are described by Hnatowich, D. J. et al. (1983) J. Immul. Methods, 65:147-157, Hnatowich, D. et al. (1984) J. Applied Radiation, 35:554-557, and Buckley, R. G. et al. (1984) F.E.B.S. 166:202-204.

In the case of a radiolabeled ligand, the ligand is administered to the patient, is localized to the tumor bearing the antigen with which the ligand reacts, and is detected or “imaged” in vivo using known techniques such as radionuclear scanning using e.g., a gamma camera or emission tomography. See e.g., A. R. Bradwell et al., “Developments in Antibody Imaging”, Monoclonal Antibodies for Cancer Detection and Therapy, R. W. Baldwin et al., (eds.), pp 65-85 (Academic Press 1985). Alternatively, a positron emission transaxial tomography scanner, such as designated Pet VI located at Brookhaven National Laboratory, can be used where the radiolabel emits positrons (e.g., ¹¹C, ¹⁸F, ¹⁵O, and ¹³N).

MRI Contrast Agents. Magnetic Resonance Imaging (MRI) uses NMR to visualize internal features of living subject, and is useful for prognosis, diagnosis, treatment, and surgery. MRI can be used without radioactive tracer compounds for obvious benefit. Some MRI techniques are summarized in EP-A-0 502 814. Generally, the differences related to relaxation time constants T1 and T2 of water protons in different environments is used to generate an image. However, these differences can be insufficient to provide sharp high resolution images.

The differences in these relaxation time constants can be enhanced by contrast agents. Examples of such contrast agents include a number of magnetic agents paramagnetic agents (which primarily alter T1) and ferromagnetic or superparamagnetic (which primarily alter T2 response). Chelates (e.g., EDTA, DTPA and NTA chelates) can be used to attach (and reduce toxicity) of some paramagnetic substances (e.g., . Fe⁺³, Mn⁺², Gd⁺³). Other agents can be in the form of particles, e.g., less than 10 μm to about 10 nM in diameter). Particles can have ferromagnetic, antiferromagnetic or superparamagnetic properties. Particles can include, e.g., magnetite (Fe₃O₄), γ-Fe₂O₃, ferrites, and other magnetic mineral compounds of transition elements. Magnetic particles may include: one or more magnetic crystals with and without nonmagnetic material. The nonmagnetic material can include synthetic or natural polymers (such as sepharose, dextran, dextrin, starch and the like

The anti-PAPP-A ligands can also be labeled with an indicating group containing of the NMR-active ¹⁹F atom, or a plurality of such atoms inasmuch as (i) substantially all of naturally abundant fluorine atoms are the ¹⁹F isotope and, thus, substantially all fluorine-containing compounds are NMR-active; (ii) many chemically active polyfluorinated compounds such as trifluoracetic anhydride are commercially available at relatively low cost, and (iii) many fluorinated compounds have been found medically acceptable for use in humans such as the perfluorinated polyethers utilized to carry oxygen as hemoglobin replacements. After permitting such time for incubation, a whole body MRI is carried out using an apparatus such as one of those described by Pykett (1982) Scientific American, 246:78-88 to locate and image cancerous tissues.

Also within the scope of the invention are kits comprising the protein ligand that binds to PAPP-A and instructions for diagnostic use, e.g., the use of the anti-PAPP-A ligand (e.g., antibody or antigen-binding fragment thereof, or other polypeptide or peptide) to detect PAPP-A, in vitro, e.g., in a sample, e.g., a biopsy or cells from a patient having a cancer, neoplastic disorder, cardiovascular or inflammatory disorder, or in vivo, e.g., by imaging a subject. The kit can further contain a least one additional reagent, such as a label or additional diagnostic agent. For in vivo use the ligand can be formulated as a pharmaceutical composition.

For example, an anti-PAPP-A ligand can be used to localize a PAPP-A-containing structure (e.g., a plaque) in a subject. The ligand can be administered, e.g., before, during, or after, a cardiovascular event, e.g., a myocardial infarction, or angina, e.g., within 2, 4, 6, 10, 12, or 24 hours after such an event.

### EXAMPLES

The following are non-limiting examples of PAPP-A ligands:

| | |
|---|---|
| AB a01 | |
| Light Chain amino acid sequence: | |
| QSVLTQPPSASGTPGQRVTISCSGSSSNIESNTV | (SEQ ID NO:78) |
| TWYQQLPGTAPKLLIYSDDQRPSGVPDRFSGSKS | |
| GTSASLAISGLQSEDEADYYCATWDNTLRGVVFG | |
| GGTKLTVL | |
| Heavy Chain amino acid sequence: | |
| EVQLLESGGGLVQPGGSLRLSCAASGFTFSPYRM | (SEQ ID NO:79) |
| DWVRQAPGKGLEWVSYIYPSGGFTPYADSVKGRF | |
| TISRDNSKNTFYLQMNSLRAEDTAVYYCAKGSTG | |
| YRYYYGMDVWGQGTTVTVSSASTKGPSVFP | |

| | | |
|---|---|---|
| LC CDR1: | SGSSSNIESNTVT | (SEQ ID NO:80) |
| LC CDR2: | SDDQRPS | (SEQ ID NO:81) |
| LC CDR3: | ATWDNTLRGVV | (SEQ ID NO:82) |
| HC CDR1: | PYRMD | (SEQ ID NO:83) |
| HC CDR2: | YIYPSGGFTPYADSVKG | (SEQ ID NO:84) |
| HC CDR3: | GSTGYRYYYGMDV | (SEQ ID NO:85) |

| | |
|---|---|
| AB a02 | |
| Light Chain amino acid sequence: | |
| QDIVMTQTPPSLPVNPGEPASISCKSSQSLLQSN | (SEQ ID NO:86) |
| GYNYLDWYLQKPGQSPQLLIYLGSNRASGVPDRF | |
| SGSGSGTDFTLKISRVEAEDVGIYYCMQALHTPP | |
| FGQGTRLEIKRTVAAPSV | |
| Heavy Chain amino acid sequence: | |
| EVQLLESGGGLVQPGGSLRLSCAASGFTFSWYWM | (SEQ ID NO:87) |
| NWVRQAPGKGLEWVSSIYSSGGYTSYADSVKGRF | |
| TISRDNSKNTLYLQMNSLRAEDTAVYYCARVRDI | |
| LTGPYYFDYWGQGTLVTVSSASTKGPSVFP | |

| | | |
|---|---|---|
| LC CDR1: | KSSQSLLQSNGYNYLD | (SEQ ID NO:88) |
| LC CDR2: | LGSNRAS | (SEQ ID NO:89) |
| LC CDR3: | MQALHTPP | (SEQ ID NO:90) |
| HC CDR1: | WYWMN | (SEQ ID NO:91) |
| HC CDR2: | SIYSSGGYTSYADSVKG | (SEQ ID NO:92) |
| HC CDR3: | VTRDILTGPYYFDY | (SEQ ID NO:93) |

| | |
|---|---|
| AB a03 | |
| Light Chain amino acid sequence: | |
| QDIQMTQSPSSLSASVGDRVTITCRASQGIRHYL | (SEQ ID NO:94) |
| GWYQQKPGKAPKRLIYAASSLQFGVPARFSGSGS | |
| GTEFTLTISSLQPEDFATYYCLQHNSFPPAFGQG | |
| TKVEIKRTVAAPSV | |
| Heavy Chain amino acid sequence: | |
| EVQLLESGGGLVQPGGSLRLSCAASGFTFSPYDM | (SEQ ID NO:95) |
| WWVRQAPGKGLEWVSYISSSGGKTMYADSVKGRF | |
| TISRDNSKISITLYLQMNSLRAEDTAVYYCARLG | |
| GNSHYYYGMDVWGQGTTVTVSSASTKGPSVFP | |

| | | |
|---|---|---|
| LC CDR1: | RASQGIRHYLG | (SEQ ID NO:96) |
| LC CDR2: | AASSLQF | (SEQ ID NO:97) |
| LC CDR3: | LQHNSFPPA (SEQ ID NO:98) | |
| HC CDR1: | PYDMW | (SEQ ID NO:99) |
| HC CDR2: | YISSSGGKTMYADSVKG | (SEQ ID NO:100) |
| HC CDR3: | LGGNSHYYYGMDV | (SEQ ID NO:101) |

| | |
|---|---|
| AB a04 | |
| Light Chain amino acid sequence: | |
| QDIQMTQSPSSVSASVGDRIAITCRASQGISTWL | (SEQ ID NO:102) |
| AWYQQRPGRAPKLLIYAASTLQSGVPSRFSGSGS | |
| GTDFTLTISSLQPEDFATYFCQQADSFPLTFGQG | |
| TKLEIKRTVAAPSV | |
| Heavy Chain amino acid sequence: | |
| EVQLLESGGGLVQPGGSLRLSCAASGFTFSNYAM | (SEQ ID NO:103) |
| DWVRQAPGKGLEWVSYISPSGGYTRYADSVKGRF | |
| TISRDNSKNTLYLQMNSLRAEDTAVYYCASDFGS | |
| WGQGTLVTVSSASTKGPSVFP | |

| | | |
|---|---|---|
| LC CDR1: | RASQGISTWLA | (SEQ ID NO:104) |
| LC CDR2: | AASTLQS | (SEQ ID NO:105) |
| LC CDR3: | QQADSFPLT | (SEQ ID NO:106) |
| HC CDR1: | NYAMD | (SEQ ID NO:107) |
| HC CDR2: | YISPSGGYTRYADSVKG | (SEQ ID NO:108) |
| HC CDR3: | DFGS | (SEQ ID NO:109) |

| | |
|---|---|
| AB a05 | |
| Light Chain amino acid sequence: | |
| QDIQMTQSPGTLSLSPGERATLSCRASQSISSSY | (SEQ ID NO:110) |
| LAWYQQKPGQAPRLLIYAAASRATGIPDRFSGIG | |
| SGTDFTLTISSLEPEDFAVYYCQQRSNWPLTFGG | |
| GTKVEIKRTVAAPSV | |
| Heavy Chain amino acid sequence: | |
| EVQLLESGGGLVQPGGSLRLSCAASGFTFSRYHM | (SEQ ID NO:111) |
| EWVRQAHGKGLEWVSYISPSGGKTLYADSVKGRF | |
| TISRDNSKNTLYLQMNSLRAEDTAVYYCARHLGY | |
| GSGSYFDYWGQGTLVTVSSASTKGPSVFP | |

| | | |
|---|---|---|
| LC CDR1: | RASQSISSSYLA | (SEQ ID NO:112) |
| LC CDR2: | AAASRAT | (SEQ ID NO:113) |
| LC CDR3: | QQRSNWPLT | (SEQ ID NO:114) |
| HC CDR1: | RYHME | (SEQ ID NO:115) |
| HC CDR2: | YISPSGGKTLYADSVKG | (SEQ ID NO:116) |
| HC CDR3: | HLGYGSGSYFDY | (SEQ ID NO:117) |

| | |
|---|---|
| AB a06 | |
| Light Chain amino acid sequence: | |
| QYELTQPPSVSVSPGQTATIICSGDKLGDKYVAW | (SEQ ID NO:118) |
| YQQKPGQSPVLVVYEDNKRPSGIPERISGSNSGN | |
| TATLTISGTQAMDDADYYCQAWDRSTDHYVFGTG | |
| TKVTVLGQPKANPT | |
| Heavy Chain amino acid sequence: | |
| EVQLLESGGGLVQPGGSLRLSCAASGFTFSNYRM | (SEQ ID NO:119) |
| PWVRQAPGKGLEWVSYIYSSGGITQYADSVKGRF | |
| TISRDNSKNTLYLQMNSLRAEDTAVYYCARSRSY | |
| YGSGSSRYWGQGTLVTVSSASTKGPSVFP | |

| | | |
|---|---|---|
| LC CDR1: | SGDKLGDKYVA | (SEQ ID NO:120) |
| LC CDR2: | EDNKRPS | (SEQ ID NO:121) |
| LC CDR3: | QAWDRSTDHYV | (SEQ ID NO:122) |
| HC CDR1: | NYRMP | (SEQ ID NO:123) |
| HC CDR2: | YIYSSGGITQYADSVKG | (SEQ ID NO:124) |
| HC CDR3: | SRSYYGSGSSRY | (SEQ ID NO:125) |

| | |
|---|---|
| AB b01 | |
| Light Chain amino acid sequence: | |
| QDIQMTQSPSSFSASTGDRVTITCRASQGISSYL | (SEQ ID NO:126) |
| AWYQQKPGKAPKLLIYAASTLQSGVPSKFSGSGS | |
| GTDFTLTISSLQPEDFATYYCQQYNSYPLTFGQG | |
| TRLEIK | |
| Heavy Chain amino acid sequence: | |
| EVQLLESGGGLVQPGGSLRLSCAASGFTFSWYTM | (SEQ ID NO:127) |
| VWVRQAPGKGLEWVSSIYSSGGFTWYADSVKGRF | |
| TISRDNSKNTLYLQMNSLRAEDTAVYYCASDFGS | |
| WGQGTLVTVSSASTKGPSVFP | |

| | | |
|---|---|---|
| LC CDR1: | RASQGISSYLA | (SEQ ID NO:128) |
| LC CDR2: | AASTLQS | (SEQ ID NO:129) |
| LC CDR3: | QQYNSYPLT | (SEQ ID NO:130) |
| HC CDR1: | WYTMV | (SEQ ID NO:131) |
| HC CDR2: | SIYSSGGFTWYADSVKG | (SEQ ID NO:132) |
| HC CDR3: | DFGS | (SEQ ID NO:133) |

| | |
|---|---|
| AB b03 | |
| Light Chain amino acid sequence: | |
| QDIQMTQSPSSLYASVGDRVTITCRASQGIRNEL | (SEQ ID NO:134) |
| GWYQQKPGKAPQRLIYDASTLQSGVPSRFSGGGS | |
| RTEFTLTISSLEPHDFGTYYCQQYASYPLTFGGG | |
| TKVEIKRTVAAPSV | |
| Heavy Chain amino acid sequence: | |
| EVQLLESGGGLVQPGGSLRLSCAASGFTFSDYKM | (SEQ ID NO:135) |
| PWVRQAPGKGLEWVSSIWSSGGTTEYADSVKGRF | |
| TISRDNSKNTLYLQMNSLRAEDTAVYYCAREEIG | |
| RYFDWFLGNYYYYGMDVWGQGTTVTVSSASTKGP | |
| SVFP | |

| | | |
|---|---|---|
| LC CDR1: | RASQGIRNELG | (SEQ ID NO:136) |
| LC CDR2: | DASTLQS | (SEQ ID NO:137) |
| LC CDR3: | QQYASYPLT | (SEQ ID NO:138) |
| HC CDR1: | DYKMP | (SEQ ID NO:139) |
| HC CDR2: | SIWSSGGTTEYADSVKG | (SEQ ID NO:140) |
| HC CDR3: | EEIGRYFDWFLGNYYYYGMDV | (SEQ ID NO:141) |

| | |
|---|---|
| AB b04 | |
| Light Chain amino acid sequence: | |
| QSALTQPPSASGTPGQRVTISCSGSSSNIGSNFV | (SEQ ID NO:142) |
| YWYHHLPGTAPKLLIYRNNQRPSGVPDRFSGSKS | |
| GTSASLAISGLRSEDEADYYCAAWDDSLSGVVFG | |
| GGTKLTVLGQPKAAPS | |
| Heavy Chain amino acid sequence: | |
| EVQLLESGGGLVQPGGSLRLSCAASGFTFSQYKM | (SEQ ID NO:143) |
| NWVRQAPGKGLEWVSYISPSGGYTAYADSVKGRF | |
| TISRDNSKNTLYLQMNSLRAEDTAVYYCARDVVA | |
| GPFDYWGQGTLVTVSSASTKGPSVFP | |

| | | |
|---|---|---|
| LC CDR1: | SGSSSNIGSNFVY | (SEQ ID NO:144) |
| LC CDR2: | RNNQRPS | (SEQ ID NO:145) |
| LC CDR3: | AAWDDSLSGVV | (SEQ ID NO:146) |
| HC CDR1: | QYKMN | (SEQ ID NO:147) |
| HC CDR2: | YISPSGGYTAYADSVKG | (SEQ ID NO:148) |
| HC CDR3: | DVVAGPFDY | (SEQ ID NO:149) |

| | |
|---|---|
| AB b05 | |
| Light Chain amino acid sequence: | |
| QDIQMTQSPSSLSASVGDRVTITCRASQDISNYL | (SEQ ID NO:150) |
| AWFQQKPGRAPKSLIYGASSLQTGVPSKFSGSGS | |
| GTEFTLTISGLQPEDVATYYCHQYNHYPPTFGGG | |
| TKVEIKRTVAAPSV | |
| Heavy Chain amino acid sequence: | |
| EVQLLESGGGLVQPGGSLRLSCAASGFTFSKYPM | (SEQ ID NO:151) |
| FWVRQAPGKGLEWVSWISPSGGKTVYADSVKGRF | |
| TISRDNSKNTLYLQMNSLRAEDTAVYYCAKDCRG | |
| GCSGGSWGQGTLVTVSSASTKGPSVFP | |

| | | |
|---|---|---|
| LC CDR1: | RASQDISNYLA | (SEQ ID NO:152) |
| LC CDR2: | GASSLQT | (SEQ ID NO:153) |
| LC CDR3: | HQYNHYPPT | (SEQ ID NO:154) |
| HC CDR1: | KYPMF | (SEQ ID NO:155) |
| HC CDR2: | WISPSGGKTVYADSVKG | (SEQ ID NO:156) |
| HC CDR3: | DCRGGCSGGS | (SEQ ID NO:157) |

| | |
|---|---|
| AB c01 | |
| Light Chain amino acid sequence: | |
| QDIQMTQSPATLSVSPGERATLSCRASQDVNRYL | (SEQ ID NO:158) |
| AWYQQKPGQPPRLLIYGASTRATGIPARISGSGS | |
| GTEFTLTISSLQSEDFAVYYCQQYHNWPLTFGGG | |
| TKVEIKRTVAAPSV | |
| Heavy Chain amino acid sequence: | |
| EVQLLESGGGLVQPGGSLRLSCAASGFTFSRYSM | (SEQ ID NO:159) |
| NWVRQAPGKGLEWVSYISPSGGMTKYADSVKGRF | |
| TISRDNSKNTLYLQMNSLRAEDTAVYYCANTLGY | |
| WGQGTLVTVSSASTKGPSVFP | |

| | | |
|---|---|---|
| LC CDR1: | RASQDVNRYLA | (SEQ ID NO:160) |
| LC CDR2: | GASTRAT | (SEQ ID NO:161) |
| LC CDR3: | QQYHNWPLT | (SEQ ID NO:162) |
| HC CDR1: | RYSMN | (SEQ ID NO:163) |
| HC CDR2: | YISPSGGMTKYADSVKG | (SEQ ID NO:164) |
| HC CDR3: | TLGY | (SEQ ID NO:165) |

| | |
|---|---|
| AB c02 | |
| Light Chain amino acid sequence: | |
| QSALTQPASVSGSPGQSITISCTGTSSDVGYYDY | (SEQ ID NO:166) |
| VSWYQHHPGKAPKLIIYDVTSRPSGVSSHFSGSK | |
| SGNTASLTISGLQADDEADYYCSSYTSGSTRYVF | |
| GPGTKVTVLGQPKANPT | |
| Heavy Chain amino acid sequence: | |
| EVQLLESGGGLVQPGGSLRLSCAASGFTFSDYYM | (SEQ ID NO:167) |
| RWVRQAPGKGLEWVSRIYPSGGHTWYADSVKGRF | |
| TISRDNSKNTLYLQMNSLRAEDTAVYYCARHRAG | |
| SSGWYSDYWGQGTLVTVSSASTKGPSVFP | |

| | | |
|---|---|---|
| LC CDR1: | TGTSSDVGYYDYVS | (SEQ ID NO:168) |
| LC CDR2: | DVTSRPS | (SEQ ID NO:169) |
| LC CDR3: | SSYTSGSTRYV | (SEQ ID NO:170) |
| HC CDR1: | DYYMR | (SEQ ID NO:171) |
| HC CDR2: | RIYPSGGHTWYADSVKG | (SEQ ID NO:172) |
| HC CDR3: | HRAGSSGWYSDY | (SEQ ID NO:173) |

| | |
|---|---|
| AB c04 | |
| Light Chain amino acid sequence: | |
| QDIQMTQSPSSLSASVGDRVTITCRASQDIRNYL | (SEQ ID NO:174) |
| AWFQQKPGEAPKSLIYAASSLQSGVSSNFSGSGS | |
| GTDFTLTISSLQPEDFATYYCQQYHRYPRTFGQG | |
| TKLEIKRTVAAPSV | |
| Heavy Chain amino acid sequence: | |
| EVQLLESGGGLVQPGGSLRLSCAASGFTFSAYNM | (SEQ ID NO:175) |
| PWVRQAPGKGLEWVSYISSSGTGYADSVKGRFTI | |
| SRDNSKNTLYLQMNSLRAEDTAVYYCARELGSGS | |
| YYPGYFQHWGQGTLVTVSSASTKGPSVFP | |

| | | |
|---|---|---|
| LC CDR1: | RASQDIRNYLA | (SEQ ID NO:176) |
| LC CDR2: | AASSLQS | (SEQ ID NO:177) |
| LC CDR3: | QQYHRYPRT | (SEQ ID NO:178) |
| HC CDR1: | AYNMP | (SEQ ID NO:179) |
| HC CDR2: | YISSSGTGYADSVKGR | (SEQ ID NO:180) |
| HC CDR3: | ELGSGSYYPGYFQH | (SEQ ID NO:181) |

| | |
|---|---|
| AB c05 | |
| Light Chain amino acid sequence: | |
| QDIQMTQSPATLYVSPGERATLSCRASQSVSRNL | (SEQ ID NO:182) |
| AWYQQKPGQAPRLLIYGASTRATGIPARFSGSGS | |
| RTEFTLTISSLQSEDFAVYHCQQYNSRPLTFGGG | |
| TKVEIKRTVAAPSV | |
| Heavy Chain amino acid sequence: | |
| EVQLLESGGGLVQPGGSLRLSCAASGFTFSWYFM | (SEQ ID NO:183) |
| NWVRQAPGKGLEWVSSIYPSGGYTMYADSVKGRF | |
| TISRDNSKKTLYLQMNSLRAEDTAVYYCASDFGS | |
| WGQGTLVTVSSASTKGPSVFP | |

| | | |
|---|---|---|
| LC CDR1: | RASQSVSRNLA | (SEQ ID NO:184) |
| LC CDR2: | GASTRAT | (SEQ ID NO:185) |
| LC CDR3: | QQYNSRPLT | (SEQ ID NO:186) |
| HC CDR1: | WYFMN | (SEQ ID NO:187) |
| HC CDR2: | SIYPSGGYTMYADSVKG | (SEQ ID NO:188) |
| HC CDR3: | DFGS | (SEQ ID NO:189) |

| | |
|---|---|
| AB c06 | |
| Light Chain amino acid sequence: | |
| QSALTQPASVSGSPGQSITISCTGTSSDVGYYDY | (SEQ ID NO:190) |
| VSWYQHHPGKAPKLIIYDVTSRPSGVSSHFSGSK | |
| SGNTASLTISGLQADDEADYYCSSYTSGSTRYVF | |
| GPGTKVTVLGQPKANPT | |
| Heavy Chain amino acid sequence: | |
| EVQLLESGGGLVQPGGSLRLSCAASGFTFSDYYM | (SEQ ID NO:191) |
| RWVRQAPGKGLEWVSRIYPSGGHTWYADSVKGRF | |
| TISRDNSKNTLYLQMNSLRAEDTAVYYCARHRAG | |
| SSGWYSDYWGQGTLVTVSSASTKGPSVFP | |

| | | |
|---|---|---|
| LC CDR1: | TGTSSDVGYYDYVS | (SEQ ID NO:192) |
| LC CDR2: | DVTSRPS | (SEQ ID NO:193) |
| LC CDR3: | SSYTSGSTRYV | (SEQ ID NO:194) |
| HC CDR1: | DYYMR | (SEQ ID NO:195) |
| HC CDR2: | RIYPSGGHTWYADSVKG | (SEQ ID NO:196) |
| HC CDR3: | HRAGSSGWYSDY | (SEQ ID NO:197) |

| | |
|---|---|
| AB d02 | |
| Light Chain amino acid sequence: | |
| QDIQMTQSPSSLSASVGDRVTITCRASQSISSYL | (SEQ ID NO:198) |
| NWYQQKPGKAPKLLIYAASSLQSGVPSRFSGSGS | |
| GTDFTLTISSLQPEDFATYYCQQSYSTRWTFGQG | |
| TKVEIKRTVAAPSV | |
| Heavy Chain amino acid sequence: | |
| EVQLLESGGGLVQPGGSLRLSCAASGFTFSTYFM | (SEQ ID NO:199) |
| RWVRQAPGKGLEWVSYIVPSGGNTLYADSVKGRF | |
| TISRDNSKNTLYLQMNSLRAEDTAVYYCAREEWD | |
| VLLWFGELSAAFDIWGQGTMVTVSSASTKGPSVF | |
| P | |

| | | |
|---|---|---|
| LC CDR1: | RASQSISSYLN | (SEQ ID NO:200) |
| LC CDR2: | AASSLQS | (SEQ ID NO:201) |
| LC CDR3: | QQSYSTRWT | (SEQ ID NO:202) |
| HC CDR1: | TYFMR | (SEQ ID NO:203) |
| HC CDR2: | YIVPSGGNTLYADSVKG | (SEQ ID NO:204) |
| HC CDR3: | EEWDVLLWFGELSAAFDI | (SEQ ID NO:205) |

| | |
|---|---|
| AB d03 | |
| Light Chain amino acid sequence: | |
| QDIQMTQSPSSLSASVGDRVTITCRASQGIRHYL | (SEQ ID NO:206) |
| GWYQQKPGKAPKRLIYAASSLQFGVPARFSGSGS | |
| GTEFTLTISSLQPEDFATYYCLQHNSFPPAFGQG | |
| TKVEIKRTVAAPSV | |
| Heavy Chain amino acid sequence: | |
| EVQLLESGGGLVQPGGSLRLSCAASGFTFSPYDM | (SEQ ID NO:207) |
| WWVRQAPGKGLEWVSYISSSGGKTMYADSVKGRF | |
| TISRDNSKNTLYLQMNSLRAEDTAVYYCARLGGN | |
| SHYYYGMDVWGQGTTVTVSSASTKGPSVFP | |

| | | |
|---|---|---|
| LC CDR1: | RASQGIRHYLG | (SEQ ID NO:208) |
| LC CDR2: | AASSLQF | (SEQ ID NO:209) |
| LC CDR3: | LQHNSFPPA | (SEQ ID NO:210) |
| HC CDR1: | PYDMW | (SEQ ID NO:211) |
| HC CDR2: | YISSSGGKTMYADSVKG | (SEQ ID NO:212) |
| HC CDR3: | LGGNSHYYYGMDV | (SEQ ID NO:213) |

| | |
|---|---|
| AB d04 | |
| Light Chain amino acid sequence: | |
| QSELTQPPSASATPGQRVTISCSGSSSNIGRNLV | (SEQ ID NO:214) |
| YWYQQLPGTAPKLLIYSNNQRPSGVPDRFSGSKS | |
| GTSASLAISGLRSEEEADYYCAAWDDSLSGWVFG | |
| GGTRLTVLGQPKAAPS | |
| Heavy Chain amino acid sequence: | |
| EVQLLESGGGLVQPGGSLRLSCAASGFTFSWYHM | (SEQ ID NO:215) |
| RWVRQAPGKGLEWVSIYPSGGVTSYADSVKGRFT | |
| ISRDNSKNTLYLQMNSLRAEDTAVYYCARETSGW | |
| YRDRWFDPWGQGTLVTVSSASTKGPSVFP | |

| | | |
|---|---|---|
| LC CDR1: | SGSSSNIGPNLVY | (SEQ ID NO:216) |
| LC CDR2: | SNNQRPS | (SEQ ID NO:217) |
| LC CDR3: | AAWDDSLSGWV | (SEQ ID NO:218) |
| HC CDR1: | WYHMR | (SEQ ID NO:219) |
| HC CDR2: | IYPSGGVTDYADSVKG | (SEQ ID NO:220) |
| HC CDR3: | ETSGWYRDRWFDP | (SEQ ID NO:221) |

| | |
|---|---|
| AB d05 | |
| Light Chain amino acid sequence: | |
| QSVLTQTASVSGSPGQSITISCTGTSSDIGDYEY | (SEQ ID NO:222) |
| VSWYQQHPGKAPKVILYEVSNRPSGVPDRFSGSK | |
| SGNTASLTISGLQAEDEADYYCGSYRKSSTPYVF | |
| GTGTKVSVLGQPKANPT | |
| Heavy Chain amino acid sequence: | |
| EVQLLESGGGLVQPGGSLRLSCAASGFTFSYYHM | (SEQ ID NO:223) |
| WWVRQAPGKGLEWVSVIVPSGGGTQYADSVKGRF | |
| TISRDNSKNTLYLQMNSLRAEDTAVYYCARDGHS | |
| SSWYGGGAHYYGMDVWGQGTTVTVSSASTKGPSV | |
| FP | |

| | | |
|---|---|---|
| LC CDR1: | TGTSSDIGDYEYVS | (SEQ ID NO:224) |
| LC CDR2: | YEVSNRPS | (SEQ ID NO:225) |
| LC CDR3: | GSYRKSSTPYV | (SEQ ID NO:226) |
| HC CDR1: | YYHMW | (SEQ ID NO:227) |
| HC CDR2: | VIVPSGGGTQYADSVKG | (SEQ ID NO:228) |
| HC CDR3: | DGHSSSWYGGGAHYYGMDV | (SEQ ID NO:229) |

| | |
|---|---|
| AB d06 | |
| Light Chain amino acid sequence: | |
| QDIQMTQSPATLSLSPGERATLSCRASQSVSSYL | (SEQ ID NO:230) |
| AWYQQKPGQAPRLLIYGASSRATGIPDRFSGSGS | |
| GTDFTLTIGRLEPEDFAVYYCQQYSSSPVTFGQG | |
| TRLEIKRTVAAPSV | |
| Heavy Chain amino acid sequence: | |
| EVQLLESGGGLVQPGGSLRLSCAASGFTFSSYRM | (SEQ ID NO:231) |
| NWVRQAPGKGLEWVSGIVPSGGKTFYADSVKGRF | |
| TISRDNSKNTLYLQMNSLRAEDTAVYYCASDFGS | |
| WGQGTLVTVSSASTKGPSVFP | |

| | | |
|---|---|---|
| LC CDR1: | RASQSVSSYLA | (SEQ ID NO:232) |
| LC CDR2: | GASSRAT | (SEQ ID NO:233) |
| LC CDR3: | QQYSSSPVT | (SEQ ID NO:234) |
| HC CDR1: | SYRMN | (SEQ ID NO:235) |
| HC CDR2: | GIVPSGGKTFYADSVKG | (SEQ ID NO:236) |
| HC CDR3: | DFGS | (SEQ ID NO:237) |

| | |
|---|---|
| AB e01 | |
| Light Chain amino acid sequence: | |
| QDIQMTQSPSSLSASVGDRVTITCRASQRISSYV | (SEQ ID NO:238) |
| NWYQQKPGKAPKLLIYSASSLQSGVPSRFSGSVS | |
| GTEFTLTISSLQPEDFATYYCQQSYRTPPFFGQG | |
| TKLEVKRTVAAPSV | |
| Heavy Chain amino acid sequence: | |
| EVQLLESGGGLVQPGGSLRLSCAASGFTFSLYQM | (SEQ ID NO:239) |
| LWVRQAPGKGLEWVSGIVSSGGLTGYADSVKGRF | |
| TISRDNSKNTLYLQMNSLRAEDTAVYYCARHNRA | |
| IGTFDYWGQGTLVTVSSASTKGPSVFP | |

| | | |
|---|---|---|
| LC CDR1: | RASQRISSYVN | (SEQ ID NO:240) |
| LC CDR2: | SASSLQS | (SEQ ID NO:241) |
| LC CDR3: | QQSYRTPPF | (SEQ ID NO:242) |
| HC CDR1: | LYQML | (SEQ ID NO:243) |
| HC CDR2: | GIVSSGGLTGYADSVKG | (SEQ ID NO:244) |
| HC CDR3: | HNRAIGTFDY | (SEQ ID NO:245) |

| | |
|---|---|
| AB e02 | |
| Light Chain amino acid sequence: | |
| QDIQMTQSPATLSLSPGERATLSCRASQSVSRYL | (SEQ ID NO:246) |
| AWYQQKPGQAPRLLIYGASTRATGIPARFSGSGS | |
| GTEFTLTISSLQSEDFAVYYCQQYNNWPSFGGGT | |
| KVEIKRTVAAPSV | |
| Heavy Chain amino acid sequence: | |
| EVQLLESGGGLVQPGGSLRLSCAASGFTFSNYSM | (SEQ ID NO:247) |
| DWVRQAPGKGLEWVSWISPSGGLTTYADSVKGRF | |
| TISRDNSKNTLYLQMNSLRAEDTAVYYCASDFGS | |
| WGQGTLVTVSSASTKGPSVFP | |

| | | |
|---|---|---|
| LC CDR1: | RASQSVSRYLA | (SEQ ID NO:248) |
| LC CDR2: | GASTRAT | (SEQ ID NO:249) |
| LC CDR3: | QQYNNWPS | (SEQ ID NO:250) |
| HC CDR1: | NYSMD | (SEQ ID NO:251) |
| HC CDR2: | WISPSGGLTTYADSVKG | (SEQ ID NO:252) |
| HC CDR3: | DFGS | (SEQ ID NO:253) |

| | |
|---|---|
| AB e03 | |
| Light Chain amino acid sequence: | |
| QSVLTQPPYASASLGASVTLTCTLSSGYSNYKVD | (SEQ ID NO:254) |
| WYQQRPGKGPQFVMRVGSGGIVGSKGDGIPDRFS | |
| VLGSGLYRYLTIKNTQEEDESDYYCGADHGRGGT | |
| FVWVFGGGTKLTVLGQPKAAPS | |
| Heavy Chain amino acid sequence: | |
| EVQLLESGGGLVQPGGSLRLSCAASGFTFSYKMM | (SEQ ID NO:255) |
| WVRQAPGKGLEWVSYISSSGGITTYADSVKGRFT | |
| ISRDNSKNTLYLQMNSLRAEDTAVYYCARDPTYD | |
| FWSGYYYYYYMDVWGKGTTVTVSSASTKGPSVFP | |

| | | |
|---|---|---|
| LC CDR1: | TLSSGYSNYKVD | (SEQ ID NO:256) |
| LC CDR2: | RVGSGGIVGSKGD | (SEQ ID NO:257) |
| LC CDR3: | GADHGRGGTFVWV | (SEQ ID NO:258) |
| HC CDR1: | SYKMM | (SEQ ID NO:259) |
| HC CDR2: | YISSSGGITTYADSVKG | (SEQ ID NO:260) |
| HC CDR3: | RDPTYDFWSGYYYYYYMDV | (SEQ ID NO:261) |

| | |
|---|---|
| AB f01 | |
| Light Chain amino acid sequence: | |
| QSALTQPSSASGTPGQRVSISCSGSSYNIGVYDV | (SEQ ID NO:262) |
| YWYQQLPGTAPKLLIYTNNQRPSGVPDRFSGSKS | |
| GTSASLAISGLQSEDEADYYCAAWDDSLSGWVFG | |
| GGTKVTVLGQPKAAPS | |
| Heavy Chain amino acid sequence: | |
| EVQLLESGGGLVQPGGSLRLSCAASGFTFSQYNM | (SEQ ID NO:263) |
| PWVRQAPGKGLEWVSSIVPSGGFTAYADSVKGRF | |
| TISRDNSKNTLYLQMNSLRAEDTAVYYCARVDCS | |
| GGSCYRGPQNYFDYWGQGTLVTVSSASTKGPSVF | |
| P | |

| | | |
|---|---|---|
| LC CDR1: | SGSSYNIGVYDVY | (SEQ ID NO:264) |
| LC CDR2: | TNNQRPS | (SEQ ID NO:265) |
| LC CDR3: | AAWDDSLSGWV | (SEQ ID NO:266) |
| HC CDR1: | QYNMP | (SEQ ID NO:267) |
| HC CDR2: | SIVPSGGFTAYADSVKG | (SEQ ID NO:268) |
| HC CDR3: | VDCSGGSCYRGPQNYFDY | (SEQ ID NO:269) |

| | |
|---|---|
| AB f03 | |
| Light Chain amino acid sequence: | |
| QYELTQPASVSGSPGQSITISCTGTSSDVGGYNY | (SEQ ID NO:270) |
| VSWYQQHPGKAPKLMIYEVSNRPSGVSNRFSGSK | |
| SDNTASLTISGLQAEDEADYYCGSYRKSSTPYVF | |
| GTGTKVSVLGQPKANPT | |
| Heavy Chain amino acid sequence: | |
| EVQLLESGGGLVQPGGSLRLSCAASGFTFSQYMM | (SEQ ID NO:271) |
| TWVRQAPGKGLEWVSYIGSSGGQTKYADSVKGRF | |
| TISRDNSKNTLYLQMNSLRAEDTAVYYCARDPGV | |
| AVAGYYYYGMDVWGQGTTVTVSSASTKGPSVFP | |

| | | |
|---|---|---|
| LC CDR1: | TGTSSDVGGYNYVS | (SEQ ID NO:272) |
| LC CDR2: | EVSNRPS | (SEQ ID NO:273) |
| LC CDR3: | GSYRKSSTPYV | (SEQ ID NO:274) |
| HC CDR1: | QYMMT | (SEQ ID NO:275) |
| HC CDR2: | YIGSSGGQTKYADSVKG | (SEQ ID NO:276) |
| HC CDR3: | DPGVAVAGYYYYGMDV | (SEQ ID NO:277) |

| | |
|---|---|
| AB f05 | |
| Light Chain amino acid sequence: | |
| QDIQMTQSPSSVSASVGDRVTITCRASRGISRWL | (SEQ ID NO:278) |
| AWYQQKPGKAPKLLIYGASTLQKGVPSRFTGSGS | |
| GTDFTLTITSLQPEDFATYYCQQGNSFPFTFGPG | |
| TKVDIKRTVAAPSV | |
| Heavy Chain amino acid sequence: | |
| EVQLLESGGGLVQPGGSLRLSCAASGFTFSGYWM | (SEQ ID NO:279) |
| SWVRQAPGKGLEWVSVIRPSGGKTGYADSVKGRF | |
| TISRDNFKNTLYLQMNSLRAEDTAVYYCARVRAP | |
| GYYYYGMDVWGQGTTVTVSSASTKGPSVFP | |

| | | |
|---|---|---|
| LC CDR1: | RASRGISRWLA | (SEQ ID NO:280) |
| LC CDR2: | GASTLQK | (SEQ ID NO:281) |
| LC CDR3: | QQGNSFPFT | (SEQ ID NO:282) |
| HC CDR1: | GYWMS | (SEQ ID NO:283) |
| HC CDR2: | VIRPSGGKTGYADSVKG | (SEQ ID NO:284) |
| HC CDR3: | VRAPGYYYYGMDV | (SEQ ID NO:285) |

| | |
|---|---|
| AB f06 | |
| Light Chain amino acid sequence: | |
| QSVLTQTASVSGSPGQSITISCTGTSSDIGDYEY | (SEQ ID NO:286) |
| VSWYQQHPGKAPKVILYEVSNRPSGVPDRFSGSK | |
| SGNTASLTISGLQAEDEADYYCGSYRKSSTPYVF | |
| GTGTKVSVLGQPKANPT | |
| Heavy Chain amino acid sequence: | |
| EVQLLESGGGLVQPGGSLRLSCAASGFTFSYYHM | (SEQ ID NO:287) |
| WWVRQAPGKGLEWVSVIVPSGGGTQYADSVKGRF | |
| TISRDNSKNTLYLQMNSLRAEDTAVYYCARDGHS | |
| SSWYGGGAHYYGMDVWGQGTTVTVSSASTKGPSV | |
| FP | |

| | | |
|---|---|---|
| LC CDR1: | TGTSSDIGDYEYVS | (SEQ ID NO:288) |
| LC CDR2: | YEVSNRPS | (SEQ ID NO:289) |
| LC CDR3: | GSYRKSSTPYV | (SEQ ID NO:290) |
| HC CDR1: | YYHMW | (SEQ ID NO:291) |
| HC CDR2: | VIVPSGGGTQYADSVKG | (SEQ ID NO:292) |
| HC CDR3: | DGHSSSWYGGGAHYYGMDV | (SEQ ID NO:293) |

| | |
|---|---|
| AB g01 | |
| Light Chain amino acid sequence: | |
| QDIQMTQSPSSLSASVGDRVTITCRASQGIRNDL | (SEQ ID NO:294) |
| GWFQQKPGKAPRRLIWGASTLQSGVPSRFSGSGS | |
| GTDFTLTISSLQPEDFATYYCLQDYNYPYTFGQG | |
| TKLEIKRTVAAPSV | |
| Heavy Chain amino acid sequence: | |
| EVQLLESGGGLVQPGGSLRLSCAASGFTFSFYGM | (SEQ ID NO:295) |
| PWVRQAPGKGLEWVSGIYPSGGVTRYADSVKGRF | |
| TISRDNSKNTLYLQMNSLRAEDTAVYYCAKTYSS | |
| SWYGWYFDYWGQGTLVTVSSASTKGPSVFP | |

| | | |
|---|---|---|
| LC CDR1: | RASQGIRNDLG | (SEQ ID NO:296) |
| LC CDR2: | GASTLQS | (SEQ ID NO:297) |
| LC CDR3: | LQDYNYPYT | (SEQ ID NO:298) |
| HC CDR1: | FYGMP | (SEQ ID NO:299) |
| HC CDR2: | GIYPSGGVTRYADSVKG | (SEQ ID NO:300) |
| HC CDR3: | TYSSSWYGWYFDY | (SEQ ID NO:301) |

| | |
|---|---|
| AB g02 | |
| Light Chain amino acid sequence: | |
| QDIQMTQSPGTLSLSPGERATLSCRASQSVSSSY | (SEQ ID NO:302) |
| LAWYQQKPGQAPRLLIYGASSRATGIPDRFSGSG | |
| SGTDFTLTISRLEPEDFAVYYCQQYGSSPWTFGQ | |
| GTKVEIKRTVAAPSV | |
| Heavy Chain amino acid sequence: | |
| EVQLLESGGGLVQPGGSLRLSCAASGFTFSFYPM | (SEQ ID NO:303) |
| PWVRQAPGKGLEWVSYISPSGGDTTYADSVKGRF | |
| TISRDNSKNTFYLQMNSLRAEDTAVYYCARGGSY | |
| SSSWYGYWGQGTLVTVSSASTKGPSVFP | |

| | | |
|---|---|---|
| LC CDR1: | RASQSVSSSYLA | (SEQ ID NO:304) |
| LC CDR2: | GASSRAT | (SEQ ID NO:305) |
| LC CDR3: | QQYGSSPWT | (SEQ ID NO:306) |
| HC CDR1: | FYPMP | (SEQ ID NO:307) |
| HC CDR2: | YISPSGGDTTYADSVKG | (SEQ ID NO:308) |
| HC CDR3: | GGSYSSSWYGY | (SEQ ID NO:309) |

| | |
|---|---|
| AB g03 | |
| Light Chain amino acid sequence: | |
| QDIQMTQSPSSVSASVGDRVTITCRASRGISRWL | (SEQ ID NO:310) |
| AWYQQKPGKAPKLLIYGASTLQKGVPSRFTGSGS | |
| GTDFTLTITSLQPEDFATYYCQQGNSFPFTFGPG | |
| TKVDIKRTVAAPSV | |
| Heavy Chain amino acid sequence: | |
| EVQLLESGGGLVQPGGSLRLSCAASGFTFSGYWM | (SEQ ID NO:311) |
| SWVRQAPGKGLEWVSVIRPSGGKTGYADSVKGRF | |
| TISRDNFKNTLYLQMNSLRAEDTAVYYCARVRAP | |
| GYYYYGMDVWGQGTTVTVSSASTKGPSVFP | |

| | | |
|---|---|---|
| LC CDR1: | RASRGISRWLA | (SEQ ID NO:312) |
| LC CDR2: | GASTLQK | (SEQ ID NO:313) |
| LC CDR3: | QQGNSFPFT | (SEQ ID NO:314) |
| HC CDR1: | GYWMS | (SEQ ID NO:315) |
| HC CDR2: | VIRPSGGKTGYADSVKG | (SEQ ID NO:316) |
| HC CDR3: | VRAPGYYYYGMDV | (SEQ ID NO:317) |

| | |
|---|---|
| AB g04 | |
| Light Chain amino acid sequence: | |
| QSVLTQPASVSGSPGQSITISCTGTSSDVGGYNY | (SEQ ID NO:318) |
| VSWYQRHPGKAPKLIIYDVTNRPSGASRHFSGSK | |
| SGNTASLTISGLQADDEADYYCVSFTNSNTFVFG | |
| SGTRVTVLGQPKANPT | |
| Heavy Chain amino acid sequence: | |
| EVQLLESGGGLVQPGGSLRLSCAASGFTFSLYHM | (SEQ ID NO:319) |
| DWVRQAPGKGLEWVSVIYPSGGGTPYADSVKGRF | |
| TISRDNSKNTLYLQMNSLRAEDTAVYYCARRVGY | |
| CSGGSCYYYYYYMDVWGKGTTVTVSSASTKGPSV | |
| FP | |

| | | |
|---|---|---|
| LC CDR1: | TGTSSDVGGYNYVS | (SEQ ID NO:320) |
| LC CDR2: | DVTNRP | (SEQ ID NO:321) |
| LC CDR3: | VSFTNSNTFV | (SEQ ID NO:322) |
| HC CDR1: | LYHMD | (SEQ ID NO:323) |
| HC CDR2: | VIYPSGGGTPYADSVKG | (SEQ ID NO:324) |
| HC CDR3: | RVGYCSGGSCYYYYYYMDV | (SEQ ID NO:325) |

| | |
|---|---|
| AB g05 | |
| Light Chain amino acid sequence: | |
| QDIQMTQSPATLSVSPGERATLSCRASQSVRSYL | (SEQ ID NO:326) |
| AWYQQKPGQAPRLLIYDASTRATGIPARFSGSGS | |
| GTEFTLTISSLQSEDFAVYYCQQYNNWPPTFGQG | |
| TKVEIKRTVAAPSV | |
| Heavy Chain amino acid sequence: | |
| EVQLLESGGGLVQPGGSLRLSCAASGFTFSWYRM | (SEQ ID NO:327) |
| NWVRQAPGKGLEWVSSIVPSGGYTRYADSVKGRF | |
| TISRDNSKNTLYLQMNSLRAEDTAVYYCASDFGS | |
| WGQGTLVTVSSASTKGPSVFP | |

| | | |
|---|---|---|
| LC CDR1: | RASQSVRSYLA | (SEQ ID NO:328) |
| LC CDR2: | DASTRAT | (SEQ ID NO:329) |
| LC CDR3: | QQYNNWPPT | (SEQ ID NO:330) |
| HC CDR1: | WYRMN | (SEQ ID NO:331) |
| HC CDR2: | SIVPSGGYTRYADSVKG | (SEQ ID NO:332) |
| HC CDR3: | DFGS | (SEQ ID NO:333) |

AB B12
Light Chain amino acid sequence:

| | |
|---|---|
| FYSHSAQSELTQPPSASGTPGQRVTISCSGSSSN | (SEQ ID NO:334) |
| IGSNTVNWYQQLPGTAPKLLIYSNNYRPSGVPDR | |
| FSGSKSGTSASLAISGLQSDDEAEYLCAAWDDSL | |
| NGPVFGGGTKVTVLGQPKAAP | |
| Heavy Chain amino acid sequence: | |
| EVQLLESGGGLVQPGGSLRLSCAASGFTFSSYVM | (SEQ ID NO:335) |

IWVRQAPGKGLEWVSWISSSGGYTSYADSVKGRF
TISRDNSKNTLYLQMNSLRAEDTAVYYCAKGPGT
RGDYWGQGTLVTVSSASTKGPSVFPLAP

| | | |
|---|---|---|
| LC CDR1: | SGSSSNIGSNTVN | (SEQ ID NO:336) |
| LC CDR2: | YSNNYRP | (SEQ ID NO:383) |
| LC CDR3: | AAWDDSLNGPV | (SEQ ID NO:384) |
| HC CDR1: | SYVMI | (SEQ ID NO:337) |
| HC CDR2: | WISSSGGYTSYADSVKG | (SEQ ID NO:338) |
| HC CDR3: | GPGTRGDY | (SEQ ID NO:339) |

| | |
|---|---|
| AB E06 | |
| Light Chain amino acid sequence: | |
| FYSHSAQSVLTQPPSASATPGQRVTFSCSGSSSN | (SEQ ID NO:340) |
| IGSNAVNWYHQLPGTAPKLLIYHNNQRPSGVPDR | |
| FSGSKSGTSASLAISGLQSEDEADYYCAAWDDSL | |
| HGYVFGPGTKVTVLGQPKANP | |
| Heavy Chain amino acid sequence: | |
| EVQLLESGGGLVQPGGSLRLSCAASGFTFSIYPM | (SEQ ID NO:341) |
| NWVRQAPGKGLEWVSGISPSGGYTGYADSVKGRF | |
| TISRDNSKNTLYLQMNSLRAEDTAVYYCARGGIS | |
| WFMDYWGQGTLVTVSSASTKGPSVFPLAP | |

| | | |
|---|---|---|
| LC CDR1: | SGSSSNIGSNAVN | (SEQ ID NO:342) |
| LC CDR2: | HNNQRPS | (SEQ ID NO:343) |
| LC CDR3: | AAWDDSLHGYV | (SEQ ID NO:344) |
| HC CDR1: | IYPMN | (SEQ ID NO:345) |
| HC CDR2: | GISPSGGYTGYADSVKG | (SEQ ID NO:346) |
| HC CDR3: | GGISWFMDY | (SEQ ID NO:347) |

| | |
|---|---|
| AB F05 | |
| Light Chain amino acid sequence: | |
| FYSHSAQSVLTQPRSVSGSPGQSVTTSCTGTSSD | (SEQ ID NO:352) |
| VGASYKFVSWYQLKPGKAPKLMLFNVRERPSGVP | |
| DRFSGSKSGNTASLTISGLQAEDEADYYCCSYAR | |
| GQTFSYVFGGGTTVTVLGQPKANP | |
| Heavy Chain amino acid sequence: | |
| EVQLLESGGGLVQPGGSLRLSCAASGFTFSRYSM | (SEQ ID NO:353) |
| GWVRQAPGKGLEWVSSIRPSGGYTRYADSVKGRF | |
| TISRDNSKNTLYLQMNSLRAEDTAVYYCAKDLEY | |
| SSGWSFDYWGQGTLVTVSSASTKGPSVFPLAP | |

| | | |
|---|---|---|
| LC CDR1: | TGTSSDVGASYKFVS | (SEQ ID NO:385) |
| LC CDR2: | FNVRERPS | (SEQ ID NO:386) |
| LC CDR3: | CSYARGQTFSYV | (SEQ ID NO:354) |
| HC CDR1: | RYSMG | (SEQ ID NO:355) |
| HC CDR2: | SIRPSGGYTRYADSVKG | (SEQ ID NO:356) |
| HC CDR3: | DLEYSSGWSFDY | (SEQ ID NO:357) |

Nucleic Acids encoding Light Chain Variable Domains

| | | |
|---|---|---|
| AB e01 | GTGAAAAAATTATTATTCGCAATTCCTTTAGTTGTTCCTTTCTATTCTCACAGTGCAC | (SEQ ID NO:3) |
| | AAGACATCCAGATGACACAGTCTCCATCCTCCCTGTCTGCATCTGTTGGAGACAGAGT | |
| | CACCATCACTTGCCGGGCTAGTCAGCGCATTAGTAGTTATGTAAATTGGTATCAACAG | |
| | AAACCAGGGAAAGCCCCTAAGCTCCTGATCTATTCTGCATCCAGTTTACAAAGTGGGG | |
| | TCCCATCAAGGTTCAGTGGCAGTGTATCTGGGACAGAGTTCACTCTCACCATCAGCAG | |
| | TCTGCAACCTGAGGATTTTGCAACTTACTACTGTCAACAGAGTTACCGTACCCCTCCT | |
| | TTTTTTGGCCAGGGGACCAAGCTGGAGGTCAAACGAACTGTGGCTGCACCATCTGTC | |
| AB c05 | GTGAAAAAATTATTATTCGCAATTCCTTTAGTTGTTCCTTTCTATTCTCACAGTGCAC | (SEQ ID NO:4) |
| | AAGACATCCAGATGACCCAGTCTCCAGCCACCCTGTATGTGTCTCCGGGGGAAAGAGC | |
| | CACCCTCTCCTGCAGGGCCAGTCAGAGTGTTAGTAGGAACTTAGCCTGGTACCAGCAG | |
| | AAACCTGGCCAGGCTCCCAGGCTCCTCATCTATGGTGCATCCACCAGGGCCACTGGTA | |
| | TCCCAGCCAGGTTCAGTGGCAGTGGGTCTCGGACAGAGTTCACTCTCACCATCAGCAG | |
| | CCTGCAGTCTGAAGATTTTGCAGTTTATCACTGTCAGCAGTATAATAGCAGGCCTCTC | |
| | ACTTTCGGCGGAGGGACCAAGGTGGAGATCAAACGAACTGTGGCTGCACCATCTGTC | |
| AB g05 | GTGAAAAAATTATTATTCGCAATTCCTTTAGTTGTTCCTTTCTATTCTCACAGTGCAC | (SEQ ID NO:5) |
| | AAGACATCCAGATGACCCAGTCTCCAGCCACCCTGTCTGTGTCTCCAGGGGAAAGAGC | |
| | CACCCTCTCCTGCAGGGCCAGTCAGAGTGTTCGCAGCTACTTAGCCTGGTACCAGCAG | |
| | AAACCAGGCCAGGCTCCCAGGCTCCTCATCTATGATGCATCCACCAGGGCCACTGGTA | |
| | TCCCAGCCAGATTCAGTGGCAGTGGGTCTGGGACAGAGTTCACTCTCACCATCAGCAG | |
| | CCTGCAGTCTGAAGATTTTGCAGTTTATTACTGTCAGCAGTATAATAACTGGCCTCCG | |
| | ACGTTCGGCCAAGGGACCAAGGTGGAAATCAAACGAACTGTGGCTGCACCATCTGTC | |
| AB c01 | GTGAAAAAATTATTATTCGCAATTCCTTTAGTTGTTCCTTTCTATTCTCACAGTGCAC | (SEQ ID NO:6) |
| | AAGACATCCAGATGACCCAGTCTCCAGCCACCCTGTCTGTGTCTCCAGGGGAAAGAGC | |
| | CACCCTCTCCTGCAGGGCCAGTCAGGATGTTAACAGATACTTAGCCTGGTACCAGCAG | |
| | AAACCTGGCCAGCCTCCCAGGCTCCTCATCTATGGTGCCTCTACCAGGGCCACTGGTA | |
| | TCCCAGCCAGGATCAGTGGCAGTGGGTCTGGGACAGAGTTCACTCTCACCATCAGCAG | |
| | CCTGCAGTCTGAAGATTTTGCAGTTTATTACTGTCAGCAGTATCATAACTGGCCCCTC | |
| | ACTTTCGGCGGAGGGACCAAGGTGGAGATCAAACGAACTGTGGCTGCACCATCTGTC | |
| AB d06 | GTGAAAAAATTATTATTCGCAATTCCTTTAGTTGTTCCTTTCTATTCTCACAGTGCAC | (SEQ ID NO:7) |
| | AAGACATCCAGATGACCCAGTCTCCAGCCACCCTGTCTTTGTCTCCAGGGGAAAGAGC | |
| | CACCCTCTCCTGCAGGGCCAGTCAGAGTGTTAGCAGCTACTTAGCCTGGTACCAACAG | |
| | AAACCTGGCCAGGCTCCCAGGCTCCTCATCTATGGTGCATCCAGCAGGGCCACTGGCA | |
| | TCCCAGACAGGTTCAGTGGCAGTGGGTCTGGGACAGACTTCACTCTCACCATCGGCAG | |
| | ACTGGAGCCTGAAGATTTTGCAGTGTATTACTGTCAGCAGTATAGTAGTTCACCGGTC | |
| | ACCTTCGGCCAAGGGACACGACTGGAGATTAAACGAACTGTGGCTGCACCATCTGTC | |
| AB e02 | GTGAAAAAATTATTATTCGCAATTCCTTTAGTTGTTCCTTTCTATTCTCACAGTGCAC | (SEQ ID NO:8) |
| | AAGACATCCAGATGACCCAGTCTCCAGCCACCCTGTCTTTGTCTCCAGGGGAAAGAGC | |
| | CACCCTCTCCTGCAGGGCCAGTCAGAGTGTTAGCAGGTACTTAGCCTGGTACCAACAG | |
| | AAACCTGGCCAGGCTCCCAGGCTCCTCATCTATGGTGCATCCACCAGGGCCACTGGTA | |
| | TCCCAGCCAGGTTCAGTGGCAGTGGGTCTGGGACAGAGTTCACTCTCACCATCAGCAG | |
| | CCTGCAGTCTGAAGATTTTGCAGTTTATTACTGTCAGCAGTATAATAACTGGCCTTCT | |
| | TTCGGCGGAGGGACCAAGGTGGAGATCAAACGAACTGTGGCTGCACCATCTGTC | |
| AB a05 | GTGAAAAAATTATTATTCGCAATTCCTTTAGTTGTTCCTTTCTATTCTCACAGTGCAC | (SEQ ID NO:9) |
| | AAGACATCCAGATGACCCAGTCTCCAGGCACCCTGTCTTTGTCTCCAGGGGAAAGAGC | |
| | CACCCTCTCCTGCAGGGCCAGTCAGAGTATTAGCAGCAGCTACTTAGCCTGGTACCAG | |
| | CAGAAACCTGGCCAGGCTCCCAGGCTCCTCATCTATGCTGCAGCCAGCAGGGCCACTG | |
| | GCATCCCAGACAGGTTCAGTGGCATTGGGTCTGGGACAGACTTCACTCTCACCATCAG | |
| | CAGCCTAGAGCCTGAAGATTTTGCAGTTTATTACTGTCAGCAGCGTAGCAACTGGCCT | |
| | CTCACTTTCGGCGGAGGGACCAAGGTGGAGATCAAACGAACTGTGGCTGCACCATCTG | |
| | TC | |
| AB g02 | GTGAAAAAATTATTATTCGCAATTCCTTTAGTTGTTCCTTTCTATTCTCACAGTGCAC | (SEQ ID NO:10) |
| | AAGACATCCAGATGACCCAGTCTCCAGGCACCCTGTCTTTGTCTCCAGGGGAAAGAGC | |
| | CACCCTCTCCTGCAGGGCCAGTCAGAGTGTTAGCAGCAGCTACTTAGCCTGGTACCAG | |
| | CAGAAACCTGGCCAGGCTCCCAGGCTCCTCATCTATGGTGCATCCAGCAGGGCCACTG | |
| | GCATCCCAGACAGGTTCAGTGGCAGTGGGTCTGGGACAGACTTCACTCTCACCATCAG | |
| | CAGACTGGAGCCTGAAGATTTTGCAGTGTATTACTGTCAGCAGTATGGTAGCTCACCG | |
| | TGGACGTTCGGCCAAGGGACCAAGGTGGAAATCAAACGAACTGTGGCTGCACCATCTG | |
| | TC | |
| AB b05 | GTGAAAAAATTATTATTCGCAATTCCTTTAGTTGTTCCTTTCTATTCTCACAGTGCAC | (SEQ ID NO:11) |
| | AAGACATCCAGATGACCCAGTCTCCATCCTCACTGTCTGCATCTGTAGGAGACAGAGT | |
| | CACCATCACTTGTCGGGCGAGTCAGGACATTAGCAATTATTTAGCCTGGTTTCAGCAG | |
| | AAACCAGGGAGAGCCCCTAAGTCCCTGATCTATGGTGCATCCAGTTTGCAAACTGGGG | |
| | TCCCATCAAAGTTCAGCGGCAGTGGATCTGGGACAGAGTTCACTCTCACCATCAGCGG | |
| | CCTGCAGCCTGAAGATGTTGCAACTTATTACTGCCATCAGTATAATCATTACCCTCCC | |
| | ACTTTCGGCGGAGGGACCAAGGTGGAGATCAAACGAACTGTGGCTGCACCATCTGTC | |
| AB c04 | GTGAAAAAATTATTATTCGCAATTCCTTTAGTTGTTCCTTTCTATTCTCACAGTGCAC | (SEQ ID NO:12) |
| | AAGACATCCAGATGACCCAGTCTCCATCCTCACTGTCTGCATCTGTAGGAGACAGAGT | |
| | CACCATCACTTGTCGGGCGAGTCAGGACATTAGGAATTATTTAGCCTGGTTTCAGCAG | |
| | AAACCAGGGGAAGCCCCTAAGTCCCTGATCTATGCTGCGTCCAGTTTGCAGAGTGGGG | |
| | TCTCATCAAACTTCAGCGGCAGTGGATCTGGGACAGATTTCACTCTCACCATCAGCAG | |
| | CCTGCAGCCTGAAGATTTTGCAACTTATTACTGCCAGCAGTATCATAGGTACCCGAGG | |
| | ACTTTTGGTCAGGGGACCAAGCTGGAGATCAAACGAACTGTGGCTGCACCATCTGTC | |
| AB b01 | GTGAAAAAATTATTATTCGCAATTCCTTTAGTTGTTCCTTTCTATTCTCACAGTGCAC | (SEQ ID NO:13) |
| | AAGACATCCAGATGACCCAGTCTCCATCCTCATTCTCTGCATCTACAGGAGACAGAGT | |
| | CACCATCACTTGTCGGGCGAGTCAGGGTATTAGCAGTTATTTAGCCTGGTATCAGCAA | |
| | AAACCAGGGAAAGCCCCTAAGCTCCTGATCTATGCTGCATCCACTTTGCAAAGTGGGG | |
| | TCCCATCAAAGTTCAGCGGCAGTGGATCTGGGACAGATTTCACTCTCACCATCAGCAG | |
| | CCTGCAGCCTGAAGATTTTGCAACTTATTACTGCCAACAGTATAATAGTTACCCCCTC | |
| | ACCTTCGGCCAAGGGACACGACTGGAGATTAAACGAACTGTGGCTGCACCATCTGTCT | |
| | TCATCTTCCCGCCATCTGATGAGCAGTTGAAATCTGGAACTGCCTCTGTTGTGTGCCT | |
| | GCTGAATAACTTCTATCCCAGAGAGGCCAAAGTACAGTGGAAGGTGGATAACGCCCTC | |
| | CAATCGGGTAACTCCCAGGAGAGTGTCACAGAGCAGGA | |
| AB b03 | GTGAAAAAATTATTATTCGCAATTCCTTTAGTTGTTCCTTTCTATTCTCACAGTGCAC | (SEQ ID NO:14) |
| | AAGACATCCAGATGACCCAGTCTCCATCCTCCCTGTATGCATCTGTAGGAGACAGAGT | |
| | CACCATCACTTGCCGGGCAAGTCAGGGCATTAGAAATGAGTTAGGTTGGTATCAGCAG | |
| | AAACCAGGGAAAGCCCCTCAGCGCCTGATCTATGATGCATCCACTTTGCAGAGTGGGG | |
| | TCCCATCAAGATTCAGCGGCGGTGGATCTAGGACAGAATTCACTCTCACCATCAGCAG | |
| | CCTGGAACCTCATGATTTTGGAACTTATTACTGCCAACAATATGCCAGTTATCCGCTC | |
| | ACTTTCGGCGGAGGGACCAAGGTGGAGATCAAACGAACTGTGGCTGCACCATCTGTC | |
| AB d02 | GTGAAAAAATTATTATTCGCAATTCCTTTAGTTGTTCCTTTCTATTCTCACAGTGCAC | (SEQ ID NO:15) |
| | AAGACATCCAGATGACCCAGTCTCCATCCTCCCTGTCTGCATCTGTAGGAGACAGAGT | |
| | CACCATCACTTGCCGGGCAAGTCAGAGCATTAGCAGCTATTTAAATTGGTATCAGCAG | |
| | AAACCAGGGAAAGCCCCTAAGCTCCTGATCTATGCTGCATCCAGTTTGCAAAGTGGGG | |
| | TCCCATCAAGGTTCAGTGGCAGTGGATCTGGGACAGATTTCACTCTCACCATCAGCAG | |
| | TCTGCAACCTGAAGATTTTGCAACTTACTACTGTCAACAGAGTTACAGTACCAGGTGG | |
| | ACGTTCGGCCAAGGGACCAAGGTGGAAATCAAACGAACTGTGGCTGCACCATCTGTC | |
| AB c03 | GTGAAAAAATTATTATTCGCAATTCCTTTAGTTGTTCCTTTCTATTCTCACAGTGCAC | (SEQ ID NO:16) |
| | AAGACATCCAGATGACCCAGTCTCCATCCTCCCTGTCTGCATCTGTAGGAGACAGAGT | |
| | CACCATCACTTGCCGGGCAAGTCAGAGCATTAGCAGCTATTTAAATTGGTATCAGCAG | |
| | AAACCAGGGAAAGCCCCTAAGCTCCTGATCTATGCTGCATCCAGTTTGCAAAGTGGGG | |
| | TCCCATCAAGGTTCAGTGGCAGTGGATCTGGGACAGATTTCACTCTCACCATCAGCAG | |
| | TCTGCAACCTGAAGATTTTGCAACTTACTACTGTCAACAGAGTTACAGTACCAGGTGG | |
| | ACGTTCGGCCAAGGGACCAAGGTGGAAATCAAACGAACTGTGGCTGCACCATCTGTC | |
| AB g01 | GTGAAAAAATTATTATTCGCAATTCCTTTAGTTGTTCCTTTCTATTCTCACAGTGCAC | (SEQ ID NO:17) |
| | AAGACATCCAGATGACCCAGTCTCCATCCTCCCTGTCTGCATCTGTAGGAGACAGAGT | |
| | CACCATCACTTGCCGGGCAAGTCAGGGCATTAGAAATGATTTAGGCTGGTTTCAGCAG | |
| | AAACCAGGGAAAGCCCCTAGGCGCCTGATCTGGGGTGCATCCACTTTACAAAGTGGGG | |
| | TCCCATCAAGGTTCAGCGGCAGTGGATCTGGCACAGATTTCACTCTCACCATCAGCAG | |
| | CCTGCAGCCTGAAGATTTTGCAACTTATTACTGTCTACAAGATTACAATTACCCGTAC | |
| | ACTTTTGGCCAGGGGACCAAGCTGGAGATCAAACGAACTGTGGCTGCACCATCTGTCT | |
| | TCATCTTCCCGCCATCTGATGAGCAGTTGAAATCTGGAACTGCCTCTGTTGTGTGCCT | |
| | GCTGAATAACTTCTATCCCAGAGAGGCCAAAGTACAGTGGAAGGTGGATAACGCCCTC | |
| | CAATCGGGTAACTCCCAGGAGAGTGTCACAGAGCAGGA | |
| AB a03 | GTGAAAAAATTATTATTCGCAATTCCTTTAGTTGTTCCTTTCTATTCTCACAGTGCAC | (SEQ ID NO:18) |
| | AAGACATCCAGATGACCCAGTCTCCATCCTCCCTGTCTGCATCTGTAGGAGACAGAGT | |
| | CACCATCACTTGCCGGGCAAGTCAGGGCATTAGACATTATTTAGGCTGGTATCAGCAG | |
| | AAACCAGGGAAAGCCCCTAAGCGCCTGATCTATGCTGCATCCAGTTTGCAATTTGGGG | |
| | TCCCAGCAAGGTTCAGCGGCAGTGGATCTGGGACGGAATTCACTCTCACAATCAGCAG | |
| | CCTGCAGCCTGAAGATTTTGCAACTTATTACTGTCTACAACACAATAGTTTCCCTCCG | |
| | GCGTTCGGCCAAGGGACCAAGGTGGAAATCAAACGAACTGTGGCTGCACCATCTGTC | |
| AB d03 | GTGAAAAAATTATTATTCGCAATTCCTTTAGTTGTTCCTTTCTATTCTCACAGTGCAC | (SEQ ID NO:19) |
| | AAGACATCCAGATGACCCAGTCTCCATCCTCCCTGTCTGCATCTGTAGGAGACAGAGT | |
| | CACCATCACTTGCCGGGCAAGTCAGGGCATTAGACATTATTTAGGCTGGTATCAGCAG | |
| | AAACCAGGGAAAGCCCCTAAGCGCCTGATCTATGCTGCATCCAGTTTGCAATTTGGGG | |
| | TCCCAGCAAGGTTCAGCGGCAGTGGATCTGGGACGGAATTCACTCTCACAATCAGCAG | |
| | CCTGCAGCCTGAAGATTTTGCAACTTATTACTGTCTACAACACAATAGTTTCCCTCCG | |
| | GCGTTCGGCCAAGGGACCAAGGTGGAAATCAAACGAACTGTGGCTGCACCATCTGTC | |
| AB b02 | GTGAAAAAATTATTATTCGCAATTCCTTTAGTTGTTCCTTTCTATTCTCACAGTGCAC | (SEQ ID NO:20) |
| | AAGACATCCAGATGACCCAGTCTCCATCCTCCCTGTCTGCATCTGTAGGAGACAGAGT | |
| | CACCATCACTTGCCGGGCAAGTCAGGGCATTAGACATTATTTAGGCTGGTATCAGCAG | |
| | AAACCAGGGAAAGCCCCTAAGCGCCTGATCTATGCTGCATCCAGTTTGCAATTTGGGG | |
| | TCCCAGCAAGGTTCAGCGGCAGTGGATCTGGGACGGAATTCACTCTCACAATCAGCAG | |
| | CCTGCAGCCTGAAGATTTTGCAACTTATTACTGTCTACAACACAATAGTTTCCCTCCG | |
| | GCGTTCGGCCAAGGGACCAAGGTGGAAATCAAACGAACTGTGGCTGCACCATCTGTC | |
| AB e06 | GTGAAAAAATTATTATTCGCAATTCCTTTAGTTGTTCCTTTCTATTCTCACAGTGCAC | (SEQ ID NO:21) |
| | AAGACATCCAGATGACCCAGTCTCCATCCTCCCTGTCTGCATCTGTAGGAGACAGAGT | |
| | CACCATCTCTTGCCGCGCAAGTCAGAACATTAGGAACTCTGTAAATTGGTATCAGCAG | |
| | AAACCAGGGAAAGCCCCTAAGCTCCTGATCTATGCTACATACGATTTGCAGAGTGGCG | |
| | CCCCATCATACTTCAGTGGCAGTGGATCTGGGACAGATTTCACTCTCACCATCACCAG | |
| | TCTGCAACCTGAAGATTTTGCAACTTACTACTGTCAACAGAGTTACAGTTTCCCTCGA | |
| | ACGTTCGGCCAAGGGACCAAGGTGGAAATCAGACGAACTGTGGCTGCACCATCTGTC | |
| AB a04 | GTGAAAAAATTATTATTCGCAATTCCTTTAGTTGTTCCTTTCTATTCTCACAGTGCAC | (SEQ ID NO:22) |
| | AAGACATCCAGATGACCCAGTCTCCATCTTCCGTGTCTGCATCTGTAGGAGACAGAAT | |
| | CGCCATCACTTGTCGGGCGAGTCAGGGTATTAGCACCTGGTTAGCCTGGTATCAGCAG | |
| | AGACCAGGGAGAGCCCCTAAGCTCCTGATCTATGCTGCATCCACTTTGCAAAGCGGAG | |
| | TCCCATCAAGGTTCAGCGGCAGTGGATCTGGGACAGATTTCACTCTCACCATCAGCAG | |
| | CCTGCAGCCTGAAGATTTTGCAACTTACTTTTGTCAACAGGCTGACAGTTTCCCCCTG | |
| | ACTTTTGGCCAGGGGACCAAACTGGAGATCAAACGAACTGTGGCTGCACCATCTGTC | |
| AB f05 | GTGAAAAAATTATTATTCGCAATTCCTTTAGTTGTTCCTTTCTATTCTCACAGTGCAC | (SEQ ID NO:23) |
| | AAGACATCCAGATGACCCAGTCTCCATCTTCCGTGTCTGCATCTGTAGGAGACAGAGT | |
| | CACCATCACTTGTCGGGCGAGTCGGGGTATTAGCAGATGGTTAGCCTGGTATCAGCAG | |
| | AAACCAGGGAAAGCCCCTAAGCTCCTGATCTATGGTGCATCCACTTTGCAAAAAGGGG | |
| | TCCCATCAAGGTTCACCGGCAGTGGATCTGGGACAGATTTCACTCTCACCATCACCAG | |
| | CCTGCAGCCTGAAGATTTTGCAACTTACTATTGTCAACAGGGTAACAGTTTCCCATTC | |
| | ACTTTCGGCCCTGGGACCAAAGTGGATATCAAACGAACTGTGGCTGCACCATCTGTC | |
| AB g03 | GTGAAAAAATTATTATTCGCAATTCCTTTAGTTGTTCCTTTCTATTCTCACAGTGCAC | (SEQ ID NO:24) |
| | AAGACATCCAGATGACCCAGTCTCCATCTTCCGTGTCTGCATCTGTAGGAGACAGAGT | |
| | CACCATCACTTGTCGGGCGAGTCGGGGTATTAGCAGATGGTTAGCCTGGTATCAGCAG | |
| | AAACCAGGGAAAGCCCCTAAGCTCCTGATCTATGGTGCATCCACTTTGCAAAAAGGGG | |
| | TCCCATCAAGGTTCACCGGCAGTGGATCTGGGACAGATTTCACTCTCACCATCACCAG | |
| | CCTGCAGCCTGAAGATTTTGCAACTTACTATTGTCAACAGGGTAACAGTTTCCCATTC | |
| | ACTTTCGGCCCTGGGACCAAAGTGGATATCAAACGAACTGTGGCTGCACCATCTGTC | |
| AB e04 | GTGAAAAAATTATTATTCGCAATTCCTTTAGTTGTTCCTTTCTATTCTCACAGTGCAC | (SEQ ID NO:25) |
| | AAGACATCCAGATGACCCAGTCTCCGTCTTCCGTGTCTGCATCTGTAGGAGACAGAGT | |
| | CACCATCACTTGTCGGGCGAGTCAGGGTATTAGCAGCTGGTTAGCCTGGTATCAGCAG | |
| | AAGCCAGGGAAAGCCCCTAAGTTGCTGATCTATGGTGCATCCAGTTTGGAAAGTGGGG | |
| | TCCCATCAAGATTCAGCGGCAGTGGATCTGGGACAGATTACACTCTCACCATCACCAG | |
| | CCTACAGCCTGAAGATTTTGCAACTTACTTTTGTCAACAGGTTAATTCTTTCCCTCGT | |
| | ACTTTTGGCCAGGGGACCAAGCTGAATATCAAACGAACTGTGGCTGCACCATCTGTC | |
| AB e05 | GTGAAAAAATTATTATTCGCAATTCCTTTAGTTGTTCCTTTCTATTCTCACAGTGCAC | (SEQ ID NO:26) |
| | AGAGCGAATTGACTCAGATAAGGGCCAGGGCTCTGGAGTCCCCAGCCGCTTCTCTGGA | |
| | TCCAAAGATGCTGCAGCCAATGCAGGGGTTTTACTCATCTCCGGCCTCCAGCCCGAGG | |
| | ATGATGCTGACTATTTTTGTATGATATGGTTAAGCAATGTACATGCGACATTCGGCGG | |
| | AGGGACCAAGCTGACCGTCCTGGGTCAGCCCAAGGCTGCCCCCTCGGTCACTCTGTTC | |
| | CCGCCCTCCTCTGAGGAGCTTCAAGCCAACAAGGCCACACTGGTGTGTCTCATAAGTG | |
| | ACTTCTACCCGGGAGCCGTGACAGTGGCCTGGAAGGCAGATAGCAGCCCCGTCAAGGC | |
| | GGGAGTGGAGACCACCACACCCTCCAAACAAAGCAACAACAAGTACGCGGCCAGCAGC | |
| | TATCTAAGCCTGACGCCTGAGCAGTGGAAGTCCCACAGAAGCTACAGCTGCCAGGTCA | |
| | CGCATGAAGGGAGCACC | |
| AB d04 | GTGAAAAAATTATTATTCGCAATTCCTTTAGTTGTTCCTTTCTATTCTCACAGTGCAC | (SEQ ID NO:27) |
| | AGAGCGAATTGACTCAGCCACCCTCAGCGTCTGCGACCCCCGGGCAGAGGGTCACCAT | |
| | CTCTTGTTCTGGAAGCAGCTCCAACATCGGACGTAATTTGGTATACTGGTACCAGCAG | |
| | CTCCCAGGAACGGCCCCCAAACTCCTCATCTATAGTAATAATCAGCGGCCCTCAGGGG | |
| | TCCCTGACCGATTCTCTGGCTCCAAGTCTGGCACCTCAGCCTCCCTGGCCATCAGTGG | |
| | GCTCCGGTCCGAGGAGGAGGCTGATTATTACTGTGCAGCATGGGATGACAGCCTGAGT | |
| | GGTTGGGTGTTCGGCGGAGGGACCAGGCTGACCGTCCTAGGTCAGCCCAAGGCTGCCC | |
| | CCTCG | |
| AB b04 | GTGAAAAAATTATTATTCGCAATTCCTTTAGTTGTTCCTTTCTATTCTCACAGTGCAC | (SEQ ID NO:28) |
| | AGAGCGCTTTGACTCAGCCACCCTCAGCGTCTGGGACCCCCGGGCAGAGGGTCACCAT | |
| | CTCTTGTTCTGGAAGCAGCTCCAACATCGGAAGTAATTTTGTATACTGGTACCACCAT | |
| | CTCCCAGGAACGGCCCCCAAACTCCTCATCTATAGGAATAATCAGCGGCCCTCAGGGG | |
| | TCCCTGACCGATTCTCTGGCTCCAAGTCTGGCACCTCAGCCTCCCTGGCCATCAGTGG | |
| | GCTCCGGTCCGAGGATGAGGCTGATTATTACTGTGCAGCATGGGATGACAGCCTGAGT | |
| | GGGGTGGTATTCGGCGGAGGGACCAAGCTGACCGTCCTAGGTCAGCCCAAGGCTGCCC | |
| | CCTCG | |
| AB c02 | GTGAAAAAATTATTATTCGCAATTCCTTTAGTTGTTCCTTTCTATTCTCACAGTGCAC | (SEQ ID NO:29) |
| | AGAGCGCTTTGACTCAGCCTGCCTCCGTGTCTGGGTCTCCTGGACAGTCGATCACCAT | |
| | CTCCTGCACTGGAACCAGCAGTGACGTTGGTTATTATGACTATGTCTCCTGGTACCAG | |
| | CACCACCCAGGCAAAGCCCCCAAACTCATCATTTATGATGTCACTTCTCGGCCCTCAG | |
| | GGGTCTCTTCTCATTTCTCTGGCTCCAAGTCTGGCAACACGGCCTCCCTGACCATCTC | |
| | TGGGCTCCAGGCTGATGACGAGGCTGATTATTACTGCAGCTCATATACAAGCGGCAGC | |
| | ACCCGTTATGTCTTCGGACCTGGGACCAAGGTCACCGTCCTAGGTCAGCCCAAGGCCA | |
| | ACCCCACT | |
| AB c06 | GTGAAAAAATTATTATTCGCAATTCCTTTAGTTGTTCCTTTCTATTCTCACAGTGCAC | (SEQ ID NO:30) |
| | AGAGCGCTTTGACTCAGCCTGCCTCCGTGTCTGGGTCTCCTGGACAGTCGATCACCAT | |
| | CTCCTGCACTGGAACCAGCAGTGACGTTGGTTATTATGACTATGTCTCCTGGTACCAG | |
| | CACCACCCAGGCAAAGCCCCCAAACTCATCATTTATGATGTCACTTCTCGGCCCTCAG | |
| | GGGTCTCTTCTCATTTCTCTGGCTCCAAGTCTGGCAACACGGCCTCCCTGACCATCTC | |
| | TGGGCTCCAGGCTGATGACGAGGCTGATTATTACTGCAGCTCATATACAAGCGGCAGC | |
| | ACCCGTTATGTCTTCGGACCTGGGACCAAGGTCACCGTCCTAGGTCAGCCCAAGGCCA | |
| | ACCCCACT | |
| AB d05 | GTGAAAAAATTATTATTCGCAATTCCTTTAGTTGTTCCTTTCTATTCTCACAGTGCAC | (SEQ ID NO:31) |
| | AGAGCGTCTTGACTCAGACTGCCTCCGTGTCTGGGTCTCCTGGACAGTCGATCACCAT | |
| | CTCCTGCACTGGAACCAGCAGTGACATTGGTGATTATGAGTATGTCTCCTGGTACCAA | |
| | CAACACCCAGGCAAAGCCCCCAAAGTCATTCTTTATGAGGTCAGTAATCGGCCCTCAG | |
| | GGGTCCCTGATCGCTTCTCTGGCTCCAAGTCTGGCAACACGGCCTCACTGACCATCTC | |
| | TGGACTCCAGGCTGAGGACGAGGCTGATTATTACTGTGGTTCATATAGAAAGAGCAGC | |
| | ACTCCTTATGTCTTCGGAACTGGGACCAAGGTCAGCGTCCTAGGTCAGCCCAAGGCCA | |
| | ACCCCACT | |
| AB f06 | GTGAAAAAATTATTATTCGCAATTCCTTTAGTTGTTCCTTTCTATTCTCACAGTGCAC | (SEQ ID NO:32) |
| | AGAGCGTCTTGACTCAGACTGCCTCCGTGTCTGGGTCTCCTGGACAGTCGATCACCAT | |
| | CTCCTGCACTGGAACCAGCAGTGACATTGGTGATTATGAGTATGTCTCCTGGTACCAA | |
| | CAACACCCAGGCAAAGCCCCCAAAGTCATTCTTTATGAGGTCAGTAATCGGCCCTCAG | |
| | GGGTCCCTGATCGCTTCTCTGGCTCCAAGTCTGGCAACACGGCCTCACTGACCATCTC | |
| | TGGACTCCAGGCTGAGGACGAGGCTGATTATTACTGTGGTTCATATAGAAAGAGCAGC | |
| | ACTCCTTATGTCTTCGGAACTGGGACCAAGGTCAGCGTCCTAGGTCAGCCCAAGGCCA | |
| | ACCCCACT | |
| AB a01 | GTGAAAAAATTATTATTCGCAATTCCTTTAGTTGTTCCTTTCTATTCTCACAGTGCAC | (SEQ ID NO:33) |
| | AGAGCGTCTTGACTCAGCCACCCTCAGCGTCTGGGACCCCCGGGCAGAGGGTCACCAT | |
| | CTCTTGTTCTGGAAGCAGCTCCAACATCGAAAGTAATACTGTAACCTGGTACCAGCAA | |
| | CTCCCAGGAACGGCCCCCAAACTCCTCATCTATAGTGATGATCAGCGGCCCTCAGGGG | |
| | TCCCTGACCGATTCTCTGGATCCAAGTCTGGCACCTCAGCCTCCCTGGCCATCAGTGG | |
| | GCTCCAGTCTGAGGATGAGGCTGACTATTACTGTGCAACATGGGATAACACCCTGAGA | |
| | GGTGTGGTTTTCGGCGGAGGGACCAAGCTGACCGTCCTGAGTCAGCCCAAGGCTGCCC | |
| | CCTCGGTCACTCTGTTCCCGCCCTCCTCTGAGGAGCTTCAAGCCAACAAGGCCACACT | |
| | GGTGTGTCTCATAAGTGACTTCTACCCGGGAGCCGTGACAGTGGCCTGGAAGGCAGAT | |
| | AGCAGCCCCGTCAAGGCGGGAGTGGAGACCACCACACC | |
| AB e03 | GTGAAAAAATTATTATTCGCAATTCCTTTAGTTGTTCCTTTCTATTCTCACAGTGCAC | (SEQ ID NO:34) |
| | AGAGCGTCTTGACTCAGCCACCTTATGCATCAGCCTCCCTGGGAGCCTCGGTCACACT | |
| | CACCTGCACCCTGAGCAGCGGCTACAGTAATTATAAAGTGGACTGGTATCAGCAAAGA | |
| | CCAGGGAAGGGCCCCCAGTTTGTGATGCGAGTGGGCAGTGGCGGGATTGTGGGATCAA | |
| | AGGGGGATGGCATCCCTGATCGCTTTTCAGTCCTGGGCTCAGGCCTGTATCGGTATCT | |
| | GACCATCAAGAACATCCAGGAAGAGGATGAGAGTGACTACTATTGTGGGGCAGACCAT | |
| | GGCAGGGGGGGCACCTTCGTGTGGGTGTTCGGCGGAGGGACCAAACTGACCGTCCTAG | |
| | GTCAGCCCAAGGCTGCCCCCTCG | |
| AB g04 | GTGAAAAAATTATTATTCGCAATTCCTTTAGTTGTTCCTTTCTATTCTCACAGTGCAC | (SEQ ID NO:35) |
| | AGAGCGTCTTGACTCAGCCTGCCTCCGTGTCTGGGTCTCCTGGACAGTCGATCACCAT | |
| | CTCCTGCACTGGAACCAGCAGTGACGTTGGTGGTTATAACTATGTCTCCTGGTACCAA | |
| | CGACACCCAGGCAAAGCCCCCAAACTCATTATTTATGATGTCACTAATCGCCCCTCAG | |
| | GGGCTTCTCGTCACTTCTCTGGCTCCAAGTCTGGCAACACGGCCTCCCTGACCATCTC | |
| | TGGTCTCCAGGCCGACGACGAGGCTGATTATTATTGCGTTTCATTTACAAACAGCAAT | |
| | ACTTTCGTCTTCGGAAGTGGGACCAGGGTCACCGTCCTCGGTCAGCCCAAGGCCAACC | |
| | CCACT | |
| AB a02 | GTGAAAAAATTATTATTCGCAATTCCTTTAGTTGTTCCTTTCTATTCTCACAGTGCAC | (SEQ ID NO:36) |
| | AGGACATCGTCATGACTCAAACCCCTCCTAGTTTACCGGTTAACCCGGGTGAACCTGC | |
| | CTCCATCTCCTGCAAGTCTAGTCAGAGCCTCCTGCAGAGTAATGGATACAACTACTTG | |
| | GATTGGTACCTGCAGAAGCCAGGGCAGTCTCCACAGCTCCTGATCTATTTGGGTTCTA | |
| | ATCGGGCCTCCGGGGTCCCTGACAGGTTCAGTGGCAGTGGATCAGGCACAGATTTTAC | |
| | ACTGAAGATCAGCAGGGTGGAGGCTGAGGATGTTGGCATTTATTACTGCATGCAAGCT | |
| | CTACACACTCCTCCCTTCGGCCAAGGGACACGACTGGAGATTAAACGAACTGTGGCTG | |
| | CACCATCTGTC | |
| AB a06 | GTGAAAAAATTATTATTCGCAATTCCTTTAGTTGTTCCTTTCTATTCTCACAGTGCAC | (SEQ ID NO:37) |
| | AGTACGAATTGACTCAGCCACCCTCAGTGTCCGTGTCCCCGGGACAGACAGCCACCAT | |
| | TATCTGCTCTGGAGATAAATTGGGGGATAAATATGTTGCCTGGTATCAGCAGAAGCCA | |
| | GGCCAGTCCCCTGTGCTGGTCGTCTATGAAGATAACAAGCGGCCCTCAGGGATCCCTG | |
| | AGCGAATTTCTGGCTCCAACTCTGGGAACACAGCCACTCTGACCATCAGTGGGACCCA | |
| | GGCTATGGATGACGCTGACTATTACTGTCAGGCGTGGGACAGAAGCACTGACCATTAT | |
| | GTCTTCGGAACTGGGACCAAGGTCACCGTCCTAGGTCAGCCCAAGGCCAACCCCACT | |
| AB f03 | GTGAAAAAATTATTATTCGCAATTCCTTTAGTTGTTCCTTTCTATTCTCACAGTGCAC | (SEQ ID NO:38) |
| | AGTACGAATTGACTCAGCCTGCCTCCGTGTCTGGGTCTCCTGGACAGTCGATCACCAT | |
| | CTCCTGCACTGGAACCAGCAGCGACGTTGGTGGTTATAACTATGTCTCCTGGTACCAA | |
| | CAGCACCCAGGCAAAGCCCCCAAACTCATGATTTATGAGGTCAGTAATCGGCCCTCAG | |
| | GGGTTTCTAATCGCTTCTCTGGCTCCAAGTCTGACAATACGGCCTCCCTGACCATCTC | |
| | TGGACTCCAGGCTGAGGACGAGGCTGATTATTACTGTGGTTCATATAGAAAGAGCAGC | |
| | ACTCCTTATGTCTTCGGAACTGGGACCAAGGTCAGCGTCCTAGGTCAGCCCAAGGCCA | |
| | ACCCCACT | |
| AB f01 | GTGAAAAAATTATTTATTCGCAATTTCCTTTAGTTGTTCCTTTCTATTCTCACAGTGC | (SEQ ID NO:39) |
| | ACAGAGCGCTTTGACTCAGCCATCCTCAGCGTCTGGGACCCCCGGGCAGAGGGTCAGT | |
| | ATCTCTTGTTCTGGAAGCAGCTACAACATCGGAGTTTATGATGTATACTGGTACCAGC | |
| | AGCTCCCAGGAACGGCCCCCAAACTCCTCATCTATACCAATAATCAGCGGCCCTCAGG | |
| | GGTCCCTGACCGATTCTCTGGCTCCAAGTCTGGCACCTCAGCCTCCCTGGCCATCAGT | |
| | GGGCTCCAGTCTGAGGATGAGGCTGATTATTACTGTGCAGCATGGGATGACAGTCTGA | |
| | GTGGTTGGGTGTTCGGCGGAGGGACCAAGGTGACCGTCCTAGGTCAGCCCAAGGCTGC | |
| | CCCCTCG | |
| AB B12 | TTCTATTCTCACAGTGCACAGAGCGAATTGACTCAGCCACCCTCAGCGTCTGGGACCC | (SEQ ID NO:53) |
| | CCGGGCAGAGGGTCACCATCTCTTGTTCTGGAAGCAGCTCCAACATCGGAAGTAATAC | |
| | TGTAAACTGGTACCAGCAGCTCCCAGGAACGGCCCCCAAACTCCTCATCTATAGTAAT | |
| | AATTACCGGCCCTCAGGGGTCCCTGACCGATTCTCTGGCTCCAAGTCTGGCACCTCAG | |
| | CCTCCCTGGCCATCAGTGGGCTCCAGTCTGACGATGAGGCTGAATATCTCTGTGCAGC | |
| | ATGGGATGACAGTCTGAATGGTCCGGTGTTCGGTGGAGGGACCAAGGTGACCGTCCTA | |
| | GGTCAGCCCAAGGCTGCCCCC | |
| AB E06 | TTCTATTCTCACAGTGCACAGAGCGTCTTGACTCAGCCACCCTCAGCGTCTGCGACCC | (SEQ ID NO:348) |
| | CCGGGCAGAGGGTCACCTTCTCTTGTTCTGGAAGCAGCTCCAACATCGGAAGTAATGC | |
| | TGTAAACTGGTACCATCAGCTCCCAGGAACGGCCCCCAAACTCCTCATCTATCATAAT | |
| | AATCAGCGACCCTCAGGGGTCCCTGACCGATTCTCTGGCTCCAAGTCTGGCACCTCAG | |
| | CCTCCCTGGCCATCAGTGGGCTCCAGTCTGAGGATGAGGCTGATTATTACTGTGCAGC | |
| | ATGGGATGACAGCCTGCATGGTTATGTCTTCGGACCTGGGACCAAGGTCACCGTCCTA | |
| | GGTCAGCCCAAGGCCAACCCC | |
| AB F05 | TTCTATTCTCACAGTGCACAGAGCGTCTTGACTCAGCCTCGCTCAGTGTCCGGGTCTC | (SEQ ID NO:350) |
| | CTGGACAGTCAGTCACCATCTCCTGCACTGGAACCAGTAGTGATGTTGGTGCTAGTTA | |
| | TAAGTTTGTCTCCTGGTACCAACTAAAGCCAGGCAAAGCCCCCAAACTCATGCTTTTT | |
| | AATGTCCGTGAGCGGCCCTCAGGGGTCCCTGATCGCTTTTCTGGGTCCAAGTCCGGCA | |
| | ACACGGCCTCCCTGACCATCTCTGGGCTCCAGGCTGAGGATGAGGCTGACTATTACTG | |
| | CTGTTCCTATGCACGCGGCCAGACTTTCTCTTATGTCTTCGGAGGTGGGACCACGGTC | |
| | ACCGTCCTAGGTCAGCCCAAGGCCAACCCC | |

Nucleic Acids encoding Heavy Chain Variable Domains

| | |
|---|---|
| AB e01 | GAAGTTCAATTGTTAGAGTCTGGTGGCGGTCTTGTTCAGCCTGGTGGTTCTTTACGTC |
| | TTTCTTGCGCTGCTTCCGGATTCACTTTCTCTCTTTACCAGATGCTTTGGGTTCGCCA |
| | AGCTCCTGGTAAAGGTTTGGAGTGGGTTTCTGGTATCGTTTCTTCTGGTGGCCTTACT |
| | GGTTATGCTGACTCCGTTAAAGGTCGCTTCACTATCTCTAGAGACAACTCTAAGAATA |
| | CTCTCTACTTGCAGATGAACAGCTTAAGGGCTGAGGACACTGCAGTCTACTATTGTGC |
| | GAGACATAATAGGGCTATTGGCACCTTTGACTACTGGGGCCAGGGAACCCTGGTCACC |
| | GTCTCAAGCGCCTCCACCAAGGGCCCATCGGTCTTCCCG (SEQ ID NO:40) |
| AB c05 | GAAGTTCAATTGTTAGAGTCTGGTGGCGGTCTTGTTCAGCCTGGTGGTTCTTTACGTC |
| | TTTCTTGCGCTGCTTCCGGATTCACTTTCTCTTGGTACTTTATGAATTGGGTTCGCCA |
| | AGCTCCTGGTAAAGGTTTGGAGTGGGTTTCTTCTATCTATCCTTCTGGTGGCTATACT |
| | ATGTATGCTGACTCTGTTAAAGGTCGCTTCACTATCTCTAGAGACAACTCTAAGAAGA |
| | CTCTCTACTTGCAGATGAACAGCTTAAGGGCTGAGGACACTGCAGTCTACTATTGTGC |
| | GAGTGACTTTGGTAGCTGGGGCCAGGGAACCCTGGTCACCGTCTCAAGCGCCTCCACC |
| | AAGGGCCCATCGGTCTTCCCG (SEQ ID NO:41) |
| AB g05 | GAAGTTCAATTGTTAGAGTCTGGTGGCGGTCTTGTTCAGCCTGGTGGTTCTTTACGTC |
| | TTTCTTGCGCTGCTTCCGGATTCACTTTCTCTTGGTACCGTATGAATTGGGTTCGCCA |
| | AGCTCCTGGTAAAGGTTTGGAGTGGGTTTCTTCTATCGTTCCTTCTGGTGGCTATACT |
| | CGTTATGCTGACTCCGTTAAAGGTCGCTTCACTATCTCTAGAGACAACTCTAAGAATA |
| | CTCTCTACTTGCAGATGAACAGCTTAAGGGCTGAGGACACTGCAGTCTACTATTGTGC |
| | GAGTGACTTTGGTAGCTGGGGCCAGGGAACCCTGGTCACCGTCTCAAGCGCCTCCACC |
| | AAGGGCCCATCGGTCTTCCCG (SEQ ID NO:42) |
| AB C01 | GAAGTTCAATTGTTAGAGTCTGGTGGCGGTCTTGTTCAGCCTGGTGGTTCTTTACGTC |
| | TTTCTTGCGCTGCTTCCGGATTCACTTTCTCTCGTTACTCTATGAATTGGGTTCGCCA |
| | AGCTCCTGGTAAAGGTTTGGAGTGGGTTTCTTATATCTCTCCTTCTGGTGGCATGACT |
| | AAGTATGCTGACTCCGTTAAAGGTCGCTTCACTATCTCTAGAGACAACTCTAAGAATA |
| | CTCTCTACTTGCAGATGAACAGCTTAAGGGCTGAGGACACTGCAGTCTACTATTGTGC |
| | GAATACCCTTGGCTACTGGGGCCAGGGAACCCTGGTCACCGTCTCAAGCGCCTCCACC |
| | AAGGGCCCATCGGTCTTCCCG (SEQ ID NO:43) |
| AB d06 | GAAGTTCAATTGTTAGAGTCTGGTGGCGGTCTTGTTCAGCCTGGTGGTTCTTTACGTC |
| | TTTCTTGCGCTGCTTCCGGATTCACTTTCTCTTCTTACCGTATGAATTGGGTTCGCCA |
| | AGCTCCTGGTAAAGGTTTGGAGTGGGTTTCTGGTATCGTTCCTTCTGGTGGCAAGACT |
| | TTTTATGCTGACTCCGTTAAAGGTCGCTTCACTATCTCTAGAGACAACTCTAAGAATA |
| | CTCTCTACTTGCAGATGAACAGCTTAAGGGCTGAGGACACTGCAGTCTACTATTGTGC |
| | GAGTGACTTTGGTAGCTGGGGCCAGGGAACCCTGGTCACCGTCTCAAGCGCCTCCACC |
| | AAGGGCCCATCGGTCTTCCCG (SEQ ID NO:44) |
| AB e02 | GAAGTTCAATTGTTAGAGTCTGGTGGCGGTCTTGTTCAGCCTGGTGGTTCTTTACGTC |
| | TTTCTTGCGCTGCTTCCGGATTCACTTTCTCTAATTACTCTATGGATTGGGTTCGCCA |
| | AGCTCCTGGTAAAGGTTTGGAGTGGGTTTCTTGGATCTCTCCTTCTGGTGGCCTTACT |
| | ACTTATGCTGACTCCGTTAAAGGTCGCTTCACTATCTCTAGAGACAACTCTAAGAATA |
| | CTCTCTACTTGCAGATGAACAGCTTAAGGGCTGAGGACACTGCAGTCTACTATTGTGC |
| | GAGTGACTTTGGTAGCTGGGGCCAGGGAACCCTGGTCACCGTCTCAAGCGCCTCCACC |
| | AAGGGCCCATCGGTCTTCCCG (SEQ ID NO:45) |
| AB a05 | GAAGTTCAATTGTTAGAGTCTGGTGGCGGTCTTGTTCAGCCTGGTGGTTCTTTACGTC |
| | TTTCTTGCGCTGCTTCCGGATTCACTTTCTCTCGTTACCATATGGAGTGGGTTCGCCA |
| | AGCTCATGGTAAAGGTTTGGAGTGGGTTTCTTATATCTCTCCTTCTGGTGGCAAGACT |
| | CTTTATGCTGACTCCGTTAAAGGTCGCTTCACTATCTCTAGAGACAACTCTAAGAATA |
| | CTCTCTACTTGCAGATGAACAGCTTAAGGGCTGAGGACACTGCAGTCTACTATTGTGC |
| | GAGACATTTGGGATATGGTTCGGGGAGTTACTTTGACTACTGGGGCCAGGGAACCCTG |
| | GTCACCGTCTCAAGCGCCTCCACCAAGGGCCCATCGGTCTTCCCG |
| | (SEQ ID NO:46) |
| AB g02 | GAAGTTCAATTGTTAGAGTCTGGTGGCGGTCTTGTTCAGCCTGGTGGTTCTTTACGTC |
| | TTTCTTGCGCTGCTTCCGGATTCACTTTCTCTTTTTACCCTATGCCTTGGGTTCGCCA |
| | AGCTCCTGGTAAAGGTTTGGAGTGGGTTTCTTATATCTCTCCTTCTGGTGGCGATACT |
| | ACTTATGCTGACTCCGTTAAAGGTCGCTTCACTATCTCTAGAGACAACTCTAAGAATA |
| | CTTTCTACTTGCAGATGAACAGCTTAAGGGCTGAGGACACTGCAGTCTACTATTGTGC |
| | GAGAGGGGGGTCCTATAGCAGCAGTTGGTACGGCTACTGGGGCCAGGGAACCCTGGTC |
| | ACCGTCTCAAGCGCCTCCACCAAGGGCCCATCGGTCTTCCCG (SEQ ID NO:47) |
| AB b05 | GAAGTTCAATTGTTAGAGTCTGGTGGCGGTCTTGTTCAGCCTGGTGGTTCTTTACGTC |
| | TTTCTTGCGCTGCTTCCGGATTCACTTTCTCTAAGTACCCTATGTTTTGGGTTCGCCA |
| | AGCTCCTGGTAAAGGTTTGGAGTGGGTTTCTTGGATCTCTCCTTCTGGTGGCAAGACT |
| | GTTTATGCTGACTCCGTTAAAGGTCGCTTCACTATCTCTAGAGACAACTCTAAGAATA |
| | CTCTCTACTTGCAGATGAACAGCTTAAGGGCTGAGGACACTGCAGTCTACTATTGTGC |
| | GAAAGATTGCAGAGGGGGTTGCAGTGGTGGAAGTTGGGGCCAGGGAACCCTGGTCACC |
| | GTCTCAAGCGCCTCCACCAAGGGCCCATCGGTCTTCCCG (SEQ ID NO:48) |
| AB c04 | GAAGTTCAATTGTTAGAGTCTGGTGGCGGTCTTGTTCAGCCTGGTGGTTCTTTACGTC |
| | TTTCTTGCGCTGCTTCCGGATTCACTTTCTCTGCTTACAATATGCCTTGGGTTCGCCA |
| | AGCTCCTGGTAAAGGTTTGGAGTGGGTTTCTTATATCTCTTCTTCTGGTACTGGTTAT |
| | GCTGACTCCGTTAAAGGTCGCTTCACTATCTCTAGAGACAACTCTAAGAATACTCTCT |
| | ACTTGCAGATGAACAGCTTAAGGGCTGAGGACACTGCAGTCTACTATTGTGCGAGAGA |
| | ACTGGGTAGTGGGAGCTACTACCCGGGATACTTCCAGCACTGGGGCCAGGGCACCCTG |
| | GTCACCGTCTCAAGCGCCTCCACCAAGGGCCCATCGGTCTTCCCG |
| | (SEQ ID NO:49) |
| AB b01 | GAAGTTCAATTGTTAGAGTCTGGTGGCGGTCTTGTTCAGCCTGGTGGTTCTTTACGTC |
| | TTTCTTGCGCTGCTTCCGGATTCACTTTCTCTTGGTACACTATGGTTTGGGTTCGCCA |
| | AGCTCCTGGTAAAGGTTTGGAGTGGGTTTCTTCTATCTATTCTTCTGGTGGCTTTACT |
| | TGGTATGCTGACTCCGTTAAAGGTCGCTTCACTATCTCTAGAGACAACTCTAAGAATA |
| | CTCTCTACTTGCAGATGAACAGCTTAAGGGCTGAGGACACTGCAGTCTACTATTGTGC |
| | GAGTGACTTTGGTAGCTGGGGCCAGGGAACCCTGGTCACCGTCTCAAGCGCCTCCACC |
| | AAGGGCCCATCGGTCTTCCCG (SEQ ID NO:50) |
| AB b03 | GAAGTTCAATTGTTAGAGTCTGGTGGCGGTCTTGTTCAGCCTGGTGGTTCTTTACGTC |
| | TTTCTTGCGCTGCTTCCGGATTCACTTTCTCTGATTACAAGATGCCTTGGGTTCGCCA |
| | AGCTCCTGGTAAAGGTTTGGAGTGGGTTTCTTCTATCTGGTCTTCTGGTGGCACTACT |
| | GAGTATGCTGACTCCGTTAAAGGTCGCTTCACTATCTCTAGAGACAACTCTAAGAATA |
| | CTCTCTACTTGCAGATGAACAGCTTAAGGGCTGAGGACACTGCAGTCTACTATTGTGC |
| | GAGAGAGGAAATTGGACGATATTTTGACTGGTTTTTAGGGAACTACTACTACTACGGT |
| | ATGGACGTCTGGGGCCAAGGGACCACGGTCACCGTCTCAAGCGCCTCCACCAAGGGCC |
| | CATCGGTCTTCCCG (SEQ ID NO:51) |
| AB d02 | GAAGTTCAATTGTTAGAGTCTGGTGGCGGTCTTGTTCAGCCTGGTGGTTCTTTACGTC |
| | TTTCTTGCGCTGCTTCCGGATTCACTTTCTCTACTTACTTTATGCGTTGGGTTCGCCA |
| | AGCTCCTGGTAAAGGTTTGGAGTGGGTTTCTTATATCGTTCCTTCTGGTGGCAATACT |
| | CTTTATGCTGACTCCGTTAAAGGTCGCTTCACTATCTCTAGAGACAACTCTAAGAATA |
| | CTCTCTACTTGCAGATGAACAGCTTAAGGGCTGAGGACACTGCAGTCTACTATTGTGC |
| | AAGAGAAGAGTGGGACGTATTACTATGGTTCGGGGAGTTAAGTGCTGCTTTTGATATC |
| | TGGGGCCAAGGGACAATGGTCACCGTCTCAAGCGCCTCCACCAAGGGCCCATCGGTCT |
| | TCCCG (SEQ ID NO:52) |
| AB g01 | GAAGTTCAATTGTTAGAGTCTGGTGGCGGTCTTGTTCAGCCTGGTGGTTCTTTACGTC |
| | TTTCTTGCGCTGCTTCCGGATTCACTTTCTCTTTTTACGGTATGCCTTGGGTTCGCCA |
| | AGCTCCTGGTAAAGGTTTGGAGTGGGTTTCTGGTATCTATCCTTCTGGTGGCGTTACT |
| | CGTTATGCTGACTCCGTTAAAGGTCGCTTCACTATCTCTAGAGACAACTCTAAGAATA |
| | CTCTCTACTTGCAGATGAACAGCTTAAGGGCTGAGGACACTGCAGTCTACTATTGTGC |
| | GAAGACGTATAGCAGCAGCTGGTACGGGTGGTACTTTGACTACTGGGGCCAGGGAACC |
| | CTGGTCACCGTCTCAAGCGCCTCCACCAAGGGCCCATCGGTCTTCCCG |
| | (SEQ ID NO:54) |
| AB a03 | GAAGTTCAATTGTTAGAGTCTGGTGGCGGTCTTGTTCAGCCTGGTGGTTCTTTACGTC |
| | TTTCTTGCGCTGCTTCCGGATTCACTTTCTCTCCTTACGATATGTGGTGGGTTCGCCA |
| | AGCTCCTGGTAAAGGTTTGGAGTGGGTTTCTTATATCTCTTCTTCTGGTGGCAAGACT |
| | ATGTATGCTGACTCCGTTAAAGGTCGCTTCACTATCTCTAGAGACAACTCTAAGAATA |
| | CTCTCTACTTGCAGATGAACAGCTTAAGGGCTGAGGACACTGCAGTCTACTATTGTGC |
| | GAGATTAGGTGGTAACTCCCACTACTACTACGGTATGGACGTCTGGGGCCAAGGGACC |
| | ACGGTCACCGTCTCAAGCGCCTCCACCAAGGGCCCATCGGTCTTCCCG |
| | (SEQ ID NO:55) |
| AB d03 | GAAGTTCAATTGTTAGAGTCTGGTGGCGGTCTTGTTCAGCCTGGTGGTTCTTTACGTC |
| | TTTCTTGCGCTGCTTCCGGATTCACTTTCTCTCCTTACGATATGTGGTGGGTTCGCCA |
| | AGCTCCTGGTAAAGGTTTGGAGTGGGTTTCTTATATCTCTTCTTCTGGTGGCAAGACT |
| | ATGTATGCTGACTCCGTTAAAGGTCGCTTCACTATCTCTAGAGACAACTCTAAGAATA |
| | CTCTCTACTTGCAGATGAACAGCTTAAGGGCTGAGGACACTGCAGTCTACTATTGTGC |
| | GAGATTAGGTGGTAACTCCCACTACTACTACGGTATGGACGTCTGGGGCCAAGGGACC |
| | ACGGTCACCGTCTCAAGCGCCTCCACCAAGGGCCCATCGGTCTTCCCG |
| | (SEQ ID NO:56) |
| AB e06 | GAAGTTCAATTGTTAGAGTCTGGTGGCGGTCTTGTTCAGCCTGGTGGTTCTTTACGTC |
| | TTTCTTGCGCTGCTTCCGGATTCACTTTCTCTCCTTACACTATGAATTGGGTTCGCCA |
| | AGCTCCTGGTAAAGGTTTGGAGTGGGTTTCTCGTATCGGTTCTTCTGGTGTTTACTCA |
| | TTATGCTGACTCCGTTAAAGGTCGCTTCACTATCTCTAGAGACAACTCTAAGAATACT |
| | CTCTACTTGCAGATGAACAGCTTAAGGGCTGAGGACACTGCAGTCTACTATTGTGCGA |
| | GACCCACCCTCTATTGGTATGGTTCGGGGAGCTATTACTACTTTGACTACTGGGGCCA |
| | GGG (SEQ ID NO:58) |
| AB a04 | GAAGTTCAATTGTTAGAGTCTGGTGGCGGTCTTGTTCAGCCTGGTGGTTCTTTACGTC |
| | TTTCTTGCGCTGCTTCCGGATTCACTTTCTCTAATTACGCTATGGATTGGGTTCGCCA |
| | AGCTCCTGGTAAAGGTTTGGAGTGGGTTTCTTATATCTCTCCTTCTGGTGGCTATACT |
| | CGTTATGCTGACTCCGTTAAAGGTCGCTTCACTATCTCTAGAGACAACTCTAAGAATA |
| | CTCTCTACTTGCAGATGAACAGCTTAAGGGCTGAGGACACTGCAGTCTACTATTGTGC |
| | GAGTGACTTTGGTAGCTGGGGCCAGGGAACCCTGGTCACCGTCTCAAGCGCCTCCACC |
| | AAGGGCCCATCGGTCTTCCCG (SEQ ID NO:59) |
| AB f05 | GAAGTTCAATTGTTAGAGTCTGGTGGCGGTCTTGTTCAGCCTGGTGGTTCTTTACGTC |
| | TTTCTTGCGCTGCTTCCGGATTCACTTTCTCTGGTTACTGGATGTCTTGGGTTCGCCA |
| | AGCTCCTGGTAAAGGTTTGGAGTGGGTTTCTGTTATCCGTCCTTCTGGTGGCAAGACT |
| | GGTTATGCTGACTCCGTTAAAGGTCGCTTCACTATCTCTAGAGACAACTTTAAGAATA |
| | CTCTCTACTTGCAGATGAACAGCTTAAGGGCTGAGGACACTGCAGTCTACTATTGTGC |
| | GAGAGTAAGGGCGCCCGGCTACTACTACTACGGTATGGACGTCTGGGGCCAAGGGACC |
| | ACGGTCACCGTCTCAAGCGCCTCCACCAAGGGCCCATCGGTCTTCCCG |
| | (SEQ ID NO:60) |
| AB g03 | GAAGTTCAATTGTTAGAGTCTGGTGGCGGTCTTGTTCAGCCTGGTGGTTCTTTACGTC |
| | TTTCTTGCGCTGCTTCCGGATTCACTTTCTCTGGTTACTGGATGTCTTGGGTTCGCCA |
| | AGCTCCTGGTAAAGGTTTGGAGTGGGTTTCTGTTATCCGTCCTTCTGGTGGCAAGACT |
| | GGTTATGCTGACTCCGTTAAAGGTCGCTTCACTATCTCTAGAGACAACTTTAAGAATA |
| | CTCTCTACTTGCAGATGAACAGCTTAAGGGCTGAGGACACTGCAGTCTACTATTGTGC |
| | GAGAGTAAGGGCGCCCGGCTACTACTACTACGGTATGGACGTCTGGGGCCAAGGGACC |
| | ACGGTCACCGTCTCAAGCGCCTCCACCAAGGGCCCATCGGTCTTCCCG |
| | (SEQ ID NO:61) |
| AB e04 | GAAGTTCAATTGTTAGAGTCTGGTGGCGGTCTTGTTCAGCCTGGTGGTTCTTTACGTC |
| | TTTCTTGCGCTGCTTCCGGATTCACTTTCTCTTCTTACCTTATGACTTGGGTTGCCAA |
| | GCTCCTGGTAAAGGTTTGGAGTGGGTTTCTTCTATCTATCCTTCTGGTGGCCATACTG |
| | GTTATGCTGACTCCGTTAAAGGTCGCTTCACTATCTCTAGAGACAACTCTAAGAATAC |
| | TCTCTACTTGCAGATGAACAGCTTAAGGGCTGAGGACACTGCAGTCTACTATTGTGCG |
| | AGAGAGGGGGGATATTGTAGTAGTACCAGCTGCTATGTTGACTACTGGGGCCAGGGAA |
| | CCC (SEQ ID NO:62) |
| AB e05 | GAAGTTCAATTGTTAGAGTCTGGTGGCGGTCTTGTTCAGCCTGGTGGTTCTTTACGTC |
| | TTTCTTGCGCTGCTTCCGGATTCACTTTCTCTCGTTACGGTATGAAGTGGGTTCGCCA |
| | AGCTCCTGGTAAAGGTTTGGAGTGGGTTTCTTATATCTATCCTTCTGGTGGCTATACT |
| | CGTTATGCTGACTCCGTTAAAGGTCGCTTCACTATCTCTAGAGACAACTCTAAGAATA |
| | CTCTCTACTTGCAGATGAACAGCTTAAGGGCTGAGGACACTGCAGTCTACTATTGTGC |
| | GAGAGCCCGCGGGCATAGCAGCAGCTGGTACAATCATTACTACTACTACTACATGGAC |
| | GTCTGGGGCAAAGGGACCACGGTCACCGTCTCAAGCGCCTCCACCAAGGGCCCATCGG |
| | TCTTCCCG (SEQ ID NO:63) |
| AB d04 | GAAGTTCAATTGTTAGAGTCTGGTGGCGGTCTTGTTCAGCCTGGTGGTTCTTTACGTC |
| | TTTCTTGCGCTGCTTCCGGATTCACTTTCTCTTGGTACCATATGCGTTGGGTTCGCCA |
| | AGCTCCTGGTAAAGGTTTGGAGTGGGTTTCTATCTATCCTTCTGGTGGCGTTACTTCT |
| | TATGCTGACTCCGTTAAAGGTCGCTTCACTATCTCTAGAGACAACTCTAAGAATACTC |
| | TCTACTTGCAGATGAACAGCTTAAGGGCTGAAGACACTGCAGTCTACTATTGTGCGAG |
| | AGAAACAAGTGGCTGGTATAGGGATCGCTGGTTCGACCCCTGGGGCCAGGGAACCCTG |
| | GTCACCGTCTCAAGCGCCTCCACCAAGGGCCCATCGGTCTTCCCG |
| | (SEQ ID NO:64) |
| AB b04 | GAAGTTCAATTGTTAGAGTCTGGTGGCGGTCTTGTTCAGCCTGGTGGTTCTTTACGTC |
| | TTTCTTGCGCTGCTTCCGGATTCACTTTCTCTCAGTACAAGATGAATTGGGTTCGCCA |
| | AGCTCCTGGTAAAGGTTTGGAGTGGGTTTCTTATATCTCTCCTTCTGGTGGCTATACT |
| | GCTTATGCTGACTCCGTTAAAGGTCGCTTCACTATCTCTAGAGACAACTCTAAGAATA |
| | CTCTCTACTTGCAGATGAACAGCTTAAGGGCTGAGGACACTGCAGTCTACTATTGTGC |
| | GAGAGATGTAGTGGCTGGGCCGTTTGACTACTGGGGCCAGGGAACCCTGGTCACCGTC |
| | TCAAGCGCCTCCACCAAGGGCCCATCGGTCTTCCCG (SEQ ID NO:65) |
| AB C02 | GAAGTTCAATTGTTAGAGTCTGGTGGCGGTCTTGTTCAGCCTGGTGGTTCTTTACGTC |
| | TTTCTTGCGCTGCTTCCGGATTCACTTTCTCTGATTACTATATGCGTTGGGTTCGCCA |
| | AGCTCCTGGTAAAGGTTTGGAGTGGGTTTCTCGTATCTATCCTTCTGGTGGCCATACT |
| | TGGTATGCTGACTCCGTTAAAGGTCGCTTCACTATCTCTAGAGACAACTCTAAGAATA |
| | CTCTCTACTTGCAGATGAACAGCTTAAGGGCTGAGGACACTGCAGTCTACTATTGTGC |
| | GAGACATAGGGCGGGTAGCAGTGGCTGGTACTCTGACTACTGGGGCCAGGGAACCCTG |
| | GTCACCGTCTCAAGCGCCTCCACCAAGGGCCCATCGGTCTTCCCG |
| | (SEQ ID NO:66) |
| AB C06 | GAAGTTCAATTGTTAGAGTCTGGTGGCGGTCTTGTTCAGCCTGGTGGTTCTTTACGTC |
| | TTTCTTGCGCTGCTTCCGGATTCACTTTCTCTGATTACTATATGCGTTGGGTTCGCCA |
| | AGCTCCTGGTAAAGGTTTGGAGTGGGTTTCTCGTATCTATCCTTCTGGTGGCCATACT |
| | TGGTATGCTGACTCCGTTAAAGGTCGCTTCACTATCTCTAGAGACAACTCTAAGAATA |
| | CTCTCTACTTGCAGATGAACAGCTTAAGGGCTGAGGACACTGCAGTCTACTATTGTGC |
| | GAGACATAGGGCGGGTAGCAGTGGCTGGTACTCTGACTACTGGGGCCAGGGAACCCTG |
| | GTCACCGTCTCAAGCGCCTCCACCAAGGGCCCATCGGTCTTCCCG |
| | (SEQ ID NO:67) |
| AB d05 | GAAGTTCAATTGTTAGAGTCTGGTGGCGGTCTTGTTCAGCCTGGTGGTTCTTTACGTC |
| | TTTCTTGCGCTGCTTCCGGATTCACTTTCTCTTATTACCATATGTGGTGGGTTCGCCA |
| | AGCTCCTGGTAAAGGTTTGGAGTGGGTTTCTGTTATCGTTCCTTCTGGTGGCGGTACT |
| | CAGTATGCTGACTCCGTTAAAGGTCGCTTCACTATCTCTAGAGACAACTCTAAGAATA |
| | CTCTCTACTTGCAGATGAACAGCTTAAGGGCTGAGGACACTGCAGTCTACTATTGTGC |
| | GAGAGATGGACATAGCAGCAGCTGGTACGGTGGGGGAGCCCACTACTACGGTATGGAC |
| | GTCTGGGGCCAAGGGACCACGGTCACCGTCTCAAGCGCCTCCACCAAGGGCCCATCGG |
| | TCTTCCCG (SEQ ID NO:68) |
| AB f06 | GAAGTTCAATTGTTAGAGTCTGGTGGCGGTCTTGTTCAGCCTGGTGGTTCTTTACGTC |
| | TTTCTTGCGCTGCTTCCGGATTCACTTTCTCTTATTACCATATGTGGTGGGTTCGCCA |
| | AGCTCCTGGTAAAGGTTTGGAGTGGGTTTCTGTTATCGTTCCTTCTGGTGGCGGTACT |
| | CAGTATGCTGACTCCGTTAAAGGTCGCTTCACTATCTCTAGAGACAACTCTAAGAATA |
| | CTCTCTACTTGCAGATGAACAGCTTAAGGGCTGAGGACACTGCAGTCTACTATTGTGC |
| | GAGAGATGGACATAGCAGCAGCTGGTACGGTGGGGGAGCCCACTACTACGGTATGGAC |
| | GTCTGGGGCCAAGGGACCACGGTCACCGTCTCAAGCGCCTCCACCAAGGGCCCATCGG |
| | TCTTCCCG (SEQ ID NO:69) |
| AB a01 | GAAGTTCAATTGTTAGAGTCTGGTGGCGGTCTTGTTCAGCCTGGTGGTTCTTTACGTC |
| | TTTCTTGCGCTGCTTCCGGATTCACTTTCTCTCCTTACCGTATGGATTGGGTTCGCCA |
| | AGCTCCTGGTAAAGGTTTGGAGTGGGTTTCTTATATCTATCCTTCTGGTGGCTTTACT |
| | CCTTATGCTGACTCCGTTAAAGGTCGCTTCACTATCTCTAGAGACAACTCTAAGAATA |
| | CTTTCTACTTGCAGATGAACAGCTTAAGGGCTGAGGACACTGCAGTCTACTATTGTGC |
| | GAAAGGTTCAACGGGATACCGCTACTACTACGGTATGGACGTCTGGGGCCAAGGGACC |
| | ACGGTCACCGTCTCAAGCGCCTCCACCAAGGGCCCATCGGTCTTCCCG |
| | (SEQ ID NO:70) |
| AB e03 | GAAGTTCAATTGTTAGAGTCTGGTGGCGGTCTTGTTCAGCCTGGTGGTTCTTTACGTC |
| | TTTCTTGCGCTGCTTCCGGATTCACTTTCTCTTACAAGATGATGTGGGTTCGCCAAGC |
| | TCCTGGTAAAGGTTTGGAGTGGGTTTCTTATATCTCTTCTTCTGGTGGCATTACTACT |
| | TATGCTGACTCCGTTAAAGGTCGCTTCACTATCTCTAGAGACAACTCTAAGAATACTC |
| | TCTACTTGCAGATGAACAGCTTAAGGGCTGAGGACACTGCAGTCTACTATTGTGCGAG |
| | AGACCCGACTTACGATTTTTGGAGTGGTTATTACTACTACTACTACATGGACGTCTGG |
| | GGCAAAGGGACCACGGTCACCGTCTCAAGCGCCTCCACCAAGGGCCCATCGGTCTTCC |
| | CG (SEQ ID NO:71) |
| AB g04 | GAAGTTCAATTGTTAGAGTCTGGTGGCGGTCTTGTTCAGCCTGGTGGTTCTTTACGTC |
| | TTTCTTGCGCTGCTTCCGGATTCACTTTCTCTCTTTACCATATGGATTGGGTTCGCCA |
| | AGCTCCTGGTAAAGGTTTGGAGTGGGTTTCTGTTATCTATCCTTCTGGTGGCGGTACT |
| | CCTTATGCTGACTCCGTTAAAGGTCGCTTCACTATCTCTAGAGACAACTCTAAGAATA |
| | CTCTCTACTTGCAGATGAACAGCTTAAGGGCTGAGGACACTGCAGTCTACTATTGTGC |
| | GAGACGGGTAGGATATTGTAGTGGTGGTAGCTGCTACTACTACTACTACTACATGGAC |
| | GTCTGGGGCAAAGGGACCACGGTCACCGTCTCAAGCGCCTCCACCAAGGGCCCATCGG |
| | TCTTCCCG (SEQ ID NO:72) |
| AB a02 | GAAGTTCAATTGTTAGAGTCTGGTGGCGGTCTTGTTCAGCCTGGTGGTTCTTTACGTC |
| | TTTCTTGCGCTGCTTCCGGATTCACTTTCTCTTGGTACTGGATGAATTGGGTTCGCCA |
| | AGCTCCTGGTAAAGGTTTGGAGTGGGTTTCTTCTATCTATTCTTCTGGTGGCTATACT |
| | TCTTATGCTGACTCCGTTAAAGGTCGCTTCACTATCTCTAGAGACAACTCTAAGAATA |
| | CTCTCTACTTGCAGATGAACAGCTTAAGGGCTGAGGACACTGCAGTCTACTATTGTGC |
| | GAGAGTTCGGGATATTTTGACTGGTCCCTACTACTTTGACTACTGGGGCCAGGGAACC |
| | CTGGTCACCGTCTCAAGCGCCTCCACCAAGGGCCCATCGGTCTTCCCG |
| | (SEQ ID NO:73) |
| AB a06 | GAAGTTCAATTGTTAGAGTCTGGTGGCGGTCTTGTTCAGCCTGGTGGTTCTTTACGTC |
| | TTTCTTGCGCTGCTTCCGGATTCACTTTCTCTAATTACCGTATGCCTTGGGTTCGCCA |
| | AGCTCCTGGTAAAGGTTTGGAGTGGGTTTCTTATATCTATTCTTCTGGTGGCATTACT |
| | CAGTATGCTGACTCCGTTAAAGGTCGCTTCACTATCTCTAGAGACAACTCTAAGAATA |
| | CTCTCTACTTGCAGATGAACAGCTTAAGGGCTGAGGACACTGCAGTCTACTATTGTGC |
| | GAGATCGCGATCTTACTATGGTTCGGGGTCGTCGCGGTACTGGGGCCAGGGAACCCTG |
| | GTCACCGTCTCAAGCGCCTCCACCAAGGGCCCATCGGTCTTCCCG |
| | (SEQ ID NO:74) |
| AB f03 | GAAGTTCAATTGTTAGAGTCTGGTGGCGGTCTTGTTCAGCCTGGTGGTTCTTTACGTC |
| | TTTCTTGCGCTGCTTCCGGATTCACTTTCTCTCAGTACATGATGACTTGGGTTCGCCA |
| | AGCTCCTGGTAAAGGTTTGGAGTGGGTTTCTTATATCGGTTCTTCTGGTGGCCAGACT |
| | AAGTATGCTGACTCCGTTAAAGGTCGCTTCACTATCTCTAGAGACAACTCTAAGAATA |
| | CTCTCTACTTGCAGATGAACAGCTTAAGGGCTGAGGACACTGCAGTCTACTATTGTGC |
| | GAGGGATCCAGGGGTAGCAGTGGCTGGGTACTACTACTACGGTATGGACGTCTGGGGC |
| | CAAGGGACCACGGTCACCGTCTCAAGCGCCTCCACCAAGGGCCCATCGGTCTTCCCG |
| | (SEQ ID NO:75) |
| AB f01 | GAAGTTCAATTGTTAGAGTCTGGTGGCGGTCTTGTTCAGCCTGGTGGTTCTTTACGTC |
| | TTTCTTGCGCTGCTTCCGGATTCACTTTCTCTCAGTACAATATGCCTTGGGTTCGCCA |
| | AGCTCCTGGTAAAGGTTTGGAGTGGGTTTCTTCTATCGTTCCTTCTGGTGGCTTTACT |
| | GCTTATGCTGACTCCGTTAAAGGTCGCTTCACTATCTCTAGAGACAACTCTAAGAATA |
| | CTCTCTACTTGCAGATGAACAGCTTAAGGGCTGAGGACACTGCAGTCTACTATTGTGC |
| | GAGAGTCGATTGTAGTGGTGGTAGCTGCTACCGGGGTCCCCAAAACTACTTTGACTAC |
| | TGGGGCCAGGGAACCCTGGTCACCGTCTCAAGCGCCTCCACCAAGGGCCCATCGGTCT |
| | TCCCG (SEQ ID NO:76) |
| AB f02 | GAAGTTCAATTGTTAGAGTCTGGTGGCGGTCTTGTTCAGCCTGGTGGTTCTTTACGTC |
| | TTTCTTGCGCTGCTTCCGGATTCACTTTCTCTATGTACTATATGTTTTGGGTTCGCCA |
| | AGCTCCTGGTAAGGTTTGGAGTGGGTTTCTGTTATCGTTTCTTCTGGTGGCACTACTG |
| | AGTATGCTGACTCCGTTAAAGGTCGCTTCACTATCTCTAGAGACAACTCTAAGAATAC |
| | TCTCTACTTGCAGATGAACAGCTTAAGGGCTGAGGACACTGCAGTCTACTATTGTGCG |
| | AGAGGGGGATATTGTAGTGGTGGCAGGTGTTACACCTGGCTCGAAGACTACTGGGGCC |
| | AGG (SEQ ID NO:77) |
| AB B12 | GAAGTTCAATTGTTAGAGTCTGGTGGCGGTCTTGTTCAGCCTGGTGGTTCTTTACGTC |
| | TTTCTTGCGCTGCTTCCGGATTCACTTTCTCTTCTTACGTTATGATTTGGGTTCGCCA |
| | AGCTCCTGGTAAAGGTTTGGAGTGGGTTTCTTGGATCTCTTCTTCTGGTGGCTATACT |
| | TCTTATGCTGACTCCGTTAAAGGTCGCTTCACTATCTCTAGAGACAACTCTAAGAATA |
| | CTCTCTACTTGCAGATGAACAGCTTAAGGGCTGAGGACACTGCAGTCTACTACTGTGC |
| | GAAAGGGCCCGGGACCCGGGGTGACTACTGGGGCCAGGGAACCCTGGTCACCGTCTCA |
| | AGCGCCTCCACCAAGGGCCCATCGGTCTTCCCGCTAGCACCC (SEQ ID NO:57) |
| AB E06 | GAAGTTCAATTGTTAGAGTCTGGTGGCGGTCTTGTTCAGCCTGGTGGTTCTTTACGTC |
| | TTTCTTGCGCTGCTTCCGGATTCACTTTCTCTATTTACCCTATGAATTGGGTTCGCCA |
| | AGCTCCTGGTAAAGGTTTGGAGTGGGTTTCTGGTATCTCTCCTTCTGGTGGCTATACT |
| | GGTTATGCTGACTCCGTTAAAGGTCGCTTCACTATCTCTAGAGACAACTCTAAGAATA |
| | CTCTCTACTTGCAGATGAACAGCTTAAGGGCTGAGGACACTGCAGTCTACTATTGTGC |
| | GAGAGGGGGCATCAGCTGGTTTATGGACTACTGGGGCCAGGGAACCCTGGTCACCGTC |
| | TCAAGCGCCTCCACCAAGGGCCCATCGGTCTTCCCGCTAGCA |
| | (SEQ ID NO:349) |
| AB F05 | GAAGTTCAATTGTTAGAGTCTGGTGGCGGTCTTGTTCAGCCTGGTGGTTCTTTACGTC |
| | TTTCTTGCGCTGCTTCCGGATTCACTTTCTCTCGTTACTCTATGGGGTGGGTTCGCCA |
| | AGCTCCTGGTAAAGGTTTGGAGTGGGTTTCTTCTATCCGTCCTTCTGGTGGCTATACT |
| | CGTTATGCTGACTCCGTTAAAGGTCGCTTCACTATCTCTAGAGACAACTCTAAGAATA |
| | CTCTCTACTTGCAGATGAACAGCTTAAGGGCTGAGGACACTGCAGTCTACTATTGTGC |
| | GAAAGATCTGGAGTATAGCAGTGGCTGGTCATTTGACTACTGGGGCCAGGGAACCCTG |
| | GTCACCGTCTCAAGCGCCTCCACCAAGGGCCCATCGGTCTTCCCGCTAGCACCC |
| | (SEQ ID NO:351) |

The contents of all references, pending patent applications and published patents, cited throughout this application are hereby expressly incorporated by reference.

1. An isolated protein comprising a first and second immunoglobulin variable domain sequence, wherein the isolated protein binds to a PAPP-A molecule, and the first and/or second immunoglobulin domain is at least 85% identical to an immunoglobulin variable domain sequence of a01, a02, a03, a04, a05, a06, b01, b03, b04, b05, c01, c02, c04, c05, c06, d02, d03, d04, d05, d06, e01, e02, e03, f01, f03, f05, f06, g01, g02, g03, g04, g05, B12, E06, or F05.

2. The protein of claim 1 wherein the first and/or second immunoglobulin variable domain sequence comprises at least one CDR of an immunoglobulin variable domain sequence of a01, a02, a03, a04, a05, a06, b01, b03, b04, b05,c01, c02, c04, c05, c06, d02, d03, d04, d05, d06, e01, e02, e03, f01, f03, f05, f06, g01, g02, g03, g04, g05, B12, E06, or F05.

3. The protein of claim 1 wherein the first and/or second immunoglobulin variable domain sequence comprises CDRs that have an amino acid sequence that differs by no more than 3 substitutions, insertions or deletions for every 10 amino acids relative to corresponding CDRs of an immunoglobulin variable domain sequence of a01, a02, a03, a04, a05, a06, b01, b03, b04, b05, c01, c02, c04, c05, c06, d02, d03, d04, d05, d06, e01, e02, e03, f01, f03, f05, f06, g01, g02, g03, g04, g05, B12, E06, or F05.

4. The protein of claim 1 wherein the first and/or second immunoglobulin variable domain sequence is at least 85% identical in the CDR regions to an immunoglobulin variable domain sequence of a01, a02, a03, a04, a05, a06, b01, b03, b04, b05, c01, c02, c04, c05, c06, d02, d03, d04, d05, d06, e01, e02, e03, f01, f03, f05, f06, g01, g02, g03, g04, g05, B12, E06, or F05.

5. The protein of claim 1, wherein the first and second immunoglobulin domain sequences are identical to respective immunoglobulin variable domain sequences of a01, a02, a03, a04, a05, a06, b01, b03, b04, b05, c01, c02, c04, c05, c06, d02, d03, d04, d05, d06, e01, e02, e03, f01, f03, f05, f06, g01, g02, g03, g04, g05, B12, E06, F05.

6. The protein of 1 wherein the first and second immunoglobulin domain are components of separate polypeptide chains.

7. The protein of claim 1 herein the protein is labeled.

8. The protein of claim 1 that further comprises a cytotoxic agent.

9. The protein of claim 1 wherein the cytotoxic agent comprises an Fc domain.

10. The protein of claim 1 wherein the protein can inhibit a PAPP-A-mediated activity.

11. The protein of claim 1 wherein at least 70% of the CDR amino acid residues that are not identical to residues in the reference CDR sequences are identical to residues at corresponding positions in a human germline sequence.

12. The protein of claim 1 wherein at least 70% of the FR regions are identical to FR sequence from a human germline sequence.

13. An antibody that binds to PAPP-A and competes with, competitively inhibits binding, or binds to the same or an overlapping epitope as a01, a02, a03, a04, a05, a06, b01, b03, b04, b05, c01, c02, c04, c05, c06, d02, d03, d04, d05, d06, e01, e02, e03, f01, f03, f05, f06, g01, g02, g03, g04, g05, B12, E06, or F05.

14. A method of detecting PAPP-A, the method comprising:
providing the protein of claim 1; and
detecting binding of the protein to a sample.

15. A method of evaluating a subject, the method comprising:
administering the protein of claim 1 to a subject; and
detecting location of the protein within the subject.

16. A method of treating a subject, the method comprising:
identifying a subject in need of a PAPP-A binding protein;
administering a pharmaceutical composition comprising the protein of claim 1 to a subject in an amount effective to treat a disease or disorder.

17. The method of claim 16 wherein the disease or disorder is a proliferative disease.

18. The method of claim 16 wherein the disease or disorder comprises IGF-1 regulated growth.

19. The method of claim 16 wherein the subject has a glioblastoma.

20. The method of claim 16 wherein the subject has an osteosarcoma.

21. The method of claim 16 wherein the protein is administered by application during a surgical procedure.

22. The method of claim 16 wherein the protein is administered to a lumbar puncture.

23. The method of claim 16 wherein the subject has experienced a cardiovascular event within the previous 72 hours.

24. The method of claim 16 wherein the subject is at risk for restenosis or has restenosis.

25. The method of claim 16 wherein the subject has experienced an angioplasty within the previous 72 hours.

26. A method of modulating IGF activity in a subject, the method comprising:
providing a pharmaceutical composition comprising the protein of claim 1;
identifying a subject having a disease or disorder associated with aberrant IGF activity; and
administering the pharmaceutical composition to a subject in an amount effective to modulate IGF activity in the subject.

27. A method of reducing activity of PAPP-A in a subject, the method comprising:
identifying a subject having a disease or disorder associated with aberrant PAPP-A activity; and
administering a pharmaceutical composition comprising the protein of claim 1 to the subject in an amount effective to reduce PAPP-A activity in the subject.

28. A method of altering a cellular activity, the method comprising: providing the protein of claim 1 to the extracellular milieu of a cell, under conditions that enable the protein to interact with PAPP-A to thereby alter the IGF signalling in the cell.

29. An isolated protein comprising a light chain (LC) and heavy chain (HC) immunoglobulin variable domain sequences, wherein the isolated protein binds to a PAPP-A molecule, and comprises one or more of the following features, (A), (B), (C), (D), (E), or (F), wherein
(A) CDR1 of the LC variable region comprises:

| | |
|---|---|
| R-A-S-[QR]-[DGRS]-[VI]-[RSN]-[NRHST]-[YDEWNS]-[LVY]-[AGNL]; | (SEQ ID NO:358) |
| R-A-S-Q-X1-[VI]-X2-X3-[YDEWNS]-X4, | (SEQ ID NO:359) |

wherein X1, X2, and X3 are any amino acid, e.g., a hydrophilic amino acid and X4 is hydrophobic, e.g., aliphatic;
X1-X2-X3-X4-X5-X6-X7-X8 (SEQ ID NO:392), wherein X1 is N, Q, R, or K, X2 is hydrophilic, A, or G, X3 is aliphatic, X4 and X5 are hydrophilic, X6 is any amino acid, or aromatic or hydrophilic, and X7 is hydrophobic;

| | |
|---|---|
| S-G-S-S-S-N-I-[GEDA]-[SRV]-[NY]-[TLFD]-V-[YT]; | (SEQ ID NO:360) |
| S-G-S-S-S-N-I-[GEDA]-[SRV]-[ANY]-[TLFD]-V-[NYT]; | (SEQ ID NO:389) |
| T-G-T-S-S-D-[IV]-G-[DGY]-Y-[NED]-Y-V-S; | (SEQ ID NO:361) |
| T-G-T-S-S-D-[IV]-G-[ADGY]-Y-[NKED]-[YF]-V-S; | (SEQ ID NO:387) |
| or | |
| X1-X2-X3-G-X4-Y-X5-X6-X7-X8, | (SEQ ID NO:393) |

wherein X1 is T or S, X2 is D or E, X3 is aliphatic, X4 is hydrophilic or G, and X5 is hydrophilic or N, E, D, or Q;
(B) CDR2 of the LC variable region comprises:

| | |
|---|---|
| [ADEG]-[AVDNE]-[ASTNV]-[STNQ]-[LRN]-[AQPR]-[TFSKP]; | (SEQ ID NO:384) |
| [ADENG]-[AVDNE]-[ASTRNV]-[STENQ]-[LRN]-[AQPR]-[TFSKP]; | (SEQ ID NO:385) |
| [ADE]-[AV]-[AST]-[ST]-[LR]-[AQ]-[TFSK]; | (SEQ ID NO:386) |

[ST]-X1-X2-X3-[LRN]-[PRQ]-S (SEQ ID NO:382), wherein X1, X2, and X3 are hydrophilic;
[NST]-X1-X2-X3-[LRN]-[PRQ]-S (SEQ ID NO:388), wherein X1, X2, and X3 are hydrophilic

| | |
|---|---|
| [ST]-[DN]-[DN]-Q-R-P-S; | (SEQ ID NO:362) |
| or | |
| G-A-S-[ST]-[LR]-[QA]; | (SEQ ID NO:363) |

(C) CDR3 of the LC variable region comprises:
[QL]-Q-X1-X2-X3-X4-P-X5 (SEQ ID NO:364), wherein X1, X2, X3, X4, and X5 are any amino acid, or X1 is hydrophilic, A, or G, X2 is hydrophilic, X3 is hydrophilic, X4 is aromatic, T, R, or K, X5 is hydrophobic, and the sequence can optionally be followed by T;
Q-Q-Y-X1-X2-X3-P-[PLR]-T (SEQ ID NO:365), wherein X1 and X2 are any amino acid, and X3 is hydrophobic (e.g., aromatic);
[AGQSV]-[ATS]-X1-X2-X3-[STGA]-X4-[STRG]-[GPNF]-X5-V (SEQ ID NO:381), wherein X2, X3, and X4 are any amino acid, and X1 is aromatic, or X2 is E, D, R, T, or S, X3 is D, N, Q, K, R, or S, and X4 is S, L, T, or N;
A-W-D-D-S-L-S-G-X1-V (SEQ ID NO:366), wherein X1 is hydrophobic;
A-W-D-D-S-L-S-G-[VW]-V (SEQ ID NO:367);
A-[AT]-W-D-[DNEQ]-[ST]-L-X1-G-X2-V (SEQ ID NO:391), wherein X1 is any amino acid (e.g., S, R, T, H, N) and X2 is any amino acid, e.g., hydrophobic, e.g., V, Y, or W; or

| | |
|---|---|
| A-[AT]-W-D-[DNEQ]-[ST]-L-[SRT]-G- | (SEQ ID NO:368) |
| [VW]-V; | |

(D) CDR1 of the HC variable region comprises:
Y-X1-M-X2 (SEQ ID NO:369), wherein X1 and X2 are any amino acid, or X1 is W, D, K, T, R, H, or P, and X2 is N, W, D, E, P, T, R, S, V, or F;
X1-Y-X2-M-X3 (SEQ ID NO:370), wherein X1 is aromatic, X2 is any amino acid, and X3 is N, W, D, E, P, T, R, S, V, or F;
W-Y-X1-M (SEQ ID NO:371), wherein X1 is any amino acid, or X1 is W, H, or T; or
Q-Y-X1-M (SEQ ID NO:372), wherein X1 is any amino acid;
(E) CDR2 of the HC variable region comprises
I-X1-[PS]-S-G-G (SEQ ID NO:373), wherein X1 is any amino acid, hydrophobic or V, Y, W, R, S, or G;
I-X1-[PS]-S-G-G-X2-T (SEQ ID NO:374), wherein X1 and X2 are any amino acid;
I-X1-[PS]-S-G-G-X2-T (SEQ ID NO:375), wherein X1 is S, V, Y, W, R, or G, and X2 is G, K, L, R, H, F, Y, T, G, Q, D, M, I, or N; or
I-X1-[PS]-S-G-G-X2-T-X3-Y-A-D-S-V-K-G (SEQ ID NO:376), wherein X1 is S, V, Y, W, R, or G, and X2 and X3 are any amino acid; or
(F) CDR3 of the HC variable region comprises:

| | |
|---|---|
| D-F-G-S; | (SEQ ID NO:394) |

at least two, three, or four consecutive tyrosines;

| | |
|---|---|
| [SG]-[SG]-W-Y; | (SEQ ID NO:377) |
| S-S-[SG]-W-Y; | (SEQ ID NO:378) |
| S-S-[SG]-W-[SY] | (SEQ ID NO:383) |
| [RHWY]-Y-Y-Y-G-M; | (SEQ ID NO:379) |
| or | |
| [YSG]-[RHWY]-Y-Y-Y-G-M-D. | (SEQ ID NO:380) |

30. A protein comprising a first and second immunoglobulin variable domain sequences, wherein the first immunoglobulin variable domain sequence comprises the light chain variable domain of B12 and the second immunoglobulin variable domain sequence comprises the heavy chain variable domain of B12.

31. A protein comprising a first and second immunoglobulin variable domain sequences, wherein the first immunoglobulin variable domain sequence comprises the light chain variable domain of F05and the second immunoglobulin variable domain comprises the heavy chain variable domain of F05.

32. A nucleic acid comprising a sequence encoding at least one variable domain of the protein of claim 1 or a nucleic acid that hybridizes under stringent conditions to such a coding sequence.

33. A nucleic acid comprising a sequence encoding at least one variable domain of the protein of claim 1 or a nucleic acid that (i) hybridizes under stringent conditions to a sequence encoding at least one variable domain of the protein of claim 1, and (ii) encodes an immunoglobulin variable domain.

34. A host cell comprising a nucleic acid comprising a sequence encoding at least one variable domain of the protein of claim 1, operably linked to a promoter.

35. A host cell comprising a nucleic acid comprising a coding sequence that (i) hybridizes under stringent conditions to a sequence encoding at least one variable domain of the protein of claim 1, and (ii) encodes an immunoglobulin variable domain, wherein the sequence is operably linked to a promoter.

36. A host cell comprising a nucleic acid comprising a first coding sequence that encodes a light chain of a PAPP-A binding antibody, and a second coding sequence that encodes a heavy chain of the antibody, wherein the antibody is selected from the group consisting of a01, a02, a03, a04, a05, a06, b01, b03, b04, b05, c01, c02, c04, c05, c06, d02, d03, d04, d05, d06, e01, e02, e03, f01, f03, f05, f06, g01, g02, g03, g04, g05,B12, E06, and F05.

37. The host cell of claim 36 wherein the antibody is a full length IgG.


SEQUENCE LISTING (394 sequences)

SEQ ID NO: 1 - protein, 1627 aa, Homo sapiens
MRLWSWVLHLGLLSAALGCGLAERPRRARRDPRAGRPPRPAAGPATCATRGPRPPRLAAAAAAAGRAWEAVRVPRRRQQREARGATEEPSPPSRALYFSGRGEQLRVLRADLELPRDAFTLQVWLRAEGGQRSPAVITGLYDKCSYISRDRGWVVGIHTISDQDNKDPRYFFSLKTDRARQVTTINAHRSYLPGQWVYLAATYDGQFMKLYVNGAQVATSGEQVGGIFSPLTQKCKVLMLGGSALNHNYRGYIEHFSLWKVARTQREILSDMETHGAHTALPQLLLQENWDNVKHAWSPMKDGSSPKVEFSNAHGFLLDTSLEPPLCGQTLCDNTEVIASYNQLSSFRQPKVVRYRVVNLYEDDHKNPTVTREQVDFQHHQLAEAFKQYNISWELDVLEVSNSSLRRRLILANCDISKIGDENCDPECNHTLTGHDGGDCRHLRHPAFVKKQHNGVCDMDCNYERFNFDGGECCDPEITNVTQTCFDPDSPHRAYLDVNELKNILKLDGSTHLNIFFAKSSEEELAGVATWPWDKEALMHLGGIVLNPSFYGMPGHTHTMIHEIGHSLGLYHVFRGISEIQSCSDPCMETEPSFETGDLCNDTNPAPKHKSCGDPGPGNDTCGFHSFFNTPYNNFMSYADDDCTDSFTPNQVARMHCYLDLVYQGWQPSRKPAPVALAPQVLGHTTDSVTLEWFPPIDGHFFERELGSACHLCLEGRILVQYASNASSPMPCSPSGHWSPREAEGHPDVEQPCKSSVRTWSPNSAVNPHTVPPACPEPQGCYLELEFLYPLVPESLTIWVTFVSTDWDSSGAVNDIKLLAVSGKNISLGPQNVFCDVPLTIRLWDVGEEVYGIQIYTLDEHLEIDAAMLTSTADTPLCLQCKPLKYKVVRDPPLQMDVASILHLNRKFVDMDLNLGSVYQYWVITISGTEESEPSPAVTYIHGRGYCGDGIIQKDQGEQCDDMNKINGDGCSLFCRQEVSFNCIDEPSRCYFHDGDGVCEEFEQKTSIKDCGVYTPQGFLDQWASNASVSHQDQQCPGWVIIGQPAASQVCRTKVIDLSEGISQHAWYPCTISYPYSQLAQTTFWLRAYFSQPMVAAAVIVHLVTDGTYYGDQKQETISVQLLDTKDQSHDLGLHVLSCRNNPLIIPVVHDLSQPFYHSQAVRVSFSSPLVAISGVALRSFDNFDPVTLSSCQRGETYSPAEQSCVHFACEKTDCPELAVENASLNCSSSDRYHGAQCTVSCRTGYVLQIRRDDELIKSQTGPSVTVTCTEGKWNKQVACEPVDCSIPDHHQVYAASFSCPEGTTFGSQCSFQCRHPAQLKGNNSLLTCMEDGLWSFPEALCELMCLAPPPVPNADLQTARCRENKHKVGSFCKYKCKPGYHVPGSSRKSKKRAFKTQCTQDGSWQEGACVPVTCDPPPPKFHGLYQCTNGFQFNSECRIKCEDSDASQGLGSNVIHCRKDGTWNGSFHVCQEMQGQCSVPNELNSNLKLQCPDGYAIGSECATSCLDHNSESIILPMNVTVRDIPHWLNPTRVERVVCTAGLKWYPHPALIHCVKGCEPFMGDNYCDAINNRAFCNYDGGDCCTSTVKTKKVTPFPMSCDLQGDCACRDPQAQEHSRKDLRGYSHG

SEQ ID NO: 2 - protein, 1547 aa, Homo sapiens
EARGATEEPSPPSRALYFSGRGEQLRVLRADLELPRDAFTLQVWLRAEGGQRSPAVITGLYDKCSYISRDRGWVVGIHTISDQDNKDPRYFFSLKTDRARQVTTINAHRSYLPGQWVYLAATYDGQFMKLYVNGAQVATSGEQVGGIFSPLTQKCKVLMLGGSALNHNYRGYIEHFSLWKVARTQREILSDMETHGAHTALPQLLLQENWDNVKHAWSPMKDGSSPKVEFSNAHGFLLDTSLEPPLCGQTLCDNTEVIASYNQLSSFRQPKVVRYRVVNLYEDDHKNPTVTREQVDFQHHQLAEAFKQYNISWELDVLEVSNSSLRRRLILANCDISKIGDENCDPECNHTLTGHDGGDCRHLRHPAFVKKQHNGVCDMDCNYERFNFDGGECCDPEITNVTQTCFDPDSPHRAYLDVNELKNILKLDGSTHLNIFFAKSSEEELAGVATWPWDKEALMHLGGIVLNPSFYGMPGHTHTMIHEIGHSLGLYHVFRGISEIQSCSDPCMETEPSFETGDLCNDTNPAPKHKSCGDPGPGNDTCGFHSFFNTPYNNFMSYADDDCTDSFTPNQVARMHCYLDLVYQGWQPSRKPAPVALAPQVLGHTTDSVTLEWFPPIDGHFFERELGSACHLCLEGRILVQYASNASSPMPCSPSGHWSPREAEGHPDVEQPCKSSVRTWSPNSAVNPHTVPPACPEPQGCYLELEFLYPLVPESLTIWVTFVSTDWDSSGAVNDIKLLAVSGKNISLGPQNVFCDVPLTIRLWDVGEEVYGIQIYTLDEHLEIDAAMLTSTADTPLCLQCKPLKYKVVRDPPLQMDVASILHLNRKFVDMDLNLGSVYQYWVITISGTEESEPSPAVTYIHGRGYCGDGIIQKDQGEQCDDMNKINGDGCSLFCRQEVSFNCIDEPSRCYFHDGDGVCEEFEQKTSIKDCGVYTPQGFLDQWASNASVSHQDQQCPGWVIIGQPAASQVCRTKVIDLSEGISQHAWYPCTISYPYSQLAQTTFWLRAYFSQPMVAAAVIVHLVTDGTYYGDQKQETISVQLLDTKDQSHDLGLHVLSCRNNPLIIPVVHDLSQPFYHSQAVRVSFSSPLVAISGVALRSFDNFDPVTLSSCQRGETYSPAEQSCVHFACEKTDCPELAVENASLNCSSSDRYHGAQCTVSCRTGYVLQIRRDDELIKSQTGPSVTVTCTEGKWNKQVACEPVDCSIPDHHQVYAASFSCPEGTTFGSQCSFQCRHPAQLKGNNSLLTCMEDGLWSFPEALCELMCLAPPPVPNADLQTARCRENKHKVGSFCKYKCKPGYHVPGSSRKSKKRAFKTQCTQDGSWQEGACVPVTCDPPPPKFHGLYQCTNGFQFNSECRIKCEDSDASQGLGSNVIHCRKDGTWNGSFHVCQEMQGQCSVPNELNSNLKLQCPDGYAIGSECATSCLDHNSESIILPMNVTVRDIPHWLNPTRVERVVCTAGLKWYPHPALIHCVKGCEPFMGDNYCDAINNRAFCNYDGGDCCTSTVKTKKVTPFPMSCDLQGDCACRDPQAQEHSRKDLRGYSHG

SEQ ID NO: 3 - DNA, 405 bp, Unknown
  Light Chain nucleic acid sequence
gtgaaaaaattattattcgcaattcctttagttgttcctttctattctcacagtgcacaagacatccagatgacacagtctccatcctccctgtctgcatctgttggagacagagtcaccatcacttgccgggctagtcagcgcattagtagttatgtaaattggtatcaacagaaaccagggaaagcccctaagctcctgatctattctgcatccagtttacaaagtggggtcccatcaaggttcagtggcagtgtatctgggacagagttcactctcaccatcagcagtctgcaacctgaggattttgcaacttactactgtcaacagagttaccgtacccctcctttttttggccaggggaccaagctggaggtcaaacgaactgtggctgcaccatctgtc

SEQ ID NO: 4 - DNA, 405 bp, Unknown
  Light Chain nucleic acid
gtgaaaaaattattattcgcaattcctttagttgttcctttctattctcacagtgcacaagacatccagatgacccagtctccagccaccctgtatgtgtctccgggggaaagagccaccctctcctgcagggccagtcagagtgttagtaggaacttagcctggtaccagcagaaacctggccaggctcccaggctcctcatctatggtgcatccaccagggccactggtatcccagccaggttcagtggcagtgggtctcggacagagttcactctcaccatcagcagcctgcagtctgaagattttgcagtttatcactgtcagcagtataatagcaggcctctcactttcggcggagggaccaaggtggagatcaaacgaactgtggctgcaccatctgtc

SEQ ID NO: 5 - DNA, 405 bp, Unknown
  Light Chain nucleic acid sequence
gtgaaaaaattattattcgcaattcctttagttgttcctttctattctcacagtgcacaagacatccagatgacccagtctccagccaccctgtctgtgtctccaggggaaagagccaccctctcctgcagggccagtcagagtgttcgcagctacttagcctggtaccagcagaaaccaggccaggctcccaggctcctcatctatgatgcatccaccagggccactggtatcccagccagattcagtggcagtgggtctgggacagagttcactctcaccatcagcagcctgcagtctgaagattttgcagtttattactgtcagcagtataataactggcctccgacgttcggccaagggaccaaggtggaaatcaaacgaactgtggctgcaccatctgtc

SEQ ID NO: 6 - DNA, 405 bp, Artificial Sequence
  Light Chain nucleic acid sequence
gtgaaaaaattattattcgcaattcctttagttgttcctttctattctcacagtgcacaagacatccagatgacccagtctccagccaccctgtctgtgtctccaggggaaagagccaccctctcctgcagggccagtcaggatgttaacagatacttagcctggtaccagcagaaacctggccagcctcccaggctcctcatctatggtgcctctaccagggccactggtatcccagccaggatcagtggcagtgggtctgggacagagttcactctcaccatcagcagcctgcagtctgaagattttgcagtttattactgtcagcagtatcataactggcccctcactttcggcggagggaccaaggtggagatcaaacgaactgtggctgcaccatctgtc

SEQ ID NO: 7 - DNA, 405 bp, Artificial Sequence
  Light Chain nucleic acid sequence
gtgaaaaaattattattcgcaattcctttagttgttcctttctattctcacagtgcacaagacatccagatgacccagtctccagccaccctgtctttgtctccaggggaaagagccaccctctcctgcagggccagtcagagtgttagcagctacttagcctggtaccaacagaaacctggccaggctcccaggctcctcatctatggtgcatccagcagggccactggcatcccagacaggttcagtggcagtgggtctgggacagacttcactctcaccatcggcagactggagcctgaagattttgcagtgtattactgtcagcagtatagtagttcaccggtcaccttcggccaagggacacgactggagattaaacgaactgtggctgcaccatctgtc

SEQ ID NO: 8 - DNA, 402 bp, Artificial Sequence
  Light Chain nucleic acid sequence
gtgaaaaaattattattcgcaattcctttagttgttcctttctattctcacagtgcacaagacatccagatgacccagtctccagccaccctgtctttgtctccaggggaaagagccaccctctcctgcagggccagtcagagtgttagcaggtacttagcctggtaccaacagaaacctggccaggctcccaggctcctcatctatggtgcatccaccagggccactggtatcccagccaggttcagtggcagtgggtctgggacagagttcactctcaccatcagcagcctgcagtctgaagattttgcagtttattactgtcagcagtataataactggccttctttcggcggagggaccaaggtggagatcaaacgaactgtggctgcaccatctgtc

SEQ ID NO: 9 - DNA, 408 bp, Artificial Sequence
  Light Chain nucleic acid sequence
gtgaaaaaattattattcgcaattcctttagttgttcctttctattctcacagtgcacaagacatccagatgacccagtctccaggcaccctgtctttgtctccaggggaaagagccaccctctcctgcagggccagtcagagtattagcagcagctacttagcctggtaccagcagaaacctggccaggctcccaggctcctcatctatgctgcagccagcagggccactggcatcccagacaggttcagtggcattgggtctgggacagacttcactctcaccatcagcagcctagagcctgaagattttgcagtttattactgtcagcagcgtagcaactggcctctcactttcggcggagggaccaaggtggagatcaaacgaactgtggctgcaccatctgtc

SEQ ID NO: 10 - DNA, 408 bp, Artificial Sequence
  Light Chain nucleic acid sequence
gtgaaaaaattattattcgcaattcctttagttgttcctttctattctcacagtgcacaagacatccagatgacccagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcaccgtggacgttcggccaagggaccaaggtggaaatcaaacgaactgtggctgcaccatctgtc

SEQ ID NO: 11 - DNA, 405 bp, Artificial Sequence
  Light Chain nucleic acid sequence
gtgaaaaaattattattcgcaattcctttagttgttcctttctattctcacagtgcacaagacatccagatgacccagtctccatcctcactgtctgcatctgtaggagacagagtcaccatcacttgtcgggcgagtcaggacattagcaattatttagcctggtttcagcagaaaccagggagagcccctaagtccctgatctatggtgcatccagtttgcaaactggggtcccatcaaagttcagcggcagtggatctgggacagagttcactctcaccatcagcggcctgcagcctgaagatgttgcaacttattactgccatcagtataatcattaccctcccactttcggcggagggaccaaggtggagatcaaacgaactgtggctgcaccatctgtc

SEQ ID NO: 12 - DNA, 405 bp, Artificial Sequence
  Light Chain nucleic acid sequence
gtgaaaaaattattattcgcaattcctttagttgttcctttctattctcacagtgcacaagacatccagatgacccagtctccatcctcactgtctgcatctgtaggagacagagtcaccatcacttgtcgggcgagtcaggacattaggaattatttagcctggtttcagcagaaaccaggggaagcccctaagtccctgatctatgctgcgtccagtttgcagagtggggtctcatcaaacttcagcggcagtggatctgggacagatttcactctcaccatcagcagcctgcagcctgaagattttgcaacttattactgccagcagtatcataggtacccgaggacttttggtcaggggaccaagctggagatcaaacgaactgtggctgcaccatctgtc

SEQ ID NO: 13 - DNA, 560 bp, Artificial Sequence
  Light Chain nucleic acid sequence
gtgaaaaaattattattcgcaattcctttagttgttcctttctattctcacagtgcacaagacatccagatgacccagtctccatcctcattctctgcatctacaggagacagagtcaccatcacttgtcgggcgagtcagggtattagcagttatttagcctggtatcagcaaaaaccagggaaagcccctaagctcctgatctatgctgcatccactttgcaaagtggggtcccatcaaagttcagcggcagtggatctgggacagatttcactctcaccatcagcagcctgcagcctgaagattttgcaacttattactgccaacagtataatagttaccccctcaccttcggccaagggacacgactggagattaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcagga

SEQ ID NO: 14 - DNA, 405 bp, Artificial Sequence
  Light Chain nucleic acid sequence
gtgaaaaaattattattcgcaattcctttagttgttcctttctattctcacagtgcacaagacatccagatgacccagtctccatcctccctgtatgcatctgtaggagacagagtcaccatcacttgccgggcaagtcagggcattagaaatgagttaggttggtatcagcagaaaccagggaaagcccctcagcgcctgatctatgatgcatccactttgcagagtggggtcccatcaagattcagcggcggtggatctaggacagaattcactctcaccatcagcagcctggaacctcatgattttggaacttattactgccaacaatatgccagttatccgctcactttcggcggagggaccaaggtggagatcaaacgaactgtggctgcaccatctgtc

SEQ ID NO: 15 - DNA, 405 bp, Artificial Sequence
  Light Chain nucleic acid sequence
gtgaaaaaattattattcgcaattcctttagttgttcctttctattctcacagtgcacaagacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtaccaggtggacgttcggccaagggaccaaggtggaaatcaaacgaactgtggctgcaccatctgtc

SEQ ID NO: 16 - DNA, 405 bp, Artificial Sequence; identical to SEQ ID NO: 15
  Light Chain nucleic acid sequence

SEQ ID NO: 17 - DNA, 560 bp, Artificial Sequence
  Light Chain nucleic acid sequence
gtgaaaaaattattattcgcaattcctttagttgttcctttctattctcacagtgcacaagacatccagatgacccagtctccatcctccctgtctgcatctgtaggagacagagtcaccatcacttgccgggcaagtcagggcattagaaatgatttaggctggtttcagcagaaaccagggaaagcccctaggcgcctgatctggggtgcatccactttacaaagtggggtcccatcaaggttcagcggcagtggatctggcacagatttcactctcaccatcagcagcctgcagcctgaagattttgcaacttattactgtctacaagattacaattacccgtacacttttggccaggggaccaagctggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcagga

SEQ ID NO: 18 - DNA, 405 bp, Artificial Sequence
  Light Chain nucleic acid sequence
gtgaaaaaattattattcgcaattcctttagttgttcctttctattctcacagtgcacaagacatccagatgacccagtctccatcctccctgtctgcatctgtaggagacagagtcaccatcacttgccgggcaagtcagggcattagacattatttaggctggtatcagcagaaaccagggaaagcccctaagcgcctgatctatgctgcatccagtttgcaatttggggtcccagcaaggttcagcggcagtggatctgggacggaattcactctcacaatcagcagcctgcagcctgaagattttgcaacttattactgtctacaacacaatagtttccctccggcgttcggccaagggaccaaggtggaaatcaaacgaactgtggctgcaccatctgtc

SEQ ID NO: 19 - DNA, 405 bp, Artificial Sequence; identical to SEQ ID NO: 18
  Light Chain nucleic acid sequence

SEQ ID NO: 20 - DNA, 405 bp, Artificial Sequence; identical to SEQ ID NO: 18
  Light Chain nucleic acid sequence

SEQ ID NO: 21 - DNA, 405 bp, Artificial Sequence
  Light Chain nucleic acid sequence
gtgaaaaaattattattcgcaattcctttagttgttcctttctattctcacagtgcacaagacatccagatgacccagtctccatcctccctgtctgcatctgtaggagacagagtcaccatctcttgccgcgcaagtcagaacattaggaactctgtaaattggtatcagcagaaaccagggaaagcccctaagctcctgatctatgctacatacgatttgcagagtggcgccccatcatacttcagtggcagtggatctgggacagatttcactctcaccatcaccagtctgcaacctgaagattttgcaacttactactgtcaacagagttacagtttccctcgaacgttcggccaagggaccaaggtggaaatcagacgaactgtggctgcaccatctgtc

SEQ ID NO: 22 - DNA, 405 bp, Artificial Sequence
  Light Chain nucleic acid sequence
gtgaaaaaattattattcgcaattcctttagttgttcctttctattctcacagtgcacaagacatccagatgacccagtctccatcttccgtgtctgcatctgtaggagacagaatcgccatcacttgtcgggcgagtcagggtattagcacctggttagcctggtatcagcagagaccagggagagcccctaagctcctgatctatgctgcatccactttgcaaagcggagtcccatcaaggttcagcggcagtggatctgggacagatttcactctcaccatcagcagcctgcagcctgaagattttgcaacttacttttgtcaacaggctgacagtttccccctgacttttggccaggggaccaaactggagatcaaacgaactgtggctgcaccatctgtc

SEQ ID NO: 23 - DNA, 405 bp, Artificial Sequence
  Light Chain nucleic acid sequence
gtgaaaaaattattattcgcaattcctttagttgttcctttctattctcacagtgcacaagacatccagatgacccagtctccatcttccgtgtctgcatctgtaggagacagagtcaccatcacttgtcgggcgagtcggggtattagcagatggttagcctggtatcagcagaaaccagggaaagcccctaagctcctgatctatggtgcatccactttgcaaaaaggggtcccatcaaggttcaccggcagtggatctgggacagatttcactctcaccatcaccagcctgcagcctgaagattttgcaacttactattgtcaacagggtaacagtttcccattcactttcggccctgggaccaaagtggatatcaaacgaactgtggctgcaccatctgtc

SEQ ID NO: 24 - DNA, 405 bp, Artificial Sequence; identical to SEQ ID NO: 23
  Light Chain nucleic acid sequence

SEQ ID NO: 25 - DNA, 405 bp, Artificial Sequence
  Light Chain nucleic acid sequence
gtgaaaaaattattattcgcaattcctttagttgttcctttctattctcacagtgcacaagacatccagatgacccagtctccgtcttccgtgtctgcatctgtaggagacagagtcaccatcacttgtcgggcgagtcagggtattagcagctggttagcctggtatcagcagaagccagggaaagcccctaagttgctgatctatggtgcatccagtttggaaagtggggtcccatcaagattcagcggcagtggatctgggacagattacactctcaccatcaccagcctacagcctgaagattttgcaacttacttttgtcaacaggttaattctttccctcgtacttttggccaggggaccaagctgaatatcaaacgaactgtggctgcaccatctgtc

SEQ ID NO: 26 - DNA, 539 bp, Artificial Sequence
  Light Chain nucleic acid sequence
gtgaaaaaattattattcgcaattcctttagttgttcctttctattctcacagtgcacagagcgaattgactcagataagggccagggctctggagtccccagccgcttctctggatccaaagatgctgcagccaatgcaggggttttactcatctccggcctccagcccgaggatgatgctgactatttttgtatgatatggttaagcaatgtacatgcgacattcggcggagggaccaagctgaccgtcctgggtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctaagcctgacgcctgagcagtggaagtcccacagaagctacagctgccaggtcacgcatgaagggagcacc

SEQ ID NO: 27 - DNA, 411 bp, Artificial Sequence
  Light Chain nucleic acid sequence
gtgaaaaaattattattcgcaattcctttagttgttcctttctattctcacagtgcacagagcgaattgactcagccaccctcagcgtctgcgacccccgggcagagggtcaccatctcttgttctggaagcagctccaacatcggacgtaatttggtatactggtaccagcagctcccaggaacggcccccaaactcctcatctatagtaataatcagcggccctcaggggtccctgaccgattctctggctccaagtctggcacctcagcctccctggccatcagtgggctccggtccgaggaggaggctgattattactgtgcagcatgggatgacagcctgagtggttgggtgttcggcggagggaccaggctgaccgtcctaggtcagcccaaggctgccccctcg

SEQ ID NO: 28 - DNA, 411 bp, Artificial Sequence
  Light Chain nucleic acid sequence
gtgaaaaaattattattcgcaattcctttagttgttcctttctattctcacagtgcacagagcgctttgactcagccaccctcagcgtctgggacccccgggcagagggtcaccatctcttgttctggaagcagctccaacatcggaagtaattttgtatactggtaccaccatctcccaggaacggcccccaaactcctcatctataggaataatcagcggccctcaggggtccctgaccgattctctggctccaagtctggcacctcagcctccctggccatcagtgggctccggtccgaggatgaggctgattattactgtgcagcatgggatgacagcctgagtggggtggtattcggcggagggaccaagctgaccgtcctaggtcagcccaaggctgccccctcg

SEQ ID NO: 29 - DNA, 414 bp, Artificial Sequence
  Light Chain nucleic acid sequence
gtgaaaaaattattattcgcaattcctttagttgttcctttctattctcacagtgcacagagcgctttgactcagcctgcctccgtgtctgggtctcctggacagtcgatcaccatctcctgcactggaaccagcagtgacgttggttattatgactatgtctcctggtaccagcaccacccaggcaaagcccccaaactcatcatttatgatgtcacttctcggccctcaggggtctcttctcatttctctggctccaagtctggcaacacggcctccctgaccatctctgggctccaggctgatgacgaggctgattattactgcagctcatatacaagcggcagcacccgttatgtcttcggacctgggaccaaggtcaccgtcctaggtcagcccaaggccaaccccact

SEQ ID NO: 30 - DNA, 414 bp, Artificial Sequence; identical to SEQ ID NO: 29
  Light Chain nucleic acid sequence

SEQ ID NO: 31 - DNA, 414 bp, Artificial Sequence
  Light Chain nucleic acid sequence
gtgaaaaaattattattcgcaattcctttagttgttcctttctattctcacagtgcacagagcgtcttgactcagactgcctccgtgtctgggtctcctggacagtcgatcaccatctcctgcactggaaccagcagtgacattggtgattatgagtatgtctcctggtaccaacaacacccaggcaaagcccccaaagtcattctttatgaggtcagtaatcggccctcaggggtccctgatcgcttctctggctccaagtctggcaacacggcctcactgaccatctctggactccaggctgaggacgaggctgattattactgtggttcatatagaaagagcagcactccttatgtcttcggaactgggaccaaggtcagcgtcctaggtcagcccaaggccaaccccact

SEQ ID NO: 32 - DNA, 414 bp, Artificial Sequence; identical to SEQ ID NO: 31
  Light Chain nucleic acid sequence

SEQ ID NO: 33 - DNA, 560 bp, Artificial Sequence
  Light Chain nucleic acid sequence
gtgaaaaaattattattcgcaattcctttagttgttcctttctattctcacagtgcacagagcgtcttgactcagccaccctcagcgtctgggacccccgggcagagggtcaccatctcttgttctggaagcagctccaacatcgaaagtaatactgtaacctggtaccagcaactcccaggaacggcccccaaactcctcatctatagtgatgatcagcggccctcaggggtccctgaccgattctctggatccaagtctggcacctcagcctccctggccatcagtgggctccagtctgaggatgaggctgactattactgtgcaacatgggataacaccctgagaggtgtggttttcggcggagggaccaagctgaccgtcctgagtcagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacacc

SEQ ID NO: 34 - DNA, 429 bp, Artificial Sequence
  Light Chain nucleic acid sequence
gtgaaaaaattattattcgcaattcctttagttgttcctttctattctcacagtgcacagagcgtcttgactcagccaccttatgcatcagcctccctgggagcctcggtcacactcacctgcaccctgagcagcggctacagtaattataaagtggactggtatcagcaaagaccagggaagggcccccagtttgtgatgcgagtgggcagtggcgggattgtgggatcaaagggggatggcatccctgatcgcttttcagtcctgggctcaggcctgtatcggtatctgaccatcaagaacatccaggaagaggatgagagtgactactattgtggggcagaccatggcagggggggcaccttcgtgtgggtgttcggcggagggaccaaactgaccgtcctaggtcagcccaaggctgccccctcg

SEQ ID NO: 35 - DNA, 411 bp, Artificial Sequence
  Light Chain nucleic acid sequence
gtgaaaaaattattattcgcaattcctttagttgttcctttctattctcacagtgcacagagcgtcttgactcagcctgcctccgtgtctgggtctcctggacagtcgatcaccatctcctgcactggaaccagcagtgacgttggtggttataactatgtctcctggtaccaacgacacccaggcaaagcccccaaactcattatttatgatgtcactaatcgcccctcaggggcttctcgtcacttctctggctccaagtctggcaacacggcctccctgaccatctctggtctccaggccgacgacgaggctgattattattgcgtttcatttacaaacagcaatactttcgtcttcggaagtgggaccagggtcaccgtcctcggtcagcccaaggccaaccccact

SEQ ID NO: 36 - DNA, 417 bp, Artificial Sequence
  Light Chain nucleic acid sequence
gtgaaaaaattattattcgcaattcctttagttgttcctttctattctcacagtgcacaggacatcgtcatgactcaaacccctcctagtttaccggttaacccgggtgaacctgcctccatctcctgcaagtctagtcagagcctcctgcagagtaatggatacaactacttggattggtacctgcagaagccagggcagtctccacagctcctgatctatttgggttctaatcgggcctccggggtccctgacaggttcagtggcagtggatcaggcacagattttacactgaagatcagcagggtggaggctgaggatgttggcatttattactgcatgcaagctctacacactcctcccttcggccaagggacacgactggagattaaacgaactgtggctgcaccatctgtc

SEQ ID NO: 37 - DNA, 405 bp, Artificial Sequence
  Light Chain nucleic acid sequence
gtgaaaaaattattattcgcaattcctttagttgttcctttctattctcacagtgcacagtacgaattgactcagccaccctcagtgtccgtgtccccgggacagacagccaccattatctgctctggagataaattgggggataaatatgttgcctggtatcagcagaagccaggccagtcccctgtgctggtcgtctatgaagataacaagcggccctcagggatccctgagcgaatttctggctccaactctgggaacacagccactctgaccatcagtgggacccaggctatggatgacgctgactattactgtcaggcgtgggacagaagcactgaccattatgtcttcggaactgggaccaaggtcaccgtcctaggtcagcccaaggccaaccccact

SEQ ID NO: 38 - DNA, 414 bp, Artificial Sequence
  Light Chain nucleic acid sequence
gtgaaaaaattattattcgcaattcctttagttgttcctttctattctcacagtgcacagtacgaattgactcagcctgcctccgtgtctgggtctcctggacagtcgatcaccatctcctgcactggaaccagcagcgacgttggtggttataactatgtctcctggtaccaacagcacccaggcaaagcccccaaactcatgatttatgaggtcagtaatcggccctcaggggtttctaatcgcttctctggctccaagtctgacaatacggcctccctgaccatctctggactccaggctgaggacgaggctgattattactgtggttcatatagaaagagcagcactccttatgtcttcggaactgggaccaaggtcagcgtcctaggtcagcccaaggccaaccccact

SEQ ID NO: 39 - DNA, 413 bp, Artificial Sequence
  Light Chain nucleic acid sequence
gtgaaaaaattatttattcgcaatttcctttagttgttcctttctattctcacagtgcacagagcgctttgactcagccatcctcagcgtctgggacccccgggcagagggtcagtatctcttgttctggaagcagctacaacatcggagtttatgatgtatactggtaccagcagctcccaggaacggcccccaaactcctcatctataccaataatcagcggccctcaggggtccctgaccgattctctggctccaagtctggcacctcagcctccctggccatcagtgggctccagtctgaggatgaggctgattattactgtgcagcatgggatgacagtctgagtggttgggtgttcggcggagggaccaaggtgaccgtcctaggtcagcccaaggctgccccctcg

SEQ ID NO: 40 - DNA, 387 bp, Artificial Sequence
  Heavy Chain nucleic acid sequence
gaagttcaattgttagagtctggtggcggtcttgttcagcctggtggttctttacgtctttcttgcgctgcttccggattcactttctctctttaccagatgctttgggttcgccaagctcctggtaaaggtttggagtgggtttctggtatcgtttcttctggtggccttactggttatgctgactccgttaaaggtcgcttcactatctctagagacaactctaagaatactctctacttgcagatgaacagcttaagggctgaggacactgcagtctactattgtgcgagacataatagggctattggcacctttgactactggggccagggaaccctggtcaccgtctcaagcgcctccaccaagggcccatcggtcttcccg

SEQ ID NO: 41 - DNA, 369 bp, Artificial Sequence
  Heavy Chain nucleic acid sequence
gaagttcaattgttagagtctggtggcggtcttgttcagcctggtggttctttacgtctttcttgcgctgcttccggattcactttctcttggtactttatgaattgggttcgccaagctcctggtaaaggtttggagtgggtttcttctatctatccttctggtggctatactatgtatgctgactctgttaaaggtcgcttcactatctctagagacaactctaagaagactctctacttgcagatgaacagcttaagggctgaggacactgcagtctactattgtgcgagtgactttggtagctggggccagggaaccctggtcaccgtctcaagcgcctccaccaagggcccatcggtcttcccg

SEQ ID NO: 42 - DNA, 369 bp, Artificial Sequence
  Heavy Chain nucleic acid sequence
gaagttcaattgttagagtctggtggcggtcttgttcagcctggtggttctttacgtctttcttgcgctgcttccggattcactttctcttggtaccgtatgaattgggttcgccaagctcctggtaaaggtttggagtgggtttcttctatcgttccttctggtggctatactcgttatgctgactccgttaaaggtcgcttcactatctctagagacaactctaagaatactctctacttgcagatgaacagcttaagggctgaggacactgcagtctactattgtgcgagtgactttggtagctggggccagggaaccctggtcaccgtctcaagcgcctccaccaagggcccatcggtcttcccg

SEQ ID NO: 43 - DNA, 369 bp, Artificial Sequence
  Heavy Chain nucleic acid sequence
gaagttcaattgttagagtctggtggcggtcttgttcagcctggtggttctttacgtctttcttgcgctgcttccggattcactttctctcgttactctatgaattgggttcgccaagctcctggtaaaggtttggagtgggtttcttatatctctccttctggtggcatgactaagtatgctgactccgttaaaggtcgcttcactatctctagagacaactctaagaatactctctacttgcagatgaacagcttaagggctgaggacactgcagtctactattgtgcgaatacccttggctactggggccagggaaccctggtcaccgtctcaagcgcctccaccaagggcccatcggtcttcccg

SEQ ID NO: 44 - DNA, 369 bp, Artificial Sequence
  Heavy Chain nucleic acid sequence
gaagttcaattgttagagtctggtggcggtcttgttcagcctggtggttctttacgtctttcttgcgctgcttccggattcactttctcttcttaccgtatgaattgggttcgccaagctcctggtaaaggtttggagtgggtttctggtatcgttccttctggtggcaagactttttatgctgactccgttaaaggtcgcttcactatctctagagacaactctaagaatactctctacttgcagatgaacagcttaagggctgaggacactgcagtctactattgtgcgagtgactttggtagctggggccagggaaccctggtcaccgtctcaagcgcctccaccaagggcccatcggtcttcccg

SEQ ID NO: 45 - DNA, 369 bp, Artificial Sequence
  Heavy Chain nucleic acid sequence
gaagttcaattgttagagtctggtggcggtcttgttcagcctggtggttctttacgtctttcttgcgctgcttccggattcactttctctaattactctatggattgggttcgccaagctcctggtaaaggtttggagtgggtttcttggatctctccttctggtggccttactacttatgctgactccgttaaaggtcgcttcactatctctagagacaactctaagaatactctctacttgcagatgaacagcttaagggctgaggacactgcagtctactattgtgcgagtgactttggtagctggggccagggaaccctggtcaccgtctcaagcgcctccaccaagggcccatcggtcttcccg

SEQ ID NO: 46 - DNA, 393 bp, Artificial Sequence
  Heavy Chain nucleic acid sequence
gaagttcaattgttagagtctggtggcggtcttgttcagcctggtggttctttacgtctttcttgcgctgcttccggattcactttctctcgttaccatatggagtgggttcgccaagctcatggtaaaggtttggagtgggtttcttatatctctccttctggtggcaagactctttatgctgactccgttaaaggtcgcttcactatctctagagacaactctaagaatactctctacttgcagatgaacagcttaagggctgaggacactgcagtctactattgtgcgagacatttgggatatggttcggggagttactttgactactggggccagggaaccctggtcaccgtctcaagcgcctccaccaagggcccatcggtcttcccg

SEQ ID NO: 47 - DNA, 390 bp, Artificial Sequence
  Heavy Chain nucleic acid sequence
gaagttcaattgttagagtctggtggcggtcttgttcagcctggtggttctttacgtctttcttgcgctgcttccggattcactttctctttttaccctatgccttgggttcgccaagctcctggtaaaggtttggagtgggtttcttatatctctccttctggtggcgatactacttatgctgactccgttaaaggtcgcttcactatctctagagacaactctaagaatactttctacttgcagatgaacagcttaagggctgaggacactgcagtctactattgtgcgagaggggggtcctatagcagcagttggtacggctactggggccagggaaccctggtcaccgtctcaagcgcctccaccaagggcccatcggtcttcccg

SEQ ID NO: 48 - DNA, 387 bp, Artificial Sequence
  Heavy Chain nucleic acid sequence
gaagttcaattgttagagtctggtggcggtcttgttcagcctggtggttctttacgtctttcttgcgctgcttccggattcactttctctaagtaccctatgttttgggttcgccaagctcctggtaaaggtttggagtgggtttcttggatctctccttctggtggcaagactgtttatgctgactccgttaaaggtcgcttcactatctctagagacaactctaagaatactctctacttgcagatgaacagcttaagggctgaggacactgcagtctactattgtgcgaaagattgcagagggggttgcagtggtggaagttggggccagggaaccctggtcaccgtctcaagcgcctccaccaagggcccatcggtcttcccg

SEQ ID NO: 49 - DNA, 393 bp, Artificial Sequence
  Heavy Chain nucleic acid sequence
gaagttcaattgttagagtctggtggcggtcttgttcagcctggtggttctttacgtctttcttgcgctgcttccggattcactttctctgcttacaatatgccttgggttcgccaagctcctggtaaaggtttggagtgggtttcttatatctcttcttctggtactggttatgctgactccgttaaaggtcgcttcactatctctagagacaactctaagaatactctctacttgcagatgaacagcttaagggctgaggacactgcagtctactattgtgcgagagaactgggtagtgggagctactacccgggatacttccagcactggggccagggcaccctggtcaccgtctcaagcgcctccaccaagggcccatcggtcttcccg

SEQ ID NO: 50 - DNA, 369 bp, Artificial Sequence
  Heavy Chain nucleic acid sequence
gaagttcaattgttagagtctggtggcggtcttgttcagcctggtggttctttacgtctttcttgcgctgcttccggattcactttctcttggtacactatggtttgggttcgccaagctcctggtaaaggtttggagtgggtttcttctatctattcttctggtggctttacttggtatgctgactccgttaaaggtcgcttcactatctctagagacaactctaagaatactctctacttgcagatgaacagcttaagggctgaggacactgcagtctactattgtgcgagtgactttggtagctggggccagggaaccctggtcaccgtctcaagcgcctccaccaagggcccatcggtcttcccg

SEQ ID NO: 51 - DNA, 420 bp, Artificial Sequence
  Heavy Chain nucleic acid sequence
gaagttcaattgttagagtctggtggcggtcttgttcagcctggtggttctttacgtctttcttgcgctgcttccggattcactttctctgattacaagatgccttgggttcgccaagctcctggtaaaggtttggagtgggtttcttctatctggtcttctggtggcactactgagtatgctgactccgttaaaggtcgcttcactatctctagagacaactctaagaatactctctacttgcagatgaacagcttaagggctgaggacactgcagtctactattgtgcgagagaggaaattggacgatattttgactggtttttagggaactactactactacggtatggacgtctggggccaagggaccacggtcaccgtctcaagcgcctccaccaagggcccatcggtcttcccg

SEQ ID NO: 52 - DNA, 411 bp, Artificial Sequence
  Heavy Chain nucleic acid sequence
gaagttcaattgttagagtctggtggcggtcttgttcagcctggtggttctttacgtctttcttgcgctgcttccggattcactttctctacttactttatgcgttgggttcgccaagctcctggtaaaggtttggagtgggtttcttatatcgttccttctggtggcaatactctttatgctgactccgttaaaggtcgcttcactatctctagagacaactctaagaatactctctacttgcagatgaacagcttaagggctgaggacactgcagtctactattgtgcaagagaagagtgggacgtattactatggttcggggagttaagtgctgcttttgatatctggggccaagggacaatggtcaccgtctcaagcgcctccaccaagggcccatcggtcttcccg

SEQ ID NO: 53 - DNA, 369 bp, Unknown
  Light Chain nucleic acid sequence
ttctattctcacagtgcacagagcgaattgactcagccaccctcagcgtctgggacccccgggcagagggtcaccatctcttgttctggaagcagctccaacatcggaagtaatactgtaaactggtaccagcagctcccaggaacggcccccaaactcctcatctatagtaataattaccggccctcaggggtccctgaccgattctctggctccaagtctggcacctcagcctccctggccatcagtgggctccagtctgacgatgaggctgaatatctctgtgcagcatgggatgacagtctgaatggtccggtgttcggtggagggaccaaggtgaccgtcctaggtcagcccaaggctgccccc

SEQ ID NO: 54 - DNA, 396 bp, Artificial Sequence
  Heavy Chain nucleic acid sequence
gaagttcaattgttagagtctggtggcggtcttgttcagcctggtggttctttacgtctttcttgcgctgcttccggattcactttctctttttacggtatgccttgggttcgccaagctcctggtaaaggtttggagtgggtttctggtatctatccttctggtggcgttactcgttatgctgactccgttaaaggtcgcttcactatctctagagacaactctaagaatactctctacttgcagatgaacagcttaagggctgaggacactgcagtctactattgtgcgaagacgtatagcagcagctggtacgggtggtactttgactactggggccagggaaccctggtcaccgtctcaagcgcctccaccaagggcccatcggtcttcccg

SEQ ID NO: 55 - DNA, 396 bp, Artificial Sequence
  Heavy Chain nucleic acid sequence
gaagttcaattgttagagtctggtggcggtcttgttcagcctggtggttctttacgtctttcttgcgctgcttccggattcactttctctccttacgatatgtggtgggttcgccaagctcctggtaaaggtttggagtgggtttcttatatctcttcttctggtggcaagactatgtatgctgactccgttaaaggtcgcttcactatctctagagacaactctaagaatactctctacttgcagatgaacagcttaagggctgaggacactgcagtctactattgtgcgagattaggtggtaactcccactactactacggtatggacgtctggggccaagggaccacggtcaccgtctcaagcgcctccaccaagggcccatcggtcttcccg

SEQ ID NO: 56 - DNA, 396 bp, Artificial Sequence; identical to SEQ ID NO: 55
  Heavy Chain nucleic acid sequence

SEQ ID NO: 57 - DNA, 390 bp, Unknown
  Heavy Chain nucleic acid sequence
gaagttcaattgttagagtctggtggcggtcttgttcagcctggtggttctttacgtctttcttgcgctgcttccggattcactttctcttcttacgttatgatttgggttcgccaagctcctggtaaaggtttggagtgggtttcttggatctcttcttctggtggctatacttcttatgctgactccgttaaaggtcgcttcactatctctagagacaactctaagaatactctctacttgcagatgaacagcttaagggctgaggacactgcagtctactactgtgcgaaagggcccgggacccggggtgactactggggccagggaaccctggtcaccgtctcaagcgcctccaccaagggcccatcggtcttcccgctagcaccc

SEQ ID NO: 58 - DNA, 351 bp, Artificial Sequence
  Heavy Chain nucleic acid sequence
gaagttcaattgttagagtctggtggcggtcttgttcagcctggtggttctttacgtctttcttgcgctgcttccggattcactttctctccttacactatgaattgggttcgccaagctcctggtaaaggtttggagtgggtttctcgtatcggttcttctggtgtttactcattatgctgactccgttaaaggtcgcttcactatctctagagacaactctaagaatactctctacttgcagatgaacagcttaagggctgaggacactgcagtctactattgtgcgagacccaccctctattggtatggttcggggagctattactactttgactactggggccaggg

SEQ ID NO: 59 - DNA, 369 bp, Artificial Sequence
  Heavy Chain nucleic acid sequence
gaagttcaattgttagagtctggtggcggtcttgttcagcctggtggttctttacgtctttcttgcgctgcttccggattcactttctctaattacgctatggattgggttcgccaagctcctggtaaaggtttggagtgggtttcttatatctctccttctggtggctatactcgttatgctgactccgttaaaggtcgcttcactatctctagagacaactctaagaatactctctacttgcagatgaacagcttaagggctgaggacactgcagtctactattgtgcgagtgactttggtagctggggccagggaaccctggtcaccgtctcaagcgcctccaccaagggcccatcggtcttcccg

SEQ ID NO: 60 - DNA, 396 bp, Artificial Sequence
  Heavy Chain nucleic acid sequence
gaagttcaattgttagagtctggtggcggtcttgttcagcctggtggttctttacgtctttcttgcgctgcttccggattcactttctctggttactggatgtcttgggttcgccaagctcctggtaaaggtttggagtgggtttctgttatccgtccttctggtggcaagactggttatgctgactccgttaaaggtcgcttcactatctctagagacaactttaagaatactctctacttgcagatgaacagcttaagggctgaggacactgcagtctactattgtgcgagagtaagggcgcccggctactactactacggtatggacgtctggggccaagggaccacggtcaccgtctcaagcgcctccaccaagggcccatcggtcttcccg

SEQ ID NO: 61 - DNA, 396 bp, Artificial Sequence; identical to SEQ ID NO: 60
  Heavy Chain nucleic acid sequence

SEQ ID NO: 62 - DNA, 351 bp, Artificial Sequence
  Heavy Chain nucleic acid sequence
gaagttcaattgttagagtctggtggcggtcttgttcagcctggtggttctttacgtctttcttgcgctgcttccggattcactttctcttcttaccttatgacttgggttgccaagctcctggtaaaggtttggagtgggtttcttctatctatccttctggtggccatactggttatgctgactccgttaaaggtcgcttcactatctctagagacaactctaagaatactctctacttgcagatgaacagcttaagggctgaggacactgcagtctactattgtgcgagagaggggggatattgtagtagtaccagctgctatgttgactactggggccagggaaccc

SEQ ID NO: 63 - DNA, 414 bp, Artificial Sequence
  Heavy Chain nucleic acid sequence
gaagttcaattgttagagtctggtggcggtcttgttcagcctggtggttctttacgtctttcttgcgctgcttccggattcactttctctcgttacggtatgaagtgggttcgccaagctcctggtaaaggtttggagtgggtttcttatatctatccttctggtggctatactcgttatgctgactccgttaaaggtcgcttcactatctctagagacaactctaagaatactctctacttgcagatgaacagcttaagggctgaggacactgcagtctactattgtgcgagagcccgcgggcatagcagcagctggtacaatcattactactactactacatggacgtctggggcaaagggaccacggtcaccgtctcaagcgcctccaccaagggcccatcggtcttcccg

SEQ ID NO: 64 - DNA, 393 bp, Artificial Sequence
  Heavy Chain nucleic acid sequence
gaagttcaattgttagagtctggtggcggtcttgttcagcctggtggttctttacgtctttcttgcgctgcttccggattcactttctcttggtaccatatgcgttgggttcgccaagctcctggtaaaggtttggagtgggtttctatctatccttctggtggcgttacttcttatgctgactccgttaaaggtcgcttcactatctctagagacaactctaagaatactctctacttgcagatgaacagcttaagggctgaagacactgcagtctactattgtgcgagagaaacaagtggctggtatagggatcgctggttcgacccctggggccagggaaccctggtcaccgtctcaagcgcctccaccaagggcccatcggtcttcccg

SEQ ID NO: 65 - DNA, 384 bp, Artificial Sequence
  Heavy Chain nucleic acid sequence
gaagttcaattgttagagtctggtggcggtcttgttcagcctggtggttctttacgtctttcttgcgctgcttccggattcactttctctcagtacaagatgaattgggttcgccaagctcctggtaaaggtttggagtgggtttcttatatctctccttctggtggctatactgcttatgctgactccgttaaaggtcgcttcactatctctagagacaactctaagaatactctctacttgcagatgaacagcttaagggctgaggacactgcagtctactattgtgcgagagatgtagtggctgggccgtttgactactggggccagggaaccctggtcaccgtctcaagcgcctccaccaagggcccatcggtcttcccg

SEQ ID NO: 66 - DNA, 393 bp, Artificial Sequence
  Heavy Chain nucleic acid sequence
gaagttcaattgttagagtctggtggcggtcttgttcagcctggtggttctttacgtctttcttgcgctgcttccggattcactttctctgattactatatgcgttgggttcgccaagctcctggtaaaggtttggagtgggtttctcgtatctatccttctggtggccatacttggtatgctgactccgttaaaggtcgcttcactatctctagagacaactctaagaatactctctacttgcagatgaacagcttaagggctgaggacactgcagtctactattgtgcgagacatagggcgggtagcagtggctggtactctgactactggggccagggaaccctggtcaccgtctcaagcgcctccaccaagggcccatcggtcttcccg

SEQ ID NO: 67 - DNA, 393 bp, Artificial Sequence; identical to SEQ ID NO: 66
  Heavy Chain nucleic acid sequence

SEQ ID NO: 68 - DNA, 414 bp, Artificial Sequence
  Heavy Chain nucleic acid sequence
gaagttcaattgttagagtctggtggcggtcttgttcagcctggtggttctttacgtctttcttgcgctgcttccggattcactttctcttattaccatatgtggtgggttcgccaagctcctggtaaaggtttggagtgggtttctgttatcgttccttctggtggcggtactcagtatgctgactccgttaaaggtcgcttcactatctctagagacaactctaagaatactctctacttgcagatgaacagcttaagggctgaggacactgcagtctactattgtgcgagagatggacatagcagcagctggtacggtgggggagcccactactacggtatggacgtctggggccaagggaccacggtcaccgtctcaagcgcctccaccaagggcccatcggtcttcccg

SEQ ID NO: 69 - DNA, 414 bp, Artificial Sequence; identical to SEQ ID NO: 68
  Heavy Chain nucleic acid sequence

SEQ ID NO: 70 - DNA, 396 bp, Artificial Sequence
  Heavy Chain nucleic acid sequence
gaagttcaattgttagagtctggtggcggtcttgttcagcctggtggttctttacgtctttcttgcgctgcttccggattcactttctctccttaccgtatggattgggttcgccaagctcctggtaaaggtttggagtgggtttcttatatctatccttctggtggctttactccttatgctgactccgttaaaggtcgcttcactatctctagagacaactctaagaatactttctacttgcagatgaacagcttaagggctgaggacactgcagtctactattgtgcgaaaggttcaacgggataccgctactactacggtatggacgtctggggccaagggaccacggtcaccgtctcaagcgcctccaccaagggcccatcggtcttcccg

SEQ ID NO: 71 - DNA, 408 bp, Artificial Sequence
  Heavy Chain nucleic acid sequence
gaagttcaattgttagagtctggtggcggtcttgttcagcctggtggttctttacgtctttcttgcgctgcttccggattcactttctcttacaagatgatgtgggttcgccaagctcctggtaaaggtttggagtgggtttcttatatctcttcttctggtggcattactacttatgctgactccgttaaaggtcgcttcactatctctagagacaactctaagaatactctctacttgcagatgaacagcttaagggctgaggacactgcagtctactattgtgcgagagacccgacttacgatttttggagtggttattactactactactacatggacgtctggggcaaagggaccacggtcaccgtctcaagcgcctccaccaagggcccatcggtcttcccg

SEQ ID NO: 72 - DNA, 414 bp, Artificial Sequence
  Heavy Chain nucleic acid sequence
gaagttcaattgttagagtctggtggcggtcttgttcagcctggtggttctttacgtctttcttgcgctgcttccggattcactttctctctttaccatatggattgggttcgccaagctcctggtaaaggtttggagtgggtttctgttatctatccttctggtggcggtactccttatgctgactccgttaaaggtcgcttcactatctctagagacaactctaagaatactctctacttgcagatgaacagcttaagggctgaggacactgcagtctactattgtgcgagacgggtaggatattgtagtggtggtagctgctactactactactactacatggacgtctggggcaaagggaccacggtcaccgtctcaagcgcctccaccaagggcccatcggtcttcccg

SEQ ID NO: 73 - DNA, 396 bp, Artificial Sequence
  Heavy Chain nucleic acid sequence
gaagttcaattgttagagtctggtggcggtcttgttcagcctggtggttctttacgtctttcttgcgctgcttccggattcactttctcttggtactggatgaattgggttcgccaagctcctggtaaaggtttggagtgggtttcttctatctattcttctggtggctatacttcttatgctgactccgttaaaggtcgcttcactatctctagagacaactctaagaatactctctacttgcagatgaacagcttaagggctgaggacactgcagtctactattgtgcgagagttcgggatattttgactggtccctactactttgactactggggccagggaaccctggtcaccgtctcaagcgcctccaccaagggcccatcggtcttcccg

SEQ ID NO: 74 - DNA, 393 bp, Artificial Sequence
  Heavy Chain nucleic acid sequence
gaagttcaattgttagagtctggtggcggtcttgttcagcctggtggttctttacgtctttcttgcgctgcttccggattcactttctctaattaccgtatgccttgggttcgccaagctcctggtaaaggtttggagtgggtttcttatatctattcttctggtggcattactcagtatgctgactccgttaaaggtcgcttcactatctctagagacaactctaagaatactctctacttgcagatgaacagcttaagggctgaggacactgcagtctactattgtgcgagatcgcgatcttactatggttcggggtcgtcgcggtactggggccagggaaccctggtcaccgtctcaagcgcctccaccaagggcccatcggtcttcccg

SEQ ID NO: 75 - DNA, 405 bp, Artificial Sequence
  Heavy Chain nucleic acid sequence
gaagttcaattgttagagtctggtggcggtcttgttcagcctggtggttctttacgtctttcttgcgctgcttccggattcactttctctcagtacatgatgacttgggttcgccaagctcctggtaaaggtttggagtgggtttcttatatcggttcttctggtggccagactaagtatgctgactccgttaaaggtcgcttcactatctctagagacaactctaagaatactctctacttgcagatgaacagcttaagggctgaggacactgcagtctactattgtgcgagggatccaggggtagcagtggctgggtactactactacggtatggacgtctggggccaagggaccacggtcaccgtctcaagcgcctccaccaagggcccatcggtcttcccg

SEQ ID NO: 76 - DNA, 411 bp, Artificial Sequence
  Heavy Chain nucleic acid sequence
gaagttcaattgttagagtctggtggcggtcttgttcagcctggtggttctttacgtctttcttgcgctgcttccggattcactttctctcagtacaatatgccttgggttcgccaagctcctggtaaaggtttggagtgggtttcttctatcgttccttctggtggctttactgcttatgctgactccgttaaaggtcgcttcactatctctagagacaactctaagaatactctctacttgcagatgaacagcttaagggctgaggacactgcagtctactattgtgcgagagtcgattgtagtggtggtagctgctaccggggtccccaaaactactttgactactggggccagggaaccctggtcaccgtctcaagcgcctccaccaagggcccatcggtcttcccg

SEQ ID NO: 77 - DNA, 351 bp, Artificial Sequence
  Heavy Chain nucleic acid sequence
gaagttcaattgttagagtctggtggcggtcttgttcagcctggtggttctttacgtctttcttgcgctgcttccggattcactttctctatgtactatatgttttgggttcgccaagctcctggtaaggtttggagtgggtttctgttatcgtttcttctggtggcactactgagtatgctgactccgttaaaggtcgcttcactatctctagagacaactctaagaatactctctacttgcagatgaacagcttaagggctgaggacactgcagtctactattgtgcgagagggggatattgtagtggtggcaggtgttacacctggctcgaagactactggggccagg

SEQ ID NO: 78 - protein, 110 aa, Artificial Sequence
  Light Chain amino acid sequence
QSVLTQPPSASGTPGQRVTISCSGSSSNIESNTVTWYQQLPGTAPKLLIYSDDQRPSGVPDRFSGSKSGTSASLAISGLQSEDEADYYCATWDNTLRGVVFGGGTKLTVL

SEQ ID NO: 79 - protein, 132 aa, Artificial Sequence
  Heavy Chain amino acid sequence
EVQLLESGGGLVQPGGSLRLSCAASGFTFSPYRMDWVRQAPGKGLEWVSYIYPSGGFTPYADSVKGRFTISRDNSKNTFYLQMNSLRAEDTAVYYCAKGSTGYRYYYGMDVWGQGTTVTVSSASTKGPSVFP

SEQ ID NO: 80 - protein, 13 aa, Artificial Sequence
  Synthetically generated peptide
SGSSSNIESNTVT

SEQ ID NO: 81 - protein, 7 aa, Artificial Sequence
  Synthetically generated peptide
SDDQRPS

SEQ ID NO: 82 - protein, 11 aa, Artificial Sequence
  Synthetically generated peptide
ATWDNTLRGVV

SEQ ID NO: 83 - protein, 5 aa, Artificial Sequence
  Synthetically generated peptide
PYRMD

SEQ ID NO: 84 - protein, 17 aa, Artificial Sequence
  Synthetically generated peptide
YIYPSGGFTPYADSVKG

SEQ ID NO: 85 - protein, 13 aa, Artificial Sequence
  Synthetically generated peptide
GSTGYRYYYGMDV

SEQ ID NO: 86 - protein, 120 aa, Artificial Sequence
  Light Chain amino acid sequence
QDIVMTQTPPSLPVNPGEPASISCKSSQSLLQSNGYNYLDWYLQKPGQSPQLLIYLGSNRASGVPDRFSGSGSGTDFTLKISRVEAEDVGIYYCMQALHTPPFGQGTRLEIKRTVAAPSV

SEQ ID NO: 87 - protein, 132 aa, Artificial Sequence
  Heavy Chain amino acid sequence
EVQLLESGGGLVQPGGSLRLSCAASGFTFSWYWMNWVRQAPGKGLEWVSSIYSSGGYTSYADSVKGRFTISRDNSKNTLYLQMNSLRAEDTAVYYCARVRDILTGPYYFDYWGQGTLVTVSSASTKGPSVFP

SEQ ID NO: 88 - protein, 16 aa, Artificial Sequence
  Synthetically generated peptide
KSSQSLLQSNGYNYLD

SEQ ID NO: 89 - protein, 7 aa, Artificial Sequence
  Synthetically generated peptide
LGSNRAS

SEQ ID NO: 90 - protein, 8 aa, Artificial Sequence
  Synthetically generated peptide
MQALHTPP

SEQ ID NO: 91 - protein, 5 aa, Artificial Sequence
  Synthetically generated peptide
WYWMN

SEQ ID NO: 92 - protein, 17 aa, Artificial Sequence
  Synthetically generated peptide
SIYSSGGYTSYADSVKG

SEQ ID NO: 93 - protein, 13 aa, Artificial Sequence
  Synthetically generated peptide
VRDILTGPYYFDY

SEQ ID NO: 94 - protein, 116 aa, Artificial Sequence
  Light Chain amino acid sequence
QDIQMTQSPSSLSASVGDRVTITCRASQGIRHYLGWYQQKPGKAPKRLIYAASSLQFGVPARFSGSGSGTEFTLTISSLQPEDFATYYCLQHNSFPPAFGQGTKVEIKRTVAAPSV

SEQ ID NO: 95 - protein, 132 aa, Artificial Sequence
  Heavy Chain amino acid sequence
EVQLLESGGGLVQPGGSLRLSCAASGFTFSPYDMWWVRQAPGKGLEWVSYISSSGGKTMYADSVKGRFTISRDNSKNTLYLQMNSLRAEDTAVYYCARLGGNSHYYYGMDVWGQGTTVTVSSASTKGPSVFP

SEQ ID NO: 96 - protein, 11 aa, Artificial Sequence
  Synthetically generated peptide
RASQGIRHYLG

SEQ ID NO: 97 - protein, 7 aa, Artificial Sequence
  Synthetically generated peptide
AASSLQF

SEQ ID NO: 98 - protein, 9 aa, Artificial Sequence
  Synthetically generated peptide
LQHNSFPPA

SEQ ID NO: 99 - protein, 5 aa, Artificial Sequence
  Synthetically generated peptide
PYDMW

SEQ ID NO: 100 - protein, 17 aa, Artificial Sequence
  Synthetically generated peptide
YISSSGGKTMYADSVKG

SEQ ID NO: 101 - protein, 13 aa, Artificial Sequence
  Synthetically generated peptide
LGGNSHYYYGMDV

SEQ ID NO: 102 - protein, 116 aa, Artificial Sequence
  Light Chain amino acid sequence
QDIQMTQSPSSVSASVGDRIAITCRASQGISTWLAWYQQRPGRAPKLLIYAASTLQSGVPSRFSGSGSGTDFTLTISSLQPEDFATYFCQQADSFPLTFGQGTKLEIKRTVAAPSV

SEQ ID NO: 103 - protein, 123 aa, Artificial Sequence
  Heavy Chain amino acid sequence Heavy Chain amino acid sequence Heavy Chain amino acid sequence Heavy Chain amino acid sequence Heavy Chain amino acid sequence
EVQLLESGGGLVQPGGSLRLSCAASGFTFSNYAMDWVRQAPGKGLEWVSYISPSGGYTRYADSVKGRFTISRDNSKNTLYLQMNSLRAEDTAVYYCASDFGSWGQGTLVTVSSASTKGPSVFP

SEQ ID NO: 104 - protein, 11 aa, Artificial Sequence
  Synthetically generated peptide
RASQGISTWLA

SEQ ID NO: 105 - protein, 7 aa, Artificial Sequence
  Synthetically generated peptide
AASTLQS

SEQ ID NO: 106 - protein, 9 aa, Artificial Sequence
  Synthetically generated peptide
QQADSFPLT

SEQ ID NO: 107 - protein, 5 aa, Artificial Sequence
  Synthetically generated peptide
NYAMD

SEQ ID NO: 108 - protein, 17 aa, Artificial Sequence
  Synthetically generated peptide
YISPSGGYTRYADSVKG

SEQ ID NO: 109 - protein, 4 aa, Artificial Sequence
  Synthetically generated peptide
DFGS

SEQ ID NO: 110 - protein, 117 aa, Artificial Sequence
  Light Chain amino acid sequence
QDIQMTQSPGTLSLSPGERATLSCRASQSISSSYLAWYQQKPGQAPRLLIYAAASRATGIPDRFSGIGSGTDFTLTISSLEPEDFAVYYCQQRSNWPLTFGGGTKVEIKRTVAAPSV

SEQ ID NO: 111 - protein, 131 aa, Artificial Sequence
  Heavy Chain amino acid sequence
EVQLLESGGGLVQPGGSLRLSCAASGFTFSRYHMEWVRQAHGKGLEWVSYISPSGGKTLYADSVKGRFTISRDNSKNTLYLQMNSLRAEDTAVYYCARHLGYGSGSYFDYWGQGTLVTVSSASTKGPSVFP

SEQ ID NO: 112 - protein, 12 aa, Artificial Sequence
  Synthetically generated peptide
RASQSISSSYLA

SEQ ID NO: 113 - protein, 7 aa, Artificial Sequence
  Synthetically generated peptide
AAASRAT

SEQ ID NO: 114 - protein, 9 aa, Artificial Sequence
  Synthetically generated peptide
QQRSNWPLT

SEQ ID NO: 115 - protein, 5 aa, Artificial Sequence
  Synthetically generated peptide
RYHME

SEQ ID NO: 116 - protein, 17 aa, Artificial Sequence
  Synthetically generated peptide
YISPSGGKTLYADSVKG

SEQ ID NO: 117 - protein, 12 aa, Artificial Sequence
  Synthetically generated peptide
HLGYGSGSYFDY

SEQ ID NO: 118 - protein, 116 aa, Artificial Sequence
  Light Chain amino acid sequence
QYELTQPPSVSVSPGQTATIICSGDKLGDKYVAWYQQKPGQSPVLVVYEDNKRPSGIPERISGSNSGNTATLTISGTQAMDDADYYCQAWDRSTDHYVFGTGTKVTVLGQPKANPT

SEQ ID NO: 119 - protein, 131 aa, Artificial Sequence
  Heavy Chain amino acid sequence
EVQLLESGGGLVQPGGSLRLSCAASGFTFSNYRMPWVRQAPGKGLEWVSYIYSSGGITQYADSVKGRFTISRDNSKNTLYLQMNSLRAEDTAVYYCARSRSYYGSGSSRYWGQGTLVTVSSASTKGPSVFP

SEQ ID NO: 120 - protein, 11 aa, Artificial Sequence
  Synthetically generated peptide
SGDKLGDKYVA

SEQ ID NO: 121 - protein, 7 aa, Artificial Sequence
  Synthetically generated peptide
EDNKRPS

SEQ ID NO: 122 - protein, 11 aa, Artificial Sequence
  Synthetically generated peptide
QAWDRSTDHYV

SEQ ID NO: 123 - protein, 5 aa, Artificial Sequence
  Synthetically generated peptide
NYRMP

SEQ ID NO: 124 - protein, 17 aa, Artificial Sequence
  Synthetically generated peptide
YIYSSGGITQYADSVKG

SEQ ID NO: 125 - protein, 12 aa, Artificial Sequence
  Synthetically generated peptide
SRSYYGSGSSRY

SEQ ID NO: 126 - protein, 108 aa, Unknown
  Synthetically generated peptide
QDIQMTQSPSSFSASTGDRVTITCRASQGISSYLAWYQQKPGKAPKLLIYAASTLQSGVPSKFSGSGSGTDFTLTISSLQPEDFATYYCQQYNSYPLTFGQGTRLEIK

SEQ ID NO: 127 - protein, 123 aa, Artificial Sequence
  Heavy Chain amino acid sequence
EVQLLESGGGLVQPGGSLRLSCAASGFTFSWYTMVWVRQAPGKGLEWVSSIYSSGGFTWYADSVKGRFTISRDNSKNTLYLQMNSLRAEDTAVYYCASDFGSWGQGTLVTVSSASTKGPSVFP

SEQ ID NO: 128 - protein, 11 aa, Artificial Sequence
  Synthetically generated peptide
RASQGISSYLA

SEQ ID NO: 129 - protein, 7 aa, Artificial Sequence
  Synthetically generated peptide
AASTLQS

SEQ ID NO: 130 - protein, 9 aa, Artificial Sequence
  Synthetically generated peptide
QQYNSYPLT

SEQ ID NO: 131 - protein, 5 aa, Artificial Sequence
  Synthetically generated peptide
WYTMV

SEQ ID NO: 132 - protein, 17 aa, Artificial Sequence
  Synthetically generated peptide
SIYSSGGFTWYADSVKG

SEQ ID NO: 133 - protein, 4 aa, Artificial Sequence
  Synthetically generated peptide
DFGS

SEQ ID NO: 134 - protein, 116 aa, Artificial Sequence
  Light Chain amino acid sequence
QDIQMTQSPSSLYASVGDRVTITCRASQGIRNELGWYQQKPGKAPQRLIYDASTLQSGVPSRFSGGGSRTEFTLTISSLEPHDFGTYYCQQYASYPLTFGGGTKVEIKRTVAAPSV

SEQ ID NO: 135 - protein, 140 aa, Artificial Sequence
  Heavy Chain amino acid sequence
EVQLLESGGGLVQPGGSLRLSCAASGFTFSDYKMPWVRQAPGKGLEWVSSIWSSGGTTEYADSVKGRFTISRDNSKNTLYLQMNSLRAEDTAVYYCAREEIGRYFDWFLGNYYYYGMDVWGQGTTVTVSSASTKGPSVFP

SEQ ID NO: 136 - protein, 11 aa, Artificial Sequence
  Light Chain amino acid sequence
RASQGIRNELG

SEQ ID NO: 137 - protein, 7 aa, Artificial Sequence
  Light Chain amino acid sequence
DASTLQS

SEQ ID NO: 138 - protein, 9 aa, Artificial Sequence
  Light Chain amino acid sequence
QQYASYPLT

SEQ ID NO: 139 - protein, 5 aa, Artificial Sequence
  Heavy Chain amino acid sequence
DYKMP

SEQ ID NO: 140 - protein, 17 aa, Artificial Sequence
  Heavy Chain amino acid sequence
SIWSSGGTTEYADSVKG

SEQ ID NO: 141 - protein, 21 aa, Artificial Sequence
  Heavy Chain amino acid sequence
EEIGRYFDWFLGNYYYYGMDV

SEQ ID NO: 142 - protein, 118 aa, Artificial Sequence
  Light Chain amino acid sequence
QSALTQPPSASGTPGQRVTISCSGSSSNIGSNFVYWYHHLPGTAPKLLIYRNNQRPSGVPDRFSGSKSGTSASLAISGLRSEDEADYYCAAWDDSLSGVVFGGGTKLTVLGQPKAAPS

SEQ ID NO: 143 - protein, 128 aa, Artificial Sequence
  Heavy Chain amino acid sequence
EVQLLESGGGLVQPGGSLRLSCAASGFTFSQYKMNWVRQAPGKGLEWVSYISPSGGYTAYADSVKGRFTISRDNSKNTLYLQMNSLRAEDTAVYYCARDVVAGPFDYWGQGTLVTVSSASTKGPSVFP

SEQ ID NO: 144 - protein, 13 aa, Artificial Sequence
  Light Chain amino acid sequence
SGSSSNIGSNFVY

SEQ ID NO: 145 - protein, 7 aa, Artificial Sequence
  Light Chain amino acid sequence
RNNQRPS

SEQ ID NO: 146 - protein, 11 aa, Artificial Sequence
  Light Chain amino acid sequence
AAWDDSLSGVV

SEQ ID NO: 147 - protein, 5 aa, Artificial Sequence
  Heavy Chain amino acid sequence
QYKMN

SEQ ID NO: 148 - protein, 17 aa, Artificial Sequence
  Heavy Chain amino acid sequence
YISPSGGYTAYADSVKG

SEQ ID NO: 149 - protein, 9 aa, Artificial Sequence
  Heavy  Chain amino acid sequence
DVVAGPFDY

SEQ ID NO: 150 - protein, 116 aa, Artificial Sequence
  Light Chain amino acid sequence
QDIQMTQSPSSLSASVGDRVTITCRASQDISNYLAWFQQKPGRAPKSLIYGASSLQTGVPSKFSGSGSGTEFTLTISGLQPEDVATYYCHQYNHYPPTFGGGTKVEIKRTVAAPSV

SEQ ID NO: 151 - protein, 129 aa, Artificial Sequence
  Heavy Chain amino acid sequence
EVQLLESGGGLVQPGGSLRLSCAASGFTFSKYPMFWVRQAPGKGLEWVSWISPSGGKTVYADSVKGRFTISRDNSKNTLYLQMNSLRAEDTAVYYCAKDCRGGCSGGSWGQGTLVTVSSASTKGPSVFP

SEQ ID NO: 152 - protein, 11 aa, Artificial Sequence
  Light Chain amino acid sequence
RASQDISNYLA

SEQ ID NO: 153 - protein, 7 aa, Artificial Sequence
  Light Chain amino acid sequence
GASSLQT

SEQ ID NO: 154 - protein, 9 aa, Artificial Sequence
  Light Chain amino acid sequence
HQYNHYPPT

SEQ ID NO: 155 - protein, 5 aa, Artificial Sequence
  Heavy Chain amino acid sequence
KYPMF

SEQ ID NO: 156 - protein, 17 aa, Artificial Sequence
  Heavy Chain amino acid sequence
WISPSGGKTVYADSVKG

SEQ ID NO: 157 - protein, 10 aa, Artificial Sequence
  Heavy Chain amino acid sequence
DCRGGCSGGS

SEQ ID NO: 158 - protein, 116 aa, Artificial Sequence
  Light Chain amino acid sequence
QDIQMTQSPATLSVSPGERATLSCRASQDVNRYLAWYQQKPGQPPRLLIYGASTRATGIPARISGSGSGTEFTLTISSLQSEDFAVYYCQQYHNWPLTFGGGTKVEIKRTVAAPSV

SEQ ID NO: 159 - protein, 123 aa, Artificial Sequence
  Heavy Chain amino acid sequence
EVQLLESGGGLVQPGGSLRLSCAASGFTFSRYSMNWVRQAPGKGLEWVSYISPSGGMTKYADSVKGRFTISRDNSKNTLYLQMNSLRAEDTAVYYCANTLGYWGQGTLVTVSSASTKGPSVFP

SEQ ID NO: 160 - protein, 11 aa, Artificial Sequence
  Light Chain amino acid sequence
RASQDVNRYLA

SEQ ID NO: 161 - protein, 7 aa, Artificial Sequence
  Light Chain amino acid sequence
GASTRAT

SEQ ID NO: 162 - protein, 9 aa, Artificial Sequence
  Light Chain amino acid sequence
QQYHNWPLT

SEQ ID NO: 163 - protein, 5 aa, Artificial Sequence
  Heavy Chain amino acid sequence
RYSMN

SEQ ID NO: 164 - protein, 17 aa, Artificial Sequence
  Heavy Chain amino acid sequence
YISPSGGMTKYADSVKG

SEQ ID NO: 165 - protein, 4 aa, Artificial Sequence
  Heavy Chain amino acid sequence
TLGY

SEQ ID NO: 166 - protein, 119 aa, Artificial Sequence
  Light Chain amino acid sequence
QSALTQPASVSGSPGQSITISCTGTSSDVGYYDYVSWYQHHPGKAPKLIIYDVTSRPSGVSSHFSGSKSGNTASLTISGLQADDEADYYCSSYTSGSTRYVFGPGTKVTVLGQPKANPT

SEQ ID NO: 167 - protein, 131 aa, Artificial Sequence
  Heavy Chain amino acid sequence
EVQLLESGGGLVQPGGSLRLSCAASGFTFSDYYMRWVRQAPGKGLEWVSRIYPSGGHTWYADSVKGRFTISRDNSKNTLYLQMNSLRAEDTAVYYCARHRAGSSGWYSDYWGQGTLVTVSSASTKGPSVFP

SEQ ID NO: 168 - protein, 14 aa, Artificial Sequence
  Light Chain amino acid sequence
TGTSSDVGYYDYVS

SEQ ID NO: 169 - protein, 7 aa, Artificial Sequence
  Light Chain amino acid sequence
DVTSRPS

SEQ ID NO: 170 - protein, 11 aa, Artificial Sequence
  Light Chain amino acid sequence
SSYTSGSTRYV

SEQ ID NO: 171 - protein, 5 aa, Artificial Sequence
  Heavy Chain amino acid sequence
DYYMR

SEQ ID NO: 172 - protein, 17 aa, Artificial Sequence
  Heavy Chain amino acid sequence
RIYPSGGHTWYADSVKG

SEQ ID NO: 173 - protein, 12 aa, Artificial Sequence
  Heavy Chain amino acid sequence
HRAGSSGWYSDY

SEQ ID NO: 174 - protein, 116 aa, Artificial Sequence
  Light Chain amino acid sequence
QDIQMTQSPSSLSASVGDRVTITCRASQDIRNYLAWFQQKPGEAPKSLIYAASSLQSGVSSNFSGSGSGTDFTLTISSLQPEDFATYYCQQYHRYPRTFGQGTKLEIKRTVAAPSV

SEQ ID NO: 175 - protein, 131 aa, Artificial Sequence
  Heavy Chain amino acid sequence
EVQLLESGGGLVQPGGSLRLSCAASGFTFSAYNMPWVRQAPGKGLEWVSYISSSGTGYADSVKGRFTISRDNSKNTLYLQMNSLRAEDTAVYYCARELGSGSYYPGYFQHWGQGTLVTVSSASTKGPSVFP

SEQ ID NO: 176 - protein, 11 aa, Artificial Sequence
  Light Chain amino acid sequence
RASQDIRNYLA

SEQ ID NO: 177 - protein, 7 aa, Artificial Sequence
  Light Chain amino acid sequence
AASSLQS

SEQ ID NO: 178 - protein, 9 aa, Artificial Sequence
  Light Chain amino acid sequence
QQYHRYPRT

SEQ ID NO: 179 - protein, 5 aa, Artificial Sequence
  Heavy Chain amino acid sequence
AYNMP

SEQ ID NO: 180 - protein, 16 aa, Artificial Sequence
  Heavy Chain amino acid sequence
YISSSGTGYADSVKGR

SEQ ID NO: 181 - protein, 14 aa, Artificial Sequence
  Heavy Chain amino acid sequence
ELGSGSYYPGYFQH

SEQ ID NO: 182 - protein, 116 aa, Artificial Sequence
  Light Chain amino acid sequence
QDIQMTQSPATLYVSPGERATLSCRASQSVSRNLAWYQQKPGQAPRLLIYGASTRATGIPARFSGSGSRTEFTLTISSLQSEDFAVYHCQQYNSRPLTFGGGTKVEIKRTVAAPSV

SEQ ID NO: 183 - protein, 123 aa, Artificial Sequence
  Heavy Chain amino acid sequence
EVQLLESGGGLVQPGGSLRLSCAASGFTFSWYFMNWVRQAPGKGLEWVSSIYPSGGYTMYADSVKGRFTISRDNSKKTLYLQMNSLRAEDTAVYYCASDFGSWGQGTLVTVSSASTKGPSVFP

SEQ ID NO: 184 - protein, 11 aa, Artificial Sequence
  Light Chain amino acid sequence
RASQSVSRNLA

SEQ ID NO: 185 - protein, 7 aa, Artificial Sequence
  Light Chain amino acid sequence
GASTRAT

SEQ ID NO: 186 - protein, 9 aa, Artificial Sequence
  Light Chain amino acid sequence
QQYNSRPLT

SEQ ID NO: 187 - protein, 5 aa, Artificial Sequence
  Heavy Chain amino acid sequence
WYFMN

SEQ ID NO: 188 - protein, 17 aa, Artificial Sequence
  Heavy Chain amino acid sequence
SIYPSGGYTMYADSVKG

SEQ ID NO: 189 - protein, 4 aa, Artificial Sequence
  Heavy Chain amino acid sequence
DFGS

SEQ ID NO: 190 - protein, 119 aa, Artificial Sequence; identical to SEQ ID NO: 166
  Light Chain amino acid sequence

SEQ ID NO: 191 - protein, 131 aa, Artificial Sequence; identical to SEQ ID NO: 167
  Heavy Chain amino acid sequence

SEQ ID NO: 192 - protein, 14 aa, Artificial Sequence
  Light Chain amino acid sequence
TGTSSDVGYYDYVS

SEQ ID NO: 193 - protein, 7 aa, Artificial Sequence
  Light Chain amino acid sequence
DVTSRPS

SEQ ID NO: 194 - protein, 11 aa, Artificial Sequence
  Light Chain amino acid sequence
SSYTSGSTRYV

SEQ ID NO: 195 - protein, 5 aa, Artificial Sequence
  Heavy Chain amino acid sequence
DYYMR

SEQ ID NO: 196 - protein, 17 aa, Artificial Sequence
  Heavy Chain amino acid sequence
RIYPSGGHTWYADSVKG

SEQ ID NO: 197 - protein, 12 aa, Artificial Sequence
  Heavy Chain amino acid sequence
HRAGSSGWYSDY

SEQ ID NO: 198 - protein, 116 aa, Artificial Sequence
  Light Chain amino acid sequence
QDIQMTQSPSSLSASVGDRVTITCRASQSISSYLNWYQQKPGKAPKLLIYAASSLQSGVPSRFSGSGSGTDFTLTISSLQPEDFATYYCQQSYSTRWTFGQGTKVEIKRTVAAPSV

SEQ ID NO: 199 - protein, 137 aa, Artificial Sequence
  Heavy Chain amino acid sequence
EVQLLESGGGLVQPGGSLRLSCAASGFTFSTYFMRWVRQAPGKGLEWVSYIVPSGGNTLYADSVKGRFTISRDNSKNTLYLQMNSLRAEDTAVYYCAREEWDVLLWFGELSAAFDIWGQGTMVTVSSASTKGPSVFP

SEQ ID NO: 200 - protein, 11 aa, Artificial Sequence
  Light Chain amino acid sequence
RASQSISSYLN

SEQ ID NO: 201 - protein, 7 aa, Artificial Sequence
  Light Chain amino acid sequence
AASSLQS

SEQ ID NO: 202 - protein, 9 aa, Artificial Sequence
  Light Chain amino acid sequence
QQSYSTRWT

SEQ ID NO: 203 - protein, 5 aa, Artificial Sequence
  Heavy Chain amino acid sequence
TYFMR

SEQ ID NO: 204 - protein, 17 aa, Artificial Sequence
  Heavy Chain amino acid sequence
YIVPSGGNTLYADSVKG

SEQ ID NO: 205 - protein, 18 aa, Artificial Sequence
  Heavy Chain amino acid sequence
EEWDVLLWFGELSAAFDI

SEQ ID NO: 206 - protein, 116 aa, Artificial Sequence; identical to SEQ ID NO: 94
  Light Chain amino acid sequence

SEQ ID NO: 207 - protein, 132 aa, Artificial Sequence; identical to SEQ ID NO: 95
  Heavy Chain amino acid sequence

SEQ ID NO: 208 - protein, 11 aa, Artificial Sequence
  Light Chain amino acid sequence
RASQGIRHYLG

SEQ ID NO: 209 - protein, 7 aa, Artificial Sequence
  Light Chain amino acid sequence
AASSLQF

SEQ ID NO: 210 - protein, 9 aa, Artificial Sequence
  Light Chain amino acid sequence
LQHNSFPPA

SEQ ID NO: 211 - protein, 5 aa, Artificial Sequence
  Heavy Chain amino acid sequence
PYDMW

SEQ ID NO: 212 - protein, 17 aa, Artificial Sequence
  Heavy Chain amino acid sequence
YISSSGGKTMYADSVKG

SEQ ID NO: 213 - protein, 13 aa, Artificial Sequence
  Heavy Chain amino acid sequence
LGGNSHYYYGMDV

SEQ ID NO: 214 - protein, 118 aa, Artificial Sequence
  Light Chain amino acid sequence
QSELTQPPSASATPGQRVTISCSGSSSNIGRNLVYWYQQLPGTAPKLLIYSNNQRPSGVPDRFSGSKSGTSASLAISGLRSEEEADYYCAAWDDSLSGWVFGGGTRLTVLGQPKAAPS

SEQ ID NO: 215 - protein, 131 aa, Artificial Sequence
  Heavy Chain amino acid sequence
EVQLLESGGGLVQPGGSLRLSCAASGFTFSWYHMRWVRQAPGKGLEWVSIYPSGGVTSYADSVKGRFTISRDNSKNTLYLQMNSLRAEDTAVYYCARETSGWYRDRWFDPWGQGTLVTVSSASTKGPSVFP

SEQ ID NO: 216 - protein, 13 aa, Artificial Sequence
  Light Chain amino acid sequence
SGSSSNIGRNLVY

SEQ ID NO: 217 - protein, 7 aa, Artificial Sequence
  Light Chain amino acid sequence
SNNQRPS

SEQ ID NO: 218 - protein, 11 aa, Artificial Sequence
  Light Chain amino acid sequence
AAWDDSLSGWV

SEQ ID NO: 219 - protein, 5 aa, Artificial Sequence
  Heavy Chain amino acid sequence
WYHMR

SEQ ID NO: 220 - protein, 16 aa, Artificial Sequence
  Heavy Chain amino acid sequence
IYPSGGVTDYADSVKG

SEQ ID NO: 221 - protein, 13 aa, Artificial Sequence
  Heavy Chain amino acid sequence
ETSGWYRDRWFDP

SEQ ID NO: 222 - protein, 119 aa, Artificial Sequence
  Light Chain amino acid sequence
QSVLTQTASVSGSPGQSITISCTGTSSDIGDYEYVSWYQQHPGKAPKVILYEVSNRPSGVPDRFSGSKSGNTASLTISGLQAEDEADYYCGSYRKSSTPYVFGTGTKVSVLGQPKANPT

SEQ ID NO: 223 - protein, 138 aa, Artificial Sequence
  Heavy Chain amino acid sequence
EVQLLESGGGLVQPGGSLRLSCAASGFTFSYYHMWWVRQAPGKGLEWVSVIVPSGGGTQYADSVKGRFTISRDNSKNTLYLQMNSLRAEDTAVYYCARDGHSSSWYGGGAHYYGMDVWGQGTTVTVSSASTKGPSVFP

SEQ ID NO: 224 - protein, 14 aa, Artificial Sequence
  Light Chain amino acid sequence
TGTSSDIGDYEYVS

SEQ ID NO: 225 - protein, 8 aa, Artificial Sequence
  Light Chain amino acid sequence
YEVSNRPS

SEQ ID NO: 226 - protein, 11 aa, Artificial Sequence
  Light Chain amino acid sequence
GSYRKSSTPYV

SEQ ID NO: 227 - protein, 5 aa, Artificial Sequence
  Heavy Chain amino acid sequence
YYHMW

SEQ ID NO: 228 - protein, 17 aa, Artificial Sequence
  Heavy Chain amino acid sequence
VIVPSGGGTQYADSVKG

SEQ ID NO: 229 - protein, 19 aa, Artificial Sequence
  Heavy Chain amino acid sequence
DGHSSSWYGGGAHYYGMDV

SEQ ID NO: 230 - protein, 116 aa, Artificial Sequence
  Light Chain amino acid sequence
QDIQMTQSPATLSLSPGERATLSCRASQSVSSYLAWYQQKPGQAPRLLIYGASSRATGIPDRFSGSGSGTDFTLTIGRLEPEDFAVYYCQQYSSSPVTFGQGTRLEIKRTVAAPSV

SEQ ID NO: 231 - protein, 123 aa, Artificial Sequence
  Heavy Chain amino acid sequence
EVQLLESGGGLVQPGGSLRLSCAASGFTFSSYRMNWVRQAPGKGLEWVSGIVPSGGKTFYADSVKGRFTISRDNSKNTLYLQMNSLRAEDTAVYYCASDFGSWGQGTLVTVSSASTKGPSVFP

SEQ ID NO: 232 - protein, 11 aa, Artificial Sequence
  Light Chain amino acid sequence
RASQSVSSYLA

SEQ ID NO: 233 - protein, 7 aa, Artificial Sequence
  Light Chain amino acid sequence
GASSRAT

SEQ ID NO: 234 - protein, 9 aa, Artificial Sequence
  Light Chain amino acid sequence
QQYSSSPVT

SEQ ID NO: 235 - protein, 5 aa, Artificial Sequence
  Heavy Chain amino acid sequence
SYRMN

SEQ ID NO: 236 - protein, 17 aa, Artificial Sequence
  Heavy Chain amino acid sequence
GIVPSGGKTFYADSVKG

SEQ ID NO: 237 - protein, 4 aa, Artificial Sequence
  Heavy Chain amino acid sequence
DFGS

SEQ ID NO: 238 - protein, 116 aa, Artificial Sequence
  Light Chain amino acid sequence
QDIQMTQSPSSLSASVGDRVTITCRASQRISSYVNWYQQKPGKAPKLLIYSASSLQSGVPSRFSGSVSGTEFTLTISSLQPEDFATYYCQQSYRTPPFFGQGTKLEVKRTVAAPSV

SEQ ID NO: 239 - protein, 129 aa, Artificial Sequence
  Heavy Chain amino acid sequence
EVQLLESGGGLVQPGGSLRLSCAASGFTFSLYQMLWVRQAPGKGLEWVSGIVSSGGLTGYADSVKGRFTISRDNSKNTLYLQMNSLRAEDTAVYYCARHNRAIGTFDYWGQGTLVTVSSASTKGPSVFP

SEQ ID NO: 240 - protein, 11 aa, Artificial Sequence
  Light Chain amino acid sequence
RASQRISSYVN

SEQ ID NO: 241 - protein, 7 aa, Artificial Sequence
  Light Chain amino acid sequence
SASSLQS

SEQ ID NO: 242 - protein, 9 aa, Artificial Sequence
  Light Chain amino acid sequence
QQSYRTPPF

SEQ ID NO: 243 - protein, 5 aa, Artificial Sequence
  Heavy Chain amino acid sequence
LYQML

SEQ ID NO: 244 - protein, 17 aa, Artificial Sequence
  Heavy Chain amino acid sequence
GIVSSGGLTGYADSVKG

SEQ ID NO: 245 - protein, 10 aa, Artificial Sequence
  Heavy Chain amino acid sequence
HNRAIGTFDY

SEQ ID NO: 246 - protein, 115 aa, Artificial Sequence
  Light Chain amino acid sequence
QDIQMTQSPATLSLSPGERATLSCRASQSVSRYLAWYQQKPGQAPRLLIYGASTRATGIPARFSGSGSGTEFTLTISSLQSEDFAVYYCQQYNNWPSFGGGTKVEIKRTVAAPSV

SEQ ID NO: 247 - protein, 123 aa, Artificial Sequence
  Heavy Chain amino acid sequence
EVQLLESGGGLVQPGGSLRLSCAASGFTFSNYSMDWVRQAPGKGLEWVSWISPSGGLTTYADSVKGRFTISRDNSKNTLYLQMNSLRAEDTAVYYCASDFGSWGQGTLVTVSSASTKGPSVFP

SEQ ID NO: 248 - protein, 11 aa, Artificial Sequence
  Light Chain amino acid sequence
RASQSVSRYLA

SEQ ID NO: 249 - protein, 7 aa, Artificial Sequence
  Light Chain amino acid sequence
GASTRAT

SEQ ID NO: 250 - protein, 8 aa, Artificial Sequence
  Light Chain amino acid sequence
QQYNNWPS

SEQ ID NO: 251 - protein, 5 aa, Artificial Sequence
  Heavy Chain amino acid sequence
NYSMD

SEQ ID NO: 252 - protein, 17 aa, Artificial Sequence
  Heavy Chain amino acid sequence
WISPSGGLTTYADSVKG

SEQ ID NO: 253 - protein, 4 aa, Artificial Sequence
  Heavy Chain amino acid sequence
DFGS

SEQ ID NO: 254 - protein, 124 aa, Artificial Sequence
  Light Chain amino acid sequence
QSVLTQPPYASASLGASVTLTCTLSSGYSNYKVDWYQQRPGKGPQFVMRVGSGGIVGSKGDGIPDRFSVLGSGLYRYLTIKNIQEEDESDYYCGADHGRGGTFVWVFGGGTKLTVLGQPKAAPS

SEQ ID NO: 255 - protein, 136 aa, Artificial Sequence
  Heavy Chain amino acid sequence
EVQLLESGGGLVQPGGSLRLSCAASGFTFSYKMMWVRQAPGKGLEWVSYISSSGGITTYADSVKGRFTISRDNSKNTLYLQMNSLRAEDTAVYYCARDPTYDFWSGYYYYYYMDVWGKGTTVTVSSASTKGPSVFP

SEQ ID NO: 256 - protein, 12 aa, Artificial Sequence
  Light Chain amino acid sequence
TLSSGYSNYKVD

SEQ ID NO: 257 - protein, 13 aa, Artificial Sequence
  Light Chain amino acid sequence
RVGSGGIVGSKGD

SEQ ID NO: 258 - protein, 13 aa, Artificial Sequence
  Light Chain amino acid sequence
GADHGRGGTFVWV

SEQ ID NO: 259 - protein, 5 aa, Artificial Sequence
  Heavy Chain amino acid sequence
SYKMM

SEQ ID NO: 260 - protein, 17 aa, Artificial Sequence
  Heavy Chain amino acid sequence
YISSSGGITTYADSVKG

SEQ ID NO: 261 - protein, 19 aa, Artificial Sequence
  Heavy Chain amino acid sequence
RDPTYDFWSGYYYYYYMDV

SEQ ID NO: 262 - protein, 118 aa, Artificial Sequence
  Light Chain amino acid sequence
QSALTQPSSASGTPGQRVSISCSGSSYNIGVYDVYWYQQLPGTAPKLLIYTNNQRPSGVPDRFSGSKSGTSASLAISGLQSEDEADYYCAAWDDSLSGWVFGGGTKVTVLGQPKAAPS

SEQ ID NO: 263 - protein, 137 aa, Artificial Sequence
  Heavy Chain amino acid sequence
EVQLLESGGGLVQPGGSLRLSCAASGFTFSQYNMPWVRQAPGKGLEWVSSIVPSGGFTAYADSVKGRFTISRDNSKNTLYLQMNSLRAEDTAVYYCARVDCSGGSCYRGPQNYFDYWGQGTLVTVSSASTKGPSVFP

SEQ ID NO: 264 - protein, 13 aa, Artificial Sequence
  Light Chain amino acid sequence
SGSSYNIGVYDVY

SEQ ID NO: 265 - protein, 7 aa, Artificial Sequence
  Light Chain amino acid sequence
TNNQRPS

SEQ ID NO: 266 - protein, 11 aa, Artificial Sequence
  Light Chain amino acid sequence
AAWDDSLSGWV

SEQ ID NO: 267 - protein, 5 aa, Artificial Sequence
  Light Chain amino acid sequence
QYNMP

SEQ ID NO: 268 - protein, 17 aa, Artificial Sequence
  Heavy Chain amino acid sequence
SIVPSGGFTAYADSVKG

SEQ ID NO: 269 - protein, 18 aa, Artificial Sequence
  Light Chain amino acid sequence
VDCSGGSCYRGPQNYFDY

SEQ ID NO: 270 - protein, 119 aa, Artificial Sequence
  Light Chain amino acid sequence
QYELTQPASVSGSPGQSITISCTGTSSDVGGYNYVSWYQQHPGKAPKLMIYEVSNRPSGVSNRFSGSKSDNTASLTISGLQAEDEADYYCGSYRKSSTPYVFGTGTKVSVLGQPKANPT

SEQ ID NO: 271 - protein, 135 aa, Artificial Sequence
  Heavy Chain amino acid sequence
EVQLLESGGGLVQPGGSLRLSCAASGFTFSQYMMTWVRQAPGKGLEWVSYIGSSGGQTKYADSVKGRFTISRDNSKNTLYLQMNSLRAEDTAVYYCARDPGVAVAGYYYYGMDVWGQGTTVTVSSASTKGPSVFP

SEQ ID NO: 272 - protein, 14 aa, Artificial Sequence
  Light Chain amino acid sequence
TGTSSDVGGYNYVS

SEQ ID NO: 273 - protein, 7 aa, Artificial Sequence
  Light Chain amino acid sequence
EVSNRPS

SEQ ID NO: 274 - protein, 11 aa, Artificial Sequence
  Light Chain amino acid sequence
GSYRKSSTPYV

SEQ ID NO: 275 - protein, 5 aa, Artificial Sequence
  Heavy Chain amino acid sequence
QYMMT

SEQ ID NO: 276 - protein, 17 aa, Artificial Sequence
  Heavy Chain amino acid sequence
YIGSSGGQTKYADSVKG

SEQ ID NO: 277 - protein, 16 aa, Artificial Sequence
  Heavy Chain amino acid sequence
DPGVAVAGYYYYGMDV

SEQ ID NO: 278 - protein, 116 aa, Artificial Sequence
  Light Chain amino acid sequence
QDIQMTQSPSSVSASVGDRVTITCRASRGISRWLAWYQQKPGKAPKLLIYGASTLQKGVPSRFTGSGSGTDFTLTITSLQPEDFATYYCQQGNSFPFTFGPGTKVDIKRTVAAPSV

SEQ ID NO: 279 - protein, 132 aa, Artificial Sequence
  Heavy Chain amino acid sequence
EVQLLESGGGLVQPGGSLRLSCAASGFTFSGYWMSWVRQAPGKGLEWVSVIRPSGGKTGYADSVKGRFTISRDNFKNTLYLQMNSLRAEDTAVYYCARVRAPGYYYYGMDVWGQGTTVTVSSASTKGPSVFP

SEQ ID NO: 280 - protein, 11 aa, Artificial Sequence
  Light Chain amino acid sequence
RASRGISRWLA

SEQ ID NO: 281 - protein, 7 aa, Artificial Sequence
  Light Chain amino acid sequence
GASTLQK

SEQ ID NO: 282 - protein, 9 aa, Artificial Sequence
  Light Chain amino acid sequence
QQGNSFPFT

SEQ ID NO: 283 - protein, 5 aa, Artificial Sequence
  Heavy Chain amino acid sequence
GYWMS

SEQ ID NO: 284 - protein, 17 aa, Artificial Sequence
  Heavy Chain amino acid sequence
VIRPSGGKTGYADSVKG

SEQ ID NO: 285 - protein, 13 aa, Artificial Sequence
  Heavy Chain amino acid sequence
VRAPGYYYYGMDV

SEQ ID NO: 286 - protein, 119 aa, Artificial Sequence; identical to SEQ ID NO: 222
  Light Chain amino acid sequence

SEQ ID NO: 287 - protein, 138 aa, Artificial Sequence; identical to SEQ ID NO: 223
  Light Chain amino acid sequence

SEQ ID NO: 288 - protein, 14 aa, Artificial Sequence
  Light Chain amino acid sequence
TGTSSDIGDYEYVS

SEQ ID NO: 289 - protein, 8 aa, Artificial Sequence
  Light Chain amino acid sequence
YEVSNRPS

SEQ ID NO: 290 - protein, 11 aa, Artificial Sequence
  Light Chain amino acid sequence
GSYRKSSTPYV

SEQ ID NO: 291 - protein, 5 aa, Artificial Sequence
  Heavy Chain amino acid sequence
YYHMW

SEQ ID NO: 292 - protein, 17 aa, Artificial Sequence
  Heavy Chain amino acid sequence
VIVPSGGGTQYADSVKG

SEQ ID NO: 293 - protein, 19 aa, Artificial Sequence
  Heavy Chain amino acid sequence
DGHSSSWYGGGAHYYGMDV

SEQ ID NO: 294 - protein, 116 aa, Artificial Sequence
  Light Chain amino acid sequence
QDIQMTQSPSSLSASVGDRVTITCRASQGIRNDLGWFQQKPGKAPRRLIWGASTLQSGVPSRFSGSGSGTDFTLTISSLQPEDFATYYCLQDYNYPYTFGQGTKLEIKRTVAAPSV

SEQ ID NO: 295 - protein, 132 aa, Artificial Sequence
  Heavy Chain amino acid sequence
EVQLLESGGGLVQPGGSLRLSCAASGFTFSFYGMPWVRQAPGKGLEWVSGIYPSGGVTRYADSVKGRFTISRDNSKNTLYLQMNSLRAEDTAVYYCAKTYSSSWYGWYFDYWGQGTLVTVSSASTKGPSVFP

SEQ ID NO: 296 - protein, 11 aa, Artificial Sequence
  Light Chain amino acid sequence
RASQGIRNDLG

SEQ ID NO: 297 - protein, 7 aa, Artificial Sequence
  Light Chain amino acid sequence
GASTLQS

SEQ ID NO: 298 - protein, 9 aa, Artificial Sequence
  Light Chain amino acid sequence
LQDYNYPYT

SEQ ID NO: 299 - protein, 5 aa, Artificial Sequence
  Heavy Chain amino acid sequence
FYGMP

SEQ ID NO: 300 - protein, 17 aa, Artificial Sequence
  Heavy Chain amino acid sequence
GIYPSGGVTRYADSVKG

SEQ ID NO: 301 - protein, 13 aa, Unknown
  Heavy Chain amino acid sequence
TYSSSWYGWYFDY

SEQ ID NO: 302 - protein, 117 aa, Unknown
  Light Chain amino acid sequence
QDIQMTQSPGTLSLSPGERATLSCRASQSVSSSYLAWYQQKPGQAPRLLIYGASSRATGIPDRFSGSGSGTDFTLTISRLEPEDFAVYYCQQYGSSPWTFGQGTKVEIKRTVAAPSV

SEQ ID NO: 303 - protein, 130 aa, Unknown
  Heavy Chain amino acid sequence
EVQLLESGGGLVQPGGSLRLSCAASGFTFSFYPMPWVRQAPGKGLEWVSYISPSGGDTTYADSVKGRFTISRDNSKNTFYLQMNSLRAEDTAVYYCARGGSYSSSWYGYWGQGTLVTVSSASTKGPSVFP

SEQ ID NO: 304 - protein, 12 aa, Unknown
  Light Chain amino acid sequence
RASQSVSSSYLA

SEQ ID NO: 305 - protein, 7 aa, Unknown
  Light Chain amino acid sequence
GASSRAT

SEQ ID NO: 306 - protein, 9 aa, Unknown
  Light Chain amino acid sequence
QQYGSSPWT

SEQ ID NO: 307 - protein, 5 aa, Unknown
  Heavy Chain amino acid sequence
FYPMP

SEQ ID NO: 308 - protein, 17 aa, Unknown
  Heavy Chain amino acid sequence
YISPSGGDTTYADSVKG

SEQ ID NO: 309 - protein, 11 aa, Unknown
  Heavy Chain amino acid sequence
GGSYSSSWYGY

SEQ ID NO: 310 - protein, 116 aa, Unknown; identical to SEQ ID NO: 278
  Light Chain amino acid sequence

SEQ ID NO: 311 - protein, 132 aa, Unknown; identical to SEQ ID NO: 279
  Heavy Chain amino acid sequence

SEQ ID NO: 312 - protein, 11 aa, Unknown
  Light Chain amino acid sequence
RASRGISRWLA

SEQ ID NO: 313 - protein, 7 aa, Unknown
  Light Chain amino acid sequence
GASTLQK

SEQ ID NO: 314 - protein, 9 aa, Unknown
  Light Chain amino acid sequence
QQGNSFPFT

SEQ ID NO: 315 - protein, 5 aa, Unknown
  Heavy Chain amino acid sequence
GYWMS

SEQ ID NO: 316 - protein, 17 aa, Unknown
  Heavy Chain amino acid sequence
VIRPSGGKTGYADSVKG

SEQ ID NO: 317 - protein, 13 aa, Unknown
  Heavy Chain amino acid sequence
VRAPGYYYYGMDV

SEQ ID NO: 318 - protein, 118 aa, Unknown
  Light Chain amino acid sequence
QSVLTQPASVSGSPGQSITISCTGTSSDVGGYNYVSWYQRHPGKAPKLIIYDVTNRPSGASRHFSGSKSGNTASLTISGLQADDEADYYCVSFTNSNTFVFGSGTRVTVLGQPKANPT

SEQ ID NO: 319 - protein, 138 aa, Unknown
  Heavy Chain amino acid sequence
EVQLLESGGGLVQPGGSLRLSCAASGFTFSLYHMDWVRQAPGKGLEWVSVIYPSGGGTPYADSVKGRFTISRDNSKNTLYLQMNSLRAEDTAVYYCARRVGYCSGGSCYYYYYYMDVWGKGTTVTVSSASTKGPSVFP

SEQ ID NO: 320 - protein, 14 aa, Unknown
  Light Chain amino acid sequence
TGTSSDVGGYNYVS

SEQ ID NO: 321 - protein, 6 aa, Unknown
  Light Chain amino acid sequence
DVTNRP

SEQ ID NO: 322 - protein, 10 aa, Unknown
  Light Chain amino acid sequence
VSFTNSNTFV

SEQ ID NO: 323 - protein, 5 aa, Unknown
  Heavy Chain amino acid sequence
LYHMD

SEQ ID NO: 324 - protein, 17 aa, Unknown
  Heavy Chain amino acid sequence
VIYPSGGGTPYADSVKG

SEQ ID NO: 325 - protein, 19 aa, Unknown
  Heavy Chain amino acid sequence
RVGYCSGGSCYYYYYYMDV

SEQ ID NO: 326 - protein, 116 aa, Unknown
  Light Chain amino acid sequence
QDIQMTQSPATLSVSPGERATLSCRASQSVRSYLAWYQQKPGQAPRLLIYDASTRATGIPARFSGSGSGTEFTLTISSLQSEDFAVYYCQQYNNWPPTFGQGTKVEIKRTVAAPSV

SEQ ID NO: 327 - protein, 123 aa, Unknown
  Heavy Chain amino acid sequence
EVQLLESGGGLVQPGGSLRLSCAASGFTFSWYRMNWVRQAPGKGLEWVSSIVPSGGYTRYADSVKGRFTISRDNSKNTLYLQMNSLRAEDTAVYYCASDFGSWGQGTLVTVSSASTKGPSVFP

SEQ ID NO: 328 - protein, 11 aa, Unknown
  Light Chain amino acid sequence
RASQSVRSYLA

SEQ ID NO: 329 - protein, 7 aa, Unknown
  Light Chain amino acid sequence
DASTRAT

SEQ ID NO: 330 - protein, 9 aa, Unknown
  Light Chain amino acid sequence
QQYNNWPPT

SEQ ID NO: 331 - protein, 5 aa, Unknown
  Heavy Chain amino acid sequence
WYRMN

SEQ ID NO: 332 - protein, 17 aa, Unknown
  Heavy Chain amino acid sequence
SIVPSGGYTRYADSVKG

SEQ ID NO: 333 - protein, 4 aa, Unknown
  Heavy Chain amino acid sequence
DFGS

SEQ ID NO: 334 - protein, 123 aa, Unknown
  Light Chain amino acid sequence
FYSHSAQSELTQPPSASGTPGQRVTISCSGSSSNIGSNTVNWYQQLPGTAPKLLIYSNNYRPSGVPDRFSGSKSGTSASLAISGLQSDDEAEYLCAAWDDSLNGPVFGGGTKVTVLGQPKAAP

SEQ ID NO: 335 - protein, 130 aa, Unknown
  Heavy Chain amino acid sequence
EVQLLESGGGLVQPGGSLRLSCAASGFTFSSYVMIWVRQAPGKGLEWVSWISSSGGYTSYADSVKGRFTISRDNSKNTLYLQMNSLRAEDTAVYYCAKGPGTRGDYWGQGTLVTVSSASTKGPSVFPLAP

SEQ ID NO: 336 - protein, 13 aa, Unknown
  Light Chain amino acid sequence
SGSSSNIGSNTVN

SEQ ID NO: 337 - protein, 5 aa, Unknown
  Heavy Chain amino acid sequence
SYVMI

SEQ ID NO: 338 - protein, 17 aa, Unknown
  Heavy Chain amino acid sequence
WISSSGGYTSYADSVKG

SEQ ID NO: 339 - protein, 8 aa, Unknown
  Heavy Chain amino acid sequence
GPGTRGDY

SEQ ID NO: 340 - protein, 123 aa, Unknown
  Light Chain amino acid sequence
FYSHSAQSVLTQPPSASATPGQRVTFSCSGSSSNIGSNAVNWYHQLPGTAPKLLIYHNNQRPSGVPDRFSGSKSGTSASLAISGLQSEDEADYYCAAWDDSLHGYVFGPGTKVTVLGQPKANP

SEQ ID NO: 341 - protein, 131 aa, Unknown
  Heavy Chain amino acid sequence
EVQLLESGGGLVQPGGSLRLSCAASGFTFSIYPMNWVRQAPGKGLEWVSGISPSGGYTGYADSVKGRFTISRDNSKNTLYLQMNSLRAEDTAVYYCARGGISWFMDYWGQGTLVTVSSASTKGPSVFPLAP

SEQ ID NO: 342 - protein, 13 aa, Unknown
  Light Chain amino acid sequence
SGSSSNIGSNAVN

SEQ ID NO: 343 - protein, 7 aa, Unknown
  Light Chain amino acid sequence
HNNQRPS

SEQ ID NO: 344 - protein, 11 aa, Unknown
  Light Chain amino acid sequence
AAWDDSLHGYV

SEQ ID NO: 345 - protein, 5 aa, Unknown
  Heavy Chain amino acid sequence
IYPMN

SEQ ID NO: 346 - protein, 17 aa, Unknown
  Heavy Chain amino acid sequence
GISPSGGYTGYADSVKG

SEQ ID NO: 347 - protein, 9 aa, Unknown
  Heavy Chain amino acid sequence
GGISWFMDY

SEQ ID NO: 348 - DNA, 369 bp, Unknown
  Light Chain nucleic acid sequence
ttctattctcacagtgcacagagcgtcttgactcagccaccctcagcgtctgcgacccccgggcagagggtcaccttctcttgttctggaagcagctccaacatcggaagtaatgctgtaaactggtaccatcagctcccaggaacggcccccaaactcctcatctatcataataatcagcgaccctcaggggtccctgaccgattctctggctccaagtctggcacctcagcctccctggccatcagtgggctccagtctgaggatgaggctgattattactgtgcagcatgggatgacagcctgcatggttatgtcttcggacctgggaccaaggtcaccgtcctaggtcagcccaaggccaacccc

SEQ ID NO: 349 - DNA, 393 bp, Unknown
  Heavy Chain nucleic acid sequence
gaagttcaattgttagagtctggtggcggtcttgttcagcctggtggttctttacgtctttcttgcgctgcttccggattcactttctctatttaccctatgaattgggttcgccaagctcctggtaaaggtttggagtgggtttctggtatctctccttctggtggctatactggttatgctgactccgttaaaggtcgcttcactatctctagagacaactctaagaatactctctacttgcagatgaacagcttaagggctgaggacactgcagtctactattgtgcgagagggggcatcagctggtttatggactactggggccagggaaccctggtcaccgtctcaagcgcctccaccaagggcccatcggtcttcccgctagcaccc

SEQ ID NO: 350 - DNA, 378 bp, Unknown
  Light Chain nucleic acid sequence
ttctattctcacagtgcacagagcgtcttgactcagcctcgctcagtgtccgggtctcctggacagtcagtcaccatctcctgcactggaaccagtagtgatgttggtgctagttataagtttgtctcctggtaccaactaaagccaggcaaagcccccaaactcatgctttttaatgtccgtgagcggccctcaggggtccctgatcgcttttctgggtccaagtccggcaacacggcctccctgaccatctctgggctccaggctgaggatgaggctgactattactgctgttcctatgcacgcggccagactttctcttatgtcttcggaggtgggaccacggtcaccgtcctaggtcagcccaaggccaacccc

SEQ ID NO: 351 - DNA, 402 bp, Unknown
  Heavy Chain nucleic acid sequence
gaagttcaattgttagagtctggtggcggtcttgttcagcctggtggttctttacgtctttcttgcgctgcttccggattcactttctctcgttactctatggggtgggttcgccaagctcctggtaaaggtttggagtgggtttcttctatccgtccttctggtggctatactcgttatgctgactccgttaaaggtcgcttcactatctctagagacaactctaagaatactctctacttgcagatgaacagcttaagggctgaggacactgcagtctactattgtgcgaaagatctggagtatagcagtggctggtcatttgactactggggccagggaaccctggtcaccgtctcaagcgcctccaccaagggcccatcggtcttcccgctagcaccc

SEQ ID NO: 352 - protein, 126 aa, Unknown
  Light Chain amino acid sequence
FYSHSAQSVLTQPRSVSGSPGQSVTISCTGTSSDVGASYKFVSWYQLKPGKAPKLMLFNVRERPSGVPDRFSGSKSGNTASLTISGLQAEDEADYYCCSYARGQTFSYVFGGGTTVTVLGQPKANP

SEQ ID NO: 353 - protein, 134 aa, Unknown
  Heavy Chain amino acid sequence
EVQLLESGGGLVQPGGSLRLSCAASGFTFSRYSMGWVRQAPGKGLEWVSSIRPSGGYTRYADSVKGRFTISRDNSKNTLYLQMNSLRAEDTAVYYCAKDLEYSSGWSFDYWGQGTLVTVSSASTKGPSVFPLAP

SEQ ID NO: 354 - protein, 12 aa, Unknown
  Light Chain amino acid sequence
CSYARGQTFSYV

SEQ ID NO: 355 - protein, 5 aa, Unknown
  Heavy Chain amino acid sequence
RYSMG

SEQ ID NO: 356 - protein, 17 aa, Unknown
  Heavy Chain amino acid sequence
SIRPSGGYTRYADSVKG

SEQ ID NO: 357 - protein, 12 aa, Unknown
  Heavy Chain amino acid sequence
DLEYSSGWSFDY

SEQ ID NO: 358 - protein, 11 aa, Artificial Sequence
  Exemplary motif
RASXXXXXXXX

SEQ ID NO: 359 - protein, 10 aa, Artificial Sequence
  Exemplary motif
RASQXXXXXX

SEQ ID NO: 360 - protein, 13 aa, Artificial Sequence
  Exemplary motif
SGSSSNIXXXXVX

SEQ ID NO: 361 - protein, 14 aa, Artificial Sequence
  Exemplary motif
TGTSSDXGXYXYVS

SEQ ID NO: 362 - protein, 7 aa, Artificial Sequence
  Exemplary motif
XXXQRPS

SEQ ID NO: 363 - protein, 6 aa, Artificial Sequence
  Exemplary motif
GASXXX

SEQ ID NO: 364 - protein, 8 aa, Artificial Sequence
  Exemplary motif
XQXXXXPX

SEQ ID NO: 365 - protein, 9 aa, Artificial Sequence
  Exemplary motif
QQYXXXPXT

SEQ ID NO: 366 - protein, 10 aa, Artificial Sequence
  Exemplary motif
AWDDSLSGXV

SEQ ID NO: 367 - protein, 10 aa, Artificial Sequence
  Exemplary motif
AWDDSLSGXV

SEQ ID NO: 368 - protein, 11 aa, Artificial Sequence
  Exemplary motif
AXWDXXLXGXV

SEQ ID NO: 369 - protein, 4 aa, Artificial Sequence
  Exemplary motif
YXMX

SEQ ID NO: 370 - protein, 5 aa, Artificial Sequence
  Exemplary motif
XYXMX

SEQ ID NO: 371 - protein, 4 aa, Artificial Sequence
  Exemplary motif
WYXM

SEQ ID NO: 372 - protein, 4 aa, Artificial Sequence
  Exemplary motif
QYXM

SEQ ID NO: 373 - protein, 6 aa, Artificial Sequence
  Exemplary motif
IXXSGG

SEQ ID NO: 374 - protein, 8 aa, Artificial Sequence
  Exemplary motif
IXXSGGXT

SEQ ID NO: 375 - protein, 8 aa, Artificial Sequence
  Exemplary motif
IXXSGGXT

SEQ ID NO: 376 - protein, 16 aa, Artificial Sequence
  Exemplary motif
IXXSGGXTXYADSVKG

SEQ ID NO: 377 - protein, 4 aa, Artificial Sequence
  Exemplary motif
XXWY

SEQ ID NO: 378 - protein, 5 aa, Artificial Sequence
  Exemplary motif
SSXWY

SEQ ID NO: 379 - protein, 6 aa, Artificial Sequence
  Exemplary motif
XYYYGM

SEQ ID NO: 380 - protein, 8 aa, Artificial Sequence
  Exemplary motif
XXYYYGMD

SEQ ID NO: 381 - protein, 11 aa, Artificial Sequence
  Exemplary motif
XXXXXXXXXXV

SEQ ID NO: 382 - protein, 7 aa, Artificial Sequence
  Exemplary motif
XXXXXXS

SEQ ID NO: 383 - protein, 5 aa, Artificial Sequence
  Exemplary motif
SSXWX

SEQ ID NO: 384 - protein, 7 aa, Artificial Sequence
  Exemplary motif
XXXXXXX

SEQ ID NO: 385 - protein, 7 aa, Artificial Sequence
  Exemplary motif
XXXXXXX

SEQ ID NO: 386 - protein, 7 aa, Artificial Sequence
  Exemplary motif
XXXXXXX

SEQ ID NO: 387 - protein, 14 aa, Artificial Sequence
  Exemplary motif
TGTSSDXGXYXXVS

SEQ ID NO: 388 - protein, 7 aa, Artificial Sequence
  Exemplary motif
XXXXXXS

SEQ ID NO: 389 - protein, 13 aa, Artificial Sequence
  Exemplary motif
SGSSSNIXXXXVX

SEQ ID NO: 390 - protein, 6 aa, Artificial Sequence
  Exemplary motif
XXXXRP

SEQ ID NO: 391 - protein, 11 aa, Artificial Sequence
  Exemplary motif
AXWDXXLXGXV

SEQ ID NO: 392 - protein, 8 aa, Artificial Sequence
  Exemplary motif
XXXXXXXX

SEQ ID NO: 393 - protein, 10 aa, Artificial Sequence
  Exemplary motif
XXXGXYXXXX

SEQ ID NO: 394 - protein, 4 aa, Artificial Sequence
  Exemplary motif
DFGS